(19) United States
(12) Patent Application Publication
(10) Pub. No.: US 2024/0368295 A1
(43) Pub. Date: Nov. 7, 2024

(54) ANTI-BCMA ANTIBODIES AND TREATMENT METHODS

(71) Applicant: SUTRO BIOPHARMA, INC., South San Francisco, CA (US)

(72) Inventors: Alice YAM, South San Francisco, CA (US); Ryan STAFFORD, South San Francisco, CA (US); Xiaofan LI, South San Francisco, CA (US); Junhao YANG, South San Francisco, CA (US); Stephanie ARMSTRONG, South San Francisco, CA (US); Abigail YU, South San Francisco, CA (US)

(21) Appl. No.: 18/638,420

(22) Filed: Apr. 17, 2024

Related U.S. Application Data
(62) Division of application No. 17/041,349, filed on Sep. 24, 2020, now Pat. No. 11,993,658.
(60) Provisional application No. 62/648,266, filed on Mar. 26, 2018.

(51) Int. Cl. C07K 16/28

(52) U.S. Cl. CPC C07K 16/2878; C07K 2317/55; C07K 2317/622; C07K 2317/73; C07K 2317/92

(57) ABSTRACT

The present disclosure relates to antibodies that selectively bind to B-cell maturation antigen (BCMA) and its isoforms and homologs, and compositions comprising the antibodies. Also provided are methods of using the antibodies, such as therapeutic and diagnostic methods.

### CROSS-REFERENCE TO RELATED APPLICATIONS

This application is a divisional application of U.S. patent application Ser. No. 17/041,349, filed on Sep. 24, 2020, which is the U.S. national stage entry of PCT Application No. PCT/US2019/023844, filed on Mar. 25, 2019, which claims the benefit of priority to U.S. Provisional Patent Application No. 62/648,266, filed on Mar. 26, 2018, the disclosures of the foregoing applications are incorporated herein by reference in their entirety.

### REFERENCE TO ELECTRONIC SEQUENCE LISTING

The application contains a Sequence Listing which has been submitted electronically in .XML format and is hereby incorporated by reference in its entirety. Said. XML copy, created on Apr. 16, 2024, is named “108843.00483.xml” and is 386,089 bytes in size. The sequence listing contained in this .XML file is part of the specification and is hereby incorporated by reference herein in its entirety.

### FIELD OF THE INVENTION

The present disclosure generally relates to antibodies with binding specificity for B-cell maturation antigen (BCMA) and compositions comprising the antibodies, including pharmaceutical compositions, diagnostic compositions, and kits. Also provided are methods of making anti-BCMA antibodies, and methods of using anti-BCMA antibodies, for example, for therapeutic purposes, diagnostic purposes, and research purposes.

### BACKGROUND

B-cell maturation antigen (BCMA) is a member of the tumor necrosis factor (TNF) receptor superfamily which recognizes B-cell activating factor. The protein in humans is encoded by the tumor necrosis factor receptor superfamily member 17 (TNFRSF17) gene and is preferentially expressed in mature B lymphocytes.

BCMA plays an important role in regulating B-cell maturation and differentiation into plasma cells. It is closely related to BAFF receptor (BAFF-R) and transmembrane activator and calcium modulator and cyclophilin ligand interactor (TACI). While BCMA, BAFF-R, and TACI are type III transmembrane proteins that promote B-cell survival at distinct stages of development, BCMA is expressed exclusively in B-cell linage cells, such as, for example, plasmablasts and differentiated plasma cells (Avery et al. (2003) J. Clin. Invest. 112 (2): 286-297; O'Connor et al. (2004) J. Exp. Med. 199 (1): 91-98). It is selectively induced during plasma cell differentiation, which occurs concurrently with loss of BAFF-R expression in the differentiated cells (Darce et al. (2007) J. Immunol. 178 (9): 5612-5622). BCMA expression appears to support the survival of normal plasma cells and plasmablasts but is typically absent on naïve and most memory B cells. Thus, it does not appear to be needed for overall B-cell homeostasis but is required for optimal survival of long-lived plasma cells in the bone marrow (O'Connor et al. (2004) supra; Xu, S. and K. P. Lam (2001) Mol. Cell. Biol. 21 (12): 4067-4074).

In multiple myeloma, BCMA has been shown to be universally and widely expressed in malignant plasma cells at elevated levels; however, it is typically undetected on normal human tissues except for plasma cells. Due to its selective expression as a cell-surface receptor on multiple myeloma cell lines, BCMA can potentially be targeted in therapies to treat multiple myeloma. BCMA expression is also associated with leukemia and lymphoma. Accordingly, there is a need for improved methods of targeting and/or modulating the activity of BCMA.

### SUMMARY

Provided herein are antibodies that selectively bind BCMA. In some embodiments, the antibodies bind human BCMA. In some embodiments, the antibodies also bind homologs of human BCMA.

In some embodiments, the antibodies comprise at least one CDR sequence defined by a consensus sequence provided in this disclosure. In some embodiments, the antibodies comprise an illustrative CDR, V_{H}, or V_{L} sequence provided in this disclosure, or a variant thereof. In some aspects, the variant is a variant with one or more conservative amino acid substitutions.

Also provided are compositions and kits comprising the antibodies. In some embodiments, the compositions are pharmaceutical compositions. Any suitable pharmaceutical composition may be used. In some embodiments, the pharmaceutical composition is a composition for parenteral administration.

This disclosure also provides methods of using the anti-BCMA antibodies provided herein. In some embodiments, the method is a method of treatment. In some embodiments, the method is a diagnostic method. In some embodiments, the method is an analytical method. In some embodiments, the method is a method of purifying and/or quantifying BCMA.

In some embodiments, the antibodies are used to treat a disease or condition. In some aspects, the disease or condition is selected from a cancer, autoimmune disease, and infection. In some embodiments, the disease or condition is leukemia, lymphoma, or multiple myeloma.

These and other embodiments of the invention along with many of its features are described in more detail in conjunction with the text below and attached figures.

### BRIEF DESCRIPTION OF THE FIGURES

FIG. 1 provides a comparison of the Kabat and Chothia numbering systems for CDR-H1. Adapted from Martin A. C. R. (2010). Protein Sequence and Structure Analysis of Antibody Variable Domains. In R. Kontermann & S. Dübel (Eds.), Antibody Engineering vol. 2 (pp. 33-51). Springer-Verlag, Berlin Heidelberg.

FIGS. 2 and 3 provide alignments of the VH sequences (SEQ ID NOs: 167-216) from the variant antibodies provided herein. CDRs according to Chothia are highlighted, and CDRs according to Kabat are in boxes.

FIG. 4 provides alignments of the VL sequences (SEQ ID NOs: 217-238) from trastuzumab and the variant antibodies provided herein. CDRs according to Chothia are highlighted, and CDRs according to Kabat are in boxes.

### DETAILED DESCRIPTION OF THE EMBODIMENTS

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Green & Sambrook, Molecular Cloning: A Laboratory Manual 4^{th} ed. (2012), Cold Spring Harbor Laboratory Press, Cold Spring Harbor, NY; and Ausubel et al., Current Protocols in Molecular Biology, John Wiley & Sons. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value±10%, ±5%, or ±1%. In certain embodiments, the term “about” indicates the designated value±one standard deviation of that value.

The term “combinations thereof” includes every possible combination of elements to which the term refers to. For example, a sentence stating that “if α₂ is A, then α₃ is not D; as is not S; or as is not S; or combinations thereof” includes the following combinations when α₂ is A: (1) α₃ is not D; (2) as is not S; (3) α₆ is not S; (4) α₃ is not D; α₅ is not S; and α₆ is not S; (5) α₃ is not D and as is not S; (6) α₃ is not D and as is not S; and (7) as is not S and α₆ is not S.

The terms “BCMA” and “B-cell maturation antigen” are used interchangeably herein. BCMA is also known by synonyms, including BCM, tumor necrosis factor receptor superfamily member 17 (“TNFRSF17”), CD269, TNFRSF13A, and TNF receptor superfamily member 17, among others. Unless specified otherwise, the terms include any variants, isoforms and species homologs of human BCMA that are naturally expressed by cells, or that are expressed by cells transfected with a BCMA or BCMA gene. BCMA proteins include, for example, human BCMA isoform 1 (SEQ ID NO: 1) and human BCMA isoform 2 (SEQ ID NO: 2). In some embodiments, BCMA proteins include cynomolgus monkey BCMA (SEQ ID NO: 3). In some embodiments, BCMA proteins include murine BCMA (SEQ ID NO: 4).

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H} or VH) and a heavy chain constant region (C_{H} or CH). The heavy chain constant region typically comprises three domains, abbreviated C_{H}1 (or CH1), C_{H}2 (or CH2), and C_{H}3 (or CH3). Each light chain typically comprises a light chain variable region (V_{L} or VL) and a light chain constant region. The light chain constant region typically comprises one domain, abbreviated C_{L} or CL.

The term “antibody” describes a type of immunoglobulin molecule and is used herein in its broadest sense. An antibody specifically includes intact antibodies (e.g., intact immunoglobulins), and antibody fragments. Antibodies comprise at least one antigen-binding domain. One example of an antigen-binding domain is an antigen binding domain formed by a V_{H}-V_{L} dimer. A “BCMA antibody,” “anti-BCMA antibody,” “BCMA Ab,” “BCMA-specific antibody,” “anti-BCMA Ab,” “BCMA antibody,” “anti-BCMA antibody,” “BCMA Ab,” “BCMA-specific antibody,” or “anti-BCMA Ab,” or any iteration of these phrases where “BCMA” is substituted by “TNFSF17,” is an antibody, as described herein, which binds specifically to BCMA. In some embodiments, the antibody binds the extracellular domain of BCMA.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2 FR3-CDR3-FR4. The CDRs are involved in antigen binding, and influence antigen specificity and binding affinity of the antibody. Sec Kabat et al., Sequences of Proteins of Immunological Interest 5th ed. (1991) Public Health Service, National Institutes of Health, Bethesda, MD, incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated a, 8, ¿, Y, and u, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme), each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

TABLE 1
Residues in CDRs according to Kabat and Chothia
numbering schemes.

| CDR | Kabat | Chothia |
|---|---|---|
| Ll | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

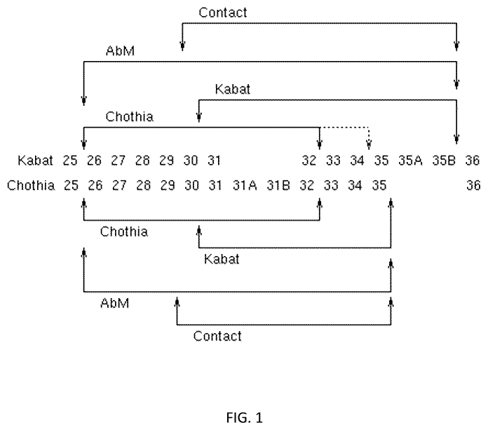

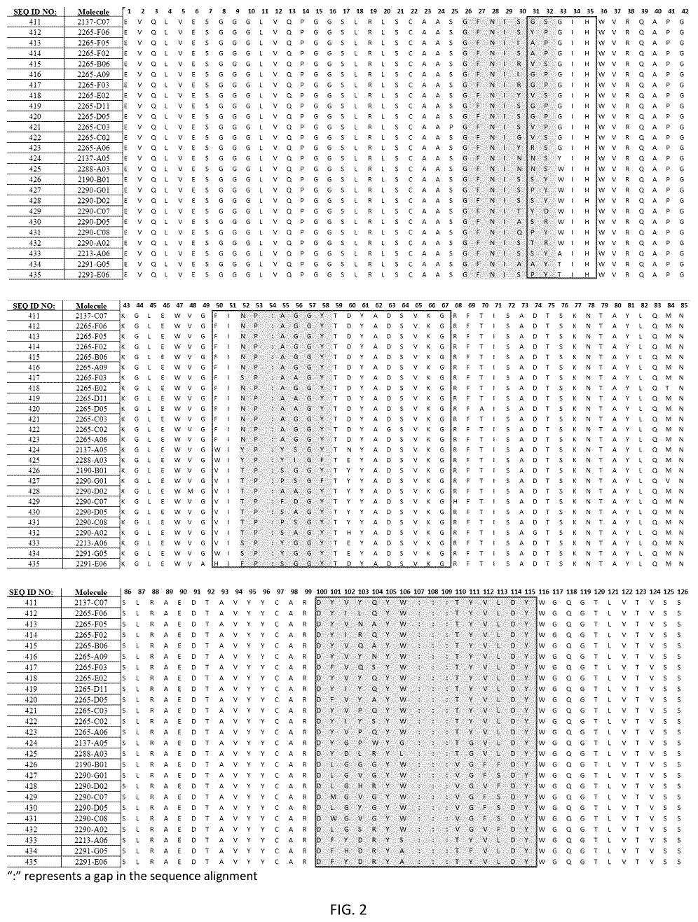

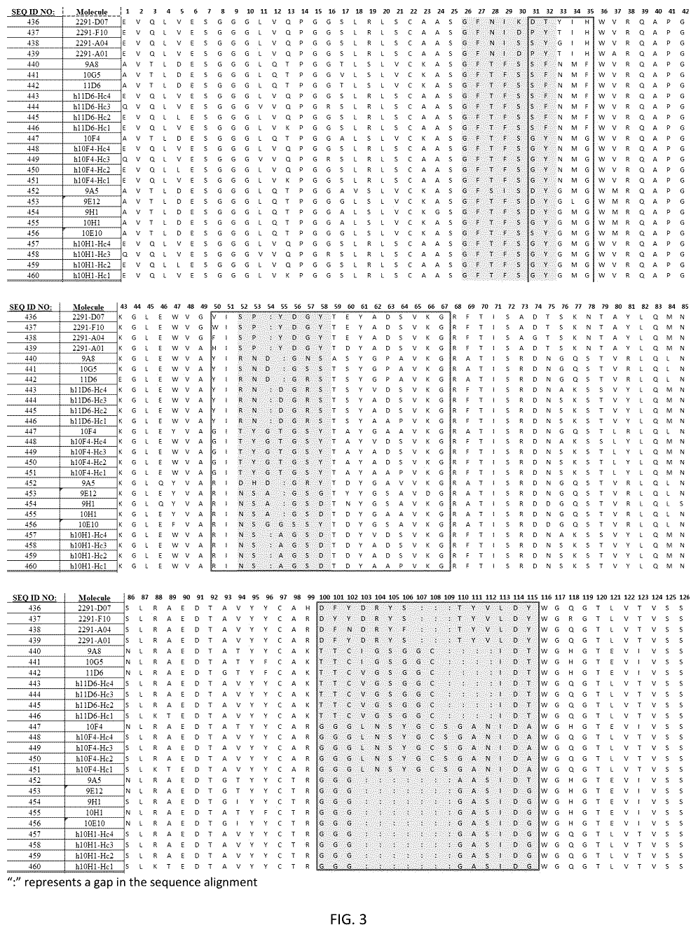

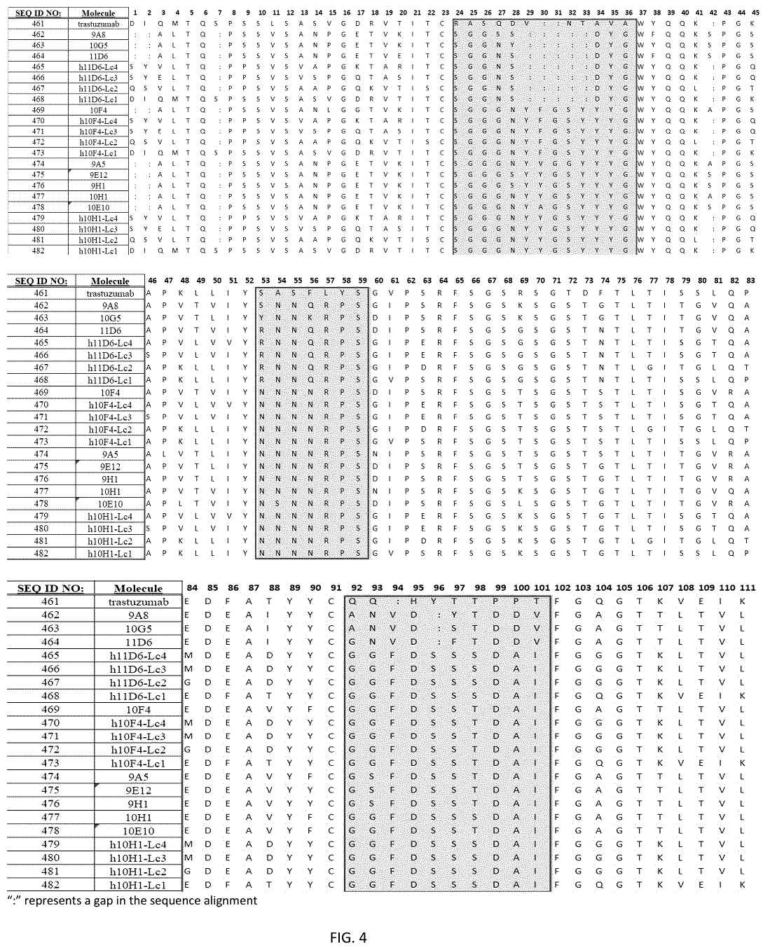

*The C-terminus of CDR-H1 when numbered using the Kabat numbering convention, varies between H32 and H34, depending on the length of the CDR, as illustrated in FIG. 1.

Unless otherwise specified, the numbering scheme used for identification of a particular CDR herein is the Kabat/Chothia numbering scheme. Where the residues encompassed by these two numbering schemes diverge (e.g., CDR-H1 and/or CDR-H2), the numbering scheme is specified as either Kabat or Chothia. For convenience, CDR-H3 is sometimes referred to herein as either Kabat or Chothia. However, this is not intended to imply differences in sequence where they do not exist, and one of skill in the art can readily confirm whether the sequences are the same or different by examining the sequences.

CDRs may be assigned, for example, using antibody numbering software, such as Abnum, available at bioinf.org.uk/abs/abnum/, and described in Abhinandan and Martin, Immunology, 2008, 45:3832-3839, incorporated by reference in its entirety.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (CH1) of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with B-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Pluckthun A. (1994). In some embodiments, the linker is SEQ ID NO: 246. In some embodiments, the linker is SEQ ID NO: 247. Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminus of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG1 Fc domain. In some embodiments, the IgG1 Fc domain comprises SEQ ID NO: 239, or a portion thereof. SEQ ID NO: 239 provides the sequence of C_{H}1, C_{H}2, and C_{H}3 of the human IgG1 constant region.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” is one that has been separated and/or recovered from a component of its natural environment. Components of the natural environment may include enzymes, hormones, and other proteinaccous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. An isolated antibody includes an antibody in situ within recombinant cells, since at least one component of the antibody's natural environment is not present. In some aspects, an isolated antibody is prepared by at least one purification step.

In some embodiments, an isolated antibody is purified to at least 80%, 85%, 90%, 95%, or 99% by weight. In some embodiments, an isolated antibody is purified to at least 80%, 85%, 90%, 95%, or 99% by volume. In some embodiments, an isolated antibody is provided as a solution comprising at least 85%, 90%, 95%, 98%, 99% to 100% by weight. In some embodiments, an isolated antibody is provided as a solution comprising at least 85%, 90%, 95%, 98%, 99% to 100% by volume.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can be represented by the dissociation constant (K_{D}). Affinity can be measured by common methods known in the art, including those described herein. Affinity can be determined, for example, using surface plasmon resonance (SPR) technology, such as a Biacore® instrument. In some embodiments, the affinity is determined at 25° C.

With regard to the binding of an antibody to a target molecule, the terms “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction. Specific binding can be measured, for example, by determining binding of a molecule compared to binding of a control molecule. Specific binding can also be determined by competition with a control molecule that mimics the antibody binding site on the target. In that case, specific binding is indicated if the binding of the antibody to the target is competitively inhibited by the control molecule.

The term “k_{d}” or “kd” (sec⁻¹), as used herein, refers to the dissociation rate constant of a particular antibody-antigen interaction. This value is also referred to as the k_{off} value.

The term “kₐ” or “ka” (M⁻¹×sec⁻¹), as used herein, refers to the association rate constant of a particular antibody-antigen interaction. This value is also referred to as the kₒₙ value.

The term “K_{D}” (also referred to as “Kd” or “KD,” M or nM), as used herein, refers to the dissociation equilibrium constant of a particular antibody-antigen interaction. K_{D})=k_{d}/kₐ. The value of K_{D} is typically equal in magnitude to the concentration of ligand at which half the protein molecules are bound to ligand at equilibrium.

The term “K_{A}” or “Kₐ” (M⁻¹), as used herein, refers to the association equilibrium constant of a particular antibody-antigen interaction. K_{A}=kₐ/k_{d}.

An “affinity matured” antibody is one with one or more alterations in one or more CDRs or FRs that result in an improvement in the affinity of the antibody for its antigen, compared to a parent antibody which does not possess the alteration(s). In one embodiment, an affinity matured antibody has nanomolar or picomolar affinity for the target antigen. Affinity matured antibodies may be produced using a variety of methods known in the art. For example, Marks et al. (Bio/Technology, 1992, 10:779-783, incorporated by reference in its entirety) describes affinity maturation by V_{H} and V_{L} domain shuffling. Random mutagenesis of CDR and/or framework residues is described by, for example, Barbas et al. (Proc. Nat. Acad. Sci. U.S.A., 1994, 91:3809-3813); Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896, each of which is incorporated by reference in its entirety.

When used herein in the context of two or more antibodies, the term “competes with” or “cross-competes with” indicates that the two or more antibodies compete for binding to an antigen (e.g., BCMA). In one exemplary assay, BCMA is coated on a plate and allowed to bind a first antibody, after which a second, labeled antibody is added. If the presence of the first antibody reduces binding of the second antibody, then the antibodies compete. In another exemplary assay, a first antibody is coated on a plate and allowed to bind the antigen, and then the second antibody is added. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 50%, at least 60%, at least 70%, at least 80%, or at least 90%.

The term “epitope” means a portion of an antigen capable of specific binding to an antibody. Epitopes frequently consist of surface-accessible amino acid residues and/or sugar side chains and may have specific three dimensional structural characteristics, as well as specific charge characteristics. Conformational and non-conformational epitopes are distinguished in that the binding to the former but not the latter is lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to variants of BCMA with different point-mutations.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution of an amino acid with a chemically or functionally similar amino acid. Conservative substitution tables providing similar amino acids are well known in the art. Polypeptide sequences having such substitutions are known as “conservatively modified variants.” By way of example, the groups of amino acids provided in Tables 2-4 are, in some embodiments, considered conservative substitutions for one another.

TABLE 2
Selected groups of amino acids that are considered conservative
substitutions for one another, in certain embodiments.

| | |
|---|---|
| Acidic Residues | D and E |
| Basic Residues | K, R, and H |
| Hydrophilic Uncharged | S, T, N, and Q |
| Residues | |
| Aliphatic Uncharged | G, A, V, L, and I |
| Residues | |
| Non-polar Uncharged | C, M, and P |
| Residues | |
| Aromatic Residues | F, Y, and W |
| Alcohol Group- | S and T |
| Containing | |
| Residues | |
| Aliphatic Residues | I, L, V, and M |
| Cycloalkenyl-associated | F, H, W, and Y |
| Residues | |
| Hydrophobic Residues | A, C, F, G, H, I, L, M, R, T, V, W, and Y |
| Negatively Charged | D and E |
| Residues | |
| Polar Residues | C, D, E, H, K, N, Q, R, S, and T |
| Positively Charged | H, K, and R |
| Residues | |
| Small Residues | A, C, D, G, N, P, S, T, and V |
| Very Small Residues | A, G, and S |
| Residues Involved in | A, C, D, E, G, H, K, N, Q, R, S, P, and T |
| Turn Formation | |
| Flexible Residues | Q, T, K, S, G, P, D, E, and R |

TABLE 3
Additional selected groups of amino acids that are
considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that are
considered conservative substitutions
for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, NY. An antibody generated by making one or more conservative substitutions of amino acid residues in a parent antibody is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “therapeutically effective amount” or “effective amount” refers to an amount of an antibody or composition that when administered to a subject is effective to treat a disease or disorder.

As used herein, the term “subject” means a mammalian subject. Exemplary subjects include, but are not limited to humans, monkeys, dogs, cats, mice, rats, cows, horses, camels, avians, goats, and sheep. In certain embodiments, the subject is a human. In some embodiments, the subject has a disease that can be treated or diagnosed with an antibody provided herein. In some embodiments, the disease is leukemia, lymphoma, or multiple myeloma, a plasmacytoid dendritic cell tumor, a B-cell lineage malignancy, a plasma cell neoplasm, diffuse large B-cell lymophoma (DLBCL), a low-grade B-cell lymphoma, Burkitt's lymphoma, a plasmablastic lymphoma, or a follicular lymphoma.

### 2. Antibodies

Provided herein are antibodies that selectively bind human BCMA. In some aspects, the antibody selectively binds to the extracellular domain of human BCMA (human BCMA).

In some embodiments, the antibody binds to a homolog of human BCMA. In some aspects, the antibody binds to a homolog of human BCMA from a species selected from monkeys, mice, dogs, cats, rats, cows, horses, goats and sheep. In some aspects, the homolog is a cynomolgus monkey homolog. In some aspects, the homolog is a mouse or murine homolog.

In some embodiments, the antibody has one or more CDRs having particular lengths, in terms of the number of amino acid residues. In some embodiments, the Chothia CDR-H1 of the antibody is 6, 7, or 8 residues in length. In some embodiments, the Kabat CDR-H1 of the antibody is 4, 5, or 6 residues in length. In some embodiments, the Chothia CDR-H2 of the antibody is 5, 6, or 7 residues in length. In some embodiments, the Kabat CDR-H2 of the antibody is 16, 17, or 18 residues in length. In some embodiments, the Kabat/Chothia CDR-H3 of the antibody is 7, 8, 9, 10, 11, 12, 13, 14, 15, or 16 residues in length.

In some aspects, the Kabat/Chothia CDR-L1 of the antibody is 10, 11, 12, 13, 14, 15, or 16 residues in length. In some aspects, the Kabat/Chothia CDR-L2 of the antibody is 6, 7, or 8 residues in length. In some aspects, the Kabat/Chothia CDR-L3 of the antibody is 8, 9, or 10 residues in length.

In some embodiments, the antibody comprises a light chain. In some aspects, the light chain is a kappa light chain. In some aspects, the light chain is a lambda light chain.

In some embodiments, the antibody comprises a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the antibody is an antibody fragment. In some aspects, the antibody fragment is an Fv fragment. In some aspects, the antibody fragment is a Fab fragment. In some aspects, the antibody fragment is a F(ab′)₂ fragment. In some aspects, the antibody fragment is a Fab′ fragment. In some aspects, the antibody fragment is an scFv (sFv) fragment. In some aspects, the antibody fragment is an scFv-Fc fragment.

In some embodiments, the antibody is a monoclonal antibody. In some embodiments, the antibody is a polyclonal antibody.

In some embodiments, the antibody is a chimeric antibody. In some embodiments, the antibody is a humanized antibody. In some embodiments, the antibody is a human antibody.

In some embodiments, the antibody is an affinity matured antibody. In some aspects, the antibody is an affinity matured antibody derived from an illustrative sequence provided in this disclosure.

The antibodies provided herein may be useful for the treatment of a variety of diseases and conditions including cancers. In some embodiments, the antibodies provided herein may be useful for the treatment of cancers of solid tumors. For example, the antibodies provided herein can be useful for the treatment of colorectal cancer.

### 2.1 CDR-H3 Sequences

In some embodiments, the antibody comprises a CDR-H3 sequence comprising, consisting of, or consisting essentially of a CDR-H3 sequence of an illustrative antibody or V_{H} sequence provided herein. In some aspects, the CDR-H3 sequence is a CDR-H3 sequence of a V_{H} sequence provided in SEQ ID NOs: 167-216.

In some embodiments, the antibody comprises a CDR-H3 sequence comprising, consisting of, or consisting essentially of a sequence selected from SEQ ID NOs: 116-145. In some aspects, the antibody comprises a CDR-H3 sequence comprising, consisting of, or consisting essentially of SEQ ID NO: 116. In some aspects, the antibody comprises a CDR-H3 sequence comprising, consisting of, or consisting essentially of SEQ ID NO: 117. In some aspects, the antibody comprises a CDR-H3 sequence comprising, consisting of, or consisting essentially of SEQ ID NO: 118. In some aspects, the antibody comprises a CDR-H3 sequence comprising, consisting of, or consisting essentially of SEQ ID NO: 119. In some aspects, the antibody comprises a CDR-H3 sequence comprising, consisting of, or consisting essentially of SEQ ID NO: 120. In some aspects, the antibody comprises a CDR-H3 sequence comprising, consisting of, or consisting essentially of SEQ ID NO: 121. In some aspects, the antibody comprises a CDR-H3 sequence comprising, consisting of, or consisting essentially of SEQ ID NO: 122. In some aspects, the antibody comprises a CDR-H3 sequence comprising, consisting of, or consisting essentially of SEQ ID NO: 123. In some aspects, the antibody comprises a CDR-H3 sequence comprising, consisting of, or consisting essentially of SEQ ID NO: 124. In some aspects, the antibody comprises a CDR-H3 sequence comprising, consisting of, or consisting essentially of SEQ ID NO: 125. In some aspects, the antibody comprises a CDR-H3 sequence comprising, consisting of, or consisting essentially of SEQ ID NO: 126. In some aspects, the antibody comprises a CDR-H3 sequence comprising, consisting of, or consisting essentially of SEQ ID NO: 127. In some aspects, the antibody comprises a CDR-H3 sequence comprising, consisting of, or consisting essentially of SEQ ID NO: 128. In some aspects, the antibody comprises a CDR-H3 sequence comprising, consisting of, or consisting essentially of SEQ ID NO: 129. In some aspects, the antibody comprises a CDR-H3 sequence comprising, consisting of, or consisting essentially of SEQ ID NO: 130. In some aspects, the antibody comprises a CDR-H3 sequence comprising, consisting of, or consisting essentially of SEQ ID NO: 131. In some aspects, the antibody comprises a CDR-H3 sequence comprising, consisting of, or consisting essentially of SEQ ID NO: 132. In some aspects, the antibody comprises a CDR-H3 sequence comprising, consisting of, or consisting essentially of SEQ ID NO: 133. In some aspects, the antibody comprises a CDR-H3 sequence comprising, consisting of, or consisting essentially of SEQ ID NO: 134. In some aspects, the antibody comprises a CDR-H3 sequence comprising, consisting of, or consisting essentially of SEQ ID NO: 135. In some aspects, the antibody comprises a CDR-H3 sequence comprising, consisting of, or consisting essentially of SEQ ID NO: 136. In some aspects, the antibody comprises a CDR-H3 sequence comprising, consisting of, or consisting essentially of SEQ ID NO: 137. In some aspects, the antibody comprises a CDR-H3 sequence comprising, consisting of, or consisting essentially of SEQ ID NO: 138. In some aspects, the antibody comprises a CDR-H3 sequence comprising, consisting of, or consisting essentially of SEQ ID NO: 139. In some aspects, the antibody comprises a CDR-H3 sequence comprising, consisting of, or consisting essentially of SEQ ID NO: 140. In some aspects, the antibody comprises a CDR-H3 sequence comprising, consisting of, or consisting essentially of SEQ ID NO: 141. In some aspects, the antibody comprises a CDR-H3 sequence comprising, consisting of, or consisting essentially of SEQ ID NO: 142. In some aspects, the antibody comprises a CDR-H3 sequence comprising, consisting of, or consisting essentially of SEQ ID NO: 143. In some aspects, the antibody comprises a CDR-H3 sequence comprising, consisting of, or consisting essentially of SEQ ID NO: 144. In some aspects, the antibody comprises a CDR-H3 sequence comprising, consisting of, or consisting essentially of SEQ ID NO: 145.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

2.2 V_{H} Sequences Comprising Illustrative CDRs

In some embodiments, the antibody comprises a V_{H} sequence comprising one or more CDR-H3 sequences comprising, consisting of, or consisting essentially of one or more illustrative CDR-H3 sequences provided in this disclosure, and variants thereof. In some embodiments, the CDR-H3 sequences comprise, consist of, or consist essentially of one or more CDR-H3 sequences provided in a V_{H} sequence selected from SEQ ID NOs: 167-216.

### 2.2.1. V_{H} Sequences Comprising Illustrative Kabat CDRs

In some embodiments, the antibody comprises a V_{H} sequence comprising one or more Kabat CDR-H3 sequences comprising, consisting of, or consisting essentially of one or more illustrative Kabat CDR-H3 sequences provided in this disclosure, and variants thereof.

### 2.2.1.1. Kabat CDR-H3

In some embodiments, the antibody comprises a V_{H} sequence comprising a CDR-H3 sequence, wherein the CDR-H3 sequence comprises, consists of, or consists essentially of a Kabat CDR-H3 sequence of an illustrative antibody or V_{H} sequence provided herein. In some aspects, the Kabat CDR-H3 sequence is a Kabat CDR-H3 sequence of a V_{H} sequence provided in SEQ ID NOs: 167-216.

In some embodiments, the antibody comprises a V_{H} sequence comprising a Kabat CDR-H3 sequence comprising, consisting of, or consisting essentially of a sequence selected from SEQ ID NOs: 116-145. In some aspects, the antibody comprises a CDR-H3 sequence comprising, consisting of, or consisting essentially of SEQ ID NO: 116. In some aspects, the antibody comprises a CDR-H3 sequence comprising, consisting of, or consisting essentially of SEQ ID NO: 117. In some aspects, the antibody comprises a CDR-H3 sequence comprising, consisting of, or consisting essentially of SEQ ID NO: 118. In some aspects, the antibody comprises a CDR-H3 sequence comprising, consisting of, or consisting essentially of SEQ ID NO: 119. In some aspects, the antibody comprises a CDR-H3 sequence comprising, consisting of, or consisting essentially of SEQ ID NO: 120. In some aspects, the antibody comprises a CDR-H3 sequence comprising, consisting of, or consisting essentially of SEQ ID NO: 121. In some aspects, the antibody comprises a CDR-H3 sequence comprising, consisting of, or consisting essentially of SEQ ID NO: 122. In some aspects, the antibody comprises a CDR-H3 sequence comprising, consisting of, or consisting essentially of SEQ ID NO: 123. In some aspects, the antibody comprises a CDR-H3 sequence comprising, consisting of, or consisting essentially of SEQ ID NO: 124. In some aspects, the antibody comprises a CDR-H3 sequence comprising, consisting of, or consisting essentially of SEQ ID NO: 125. In some aspects, the antibody comprises a CDR-H3 sequence comprising, consisting of, or consisting essentially of SEQ ID NO: 126. In some aspects, the antibody comprises a CDR-H3 sequence comprising, consisting of, or consisting essentially of SEQ ID NO: 127. In some aspects, the antibody comprises a CDR-H3 sequence comprising, consisting of, or consisting essentially of SEQ ID NO: 128. In some aspects, the antibody comprises a CDR-H3 sequence comprising, consisting of, or consisting essentially of SEQ ID NO: 129. In some aspects, the antibody comprises a CDR-H3 sequence comprising, consisting of, or consisting essentially of SEQ ID NO: 130. In some aspects, the antibody comprises a CDR-H3 sequence comprising, consisting of, or consisting essentially of SEQ ID NO: 131. In some aspects, the antibody comprises a CDR-H3 sequence comprising, consisting of, or consisting essentially of SEQ ID NO: 132. In some aspects, the antibody comprises a CDR-H3 sequence comprising, consisting of, or consisting essentially of SEQ ID NO: 133. In some aspects, the antibody comprises a CDR-H3 sequence comprising, consisting of, or consisting essentially of SEQ ID NO: 134. In some aspects, the antibody comprises a CDR-H3 sequence comprising, consisting of, or consisting essentially of SEQ ID NO: 135. In some aspects, the antibody comprises a CDR-H3 sequence comprising, consisting of, or consisting essentially of SEQ ID NO: 136. In some aspects, the antibody comprises a CDR-H3 sequence comprising, consisting of, or consisting essentially of SEQ ID NO: 137. In some aspects, the antibody comprises a CDR-H3 sequence comprising, consisting of, or consisting essentially of SEQ ID NO: 138. In some aspects, the antibody comprises a CDR-H3 sequence comprising, consisting of, or consisting essentially of SEQ ID NO: 139. In some aspects, the antibody comprises a CDR-H3 sequence comprising, consisting of, or consisting essentially of SEQ ID NO: 140. In some aspects, the antibody comprises a CDR-H3 sequence comprising, consisting of, or consisting essentially of SEQ ID NO: 141. In some aspects, the antibody comprises a CDR-H3 sequence comprising, consisting of, or consisting essentially of SEQ ID NO: 142. In some aspects, the antibody comprises a CDR-H3 sequence comprising, consisting of, or consisting essentially of SEQ ID NO: 143. In some aspects, the antibody comprises a CDR-H3 sequence comprising, consisting of, or consisting essentially of SEQ ID NO: 144. In some aspects, the antibody comprises a CDR-H3 sequence comprising, consisting of, or consisting essentially of SEQ ID NO: 145.

### 2.2.1.2. Kabat CDR-H2

In some embodiments, the antibody comprises a V_{H} sequence comprising a CDR-H2 sequence, wherein the CDR-H2 sequence comprises, consists of, or consists essentially of a Kabat CDR-H2 sequence of an illustrative antibody or V_{H} sequence provided herein. In some aspects, the Kabat CDR-H2 sequence is a Kabat CDR-H2 sequence of a V_{H} sequence provided in SEQ ID NOs: 167-216.

In some embodiments, the antibody comprises a V_{H} sequence comprising a Kabat CDR-H2 sequence comprising, consisting of, or consisting essentially of a sequence selected from SEQ ID NOs: 79-115. In some aspects, the antibody comprises a CDR-H2 sequence comprising, consisting of, or consisting essentially of SEQ ID NO: 79. In some aspects, the antibody comprises a CDR-H2 sequence comprising, consisting of, or consisting essentially of SEQ ID NO: 80. In some aspects, the antibody comprises a CDR-H2 sequence comprising, consisting of, or consisting essentially of SEQ ID NO: 81. In some aspects, the antibody comprises a CDR-H2 sequence comprising, consisting of, or consisting essentially of SEQ ID NO: 82. In some aspects, the antibody comprises a CDR-H2 sequence comprising, consisting of, or consisting essentially of SEQ ID NO: 83. In some aspects, the antibody comprises a CDR-H2 sequence comprising, consisting of, or consisting essentially of SEQ ID NO: 84. In some aspects, the antibody comprises a CDR-H2 sequence comprising, consisting of, or consisting essentially of SEQ ID NO: 85. In some aspects, the antibody comprises a CDR-H2 sequence comprising, consisting of, or consisting essentially of SEQ ID NO: 86. In some aspects, the antibody comprises a CDR-H2 sequence comprising, consisting of, or consisting essentially of SEQ ID NO: 87. In some aspects, the antibody comprises a CDR-H2 sequence comprising, consisting of, or consisting essentially of SEQ ID NO: 88. In some aspects, the antibody comprises a CDR-H2 sequence comprising, consisting of, or consisting essentially of SEQ ID NO: 89. In some aspects, the antibody comprises a CDR-H2 sequence comprising, consisting of, or consisting essentially of SEQ ID NO: 90. In some aspects, the antibody comprises a CDR-H2 sequence comprising, consisting of, or consisting essentially of SEQ ID NO: 91. In some aspects, the antibody comprises a CDR-H2 sequence comprising, consisting of, or consisting essentially of SEQ ID NO: 92. In some aspects, the antibody comprises a CDR-H2 sequence comprising, consisting of, or consisting essentially of SEQ ID NO: 93. In some aspects, the antibody comprises a CDR-H2 sequence comprising, consisting of, or consisting essentially of SEQ ID NO: 94. In some aspects, the antibody comprises a CDR-H2 sequence comprising, consisting of, or consisting essentially of SEQ ID NO: 95. In some aspects, the antibody comprises a CDR-H2 sequence comprising, consisting of, or consisting essentially of SEQ ID NO: 96. In some aspects, the antibody comprises a CDR-H2 sequence comprising, consisting of, or consisting essentially of SEQ ID NO: 97. In some aspects, the antibody comprises a CDR-H2 sequence comprising, consisting of, or consisting essentially of SEQ ID NO: 98. In some aspects, the antibody comprises a CDR-H2 sequence comprising, consisting of, or consisting essentially of SEQ ID NO: 99. In some aspects, the antibody comprises a CDR-H2 sequence comprising, consisting of, or consisting essentially of SEQ ID NO: 100. In some aspects, the antibody comprises a CDR-H2 sequence comprising, consisting of, or consisting essentially of SEQ ID NO: 101. In some aspects, the antibody comprises a CDR-H2 sequence comprising, consisting of, or consisting essentially of SEQ ID NO: 102. In some aspects, the antibody comprises a CDR-H2 sequence comprising, consisting of, or consisting essentially of SEQ ID NO: 103. In some aspects, the antibody comprises a CDR-H2 sequence comprising, consisting of, or consisting essentially of SEQ ID NO: 104. In some aspects, the antibody comprises a CDR-H2 sequence comprising, consisting of, or consisting essentially of SEQ ID NO: 105. In some aspects, the antibody comprises a CDR-H2 sequence comprising, consisting of, or consisting essentially of SEQ ID NO: 106. In some aspects, the antibody comprises a CDR-H2 sequence comprising, consisting of, or consisting essentially of SEQ ID NO: 107. In some aspects, the antibody comprises a CDR-H2 sequence comprising, consisting of, or consisting essentially of SEQ ID NO: 108. In some aspects, the antibody comprises a CDR-H2 sequence comprising, consisting of, or consisting essentially of SEQ ID NO: 109. In some aspects, the antibody comprises a CDR-H2 sequence comprising, consisting of, or consisting essentially of SEQ ID NO: 110. In some aspects, the antibody comprises a CDR-H2 sequence comprising, consisting of, or consisting essentially of SEQ ID NO: 111. In some aspects, the antibody comprises a CDR-H2 sequence comprising, consisting of, or consisting essentially of SEQ ID NO: 112. In some aspects, the antibody comprises a CDR-H2 sequence comprising, consisting of, or consisting essentially of SEQ ID NO: 113. In some aspects, the antibody comprises a CDR-H2 sequence comprising, consisting of, or consisting essentially of SEQ ID NO: 114. In some aspects, the antibody comprises a CDR-H2 sequence comprising, consisting of, or consisting essentially of SEQ ID NO: 115.

### 2.2.1.3. Kabat CDR-H1

In some embodiments, the antibody comprises a V_{H} sequence comprising a CDR-H1 sequence, wherein the CDR-H1 sequence comprises, consists of, or consists essentially of a Kabat CDR-H1 sequence of an illustrative antibody or V_{H} sequence provided herein. In some aspects, the Kabat CDR-H1 sequence is a Kabat CDR-H1 sequence of a V_{H} sequence provided in SEQ ID NOs: 167-216.

In some embodiments, the antibody comprises a V_{H} sequence comprising a Kabat CDR-H1 sequence comprising, consisting of, or consisting essentially of a sequence selected from SEQ ID NOs: 32-56. In some aspects, the antibody comprises a CDR-H1 sequence comprising, consisting of, or consisting essentially of SEQ ID NO: 32. In some aspects, the antibody comprises a CDR-H1 sequence comprising, consisting of, or consisting essentially of SEQ ID NO: 33. In some aspects, the antibody comprises a CDR-H1 sequence comprising, consisting of, or consisting essentially of SEQ ID NO: 34. In some aspects, the antibody comprises a CDR-H1 sequence comprising, consisting of, or consisting essentially of SEQ ID NO: 35. In some aspects, the antibody comprises a CDR-H1 sequence comprising, consisting of, or consisting essentially of SEQ ID NO: 36. In some aspects, the antibody comprises a CDR-H1 sequence comprising, consisting of, or consisting essentially of SEQ ID NO: 37. In some aspects, the antibody comprises a CDR-H1 sequence comprising, consisting of, or consisting essentially of SEQ ID NO: 38. In some aspects, the antibody comprises a CDR-H1 sequence comprising, consisting of, or consisting essentially of SEQ ID NO: 39. In some aspects, the antibody comprises a CDR-H1 sequence comprising, consisting of, or consisting essentially of SEQ ID NO: 40. In some aspects, the antibody comprises a CDR-H1 sequence comprising, consisting of, or consisting essentially of SEQ ID NO: 41. In some aspects, the antibody comprises a CDR-H1 sequence comprising, consisting of, or consisting essentially of SEQ ID NO: 42. In some aspects, the antibody comprises a CDR-H1 sequence comprising, consisting of, or consisting essentially of SEQ ID NO: 43. In some aspects, the antibody comprises a CDR-H1 sequence comprising, consisting of, or consisting essentially of SEQ ID NO: 44. In some aspects, the antibody comprises a CDR-H1 sequence comprising, consisting of, or consisting essentially of SEQ ID NO: 45. In some aspects, the antibody comprises a CDR-H1 sequence comprising, consisting of, or consisting essentially of SEQ ID NO: 46. In some aspects, the antibody comprises a CDR-H1 sequence comprising, consisting of, or consisting essentially of SEQ ID NO: 47. In some aspects, the antibody comprises a CDR-H1 sequence comprising, consisting of, or consisting essentially of SEQ ID NO: 48. In some aspects, the antibody comprises a CDR-H1 sequence comprising, consisting of, or consisting essentially of SEQ ID NO: 49. In some aspects, the antibody comprises a CDR-H1 sequence comprising, consisting of, or consisting essentially of SEQ ID NO: 50. In some aspects, the antibody comprises a CDR-H1 sequence comprising, consisting of, or consisting essentially of SEQ ID NO: 51. In some aspects, the antibody comprises a CDR-H1 sequence comprising, consisting of, or consisting essentially of SEQ ID NO: 52. In some aspects, the antibody comprises a CDR-H1 sequence comprising, consisting of, or consisting essentially of SEQ ID NO: 53. In some aspects, the antibody comprises a CDR-H1 sequence comprising, consisting of, or consisting essentially of SEQ ID NO: 54. In some aspects, the antibody comprises a CDR-H1 sequence comprising, consisting of, or consisting essentially of SEQ ID NO: 55. In some aspects, the antibody comprises a CDR-H1 sequence comprising, consisting of, or consisting essentially of SEQ ID NO: 56.

### 2.2.1.4. Kabat CDR-H3+Kabat CDR-H2

In some embodiments, the antibody comprises a V_{H} sequence comprising a Kabat CDR-H3 sequence comprising, consisting of, or consisting essentially of a sequence selected from SEQ ID NOs: 116-145, and a Kabat CDR-H2 sequence comprising, consisting of, or consisting essentially of a sequence selected from SEQ ID NOs: 79-115. In some aspects, the Kabat CDR-H3 sequence and the Kabat CDR-H2 sequence are both from a single illustrative V_{H} sequence provided in this disclosure. For example, in some aspects, the Kabat CDR-H3 and Kabat CDR-H2 are both from a single illustrative V_{H} sequence selected from SEQ ID NOs: 167-216.

### 2.2.1.5. Kabat CDR-H3+Kabat CDR-H1

In some embodiments, the antibody comprises a V_{H} sequence comprising a Kabat CDR-H3 sequence comprising, consisting of, or consisting essentially of a sequence selected from SEQ ID NOs: 116-145, and a Kabat CDR-H1 sequence comprising, consisting of, or consisting essentially of a sequence selected from SEQ ID NOs: 32-56. In some aspects, the Kabat CDR-H3 sequence and the Kabat CDR-H1 sequence are both from a single illustrative V_{H} sequence provided in this disclosure. For example, in some aspects, the Kabat CDR-H3 and Kabat CDR-H1 are both from a single illustrative V_{H} sequence selected from SEQ ID NOs: 167-216.

### 2.2.1.6. Kabat CDR-H1+Kabat CDR-H2

In some embodiments, the antibody comprises a V_{H} sequence comprising a Kabat CDR-H1 sequence comprising, consisting of, or consisting essentially of a sequence selected from SEQ ID NOs: 32-56 and a Kabat CDR-H2 sequence comprising, consisting of, or consisting essentially of a sequence selected from SEQ ID NOs: 79-115. In some aspects, the Kabat CDR-H1 sequence and the Kabat CDR-H2 sequence are both from a single illustrative V_{H} sequence provided in this disclosure. For example, in some aspects, the Kabat CDR-H1 and Kabat CDR-H2 are both from a single illustrative V_{H} sequence selected from SEQ ID NOs: 167-216.

### 2.2.1.7. Kabat CDR-H1+Kabat CDR-H2+Kabat CDR-H3

In some embodiments, the antibody comprises a V_{H} sequence comprising a Kabat CDR-H1 sequence comprising, consisting of, or consisting essentially of a sequence selected from SEQ ID NOs: 32-56, a Kabat CDR-H2 sequence comprising, consisting of, or consisting essentially of a sequence selected from SEQ ID NOs: 79-115, and a Kabat CDR-H3 sequence comprising, consisting of, or consisting essentially of a sequence selected from SEQ ID NOs: 116-145. In some aspects, the Kabat CDR-H1 sequence, Kabat CDR-H2 sequence, and Kabat CDR-H3 sequence are all from a single illustrative V_{H} sequence provided in this disclosure. For example, in some aspects, the Kabat CDR-H1, Kabat CDR-H2, and Kabat CDR-H3 are all from a single illustrative V_{H} sequence selected from SEQ ID NOs: 167-216.

### 2.2.1.8. Variants of V_{H} Sequences Comprising Illustrative Kabat CDRs

In some embodiments, the V_{H} sequences provided herein comprise a variant of an illustrative Kabat CDR-H3, CDR-H2, and/or CDR-H1 sequence provided in this disclosure.

In some aspects, the Kabat CDR-H3 sequence comprises, consists of, or consists essentially of a variant of an illustrative Kabat CDR-H3 sequence provided in this disclosure. In some aspects, the Kabat CDR-H3 sequence comprises, consists of, or consists essentially of a sequence having at least 70%, 75%, 80%, 85%, 90%, or 95% identity with any of the illustrative Kabat CDR-H3 sequences provided in this disclosure. In some aspects, the Kabat CDR-H3 sequence comprises, consists of, or consists essentially of any of the illustrative Kabat CDR-H3 sequences provided in this disclosure, with 1, 2, or 3 amino acid substitutions. In some aspects, the amino acid substitutions are conservative amino acid substitutions.

In some aspects, the Kabat CDR-H2 sequence comprises, consists of, or consists essentially of a variant of an illustrative Kabat CDR-H2 sequence provided in this disclosure. In some aspects, the Kabat CDR-H2 sequence comprises, consists of, or consists essentially of a sequence having at least 70%, 75%, 80%, 85%, 90%, or 95% identity with any of the illustrative Kabat CDR-H2 sequences provided in this disclosure. In some aspects, the Kabat CDR-H2 sequence comprises, consists of, or consists essentially of any of the illustrative Kabat CDR-H2 sequences provided in this disclosure, with 1, 2, or 3 amino acid substitutions. In some aspects, the amino acid substitutions are conservative amino acid substitutions.

In some aspects, the Kabat CDR-H1 sequence comprises, consists of, or consists essentially of a variant of an illustrative Kabat CDR-H1 sequence provided in this disclosure. In some aspects, the Kabat CDR-H1 sequence comprises, consists of, or consists essentially of a sequence having at least 70%, 75%, 80%, 85%, 90%, or 95% identity with any of the illustrative Kabat CDR-H1 sequences provided in this disclosure. In some aspects, the Kabat CDR-H1 sequence comprises, consists of, or consists essentially of any of the illustrative Kabat CDR-H1 sequences provided in this disclosure, with 1, 2, or 3 amino acid substitutions. In some aspects, the amino acid substitutions are conservative amino acid substitutions.

### 2.2.2. V_{H} Sequences Comprising Illustrative Chothia CDRs

In some embodiments, the antibody comprises a V_{H} sequence comprising one or more Chothia CDR-H sequences comprising, consisting of, or consisting essentially of one or more illustrative Chothia CDR-H sequences provided in this disclosure, and variants thereof.

### 2.2.2.1. Chotia CDR-H3

In some embodiments, the antibody comprises a V_{H} sequence comprising a CDR-H3 sequence, wherein the CDR-H3 sequence comprises, consists of, or consists essentially of a Chothia CDR-H3 sequence of an illustrative antibody or V_{H} sequence provided herein. In some aspects, the Chothia CDR-H3 sequence is a Chothia CDR-H3 sequence of a V_{H} sequence provided in SEQ ID NOs: 167-216.

In some embodiments, the antibody comprises a V_{H} sequence comprising a Chothia CDR-H3 sequence comprising, consisting of, or consisting essentially of a sequence selected from SEQ ID NOs: 116-145. In some aspects, the antibody comprises a CDR-H3 sequence comprising, consisting of, or consisting essentially of SEQ ID NO: 116. In some aspects, the antibody comprises a CDR-H3 sequence comprising, consisting of, or consisting essentially of SEQ ID NO: 117. In some aspects, the antibody comprises a CDR-H3 sequence comprising, consisting of, or consisting essentially of SEQ ID NO: 118. In some aspects, the antibody comprises a CDR-H3 sequence comprising, consisting of, or consisting essentially of SEQ ID NO: 119. In some aspects, the antibody comprises a CDR-H3 sequence comprising, consisting of, or consisting essentially of SEQ ID NO: 120. In some aspects, the antibody comprises a CDR-H3 sequence comprising, consisting of, or consisting essentially of SEQ ID NO: 121. In some aspects, the antibody comprises a CDR-H3 sequence comprising, consisting of, or consisting essentially of SEQ ID NO: 122. In some aspects, the antibody comprises a CDR-H3 sequence comprising, consisting of, or consisting essentially of SEQ ID NO: 123. In some aspects, the antibody comprises a CDR-H3 sequence comprising, consisting of, or consisting essentially of SEQ ID NO: 124. In some aspects, the antibody comprises a CDR-H3 sequence comprising, consisting of, or consisting essentially of SEQ ID NO: 125. In some aspects, the antibody comprises a CDR-H3 sequence comprising, consisting of, or consisting essentially of SEQ ID NO: 126. In some aspects, the antibody comprises a CDR-H3 sequence comprising, consisting of, or consisting essentially of SEQ ID NO: 127. In some aspects, the antibody comprises a CDR-H3 sequence comprising, consisting of, or consisting essentially of SEQ ID NO: 128. In some aspects, the antibody comprises a CDR-H3 sequence comprising, consisting of, or consisting essentially of SEQ ID NO: 129. In some aspects, the antibody comprises a CDR-H3 sequence comprising, consisting of, or consisting essentially of SEQ ID NO: 130. In some aspects, the antibody comprises a CDR-H3 sequence comprising, consisting of, or consisting essentially of SEQ ID NO: 131. In some aspects, the antibody comprises a CDR-H3 sequence comprising, consisting of, or consisting essentially of SEQ ID NO: 132. In some aspects, the antibody comprises a CDR-H3 sequence comprising, consisting of, or consisting essentially of SEQ ID NO: 133. In some aspects, the antibody comprises a CDR-H3 sequence comprising, consisting of, or consisting essentially of SEQ ID NO: 134. In some aspects, the antibody comprises a CDR-H3 sequence comprising, consisting of, or consisting essentially of SEQ ID NO: 135. In some aspects, the antibody comprises a CDR-H3 sequence comprising, consisting of, or consisting essentially of SEQ ID NO: 136. In some aspects, the antibody comprises a CDR-H3 sequence comprising, consisting of, or consisting essentially of SEQ ID NO: 137. In some aspects, the antibody comprises a CDR-H3 sequence comprising, consisting of, or consisting essentially of SEQ ID NO: 138. In some aspects, the antibody comprises a CDR-H3 sequence comprising, consisting of, or consisting essentially of SEQ ID NO: 139. In some aspects, the antibody comprises a CDR-H3 sequence comprising, consisting of, or consisting essentially of SEQ ID NO: 140. In some aspects, the antibody comprises a CDR-H3 sequence comprising, consisting of, or consisting essentially of SEQ ID NO: 141. In some aspects, the antibody comprises a CDR-H3 sequence comprising, consisting of, or consisting essentially of SEQ ID NO: 142. In some aspects, the antibody comprises a CDR-H3 sequence comprising, consisting of, or consisting essentially of SEQ ID NO: 143. In some aspects, the antibody comprises a CDR-H3 sequence comprising, consisting of, or consisting essentially of SEQ ID NO: 144. In some aspects, the antibody comprises a CDR-H3 sequence comprising, consisting of, or consisting essentially of SEQ ID NO: 145.

### 2.2.2.2. Chothia CDR-H2

In some embodiments, the antibody comprises a V_{H} sequence comprising a CDR-H2 sequence, wherein the CDR-H2 sequence comprises, consists of, or consists essentially of a Chothia CDR-H2 sequence of an illustrative antibody or V_{H} sequence provided herein. In some aspects, the Chothia CDR-H2 sequence is a Chothia CDR-H2 sequence of a V_{H} sequence provided in SEQ ID NOs: 167-216.

In some embodiments, the antibody comprises a V_{H} sequence comprising a Chothia CDR-H2 sequence comprising, consisting of, or consisting essentially of a sequence selected from SEQ ID NOs: 57-78. In some aspects, the antibody comprises a CDR-H2 sequence comprising, consisting of, or consisting essentially of SEQ ID NO: 57. In some aspects, the antibody comprises a CDR-H2 sequence comprising, consisting of, or consisting essentially of SEQ ID NO: 58. In some aspects, the antibody comprises a CDR-H2 sequence comprising, consisting of, or consisting essentially of SEQ ID NO: 59. In some aspects, the antibody comprises a CDR-H2 sequence comprising, consisting of, or consisting essentially of SEQ ID NO: 60. In some aspects, the antibody comprises a CDR-H2 sequence comprising, consisting of, or consisting essentially of SEQ ID NO: 61. In some aspects, the antibody comprises a CDR-H2 sequence comprising, consisting of, or consisting essentially of SEQ ID NO: 62. In some aspects, the antibody comprises a CDR-H2 sequence comprising, consisting of, or consisting essentially of SEQ ID NO: 63. In some aspects, the antibody comprises a CDR-H2 sequence comprising, consisting of, or consisting essentially of SEQ ID NO: 64. In some aspects, the antibody comprises a CDR-H2 sequence comprising, consisting of, or consisting essentially of SEQ ID NO: 65. In some aspects, the antibody comprises a CDR-H2 sequence comprising, consisting of, or consisting essentially of SEQ ID NO: 66. In some aspects, the antibody comprises a CDR-H2 sequence comprising, consisting of, or consisting essentially of SEQ ID NO: 67. In some aspects, the antibody comprises a CDR-H2 sequence comprising, consisting of, or consisting essentially of SEQ ID NO: 68. In some aspects, the antibody comprises a CDR-H2 sequence comprising, consisting of, or consisting essentially of SEQ ID NO: 69. In some aspects, the antibody comprises a CDR-H2 sequence comprising, consisting of, or consisting essentially of SEQ ID NO: 70. In some aspects, the antibody comprises a CDR-H2 sequence comprising, consisting of, or consisting essentially of SEQ ID NO: 71. In some aspects, the antibody comprises a CDR-H2 sequence comprising, consisting of, or consisting essentially of SEQ ID NO: 72. In some aspects, the antibody comprises a CDR-H2 sequence comprising, consisting of, or consisting essentially of SEQ ID NO: 73. In some aspects, the antibody comprises a CDR-H2 sequence comprising, consisting of, or consisting essentially of SEQ ID NO: 74. In some aspects, the antibody comprises a CDR-H2 sequence comprising, consisting of, or consisting essentially of SEQ ID NO: 75. In some aspects, the antibody comprises a CDR-H2 sequence comprising, consisting of, or consisting essentially of SEQ ID NO: 76. In some aspects, the antibody comprises a CDR-H2 sequence comprising, consisting of, or consisting essentially of SEQ ID NO: 77. In some aspects, the antibody comprises a CDR-H2 sequence comprising, consisting of, or consisting essentially of SEQ ID NO: 78.

### 2.2.2.3. Chothia CDR-H1

In some embodiments, the antibody comprises a V_{H} sequence comprising a CDR-HI sequence, wherein the CDR-H1 sequence comprises, consists of, or consists essentially of a Chothia CDR-H1 sequence of an illustrative antibody or V_{H} sequence provided herein. In some aspects, the Chothia CDR-H1 sequence is a Chothia CDR-H1 sequence of a V_{H} sequence provided in SEQ ID NOs: 167-216.

In some embodiments, the antibody comprises a V_{H} sequence comprising a Chothia CDR-H1 sequence comprising, consisting of, or consisting essentially of a sequence selected from SEQ ID NOs: 5-31. In some aspects, the antibody comprises a CDR-H1 sequence comprising, consisting of, or consisting essentially of SEQ ID NO: 5. In some aspects, the antibody comprises a CDR-H1 sequence comprising, consisting of, or consisting essentially of SEQ ID NO: 6. In some aspects, the antibody comprises a CDR-H1 sequence comprising, consisting of, or consisting essentially of SEQ ID NO: 7. In some aspects, the antibody comprises a CDR-H1 sequence comprising, consisting of, or consisting essentially of SEQ ID NO: 8. In some aspects, the antibody comprises a CDR-H1 sequence comprising, consisting of, or consisting essentially of SEQ ID NO: 9. In some aspects, the antibody comprises a CDR-HI sequence comprising, consisting of, or consisting essentially of SEQ ID NO: 10. In some aspects, the antibody comprises a CDR-H1 sequence comprising, consisting of, or consisting essentially of SEQ ID NO: 11. In some aspects, the antibody comprises a CDR-H1 sequence comprising, consisting of, or consisting essentially of SEQ ID NO: 12. In some aspects, the antibody comprises a CDR-H1 sequence comprising, consisting of, or consisting essentially of SEQ ID NO: 13. In some aspects, the antibody comprises a CDR-H1 sequence comprising, consisting of, or consisting essentially of SEQ ID NO: 14. In some aspects, the antibody comprises a CDR-H1 sequence comprising, consisting of, or consisting essentially of SEQ ID NO: 15. In some aspects, the antibody comprises a CDR-H1 sequence comprising, consisting of, or consisting essentially of SEQ ID NO: 16. In some aspects, the antibody comprises a CDR-H1 sequence comprising, consisting of, or consisting essentially of SEQ ID NO: 17. In some aspects, the antibody comprises a CDR-H1 sequence comprising, consisting of, or consisting essentially of SEQ ID NO: 18. In some aspects, the antibody comprises a CDR-H1 sequence comprising, consisting of, or consisting essentially of SEQ ID NO: 19. In some aspects, the antibody comprises a CDR-H1 sequence comprising, consisting of, or consisting essentially of SEQ ID NO: 20. In some aspects, the antibody comprises a CDR-H1 sequence comprising, consisting of, or consisting essentially of SEQ ID NO: 21. In some aspects, the antibody comprises a CDR-H1 sequence comprising, consisting of, or consisting essentially of SEQ ID NO: 22. In some aspects, the antibody comprises a CDR-H1 sequence comprising, consisting of, or consisting essentially of SEQ ID NO: 23. In some aspects, the antibody comprises a CDR-H1 sequence comprising, consisting of, or consisting essentially of SEQ ID NO: 24. In some aspects, the antibody comprises a CDR-H1 sequence comprising, consisting of, or consisting essentially of SEQ ID NO: 25. In some aspects, the antibody comprises a CDR-H1 sequence comprising, consisting of, or consisting essentially of SEQ ID NO: 26. In some aspects, the antibody comprises a CDR-H1 sequence comprising, consisting of, or consisting essentially of SEQ ID NO: 27. In some aspects, the antibody comprises a CDR-H1 sequence comprising, consisting of, or consisting essentially of SEQ ID NO: 28. In some aspects, the antibody comprises a CDR-H1 sequence comprising, consisting of, or consisting essentially of SEQ ID NO: 29. In some aspects, the antibody comprises a CDR-H1 sequence comprising, consisting of, or consisting essentially of SEQ ID NO: 30. In some aspects, the antibody comprises a CDR-H1 sequence comprising, consisting of, or consisting essentially of SEQ ID NO: 31.

### 2.2.2.4. Chothia CDR-H3+Chothia CDR-H2

In some embodiments, the antibody comprises a V_{H} sequence comprising a Chothia CDR-H3 sequence comprising, consisting of, or consisting essentially of a sequence selected from SEQ ID NOs: 116-145, and a Chothia CDR-H2 sequence comprising, consisting of, or consisting essentially of a sequence selected from SEQ ID NOs: 57-78. In some aspects, the Chothia CDR-H3 sequence and the Chothia CDR-H2 sequence are both from a single illustrative V_{H} sequence provided in this disclosure. For example, in some aspects, the Chothia CDR-H3 and Chothia CDR-H2 are both from a single illustrative V_{H} sequence selected from SEQ ID NOs: 167-216.

### 2.2.2.5. Chothia CDR-H3+Chothia CDR-H1

In some embodiments, the antibody comprises a V_{H} sequence comprising a Chothia CDR-H3 sequence comprising, consisting of, or consisting essentially of a sequence selected from SEQ ID NOs: 116-145, and a Chothia CDR-H1 sequence comprising, consisting of, or consisting essentially of a sequence selected from SEQ ID NOs: 5-31. In some aspects, the Chothia CDR-H3 sequence and the Chothia CDR-H1 sequence are both from a single illustrative V_{H} sequence provided in this disclosure. For example, in some aspects, the Chothia CDR-H3 and Chothia CDR-H1 are both from a single illustrative V_{H} sequence selected from SEQ ID NOs: 167-216.

### 2.2.2.6. Chothia CDR-H1+Chothia CDR-H2

In some embodiments, the antibody comprises a V_{H} sequence comprising a Chothia CDR-H1 sequence comprising, consisting of, or consisting essentially of a sequence selected from SEQ ID NOs: 5-31 and a Chothia CDR-H2 sequence comprising, consisting of, or consisting essentially of a sequence selected from SEQ ID NOs: 57-78. In some aspects, the Chothia CDR-H1 sequence and the Chothia CDR-H2 sequence are both from a single illustrative V_{H} sequence provided in this disclosure. For example, in some aspects, the Chothia CDR-H1 and Chothia CDR-H2 are both from a single illustrative V_{H} sequence selected from SEQ ID NOs: 167-216.

### 2.2.2.7. Chothia CDR-H1+Chothia CDR-H2+Chothia CDR-H3

In some embodiments, the antibody comprises a V_{H} sequence comprising a Chothia CDR-H1 sequence comprising, consisting of, or consisting essentially of a sequence selected from SEQ ID NOs: 5-31, a Chothia CDR-H2 sequence comprising, consisting of, or consisting essentially of a sequence selected from SEQ ID NOs: 57-78, and a Chothia CDR-H3 sequence comprising, consisting of, or consisting essentially of a sequence selected from SEQ ID NOs: 116-145. In some aspects, the Chothia CDR-H1 sequence, Chothia CDR-H2 sequence, and Chothia CDR-H3 sequence are all from a single illustrative V_{H} sequence provided in this disclosure. For example, in some aspects, the Chothia CDR-H1, Chothia CDR-H2, and Chothia CDR-H3 are all from a single illustrative V_{H} sequence selected from SEQ ID NOs: 167-216.

### 2.2.2.8. Variants of V_{H} Sequences Comprising Illustrative Chothia CDRs

In some embodiments, the V_{H} sequences provided herein comprise a variant of an illustrative Chothia CDR-H3, CDR-H2, and/or CDR-H1 sequence provided in this disclosure.

In some aspects, the Chothia CDR-H3 sequence comprises, consists of, or consists essentially of a variant of an illustrative Chothia CDR-H3 sequence provided in this disclosure. In some aspects, the Chothia CDR-H3 sequence comprises, consists of, or consists essentially of a sequence having at least 70%, 75%, 80%, 85%, 90%, or 95% identity with any of the illustrative Chothia CDR-H3 sequences provided in this disclosure. In some aspects, the Chothia CDR-H3 sequence comprises, consists of, or consists essentially of any of the illustrative Chothia CDR-H3 sequences provided in this disclosure, with 1, 2, or 3 amino acid substitutions. In some aspects, the amino acid substitutions are conservative amino acid substitutions.

In some aspects, the Chothia CDR-H2 sequence comprises, consists of, or consists essentially of a variant of an illustrative Chothia CDR-H2 sequence provided in this disclosure. In some aspects, the Chothia CDR-H2 sequence comprises, consists of, or consists essentially of a sequence having at least 70%, 75%, 80%, 85%, 90%, or 95% identity with any of the illustrative Chothia CDR-H2 sequences provided in this disclosure. In some aspects, the Chothia CDR-H2 sequence comprises, consists of, or consists essentially of any of the illustrative Chothia CDR-H2 sequences provided in this disclosure, with 1, 2, or 3 amino acid substitutions. In some aspects, the amino acid substitutions are conservative amino acid substitutions.

In some aspects, the Chothia CDR-H1 sequence comprises, consists of, or consists essentially of a variant of an illustrative Chothia CDR-H1 sequence provided in this disclosure. In some aspects, the Chothia CDR-H1 sequence comprises, consists of, or consists essentially of a sequence having at least 70%, 75%, 80%, 85%, 90%, or 95% identity with any of the illustrative Chothia CDR-H1 sequences provided in this disclosure. In some aspects, the Chothia CDR-H1 sequence comprises, consists of, or consists essentially of any of the illustrative Chothia CDR-H1 sequences provided in this disclosure, with 1, 2, or 3 amino acid substitutions. In some aspects, the amino acid substitutions are conservative amino acid substitutions.

### 2.3. V_{H} Sequences

In some embodiments, the antibody comprises, consists of, or consists essentially of a V_{H} sequence provided in SEQ ID NOs: 167-216.

In some embodiments, the antibody comprises a V_{H} sequence comprising, consisting of, or consisting essentially of a sequence selected from SEQ ID NOs: 167-216. In some aspects, the antibody comprises a V_{H} sequence comprising, consisting of, or consisting essentially of SEQ ID NO: 167. In some aspects, the antibody comprises a V_{H} sequence comprising, consisting of, or consisting essentially of SEQ ID NO: 168. In some aspects, the antibody comprises a V_{H} sequence comprising, consisting of, or consisting essentially of SEQ ID NO: 169. In some aspects, the antibody comprises a V_{H} sequence comprising, consisting of, or consisting essentially of SEQ ID NO: 170. In some aspects, the antibody comprises a V_{H} sequence comprising, consisting of, or consisting essentially of SEQ ID NO: 171. In some aspects, the antibody comprises a V_{H} sequence comprising, consisting of, or consisting essentially of SEQ ID NO: 172. In some aspects, the antibody comprises a V_{H} sequence comprising, consisting of, or consisting essentially of SEQ ID NO: 173. In some aspects, the antibody comprises a V_{H} sequence comprising, consisting of, or consisting essentially of SEQ ID NO: 174. In some aspects, the antibody comprises a V_{H} sequence comprising, consisting of, or consisting essentially of SEQ ID NO: 175. In some aspects, the antibody comprises a V_{H} sequence comprising, consisting of, or consisting essentially of SEQ ID NO: 176. In some aspects, the antibody comprises a V_{H} sequence comprising, consisting of, or consisting essentially of SEQ ID NO: 177. In some aspects, the antibody comprises a V_{H} sequence comprising, consisting of, or consisting essentially of SEQ ID NO: 178. In some aspects, the antibody comprises a V_{H} sequence comprising, consisting of, or consisting essentially of SEQ ID NO: 179. In some aspects, the antibody comprises a V_{H} sequence comprising, consisting of, or consisting essentially of SEQ ID NO: 180. In some aspects, the antibody comprises a V_{H} sequence comprising, consisting of, or consisting essentially of SEQ ID NO: 181. In some aspects, the antibody comprises a V_{H} sequence comprising, consisting of, or consisting essentially of SEQ ID NO: 182. In some aspects, the antibody comprises a V_{H} sequence comprising, consisting of, or consisting essentially of SEQ ID NO: 183. In some aspects, the antibody comprises a V_{H} sequence comprising, consisting of, or consisting essentially of SEQ ID NO: 184. In some aspects, the antibody comprises a V_{H} sequence comprising, consisting of, or consisting essentially of SEQ ID NO: 185. In some aspects, the antibody comprises a V_{H} sequence comprising, consisting of, or consisting essentially of SEQ ID NO: 186. In some aspects, the antibody comprises a V_{H} sequence comprising, consisting of, or consisting essentially of SEQ ID NO: 187. In some aspects, the antibody comprises a V_{H} sequence comprising, consisting of, or consisting essentially of SEQ ID NO: 188. In some aspects, the antibody comprises a V_{H} sequence comprising, consisting of, or consisting essentially of SEQ ID NO: 189. In some aspects, the antibody comprises a V_{H} sequence comprising, consisting of, or consisting essentially of SEQ ID NO: 190. In some aspects, the antibody comprises a V_{H} sequence comprising, consisting of, or consisting essentially of SEQ ID NO: 191. In some aspects, the antibody comprises a V_{H} sequence comprising, consisting of, or consisting essentially of SEQ ID NO: 192. In some aspects, the antibody comprises a V_{H} sequence comprising, consisting of, or consisting essentially of SEQ ID NO: 193. In some aspects, the antibody comprises a V_{H} sequence comprising, consisting of, or consisting essentially of SEQ ID NO: 194. In some aspects, the antibody comprises a V_{H} sequence comprising, consisting of, or consisting essentially of SEQ ID NO: 195. In some aspects, the antibody comprises a V_{H} sequence comprising, consisting of, or consisting essentially of SEQ ID NO: 196. In some aspects, the antibody comprises a V_{H} sequence comprising, consisting of, or consisting essentially of SEQ ID NO: 197. In some aspects, the antibody comprises a V_{H} sequence comprising, consisting of, or consisting essentially of SEQ ID NO: 198. In some aspects, the antibody comprises a V_{H} sequence comprising, consisting of, or consisting essentially of SEQ ID NO: 199. In some aspects, the antibody comprises a V_{H} sequence comprising, consisting of, or consisting essentially of SEQ ID NO: 200. In some aspects, the antibody comprises a V_{H} sequence comprising, consisting of, or consisting essentially of SEQ ID NO: 201. In some aspects, the antibody comprises a V_{H} sequence comprising, consisting of, or consisting essentially of SEQ ID NO: 202. In some aspects, the antibody comprises a V_{H} sequence comprising, consisting of, or consisting essentially of SEQ ID NO: 203. In some aspects, the antibody comprises a V_{H} sequence comprising, consisting of, or consisting essentially of SEQ ID NO: 204. In some aspects, the antibody comprises a V_{H} sequence comprising, consisting of, or consisting essentially of SEQ ID NO: 205. In some aspects, the antibody comprises a V_{H} sequence comprising, consisting of, or consisting essentially of SEQ ID NO: 206. In some aspects, the antibody comprises a V_{H} sequence comprising, consisting of, or consisting essentially of SEQ ID NO: 207. In some aspects, the antibody comprises a V_{H} sequence comprising, consisting of, or consisting essentially of SEQ ID NO: 208. In some aspects, the antibody comprises a V_{H} sequence comprising, consisting of, or consisting essentially of SEQ ID NO: 209. In some aspects, the antibody comprises a V_{H} sequence comprising, consisting of, or consisting essentially of SEQ ID NO: 210. In some aspects, the antibody comprises a V_{H} sequence comprising, consisting of, or consisting essentially of SEQ ID NO: 211. In some aspects, the antibody comprises a V_{H} sequence comprising, consisting of, or consisting essentially of SEQ ID NO: 212. In some aspects, the antibody comprises a V_{H} sequence comprising, consisting of, or consisting essentially of SEQ ID NO: 213. In some aspects, the antibody comprises a V_{H} sequence comprising, consisting of, or consisting essentially of SEQ ID NO: 214. In some aspects, the antibody comprises a V_{H} sequence comprising, consisting of, or consisting essentially of SEQ ID NO: 215. In some aspects, the antibody comprises a V_{H} sequence comprising, consisting of, or consisting essentially of SEQ ID NO: 216.

### 2.3.1. Variants of V_{H} Sequences

In some embodiments, the V_{H} sequences provided herein comprise, consist of, or consist essentially of a variant of an illustrative V_{H} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 2.4. CDR-L3 Sequences

In some embodiments, the antibody comprises a CDR-L3 sequence comprising, consisting of, or consisting essentially of a CDR-L3 sequence of an illustrative antibody or V_{L} sequence provided herein. In some aspects, the CDR-L3 sequence is a CDR-L3 sequence of a V_{L} sequence provided in SEQ ID NOs: 217-238.

In some embodiments, the antibody comprises a CDR-L3 sequence comprising, consisting of, or consisting essentially of a sequence selected from SEQ ID NOs: 161-166. In some aspects, the antibody comprises a CDR-L3 sequence comprising, consisting of, or consisting essentially of SEQ ID NO: 161. In some aspects, the antibody comprises a CDR-L3 sequence comprising, consisting of, or consisting essentially of SEQ ID NO: 162. In some aspects, the antibody comprises a CDR-L3 sequence comprising, consisting of, or consisting essentially of SEQ ID NO: 163. In some aspects, the antibody comprises a CDR-L3 sequence comprising, consisting of, or consisting essentially of SEQ ID NO: 164. In some aspects, the antibody comprises a CDR-L3 sequence comprising, consisting of, or consisting essentially of SEQ ID NO: 165. In some aspects, the antibody comprises a CDR-L3 sequence comprising, consisting of, or consisting essentially of SEQ ID NO: 166.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

### 2.5. V_{L} Sequences Comprising Illustrative CDRs

In some embodiments, the antibody comprises a V_{L} sequence comprising one or more CDR-L sequences comprising, consisting of, or consisting essentially of one or more illustrative CDR-L sequences provided in this disclosure, and variants thereof.

### 2.5.1. CDR-L3

In some embodiments, the antibody comprises a V_{L} sequence comprising a CDR-L3 sequence, wherein the CDR-L3 sequence comprises, consists of, or consists essentially of a CDR-L3 sequence of an illustrative antibody or V_{L} sequence provided herein. In some aspects, the CDR-L3 sequence is a CDR-L3 sequence of a V_{L} sequence provided in SEQ ID NOs: 217-238.

In some embodiments, the antibody comprises a V_{L} sequence comprising a CDR-L3 sequence comprising, consisting of, or consisting essentially of a sequence selected from SEQ ID NOs: 161-166. In some aspects, the antibody comprises a CDR-L3 sequence comprising, consisting of, or consisting essentially of SEQ ID NO: 161. In some aspects, the antibody comprises a CDR-L3 sequence comprising, consisting of, or consisting essentially of SEQ ID NO: 162. In some aspects, the antibody comprises a CDR-L3 sequence comprising, consisting of, or consisting essentially of SEQ ID NO: 163. In some aspects, the antibody comprises a CDR-L3 sequence comprising, consisting of, or consisting essentially of SEQ ID NO: 164. In some aspects, the antibody comprises a CDR-L3 sequence comprising, consisting of, or consisting essentially of SEQ ID NO: 165. In some aspects, the antibody comprises a CDR-L3 sequence comprising, consisting of, or consisting essentially of SEQ ID NO: 166.

### 2.5.2. CDR-L2

In some embodiments, the antibody comprises a V_{L} sequence comprising a CDR-L2 sequence, wherein the CDR-L2 sequence comprises, consists of, or consists essentially of a CDR-L2 sequence of an illustrative antibody or V_{L} sequence provided herein. In some aspects, the CDR-L2 sequence is a CDR-L2 sequence of a V_{L} sequence provided in SEQ ID NOs: 217-238.

In some embodiments, the antibody comprises a V_{L} sequence comprising a CDR-L2 sequence comprising, consisting of, or consisting essentially of a sequence selected from SEQ ID NOs: 155-160. In some aspects, the antibody comprises a V_{L} sequence comprising a CDR-L2 sequence comprising, consisting of, or consisting essentially of SEQ ID NO: 155. In some aspects, the antibody comprises a V_{L} sequence comprising a CDR-L2 sequence comprising, consisting of, or consisting essentially of SEQ ID NO: 156. In some aspects, the antibody comprises a V_{L} sequence comprising a CDR-L2 sequence comprising, consisting of, or consisting essentially of SEQ ID NO: 157. In some aspects, the antibody comprises a V_{L} sequence comprising a CDR-L2 sequence comprising, consisting of, or consisting essentially of SEQ ID NO: 158. In some aspects, the antibody comprises a V_{L} sequence comprising a CDR-L2 sequence comprising, consisting of, or consisting essentially of SEQ ID NO: 159. In some aspects, the antibody comprises a V_{L} sequence comprising a CDR-L2 sequence comprising, consisting of, or consisting essentially of SEQ ID NO: 160.

### 2.5.3. CDR-L1

In some embodiments, the antibody comprises a V_{L} sequence comprising a CDR-L1 sequence, wherein the CDR-L1 sequence comprises, consists of, or consists essentially of a CDR-L1 sequence of an illustrative antibody or V_{L} sequence provided herein. In some aspects, the CDR-L1 sequence is a CDR-L1 sequence of a V_{L} sequence provided in SEQ ID NOs: 217-238.

In some embodiments, the antibody comprises a V_{L} sequence comprising a CDR-L1 sequence comprising, consisting of, or consisting essentially of a sequence selected from SEQ ID NOs: 146-154. In some aspects, the antibody comprises a V_{L} sequence comprising a CDR-L1 sequence comprising, consisting of, or consisting essentially of SEQ ID NO: 146. In some aspects, the antibody comprises a V_{L} sequence comprising a CDR-L1 sequence comprising, consisting of, or consisting essentially of SEQ ID NO: 147. In some aspects, the antibody comprises a V_{L} sequence comprising a CDR-L1 sequence comprising, consisting of, or consisting essentially of SEQ ID NO: 148. In some aspects, the antibody comprises a V_{L} sequence comprising a CDR-L1 sequence comprising, consisting of, or consisting essentially of SEQ ID NO: 149. In some aspects, the antibody comprises a V_{L} sequence comprising a CDR-L1 sequence comprising, consisting of, or consisting essentially of SEQ ID NO: 150. In some aspects, the antibody comprises a V_{L} sequence comprising a CDR-L1 sequence comprising, consisting of, or consisting essentially of SEQ ID NO: 151. In some aspects, the antibody comprises a V_{L} sequence comprising a CDR-L1 sequence comprising, consisting of, or consisting essentially of SEQ ID NO: 152. In some aspects, the antibody comprises a V_{L} sequence comprising a CDR-L1 sequence comprising, consisting of, or consisting essentially of SEQ ID NO: 153. In some aspects, the antibody comprises a V_{L} sequence comprising a CDR-L1 sequence comprising, consisting of, or consisting essentially of SEQ ID NO: 154.

### 2.5.4. CDR-L3+CDR-L2

In some embodiments, the antibody comprises a V_{L} sequence comprising a CDR-L3 sequence comprising, consisting of, or consisting essentially of a sequence selected from SEQ ID NOs: 161-166 and a CDR-L2 sequence comprising, consisting of, or consisting essentially of a sequence selected from SEQ ID NOs: 155-160. In some aspects, the CDR-L3 sequence and the CDR-L2 sequence are both from a single illustrative V_{L} sequence provided in this disclosure. For example, in some aspects, the CDR-L3 and CDR-L2 are both from a single illustrative V_{L} sequence selected from SEQ ID NOs: 217-238.

### 2.5.5. CDR-L3+CDR-L1

In some embodiments, the antibody comprises a V_{L} sequence comprising a CDR-L3 sequence comprising, consisting of, or consisting essentially of a sequence selected from SEQ ID NOs: 161-166 and a CDR-L1 sequence comprising, consisting of, or consisting essentially of a sequence selected from SEQ ID NOs: 146-154. In some aspects, the CDR-L3 sequence and the CDR-L1 sequence are both from a single illustrative V_{L} sequence provided in this disclosure. For example, in some aspects, the CDR-L3 and CDR-L1 are both from a single illustrative V_{L} sequence selected from SEQ ID NOs: 217-238.

### 2.5.6. CDR-L1+CDR-L2

In some embodiments, the antibody comprises a V_{L} sequence comprising a CDR-L1 sequence comprising, consisting of, or consisting essentially of a sequence selected from SEQ ID NOs: 146-154 and a CDR-L2 sequence comprising, consisting of, or consisting essentially of a sequence selected from SEQ ID NOs: 155-160. In some aspects, the CDR-L1 sequence and the CDR-L2 sequence are both from a single illustrative V_{L} sequence provided in this disclosure. For example, in some aspects, the CDR-L1 and CDR-L2 are both from a single illustrative V_{L} sequence selected from SEQ ID NOs: 217-238.

### 2.5.7. CDR-L1+CDR-L2+CDR-L3

In some embodiments, the antibody comprises a V_{L} sequence comprising a CDR-L1 sequence comprising, consisting of, or consisting essentially of a sequence selected from SEQ ID NOs: 146-154, a CDR-L2 sequence comprising, consisting of, or consisting essentially of a sequence selected from SEQ ID NOs: 155-160, and a CDR-L3 sequence comprising, consisting of, or consisting essentially of a sequence selected from SEQ ID NOs: 161-166. In some aspects, the CDR-L1 sequence, CDR-L2 sequence, and CDR-L3 sequence are all from a single illustrative V_{L} sequence provided in this disclosure. For example, in some aspects, the CDR-L1, CDR-L2, and CDR-L3 are all from a single illustrative V_{L} sequence selected from SEQ ID NOs: 217-238.

### 2.5.8. Variants of V_{L} Sequences Comprising Illustrative CDR-Ls

In some embodiments, the V_{L} sequences provided herein comprise a variant of an illustrative CDR-L3, CDR-L2, and/or CDR-L1 sequence provided in this disclosure.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

### 2.6. V_{L} Sequences

In some embodiments, the antibody comprises, consists of, or consists essentially of a V_{L} sequence provided in SEQ ID NOs: 217-238.

In some embodiments, the antibody comprises a V_{L} sequence comprising, consisting of, or consisting essentially of a sequence selected from SEQ ID NOs: 217-238. In some aspects, the antibody comprises a V_{L} sequence comprising, consisting of, or consisting essentially of SEQ ID NO: 217. In some aspects, the antibody comprises a V_{L} sequence comprising, consisting of, or consisting essentially of SEQ ID NO: 218. In some aspects, the antibody comprises a V_{L} sequence comprising, consisting of, or consisting essentially of SEQ ID NO: 219. In some aspects, the antibody comprises a V_{L} sequence comprising, consisting of, or consisting essentially of SEQ ID NO: 220. In some aspects, the antibody comprises a V_{L} sequence comprising, consisting of, or consisting essentially of SEQ ID NO: 221. In some aspects, the antibody comprises a V_{L} sequence comprising, consisting of, or consisting essentially of SEQ ID NO: 222. In some aspects, the antibody comprises a V_{L} sequence comprising, consisting of, or consisting essentially of SEQ ID NO: 223. In some aspects, the antibody comprises a V_{L} sequence comprising, consisting of, or consisting essentially of SEQ ID NO: 224. In some aspects, the antibody comprises a V_{L} sequence comprising, consisting of, or consisting essentially of SEQ ID NO: 225. In some aspects, the antibody comprises a V_{L} sequence comprising, consisting of, or consisting essentially of SEQ ID NO: 226. In some aspects, the antibody comprises a V_{L} sequence comprising, consisting of, or consisting essentially of SEQ ID NO: 227. In some aspects, the antibody comprises a V_{L} sequence comprising, consisting of, or consisting essentially of SEQ ID NO: 228. In some aspects, the antibody comprises a V_{L} sequence comprising, consisting of, or consisting essentially of SEQ ID NO: 229. In some aspects, the antibody comprises a V_{L} sequence comprising, consisting of, or consisting essentially of SEQ ID NO: 230. In some aspects, the antibody comprises a V_{L} sequence comprising, consisting of, or consisting essentially of SEQ ID NO: 231. In some aspects, the antibody comprises a V_{L} sequence comprising, consisting of, or consisting essentially of SEQ ID NO: 232. In some aspects, the antibody comprises a V_{L} sequence comprising, consisting of, or consisting essentially of SEQ ID NO: 233. In some aspects, the antibody comprises a V_{L} sequence comprising, consisting of, or consisting essentially of SEQ ID NO: 234. In some aspects, the antibody comprises a V_{L} sequence comprising, consisting of, or consisting essentially of SEQ ID NO: 235. In some aspects, the antibody comprises a V_{L} sequence comprising, consisting of, or consisting essentially of SEQ ID NO: 236. In some aspects, the antibody comprises a V_{L} sequence comprising, consisting of, or consisting essentially of SEQ ID NO: 237. In some aspects, the antibody comprises a V_{L} sequence comprising, consisting of, or consisting essentially of SEQ ID NO: 238.

### 2.6.1. Variants of V_{L} Sequences

In some embodiments, the V_{L} sequences provided herein comprise, consist of, or consist essentially of a variant of an illustrative V_{L} sequence provided in this disclosure.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 2.7. Pairs

### 2.7.1. CDR-H3 CDR-L3 Pairs

In some embodiments, the antibody comprises a CDR-H3 sequence and a CDR-L3 sequence. In some aspects, the CDR-H3 sequence is part of a V_{H} and the CDR-L3 sequence is part of a V_{L}.

In some aspects, the CDR-H3 sequence is a CDR-H3 sequence comprising, consisting of, or consisting essentially of any one of SEQ ID NOs: 116-145, and the CDR-L3 sequence is a CDR-L3 sequence comprising, consisting of, or consisting essentially of any one of SEQ ID NOs: 161-166.

In some aspects, the CDR-H3—CDR-L3 pairs are selected from SEQ ID NO: 116 and SEQ ID NO: 161; SEQ ID NO: 117 and SEQ ID NO: 161; SEQ ID NO: 118 and SEQ ID NO: 161; SEQ ID NO: 119 and SEQ ID NO: 161; SEQ ID NO: 120 and SEQ ID NO: 161; SEQ ID NO: 121 and SEQ ID NO: 161; SEQ ID NO: 122 and SEQ ID NO: 161; SEQ ID NO: 123 and SEQ ID NO: 161; SEQ ID NO: 124 and SEQ ID NO: 161; SEQ ID NO: 125 and SEQ ID NO: 161; SEQ ID NO: 126 and SEQ ID NO: 161; SEQ ID NO: 127 and SEQ ID NO: 161; SEQ ID NO: 128 and SEQ ID NO: 161; SEQ ID NO: 129 and SEQ ID NO: 161; SEQ ID NO: 130 and SEQ ID NO: 161; SEQ ID NO: 131 and SEQ ID NO: 161; SEQ ID NO: 132 and SEQ ID NO: 161; SEQ ID NO: 133 and SEQ ID NO: 161; SEQ ID NO: 134 and SEQ ID NO: 161; SEQ ID NO: 135 and SEQ ID NO:161; SEQ ID NO: 136 and SEQ ID NO: 161; SEQ ID NO: 137 and SEQ ID NO: 161; SEQ ID NO: 138 and SEQ ID NO: 161; SEQ ID NO: 139 and SEQ ID NO: 161; SEQ ID NO: 140 and SEQ ID NO: 161; SEQ ID NO: 141 and SEQ ID NO: 161; SEQ ID NO: 142 and SEQ ID NO: 161; SEQ ID NO: 143 and SEQ ID NO: 161; SEQ ID NO: 144 and SEQ ID NO: 161; SEQ ID NO: 145 and SEQ ID NO: 161.

In some aspects, the CDR-H3—CDR-L3 pairs are selected from SEQ ID NO: 116 and SEQ ID NO: 162; SEQ ID NO: 117 and SEQ ID NO: 162; SEQ ID NO: 118 and SEQ ID NO: 162; SEQ ID NO: 119 and SEQ ID NO: 162; SEQ ID NO: 120 and SEQ ID NO: 162; SEQ ID NO: 121 and SEQ ID NO: 162; SEQ ID NO: 122 and SEQ ID NO: 162; SEQ ID NO: 123 and SEQ ID NO: 162; SEQ ID NO: 124 and SEQ ID NO: 162; SEQ ID NO: 125 and SEQ ID NO: 162; SEQ ID NO: 126 and SEQ ID NO: 162; SEQ ID NO: 127 and SEQ ID NO: 162; SEQ ID NO: 128 and SEQ ID NO: 162; SEQ ID NO: 129 and SEQ ID NO: 162; SEQ ID NO: 130 and SEQ ID NO: 162; SEQ ID NO: 131 and SEQ ID NO: 162; SEQ ID NO: 132 and SEQ ID NO: 162; SEQ ID NO: 133 and SEQ ID NO: 162; SEQ ID NO: 134 and SEQ ID NO: 162; SEQ ID NO: 135 and SEQ ID NO:161; SEQ ID NO: 136 and SEQ ID NO: 162; SEQ ID NO: 137 and SEQ ID NO: 162; SEQ ID NO: 138 and SEQ ID NO: 162; SEQ ID NO: 139 and SEQ ID NO: 162; SEQ ID NO: 140 and SEQ ID NO: 162; SEQ ID NO: 141 and SEQ ID NO: 162; SEQ ID NO: 142 and SEQ ID NO: 162; SEQ ID NO: 143 and SEQ ID NO: 162; SEQ ID NO: 144 and SEQ ID NO: 162; SEQ ID NO: 145 and SEQ ID NO: 162.

In some aspects, the CDR-H3—CDR-L3 pairs are selected from SEQ ID NO: 116 and SEQ ID NO: 163; SEQ ID NO: 117 and SEQ ID NO: 163; SEQ ID NO: 118 and SEQ ID NO: 163; SEQ ID NO: 119 and SEQ ID NO: 163; SEQ ID NO: 120 and SEQ ID NO: 163; SEQ ID NO: 121 and SEQ ID NO: 163; SEQ ID NO: 122 and SEQ ID NO: 163; SEQ ID NO: 123 and SEQ ID NO: 163; SEQ ID NO: 124 and SEQ ID NO: 163; SEQ ID NO: 125 and SEQ ID NO: 163; SEQ ID NO: 126 and SEQ ID NO: 163; SEQ ID NO: 127 and SEQ ID NO: 163; SEQ ID NO: 128 and SEQ ID NO: 163; SEQ ID NO: 129 and SEQ ID NO: 163; SEQ ID NO: 130 and SEQ ID NO: 163; SEQ ID NO: 131 and SEQ ID NO: 163; SEQ ID NO: 132 and SEQ ID NO: 163; SEQ ID NO: 133 and SEQ ID NO: 163; SEQ ID NO: 134 and SEQ ID NO: 163; SEQ ID NO: 135 and SEQ ID NO:161; SEQ ID NO: 136 and SEQ ID NO: 163; SEQ ID NO: 137 and SEQ ID NO: 163; SEQ ID NO: 138 and SEQ ID NO: 163; SEQ ID NO: 139 and SEQ ID NO: 163; SEQ ID NO: 140 and SEQ ID NO: 163; SEQ ID NO: 141 and SEQ ID NO: 163; SEQ ID NO: 142 and SEQ ID NO: 163; SEQ ID NO: 143 and SEQ ID NO: 163; SEQ ID NO: 144 and SEQ ID NO: 163; SEQ ID NO: 145 and SEQ ID NO: 163.

In some aspects, the CDR-H3—CDR-L3 pairs are selected from SEQ ID NO: 116 and SEQ ID NO: 164; SEQ ID NO: 117 and SEQ ID NO: 164; SEQ ID NO: 118 and SEQ ID NO: 164; SEQ ID NO: 119 and SEQ ID NO: 164; SEQ ID NO: 120 and SEQ ID NO: 164; SEQ ID NO: 121 and SEQ ID NO: 164; SEQ ID NO: 122 and SEQ ID NO: 164; SEQ ID NO: 123 and SEQ ID NO: 164; SEQ ID NO: 124 and SEQ ID NO: 164; SEQ ID NO: 125 and SEQ ID NO: 164; SEQ ID NO: 126 and SEQ ID NO: 164; SEQ ID NO: 127 and SEQ ID NO: 164; SEQ ID NO: 128 and SEQ ID NO: 164; SEQ ID NO: 129 and SEQ ID NO: 164; SEQ ID NO: 130 and SEQ ID NO: 164; SEQ ID NO: 131 and SEQ ID NO: 164; SEQ ID NO: 132 and SEQ ID NO: 164; SEQ ID NO: 133 and SEQ ID NO: 164; SEQ ID NO: 134 and SEQ ID NO: 164; SEQ ID NO: 135 and SEQ ID NO:161; SEQ ID NO: 136 and SEQ ID NO: 164; SEQ ID NO: 137 and SEQ ID NO: 164; SEQ ID NO: 138 and SEQ ID NO: 164; SEQ ID NO: 139 and SEQ ID NO: 164; SEQ ID NO: 140 and SEQ ID NO: 164; SEQ ID NO: 141 and SEQ ID NO: 164; SEQ ID NO: 142 and SEQ ID NO: 164; SEQ ID NO: 143 and SEQ ID NO: 164; SEQ ID NO: 144 and SEQ ID NO: 164; SEQ ID NO: 145 and SEQ ID NO: 164.

In some aspects, the CDR-H3—CDR-L3 pairs are selected from SEQ ID NO: 116 and SEQ ID NO: 165; SEQ ID NO: 117 and SEQ ID NO: 165; SEQ ID NO: 118 and SEQ ID NO: 165; SEQ ID NO: 119 and SEQ ID NO: 165; SEQ ID NO: 120 and SEQ ID NO: 165; SEQ ID NO: 121 and SEQ ID NO: 165; SEQ ID NO: 122 and SEQ ID NO: 165; SEQ ID NO: 123 and SEQ ID NO: 165; SEQ ID NO: 124 and SEQ ID NO: 165; SEQ ID NO: 125 and SEQ ID NO: 165; SEQ ID NO: 126 and SEQ ID NO: 165; SEQ ID NO: 127 and SEQ ID NO: 165; SEQ ID NO: 128 and SEQ ID NO: 165; SEQ ID NO: 129 and SEQ ID NO: 165; SEQ ID NO: 130 and SEQ ID NO: 165; SEQ ID NO: 131 and SEQ ID NO: 165; SEQ ID NO: 132 and SEQ ID NO: 165; SEQ ID NO: 133 and SEQ ID NO: 165; SEQ ID NO: 134 and SEQ ID NO: 165; SEQ ID NO: 135 and SEQ ID NO:161; SEQ ID NO: 136 and SEQ ID NO: 165; SEQ ID NO: 137 and SEQ ID NO: 165; SEQ ID NO: 138 and SEQ ID NO: 165; SEQ ID NO: 139 and SEQ ID NO: 165; SEQ ID NO: 140 and SEQ ID NO: 165; SEQ ID NO: 141 and SEQ ID NO: 165; SEQ ID NO: 142 and SEQ ID NO: 165; SEQ ID NO: 143 and SEQ ID NO: 165; SEQ ID NO: 144 and SEQ ID NO: 165; SEQ ID NO: 145 and SEQ ID NO: 165.

In some aspects, the CDR-H3—CDR-L3 pairs are selected from SEQ ID NO: 116 and SEQ ID NO: 166; SEQ ID NO: 117 and SEQ ID NO: 166; SEQ ID NO: 118 and SEQ ID NO: 166; SEQ ID NO: 119 and SEQ ID NO: 166; SEQ ID NO: 120 and SEQ ID NO: 166; SEQ ID NO: 121 and SEQ ID NO: 166; SEQ ID NO: 122 and SEQ ID NO: 166; SEQ ID NO: 123 and SEQ ID NO: 166; SEQ ID NO: 124 and SEQ ID NO: 166; SEQ ID NO: 125 and SEQ ID NO: 166; SEQ ID NO: 126 and SEQ ID NO: 166; SEQ ID NO: 127 and SEQ ID NO: 166; SEQ ID NO: 128 and SEQ ID NO: 166; SEQ ID NO: 129 and SEQ ID NO: 166; SEQ ID NO: 130 and SEQ ID NO: 166; SEQ ID NO: 131 and SEQ ID NO: 166; SEQ ID NO: 132 and SEQ ID NO: 166; SEQ ID NO: 133 and SEQ ID NO: 166; SEQ ID NO: 134 and SEQ ID NO: 166; SEQ ID NO: 135 and SEQ ID NO:161; SEQ ID NO: 136 and SEQ ID NO: 166; SEQ ID NO: 137 and SEQ ID NO: 166; SEQ ID NO: 138 and SEQ ID NO: 166; SEQ ID NO: 139 and SEQ ID NO: 166; SEQ ID NO: 140 and SEQ ID NO: 166; SEQ ID NO: 141 and SEQ ID NO: 166; SEQ ID NO: 142 and SEQ ID NO: 166; SEQ ID NO: 143 and SEQ ID NO: 166; SEQ ID NO: 144 and SEQ ID NO: 166; SEQ ID NO: 145 and SEQ ID NO: 166.

### 2.7.1.1. Variants of CDR-H3 CDR-L3 Pairs

In some embodiments, the CDR-H3—CDR-L3 pairs provided herein comprise a variant of an illustrative CDR-H3 and/or CDR-L1 sequence provided in this disclosure.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

### 2.7.2. CDR-H1—CDR-L1 Pairs

In some embodiments, the antibody comprises a CDR-H1 sequence and a CDR-L1 sequence. In some aspects, the CDR-H1 sequence is part of a V_{H} and the CDR-L1 sequence is part of a V_{L}.

In some aspects, the CDR-H1 sequence is a Chothia CDR-H1 sequence comprising, consisting of, or consisting essentially of any one of SEQ ID NOs: 5-31, and the CDR-L1 sequence is a CDR-L1 sequence comprising, consisting of, or consisting essentially of any one of SEQ ID NOs: 146-154.

In some aspects, the CDR-H1 sequence is a Kabat CDR-H1 sequence comprising, consisting of, or consisting essentially of any one of SEQ ID NOs: 32-56, and the CDR-L1 sequence is a CDR-L1 sequence comprising, consisting of, or consisting essentially of any one of SEQ ID NOs: 146-154.

### 2.7.2.1. Variants of CDR-H1—CDR-L1 Pairs

In some embodiments, the CDR-H1—CDR-L1 pairs provided herein comprise a variant of an illustrative CDR-H1 and/or CDR-L1 sequence provided in this disclosure.

In some aspects, the CDR-H1 sequence comprises, consists of, or consists essentially of a variant of an illustrative CDR-H1 sequence provided in this disclosure. In some aspects, the CDR-H1 sequence comprises, consists of, or consists essentially of a sequence having at least 70%, 75%, 80%, 85%, 90%, or 95% identity with any of the illustrative CDR-H1 sequences provided in this disclosure. In some aspects, the CDR-H1 sequence comprises, consists of, or consists essentially of any of the illustrative CDR-H1 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

### 2.7.3. CDR-H2—CDR-L2 Pairs

In some embodiments, the antibody comprises a CDR-H2 sequence and a CDR-L2 sequence. In some aspects, the CDR-H2 sequence is part of a V_{H} and the CDR-L2 sequence is part of a V_{L}.

In some aspects, the CDR-H2 sequence is a Chothia CDR-H2 sequence comprising, consisting of, or consisting essentially of any one of SEQ ID NOs: 57-78, and the CDR-L2 sequence is a CDR-L2 sequence comprising, consisting of, or consisting essentially of any one of SEQ ID NOs: 155-160.

In some aspects, the CDR-H1 sequence is a Kabat CDR-H2 sequence comprising, consisting of, or consisting essentially of any one of SEQ ID NOs: 79-115, and the CDR-L2 sequence is a CDR-L2 sequence comprising, consisting of, or consisting essentially of any one of SEQ ID NOs: 155-160.

### 2.7.3.1. Variants of CDR-H2—CDR-L2 Pairs

In some embodiments, the CDR-H2—CDR-L2 pairs provided herein comprise a variant of an illustrative CDR-H2 and/or CDR-L2 sequence provided in this disclosure.

In some aspects, the CDR-H2 sequence comprises, consists of, or consists essentially of a variant of an illustrative CDR-H2 sequence provided in this disclosure. In some aspects, the CDR-H2 sequence comprises, consists of, or consists essentially of a sequence having at least 70%, 75%, 80%, 85%, 90%, or 95% identity with any of the illustrative CDR-H2 sequences provided in this disclosure. In some aspects, the CDR-H2 sequence comprises, consists of, or consists essentially of any of the illustrative CDR-H2 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

2.7.4. V_{H}-V_{L} Pairs

In some embodiments, the antibody comprises a V_{H} sequence and a V_{L} sequence.

In some aspects, the V_{H} sequence is a V_{H} sequence comprising, consisting of, or consisting essentially of any one of SEQ ID NOs: 167-216, and the V_{L} sequence is a V_{L} sequence comprising, consisting of, or consisting essentially of any one of SEQ ID NOs: 217-238.

In some aspects, the V_{H}-V_{L} pairs are selected from SEQ ID NO: 167 and SEQ ID NO: 217; SEQ ID NO: 168 and SEQ ID NO:217; SEQ ID NO: 169 and SEQ ID NO:217; SEQ ID NO: 170 and SEQ ID NO:217; SEQ ID NO:171 and SEQ ID NO:217; SEQ ID NO: 172 and SEQ ID NO:217; SEQ ID NO: 173 and SEQ ID NO:217; SEQ ID NO: 174 and SEQ ID NO:217; SEQ ID NO: 175 and SEQ ID NO:217; SEQ ID NO: 176 and SEQ ID NO:217; SEQ ID NO: 177 and SEQ ID NO:217; SEQ ID NO:178 and SEQ ID NO:217; SEQ ID NO:179 and SEQ ID NO: 217; SEQ ID NO:180 and SEQ ID NO:217; SEQ ID NO:181 and SEQ ID NO:217; SEQ ID NO: 182 and SEQ ID NO:217; SEQ ID NO: 183 and SEQ ID NO:217; SEQ ID NO:184 and SEQ ID NO:217; SEQ ID NO: 185 and SEQ ID NO:217; SEQ ID NO: 186 and SEQ ID NO:217; SEQ ID NO:187 and SEQ ID NO:217; SEQ ID NO: 188 and SEQ ID NO:217; SEQ ID NO:189 and SEQ ID NO:217; SEQ ID NO: 190 and SEQ ID NO:217; SEQ ID NO: 191 and SEQ ID NO: 217; SEQ ID NO: 192 and SEQ ID NO:217; SEQ ID NO: 193 and SEQ ID NO:217; SEQ ID NO: 194 and SEQ ID NO:217; SEQ ID NO: 195 and SEQ ID NO:217; SEQ ID NO: 196 and SEQ ID NO:217; SEQ ID NO: 197 and SEQ ID NO:217; SEQ ID NO: 198 and SEQ ID NO:217; SEQ ID NO: 199 and SEQ ID NO:217; SEQ ID NO:200 and SEQ ID NO:217; SEQ ID NO:201 and SEQ ID NO:217; SEQ ID NO:202 and SEQ ID NO:217; SEQ ID NO:203 and SEQ ID NO: 217; SEQ ID NO:204 and SEQ ID NO:217; SEQ ID NO:205 and SEQ ID NO:217; SEQ ID NO: 206 and SEQ ID NO:217; SEQ ID NO:207 and SEQ ID NO:217; SEQ ID NO:208 and SEQ ID NO:217; SEQ ID NO:209 and SEQ ID NO:217; SEQ ID NO:210 and SEQ ID NO:217; SEQ ID NO:211 and SEQ ID NO:217; SEQ ID NO:212 and SEQ ID NO:217; SEQ ID NO:213 and SEQ ID NO:217; SEQ ID NO:214 and SEQ ID NO:217; SEQ ID NO:215 and SEQ ID NO: 217; and SEQ ID NO:216 and SEQ ID NO:217.

In some aspects, the V_{H}-V_{L} pairs are selected from SEQ ID NO:167 and SEQ ID NO: 218; SEQ ID NO: 168 and SEQ ID NO:218; SEQ ID NO: 169 and SEQ ID NO:218; SEQ ID NO: 170 and SEQ ID NO:218; SEQ ID NO: 171 and SEQ ID NO:218; SEQ ID NO:172 and SEQ ID NO:218; SEQ ID NO: 173 and SEQ ID NO:218; SEQ ID NO: 174 and SEQ ID NO:218; SEQ ID NO: 175 and SEQ ID NO:218; SEQ ID NO: 176 and SEQ ID NO:218; SEQ ID NO: 177 and SEQ ID NO:218; SEQ ID NO: 178 and SEQ ID NO:218; SEQ ID NO: 179 and SEQ ID NO: 218; SEQ ID NO: 180 and SEQ ID NO:218; SEQ ID NO: 181 and SEQ ID NO:218; SEQ ID NO: 182 and SEQ ID NO:218; SEQ ID NO: 183 and SEQ ID NO:218; SEQ ID NO: 184 and SEQ ID NO: 218; SEQ ID NO: 185 and SEQ ID NO: 218; SEQ ID NO: 186 and SEQ ID NO:218; SEQ ID NO: 187 and SEQ ID NO:218; SEQ ID NO: 188 and SEQ ID NO:218; SEQ ID NO: 189 and SEQ ID NO:218; SEQ ID NO: 190 and SEQ ID NO:218; SEQ ID NO: 191 and SEQ ID NO: 218; SEQ ID NO: 192 and SEQ ID NO:218; SEQ ID NO: 193 and SEQ ID NO:218; SEQ ID NO: 194 and SEQ ID NO:218; SEQ ID NO: 195 and SEQ ID NO:218; SEQ ID NO: 196 and SEQ ID NO: 218; SEQ ID NO: 197 and SEQ ID NO: 218; SEQ ID NO: 198 and SEQ ID NO:218; SEQ ID NO: 199 and SEQ ID NO: 218; SEQ ID NO: 200 and SEQ ID NO:218; SEQ ID NO:201 and SEQ ID NO:218; SEQ ID NO:202 and SEQ ID NO:218; SEQ ID NO:203 and SEQ ID NO: 218; SEQ ID NO:204 and SEQ ID NO:218; SEQ ID NO:205 and SEQ ID NO:218; SEQ ID NO: 206 and SEQ ID NO:218; SEQ ID NO:207 and SEQ ID NO:218; SEQ ID NO:208 and SEQ ID NO:218; SEQ ID NO:209 and SEQ ID NO:218; SEQ ID NO:210 and SEQ ID NO:218; SEQ ID NO:211 and SEQ ID NO:218; SEQ ID NO:212 and SEQ ID NO:218; SEQ ID NO:213 and SEQ ID NO:218; SEQ ID NO:214 and SEQ ID NO:218; SEQ ID NO:215 and SEQ ID NO: 218; and SEQ ID NO:216 and SEQ ID NO:218.

In some aspects, the V_{H}-V_{L} pairs are selected from SEQ ID NO: 167 and SEQ ID NO: 219; SEQ ID NO: 168 and SEQ ID NO: 219; SEQ ID NO: 169 and SEQ ID NO:219; SEQ ID NO: 170 and SEQ ID NO:219; SEQ ID NO: 171 and SEQ ID NO: 219; SEQ ID NO: 172 and SEQ ID NO:219; SEQ ID NO: 173 and SEQ ID NO: 219; SEQ ID NO: 174 and SEQ ID NO:219; SEQ ID NO: 175 and SEQ ID NO:219; SEQ ID NO: 176 and SEQ ID NO:219; SEQ ID NO: 177 and SEQ ID NO: 219; SEQ ID NO: 178 and SEQ ID NO:219; SEQ ID NO: 179 and SEQ ID NO: 219; SEQ ID NO: 180 and SEQ ID NO:219; SEQ ID NO: 181 and SEQ ID NO:219; SEQ ID NO: 182 and SEQ ID NO:219; SEQ ID NO: 183 and SEQ ID NO:219; SEQ ID NO: 184 and SEQ ID NO: 219; SEQ ID NO: 185 and SEQ ID NO: 219; SEQ ID NO: 186 and SEQ ID NO:219; SEQ ID NO: 187 and SEQ ID NO: 219; SEQ ID NO: 188 and SEQ ID NO:219; SEQ ID NO: 189 and SEQ ID NO:219; SEQ ID NO: 190 and SEQ ID NO:219; SEQ ID NO: 191 and SEQ ID NO: 219; SEQ ID NO: 192 and SEQ ID NO: 219; SEQ ID NO: 193 and SEQ ID NO:219; SEQ ID NO: 194 and SEQ ID NO:219; SEQ ID NO: 195 and SEQ ID NO:219; SEQ ID NO: 196 and SEQ ID NO: 219; SEQ ID NO: 197 and SEQ ID NO:219; SEQ ID NO: 198 and SEQ ID NO:219; SEQ ID NO: 199 and SEQ ID NO: 219; SEQ ID NO: 200 and SEQ ID NO:219; SEQ ID NO:201 and SEQ ID NO:219; SEQ ID NO:202 and SEQ ID NO:219; SEQ ID NO:203 and SEQ ID NO: 219; SEQ ID NO:204 and SEQ ID NO: 219; SEQ ID NO:205 and SEQ ID NO:219; SEQ ID NO: 206 and SEQ ID NO: 219; SEQ ID NO: 207 and SEQ ID NO:219; SEQ ID NO:208 and SEQ ID NO: 219; SEQ ID NO:209 and SEQ ID NO:219; SEQ ID NO: 210 and SEQ ID NO:219; SEQ ID NO:211 and SEQ ID NO:219; SEQ ID NO:212 and SEQ ID NO: 219; SEQ ID NO:213 and SEQ ID NO:219; SEQ ID NO:214 and SEQ ID NO:219; SEQ ID NO:215 and SEQ ID NO: 219; and SEQ ID NO:216 and SEQ ID NO:219.

In some aspects, the V_{H}-V_{L} pairs are selected from SEQ ID NO: 167 and SEQ ID NO: 220; SEQ ID NO: 168 and SEQ ID NO:220; SEQ ID NO: 169 and SEQ ID NO:220; SEQ ID NO: 170 and SEQ ID NO:220; SEQ ID NO: 171 and SEQ ID NO:220; SEQ ID NO: 172 and SEQ ID NO: 220; SEQ ID NO: 173 and SEQ ID NO:220; SEQ ID NO: 174 and SEQ ID NO:220; SEQ ID NO: 175 and SEQ ID NO:220; SEQ ID NO: 176 and SEQ ID NO: 220; SEQ ID NO: 177 and SEQ ID NO:220; SEQ ID NO: 178 and SEQ ID NO: 220; SEQ ID NO: 179 and SEQ ID NO: 220; SEQ ID NO: 180 and SEQ ID NO:220; SEQ ID NO: 181 and SEQ ID NO:220; SEQ ID NO: 182 and SEQ ID NO:220; SEQ ID NO: 183 and SEQ ID NO:220; SEQ ID NO: 184 and SEQ ID NO: 220; SEQ ID NO: 185 and SEQ ID NO:220; SEQ ID NO: 186 and SEQ ID NO:220; SEQ ID NO: 187 and SEQ ID NO:220; SEQ ID NO: 188 and SEQ ID NO:220; SEQ ID NO: 189 and SEQ ID NO: 220; SEQ ID NO: 190 and SEQ ID NO:220; SEQ ID NO: 191 and SEQ ID NO: 220; SEQ ID NO: 192 and SEQ ID NO:220; SEQ ID NO: 193 and SEQ ID NO:220; SEQ ID NO: 194 and SEQ ID NO:220; SEQ ID NO: 195 and SEQ ID NO:220; SEQ ID NO: 196 and SEQ ID NO: 220; SEQ ID NO: 197 and SEQ ID NO: 220; SEQ ID NO: 198 and SEQ ID NO:220; SEQ ID NO: 199 and SEQ ID NO:220; SEQ ID NO: 200 and SEQ ID NO:220; SEQ ID NO:201 and SEQ ID NO:220; SEQ ID NO:202 and SEQ ID NO:220; SEQ ID NO:203 and SEQ ID NO: 220; SEQ ID NO:204 and SEQ ID NO:220; SEQ ID NO:205 and SEQ ID NO:220; SEQ ID NO: 206 and SEQ ID NO: 220; SEQ ID NO: 207 and SEQ ID NO: 220; SEQ ID NO:208 and SEQ ID NO:220; SEQ ID NO: 209 and SEQ ID NO:220; SEQ ID NO:210 and SEQ ID NO:220; SEQ ID NO:211 and SEQ ID NO:220; SEQ ID NO:212 and SEQ ID NO:220; SEQ ID NO:213 and SEQ ID NO:220; SEQ ID NO:214 and SEQ ID NO:220; SEQ ID NO:215 and SEQ ID NO: 220; and SEQ ID NO:216 and SEQ ID NO:220.

In some aspects, the V_{H}-V_{L} pairs are selected from SEQ ID NO: 167 and SEQ ID NO: 221; SEQ ID NO: 168 and SEQ ID NO:221; SEQ ID NO: 169 and SEQ ID NO:221; SEQ ID NO: 170 and SEQ ID NO:221; SEQ ID NO: 171 and SEQ ID NO:221; SEQ ID NO: 172 and SEQ ID NO:221; SEQ ID NO: 173 and SEQ ID NO:221; SEQ ID NO: 174 and SEQ ID NO:221; SEQ ID NO: 175 and SEQ ID NO:221; SEQ ID NO: 176 and SEQ ID NO: 221; SEQ ID NO: 177 and SEQ ID NO:221; SEQ ID NO:178 and SEQ ID NO:221; SEQ ID NO: 179 and SEQ ID NO: 221; SEQ ID NO: 180 and SEQ ID NO:221; SEQ ID NO: 181 and SEQ ID NO:221; SEQ ID NO: 182 and SEQ ID NO:221; SEQ ID NO: 183 and SEQ ID NO:221; SEQ ID NO: 184 and SEQ ID NO: 221; SEQ ID NO: 185 and SEQ ID NO:221; SEQ ID NO: 186 and SEQ ID NO:221; SEQ ID NO: 187 and SEQ ID NO:221; SEQ ID NO: 188 and SEQ ID NO:221; SEQ ID NO:189 and SEQ ID NO:221; SEQ ID NO: 190 and SEQ ID NO:221; SEQ ID NO: 191 and SEQ ID NO: 221; SEQ ID NO: 192 and SEQ ID NO:221; SEQ ID NO: 193 and SEQ ID NO:221; SEQ ID NO: 194 and SEQ ID NO:221; SEQ ID NO: 195 and SEQ ID NO:221; SEQ ID NO: 196 and SEQ ID NO: 221; SEQ ID NO: 197 and SEQ ID NO: 221; SEQ ID NO: 198 and SEQ ID NO:221; SEQ ID NO: 199 and SEQ ID NO: 221; SEQ ID NO:200 and SEQ ID NO:221; SEQ ID NO:201 and SEQ ID NO:221; SEQ ID NO:202 and SEQ ID NO:221; SEQ ID NO:203 and SEQ ID NO: 221; SEQ ID NO:204 and SEQ ID NO:221; SEQ ID NO:205 and SEQ ID NO:221; SEQ ID NO: 206 and SEQ ID NO:221; SEQ ID NO:207 and SEQ ID NO:221; SEQ ID NO:208 and SEQ ID NO:221; SEQ ID NO: 209 and SEQ ID NO: 221; SEQ ID NO:210 and SEQ ID NO:221; SEQ ID NO:211 and SEQ ID NO:221; SEQ ID NO:212 and SEQ ID NO:221; SEQ ID NO:213 and SEQ ID NO:221; SEQ ID NO:214 and SEQ ID NO:221; SEQ ID NO:215 and SEQ ID NO: 221; and SEQ ID NO: 216 and SEQ ID NO:221.

In some aspects, the V_{H}-V_{L} pairs are selected from SEQ ID NO: 167 and SEQ ID NO: 222; SEQ ID NO: 168 and SEQ ID NO:222; SEQ ID NO: 169 and SEQ ID NO:222; SEQ ID NO: 170 and SEQ ID NO:222; SEQ ID NO:171 and SEQ ID NO:222; SEQ ID NO: 172 and SEQ ID NO:222; SEQ ID NO: 173 and SEQ ID NO:222; SEQ ID NO: 174 and SEQ ID NO:222; SEQ ID NO: 175 and SEQ ID NO:222; SEQ ID NO: 176 and SEQ ID NO:222; SEQ ID NO: 177 and SEQ ID NO:222; SEQ ID NO: 178 and SEQ ID NO:222; SEQ ID NO: 179 and SEQ ID NO: 222; SEQ ID NO: 180 and SEQ ID NO: 222; SEQ ID NO: 181 and SEQ ID NO: 222; SEQ ID NO: 182 and SEQ ID NO:222; SEQ ID NO: 183 and SEQ ID NO:222; SEQ ID NO: 184 and SEQ ID NO:222; SEQ ID NO: 185 and SEQ ID NO:222; SEQ ID NO: 186 and SEQ ID NO:222; SEQ ID NO: 187 and SEQ ID NO:222; SEQ ID NO: 188 and SEQ ID NO:222; SEQ ID NO:189 and SEQ ID NO:222; SEQ ID NO: 190 and SEQ ID NO:222; SEQ ID NO: 191 and SEQ ID NO: 222; SEQ ID NO: 192 and SEQ ID NO:222; SEQ ID NO: 193 and SEQ ID NO: 222; SEQ ID NO: 194 and SEQ ID NO:222; SEQ ID NO: 195 and SEQ ID NO:222; SEQ ID NO: 196 and SEQ ID NO:222; SEQ ID NO: 197 and SEQ ID NO:222; SEQ ID NO: 198 and SEQ ID NO:222; SEQ ID NO: 199 and SEQ ID NO:222; SEQ ID NO: 200 and SEQ ID NO:222; SEQ ID NO:201 and SEQ ID NO:222; SEQ ID NO:202 and SEQ ID NO:222; SEQ ID NO:203 and SEQ ID NO: 222; SEQ ID NO:204 and SEQ ID NO:222; SEQ ID NO:205 and SEQ ID NO:222; SEQ ID NO: 206 and SEQ ID NO:222; SEQ ID NO:207 and SEQ ID NO:222; SEQ ID NO:208 and SEQ ID NO:222; SEQ ID NO: 209 and SEQ ID NO:222; SEQ ID NO:210 and SEQ ID NO:222; SEQ ID NO:211 and SEQ ID NO:222; SEQ ID NO:212 and SEQ ID NO:222; SEQ ID NO:213 and SEQ ID NO:222; SEQ ID NO:214 and SEQ ID NO:222; SEQ ID NO:215 and SEQ ID NO: 222; and SEQ ID NO:216 and SEQ ID NO:222.

In some aspects, the V_{H}-V_{L} pairs are selected from SEQ ID NO: 167 and SEQ ID NO: 223; SEQ ID NO: 168 and SEQ ID NO:223; SEQ ID NO: 169 and SEQ ID NO: 223; SEQ ID NO: 170 and SEQ ID NO:223; SEQ ID NO: 171 and SEQ ID NO:223; SEQ ID NO: 172 and SEQ ID NO: 223; SEQ ID NO: 173 and SEQ ID NO:223; SEQ ID NO: 174 and SEQ ID NO:223; SEQ ID NO: 175 and SEQ ID NO:223; SEQ ID NO: 176 and SEQ ID NO:223; SEQ ID NO: 177 and SEQ ID NO:223; SEQ ID NO: 178 and SEQ ID NO:223; SEQ ID NO: 179 and SEQ ID NO: 223; SEQ ID NO: 180 and SEQ ID NO:223; SEQ ID NO: 181 and SEQ ID NO:223; SEQ ID NO: 182 and SEQ ID NO:223; SEQ ID NO: 183 and SEQ ID NO:223; SEQ ID NO: 184 and SEQ ID NO:223; SEQ ID NO: 185 and SEQ ID NO:223; SEQ ID NO: 186 and SEQ ID NO:223; SEQ ID NO: 187 and SEQ ID NO:223; SEQ ID NO: 188 and SEQ ID NO:223; SEQ ID NO: 189 and SEQ ID NO:223; SEQ ID NO: 190 and SEQ ID NO:223; SEQ ID NO: 191 and SEQ ID NO: 223; SEQ ID NO: 192 and SEQ ID NO:223; SEQ ID NO: 193 and SEQ ID NO:223; SEQ ID NO: 194 and SEQ ID NO:223; SEQ ID NO: 195 and SEQ ID NO:223; SEQ ID NO: 196 and SEQ ID NO:223; SEQ ID NO: 197 and SEQ ID NO:223; SEQ ID NO: 198 and SEQ ID NO:223; SEQ ID NO: 199 and SEQ ID NO:223; SEQ ID NO: 200 and SEQ ID NO:223; SEQ ID NO:201 and SEQ ID NO:223; SEQ ID NO: 202 and SEQ ID NO:223; SEQ ID NO:203 and SEQ ID NO: 223; SEQ ID NO:204 and SEQ ID NO:223; SEQ ID NO:205 and SEQ ID NO:223; SEQ ID NO: 206 and SEQ ID NO:223; SEQ ID NO: 207 and SEQ ID NO:223; SEQ ID NO:208 and SEQ ID NO:223; SEQ ID NO:209 and SEQ ID NO:223; SEQ ID NO:210 and SEQ ID NO:223; SEQ ID NO:211 and SEQ ID NO:223; SEQ ID NO:212 and SEQ ID NO:223; SEQ ID NO:213 and SEQ ID NO:223; SEQ ID NO:214 and SEQ ID NO:223; SEQ ID NO:215 and SEQ ID NO: 223; and SEQ ID NO:216 and SEQ ID NO:223.

In some aspects, the V_{H}-V_{L} pairs are selected from SEQ ID NO:167 and SEQ ID NO: 224; SEQ ID NO: 168 and SEQ ID NO:224; SEQ ID NO: 169 and SEQ ID NO:224; SEQ ID NO: 170 and SEQ ID NO:224; SEQ ID NO: 171 and SEQ ID NO:224; SEQ ID NO: 172 and SEQ ID NO:224; SEQ ID NO: 173 and SEQ ID NO: 224; SEQ ID NO: 174 and SEQ ID NO:224; SEQ ID NO: 175 and SEQ ID NO:224; SEQ ID NO: 176 and SEQ ID NO: 224; SEQ ID NO: 177 and SEQ ID NO:224; SEQ ID NO:178 and SEQ ID NO:224; SEQ ID NO: 179 and SEQ ID NO: 224; SEQ ID NO: 180 and SEQ ID NO:224; SEQ ID NO: 181 and SEQ ID NO:224; SEQ ID NO: 182 and SEQ ID NO:224; SEQ ID NO: 183 and SEQ ID NO:224; SEQ ID NO: 184 and SEQ ID NO:224; SEQ ID NO: 185 and SEQ ID NO:224; SEQ ID NO: 186 and SEQ ID NO:224; SEQ ID NO: 187 and SEQ ID NO: 224; SEQ ID NO: 188 and SEQ ID NO:224; SEQ ID NO:189 and SEQ ID NO: 224; SEQ ID NO: 190 and SEQ ID NO:224; SEQ ID NO: 191 and SEQ ID NO: 224; SEQ ID NO: 192 and SEQ ID NO:224; SEQ ID NO: 193 and SEQ ID NO: 224; SEQ ID NO: 194 and SEQ ID NO:224; SEQ ID NO: 195 and SEQ ID NO:224; SEQ ID NO: 196 and SEQ ID NO: 224; SEQ ID NO: 197 and SEQ ID NO:224; SEQ ID NO: 198 and SEQ ID NO:224; SEQ ID NO: 199 and SEQ ID NO:224; SEQ ID NO:200 and SEQ ID NO:224; SEQ ID NO:201 and SEQ ID NO:224; SEQ ID NO:202 and SEQ ID NO:224; SEQ ID NO:203 and SEQ ID NO: 224; SEQ ID NO:204 and SEQ ID NO:224; SEQ ID NO:205 and SEQ ID NO:224; SEQ ID NO: 206 and SEQ ID NO:224; SEQ ID NO:207 and SEQ ID NO:224; SEQ ID NO:208 and SEQ ID NO:224; SEQ ID NO:209 and SEQ ID NO:224; SEQ ID NO:210 and SEQ ID NO:224; SEQ ID NO:211 and SEQ ID NO:224; SEQ ID NO:212 and SEQ ID NO:224; SEQ ID NO:213 and SEQ ID NO:224; SEQ ID NO:214 and SEQ ID NO:224; SEQ ID NO:215 and SEQ ID NO: 224; and SEQ ID NO:216 and SEQ ID NO:224.

In some aspects, the V_{H}-V_{L} pairs are selected from SEQ ID NO: 167 and SEQ ID NO: 225; SEQ ID NO: 168 and SEQ ID NO:225; SEQ ID NO: 169 and SEQ ID NO:225; SEQ ID NO: 170 and SEQ ID NO:225; SEQ ID NO: 171 and SEQ ID NO:225; SEQ ID NO: 172 and SEQ ID NO:225; SEQ ID NO: 173 and SEQ ID NO:225; SEQ ID NO: 174 and SEQ ID NO:225; SEQ ID NO: 175 and SEQ ID NO: 225; SEQ ID NO: 176 and SEQ ID NO:225; SEQ ID NO: 177 and SEQ ID NO:225; SEQ ID NO: 178 and SEQ ID NO:225; SEQ ID NO: 179 and SEQ ID NO: 225; SEQ ID NO: 180 and SEQ ID NO: 225; SEQ ID NO: 181 and SEQ ID NO:225; SEQ ID NO: 182 and SEQ ID NO:225; SEQ ID NO:183 and SEQ ID NO:225; SEQ ID NO: 184 and SEQ ID NO:225; SEQ ID NO: 185 and SEQ ID NO:225; SEQ ID NO: 186 and SEQ ID NO:225; SEQ ID NO: 187 and SEQ ID NO:225; SEQ ID NO: 188 and SEQ ID NO:225; SEQ ID NO:189 and SEQ ID NO:225; SEQ ID NO: 190 and SEQ ID NO:225; SEQ ID NO: 191 and SEQ ID NO: 225; SEQ ID NO: 192 and SEQ ID NO:225; SEQ ID NO: 193 and SEQ ID NO:225; SEQ ID NO: 194 and SEQ ID NO:225; SEQ ID NO: 195 and SEQ ID NO:225; SEQ ID NO: 196 and SEQ ID NO: 225; SEQ ID NO: 197 and SEQ ID NO:225; SEQ ID NO: 198 and SEQ ID NO:225; SEQ ID NO: 199 and SEQ ID NO:225; SEQ ID NO: 200 and SEQ ID NO:225; SEQ ID NO:201 and SEQ ID NO:225; SEQ ID NO:202 and SEQ ID NO:225; SEQ ID NO:203 and SEQ ID NO: 225; SEQ ID NO:204 and SEQ ID NO:225; SEQ ID NO:205 and SEQ ID NO:225; SEQ ID NO: 206 and SEQ ID NO:225; SEQ ID NO:207 and SEQ ID NO:225; SEQ ID NO:208 and SEQ ID NO:225; SEQ ID NO:209 and SEQ ID NO:225; SEQ ID NO:210 and SEQ ID NO:225; SEQ ID NO:211 and SEQ ID NO:225; SEQ ID NO: 212 and SEQ ID NO: 225; SEQ ID NO:213 and SEQ ID NO:225; SEQ ID NO:214 and SEQ ID NO:225; SEQ ID NO:215 and SEQ ID NO: 225; and SEQ ID NO:216 and SEQ ID NO:225.

In some aspects, the V_{H}-V_{L} pairs are selected from SEQ ID NO: 167 and SEQ ID NO: 226; SEQ ID NO: 168 and SEQ ID NO:226; SEQ ID NO: 169 and SEQ ID NO:226; SEQ ID NO: 170 and SEQ ID NO:226; SEQ ID NO: 171 and SEQ ID NO:226; SEQ ID NO: 172 and SEQ ID NO: 226; SEQ ID NO: 173 and SEQ ID NO:226; SEQ ID NO: 174 and SEQ ID NO:226; SEQ ID NO: 175 and SEQ ID NO: 226; SEQ ID NO: 176 and SEQ ID NO:226; SEQ ID NO: 177 and SEQ ID NO:226; SEQ ID NO: 178 and SEQ ID NO:226; SEQ ID NO: 179 and SEQ ID NO: 226; SEQ ID NO: 180 and SEQ ID NO:226; SEQ ID NO: 181 and SEQ ID NO:226; SEQ ID NO: 182 and SEQ ID NO:226; SEQ ID NO: 183 and SEQ ID NO:226; SEQ ID NO: 184 and SEQ ID NO:226; SEQ ID NO: 185 and SEQ ID NO:226; SEQ ID NO: 186 and SEQ ID NO:226; SEQ ID NO: 187 and SEQ ID NO:226; SEQ ID NO: 188 and SEQ ID NO:226; SEQ ID NO: 189 and SEQ ID NO:226; SEQ ID NO: 190 and SEQ ID NO:226; SEQ ID NO: 191 and SEQ ID NO: 226; SEQ ID NO: 192 and SEQ ID NO:226; SEQ ID NO: 193 and SEQ ID NO:226; SEQ ID NO: 194 and SEQ ID NO:226; SEQ ID NO: 195 and SEQ ID NO:226; SEQ ID NO: 196 and SEQ ID NO:226; SEQ ID NO: 197 and SEQ ID NO:226; SEQ ID NO: 198 and SEQ ID NO:226; SEQ ID NO: 199 and SEQ ID NO:226; SEQ ID NO:200 and SEQ ID NO:226; SEQ ID NO:201 and SEQ ID NO:226; SEQ ID NO:202 and SEQ ID NO:226; SEQ ID NO:203 and SEQ ID NO: 226; SEQ ID NO:204 and SEQ ID NO:226; SEQ ID NO:205 and SEQ ID NO:226; SEQ ID NO: 206 and SEQ ID NO:226; SEQ ID NO:207 and SEQ ID NO:226; SEQ ID NO:208 and SEQ ID NO: 226; SEQ ID NO: 209 and SEQ ID NO:226; SEQ ID NO:210 and SEQ ID NO:226; SEQ ID NO:211 and SEQ ID NO:226; SEQ ID NO:212 and SEQ ID NO:226; SEQ ID NO:213 and SEQ ID NO:226; SEQ ID NO:214 and SEQ ID NO:226; SEQ ID NO:215 and SEQ ID NO: 226; and SEQ ID NO:216 and SEQ ID NO:226.

In some aspects, the V_{H}-V_{L} pairs are selected from SEQ ID NO: 167 and SEQ ID NO: 227; SEQ ID NO:168 and SEQ ID NO:227; SEQ ID NO:169 and SEQ ID NO:227; SEQ ID NO: 170 and SEQ ID NO:227; SEQ ID NO: 171 and SEQ ID NO:227; SEQ ID NO: 172 and SEQ ID NO:227; SEQ ID NO: 173 and SEQ ID NO:227; SEQ ID NO: 174 and SEQ ID NO:227; SEQ ID NO: 175 and SEQ ID NO:227; SEQ ID NO: 176 and SEQ ID NO:227; SEQ ID NO: 177 and SEQ ID NO:227; SEQ ID NO: 178 and SEQ ID NO:227; SEQ ID NO: 179 and SEQ ID NO: 227; SEQ ID NO: 180 and SEQ ID NO:227; SEQ ID NO: 181 and SEQ ID NO:227; SEQ ID NO: 182 and SEQ ID NO:227; SEQ ID NO: 183 and SEQ ID NO:227; SEQ ID NO:184 and SEQ ID NO: 227; SEQ ID NO: 185 and SEQ ID NO:227; SEQ ID NO: 186 and SEQ ID NO:227; SEQ ID NO: 187 and SEQ ID NO:227; SEQ ID NO: 188 and SEQ ID NO:227; SEQ ID NO: 189 and SEQ ID NO:227; SEQ ID NO: 190 and SEQ ID NO:227; SEQ ID NO: 191 and SEQ ID NO: 227; SEQ ID NO: 192 and SEQ ID NO:227; SEQ ID NO: 193 and SEQ ID NO: 227; SEQ ID NO: 194 and SEQ ID NO:227; SEQ ID NO: 195 and SEQ ID NO:227; SEQ ID NO: 196 and SEQ ID NO:227; SEQ ID NO: 197 and SEQ ID NO:227; SEQ ID NO: 198 and SEQ ID NO:227; SEQ ID NO: 199 and SEQ ID NO: 227; SEQ ID NO: 200 and SEQ ID NO:227; SEQ ID NO:201 and SEQ ID NO:227; SEQ ID NO:202 and SEQ ID NO:227; SEQ ID NO:203 and SEQ ID NO: 227; SEQ ID NO:204 and SEQ ID NO:227; SEQ ID NO:205 and SEQ ID NO:227; SEQ ID NO: 206 and SEQ ID NO:227; SEQ ID NO: 207 and SEQ ID NO:227; SEQ ID NO:208 and SEQ ID NO:227; SEQ ID NO:209 and SEQ ID NO:227; SEQ ID NO:210 and SEQ ID NO:227; SEQ ID NO:211 and SEQ ID NO:227; SEQ ID NO:212 and SEQ ID NO:227; SEQ ID NO:213 and SEQ ID NO:227; SEQ ID NO:214 and SEQ ID NO:227; SEQ ID NO:215 and SEQ ID NO: 227; and SEQ ID NO: 216 and SEQ ID NO:227.

In some aspects, the V_{H}-V_{L} pairs are selected from SEQ ID NO: 167 and SEQ ID NO: 228; SEQ ID NO: 168 and SEQ ID NO:228; SEQ ID NO: 169 and SEQ ID NO:228; SEQ ID NO: 170 and SEQ ID NO:228; SEQ ID NO:171 and SEQ ID NO:228; SEQ ID NO: 172 and SEQ ID NO:228; SEQ ID NO: 173 and SEQ ID NO:228; SEQ ID NO: 174 and SEQ ID NO:228; SEQ ID NO: 175 and SEQ ID NO:228; SEQ ID NO: 176 and SEQ ID NO:228; SEQ ID NO: 177 and SEQ ID NO:228; SEQ ID NO: 178 and SEQ ID NO:228; SEQ ID NO: 179 and SEQ ID NO: 228; SEQ ID NO: 180 and SEQ ID NO: 228; SEQ ID NO: 181 and SEQ ID NO:228; SEQ ID NO: 182 and SEQ ID NO:228; SEQ ID NO: 183 and SEQ ID NO:228; SEQ ID NO: 184 and SEQ ID NO:228; SEQ ID NO: 185 and SEQ ID NO:228; SEQ ID NO: 186 and SEQ ID NO:228; SEQ ID NO: 187 and SEQ ID NO:228; SEQ ID NO: 188 and SEQ ID NO:228; SEQ ID NO: 189 and SEQ ID NO:228; SEQ ID NO: 190 and SEQ ID NO:228; SEQ ID NO: 191 and SEQ ID NO: 228; SEQ ID NO: 192 and SEQ ID NO:228; SEQ ID NO: 193 and SEQ ID NO:228; SEQ ID NO: 194 and SEQ ID NO:228; SEQ ID NO: 195 and SEQ ID NO:228; SEQ ID NO: 196 and SEQ ID NO:228; SEQ ID NO: 197 and SEQ ID NO:228; SEQ ID NO: 198 and SEQ ID NO:228; SEQ ID NO: 199 and SEQ ID NO:228; SEQ ID NO: 200 and SEQ ID NO:228; SEQ ID NO:201 and SEQ ID NO:228; SEQ ID NO:202 and SEQ ID NO:228; SEQ ID NO:203 and SEQ ID NO: 228; SEQ ID NO:204 and SEQ ID NO:228; SEQ ID NO:205 and SEQ ID NO:228; SEQ ID NO: 206 and SEQ ID NO:228; SEQ ID NO: 207 and SEQ ID NO:228; SEQ ID NO:208 and SEQ ID NO:228; SEQ ID NO: 209 and SEQ ID NO: 228; SEQ ID NO: 210 and SEQ ID NO:228; SEQ ID NO:211 and SEQ ID NO: 228; SEQ ID NO:212 and SEQ ID NO: 228; SEQ ID NO:213 and SEQ ID NO:228; SEQ ID NO:214 and SEQ ID NO:228; SEQ ID NO:215 and SEQ ID NO: 228; and SEQ ID NO:216 and SEQ ID NO:228.

In some aspects, the V_{H}-V_{L} pairs are selected from SEQ ID NO: 167 and SEQ ID NO: 229; SEQ ID NO: 168 and SEQ ID NO: 229; SEQ ID NO: 169 and SEQ ID NO:229; SEQ ID NO: 170 and SEQ ID NO: 229; SEQ ID NO: 171 and SEQ ID NO:229; SEQ ID NO:172 and SEQ ID NO: 229; SEQ ID NO: 173 and SEQ ID NO:229; SEQ ID NO: 174 and SEQ ID NO:229; SEQ ID NO: 175 and SEQ ID NO: 229; SEQ ID NO: 176 and SEQ ID NO: 229; SEQ ID NO: 177 and SEQ ID NO:229; SEQ ID NO: 178 and SEQ ID NO:229; SEQ ID NO: 179 and SEQ ID NO: 229; SEQ ID NO: 180 and SEQ ID NO: 229; SEQ ID NO: 181 and SEQ ID NO:229; SEQ ID NO: 182 and SEQ ID NO:229; SEQ ID NO: 183 and SEQ ID NO:229; SEQ ID NO: 184 and SEQ ID NO: 229; SEQ ID NO: 185 and SEQ ID NO:229; SEQ ID NO: 186 and SEQ ID NO:229; SEQ ID NO: 187 and SEQ ID NO:229; SEQ ID NO: 188 and SEQ ID NO: 229; SEQ ID NO: 189 and SEQ ID NO: 229; SEQ ID NO: 190 and SEQ ID NO:229; SEQ ID NO: 191 and SEQ ID NO: 229; SEQ ID NO: 192 and SEQ ID NO: 229; SEQ ID NO: 193 and SEQ ID NO: 229; SEQ ID NO: 194 and SEQ ID NO:229; SEQ ID NO: 195 and SEQ ID NO:229; SEQ ID NO: 196 and SEQ ID NO: 229; SEQ ID NO: 197 and SEQ ID NO:229; SEQ ID NO: 198 and SEQ ID NO:229; SEQ ID NO: 199 and SEQ ID NO:229; SEQ ID NO: 200 and SEQ ID NO:229; SEQ ID NO:201 and SEQ ID NO: 229; SEQ ID NO:202 and SEQ ID NO:229; SEQ ID NO:203 and SEQ ID NO: 229; SEQ ID NO:204 and SEQ ID NO:229; SEQ ID NO:205 and SEQ ID NO:229; SEQ ID NO: 206 and SEQ ID NO:229; SEQ ID NO:207 and SEQ ID NO:229; SEQ ID NO:208 and SEQ ID NO: 229; SEQ ID NO: 209 and SEQ ID NO: 229; SEQ ID NO:210 and SEQ ID NO:229; SEQ ID NO: 211 and SEQ ID NO: 229; SEQ ID NO:212 and SEQ ID NO:229; SEQ ID NO:213 and SEQ ID NO:229; SEQ ID NO:214 and SEQ ID NO:229; SEQ ID NO:215 and SEQ ID NO: 229; and SEQ ID NO: 216 and SEQ ID NO:229.

In some aspects, the V_{H}-V_{L} pairs are selected from SEQ ID NO: 167 and SEQ ID NO: 230; SEQ ID NO: 168 and SEQ ID NO:230; SEQ ID NO: 169 and SEQ ID NO:230; SEQ ID NO: 170 and SEQ ID NO:230; SEQ ID NO: 171 and SEQ ID NO:230; SEQ ID NO: 172 and SEQ ID NO: 230; SEQ ID NO: 173 and SEQ ID NO:230; SEQ ID NO: 174 and SEQ ID NO:230; SEQ ID NO: 175 and SEQ ID NO:230; SEQ ID NO: 176 and SEQ ID NO:230; SEQ ID NO: 177 and SEQ ID NO:230; SEQ ID NO: 178 and SEQ ID NO:230; SEQ ID NO: 179 and SEQ ID NO: 230; SEQ ID NO: 180 and SEQ ID NO: 230; SEQ ID NO: 181 and SEQ ID NO:230; SEQ ID NO: 182 and SEQ ID NO:230; SEQ ID NO: 183 and SEQ ID NO:230; SEQ ID NO: 184 and SEQ ID NO: 230; SEQ ID NO: 185 and SEQ ID NO:230; SEQ ID NO: 186 and SEQ ID NO:230; SEQ ID NO: 187 and SEQ ID NO: 230; SEQ ID NO: 188 and SEQ ID NO:230; SEQ ID NO: 189 and SEQ ID NO: 230; SEQ ID NO: 190 and SEQ ID NO:230; SEQ ID NO: 191 and SEQ ID NO: 230; SEQ ID NO: 192 and SEQ ID NO:230; SEQ ID NO: 193 and SEQ ID NO: 230; SEQ ID NO: 194 and SEQ ID NO:230; SEQ ID NO: 195 and SEQ ID NO:230; SEQ ID NO: 196 and SEQ ID NO:230; SEQ ID NO: 197 and SEQ ID NO:230; SEQ ID NO: 198 and SEQ ID NO:230; SEQ ID NO: 199 and SEQ ID NO:230; SEQ ID NO: 200 and SEQ ID NO:230; SEQ ID NO:201 and SEQ ID NO:230; SEQ ID NO:202 and SEQ ID NO:230; SEQ ID NO:203 and SEQ ID NO: 230; SEQ ID NO:204 and SEQ ID NO:230; SEQ ID NO:205 and SEQ ID NO:230; SEQ ID NO: 206 and SEQ ID NO:230; SEQ ID NO:207 and SEQ ID NO:230; SEQ ID NO:208 and SEQ ID NO: 230; SEQ ID NO:209 and SEQ ID NO:230; SEQ ID NO:210 and SEQ ID NO:230; SEQ ID NO:211 and SEQ ID NO:230; SEQ ID NO:212 and SEQ ID NO:230; SEQ ID NO:213 and SEQ ID NO:230; SEQ ID NO:214 and SEQ ID NO:230; SEQ ID NO:215 and SEQ ID NO: 230; and SEQ ID NO:216 and SEQ ID NO:230.

In some aspects, the V_{H}-V_{L} pairs are selected from SEQ ID NO: 167 and SEQ ID NO: 231; SEQ ID NO: 168 and SEQ ID NO:231; SEQ ID NO: 169 and SEQ ID NO:231; SEQ ID NO: 170 and SEQ ID NO:231; SEQ ID NO:171 and SEQ ID NO:231; SEQ ID NO: 172 and SEQ ID NO:231; SEQ ID NO: 173 and SEQ ID NO:231; SEQ ID NO: 174 and SEQ ID NO:231; SEQ ID NO: 175 and SEQ ID NO:231; SEQ ID NO: 176 and SEQ ID NO:231; SEQ ID NO: 177 and SEQ ID NO:231; SEQ ID NO: 178 and SEQ ID NO:231; SEQ ID NO: 179 and SEQ ID NO: 231; SEQ ID NO: 180 and SEQ ID NO:231; SEQ ID NO: 181 and SEQ ID NO: 231; SEQ ID NO: 182 and SEQ ID NO:231; SEQ ID NO: 183 and SEQ ID NO:231; SEQ ID NO: 184 and SEQ ID NO:231; SEQ ID NO: 185 and SEQ ID NO:231; SEQ ID NO: 186 and SEQ ID NO:231; SEQ ID NO: 187 and SEQ ID NO:231; SEQ ID NO: 188 and SEQ ID NO:231; SEQ ID NO: 189 and SEQ ID NO:231; SEQ ID NO: 190 and SEQ ID NO:231; SEQ ID NO: 191 and SEQ ID NO: 231; SEQ ID NO: 192 and SEQ ID NO:231; SEQ ID NO: 193 and SEQ ID NO:231; SEQ ID NO: 194 and SEQ ID NO:231; SEQ ID NO: 195 and SEQ ID NO:231; SEQ ID NO: 196 and SEQ ID NO:231; SEQ ID NO: 197 and SEQ ID NO:231; SEQ ID NO: 198 and SEQ ID NO:231; SEQ ID NO: 199 and SEQ ID NO:231; SEQ ID NO: 200 and SEQ ID NO:231; SEQ ID NO:201 and SEQ ID NO:231; SEQ ID NO:202 and SEQ ID NO:231; SEQ ID NO:203 and SEQ ID NO: 231; SEQ ID NO:204 and SEQ ID NO:231; SEQ ID NO:205 and SEQ ID NO:231; SEQ ID NO: 206 and SEQ ID NO: 231; SEQ ID NO:207 and SEQ ID NO:231; SEQ ID NO:208 and SEQ ID NO: 231; SEQ ID NO:209 and SEQ ID NO:231; SEQ ID NO:210 and SEQ ID NO:231; SEQ ID NO:211 and SEQ ID NO:231; SEQ ID NO: 212 and SEQ ID NO:231; SEQ ID NO:213 and SEQ ID NO:231; SEQ ID NO:214 and SEQ ID NO:231; SEQ ID NO:215 and SEQ ID NO: 231; and SEQ ID NO:216 and SEQ ID NO:231.

In some aspects, the V_{H}-V_{L} pairs are selected from SEQ ID NO: 167 and SEQ ID NO: 232; SEQ ID NO: 168 and SEQ ID NO:232; SEQ ID NO: 169 and SEQ ID NO:232; SEQ ID NO: 170 and SEQ ID NO:232; SEQ ID NO: 171 and SEQ ID NO:232; SEQ ID NO: 172 and SEQ ID NO: 232; SEQ ID NO: 173 and SEQ ID NO:232; SEQ ID NO: 174 and SEQ ID NO:232; SEQ ID NO: 175 and SEQ ID NO:232; SEQ ID NO: 176 and SEQ ID NO: 232; SEQ ID NO: 177 and SEQ ID NO:232; SEQ ID NO: 178 and SEQ ID NO:232; SEQ ID NO: 179 and SEQ ID NO: 232; SEQ ID NO: 180 and SEQ ID NO:232; SEQ ID NO: 181 and SEQ ID NO:232; SEQ ID NO: 182 and SEQ ID NO:232; SEQ ID NO: 183 and SEQ ID NO:232; SEQ ID NO: 184 and SEQ ID NO:232; SEQ ID NO: 185 and SEQ ID NO:232; SEQ ID NO: 186 and SEQ ID NO:232; SEQ ID NO: 187 and SEQ ID NO:232; SEQ ID NO: 188 and SEQ ID NO:232; SEQ ID NO: 189 and SEQ ID NO:232; SEQ ID NO: 190 and SEQ ID NO:232; SEQ ID NO: 191 and SEQ ID NO: 232; SEQ ID NO: 192 and SEQ ID NO:232; SEQ ID NO: 193 and SEQ ID NO: 232; SEQ ID NO: 194 and SEQ ID NO:232; SEQ ID NO: 195 and SEQ ID NO:232; SEQ ID NO: 196 and SEQ ID NO: 232; SEQ ID NO: 197 and SEQ ID NO: 232; SEQ ID NO: 198 and SEQ ID NO:232; SEQ ID NO: 199 and SEQ ID NO:232; SEQ ID NO: 200 and SEQ ID NO:232; SEQ ID NO:201 and SEQ ID NO:232; SEQ ID NO:202 and SEQ ID NO:232; SEQ ID NO:203 and SEQ ID NO: 232; SEQ ID NO:204 and SEQ ID NO:232; SEQ ID NO:205 and SEQ ID NO:232; SEQ ID NO: 206 and SEQ ID NO:232; SEQ ID NO:207 and SEQ ID NO:232; SEQ ID NO:208 and SEQ ID NO:232; SEQ ID NO:209 and SEQ ID NO:232; SEQ ID NO:210 and SEQ ID NO:232; SEQ ID NO:211 and SEQ ID NO:232; SEQ ID NO:212 and SEQ ID NO:232; SEQ ID NO:213 and SEQ ID NO:232; SEQ ID NO:214 and SEQ ID NO:232; SEQ ID NO:215 and SEQ ID NO: 232; and SEQ ID NO:216 and SEQ ID NO:232.

In some aspects, the V_{H}-V_{L} pairs are selected from SEQ ID NO: 167 and SEQ ID NO: 233; SEQ ID NO: 168 and SEQ ID NO:233; SEQ ID NO: 169 and SEQ ID NO:233; SEQ ID NO: 170 and SEQ ID NO:233; SEQ ID NO: 171 and SEQ ID NO:233; SEQ ID NO: 172 and SEQ ID NO:233; SEQ ID NO: 173 and SEQ ID NO: 233; SEQ ID NO: 174 and SEQ ID NO:233; SEQ ID NO: 175 and SEQ ID NO: 233; SEQ ID NO: 176 and SEQ ID NO:233; SEQ ID NO: 177 and SEQ ID NO:233; SEQ ID NO: 178 and SEQ ID NO:233; SEQ ID NO: 179 and SEQ ID NO: 233; SEQ ID NO: 180 and SEQ ID NO:233; SEQ ID NO: 181 and SEQ ID NO:233; SEQ ID NO: 182 and SEQ ID NO:233; SEQ ID NO: 183 and SEQ ID NO:233; SEQ ID NO: 184 and SEQ ID NO:233; SEQ ID NO: 185 and SEQ ID NO:233; SEQ ID NO: 186 and SEQ ID NO:233; SEQ ID NO: 187 and SEQ ID NO:233; SEQ ID NO: 188 and SEQ ID NO:233; SEQ ID NO:189 and SEQ ID NO:233; SEQ ID NO: 190 and SEQ ID NO:233; SEQ ID NO: 191 and SEQ ID NO: 233; SEQ ID NO: 192 and SEQ ID NO:233; SEQ ID NO: 193 and SEQ ID NO:233; SEQ ID NO: 194 and SEQ ID NO:233; SEQ ID NO: 195 and SEQ ID NO:233; SEQ ID NO: 196 and SEQ ID NO: 233; SEQ ID NO: 197 and SEQ ID NO:233; SEQ ID NO: 198 and SEQ ID NO:233; SEQ ID NO: 199 and SEQ ID NO: 233; SEQ ID NO: 200 and SEQ ID NO:233; SEQ ID NO:201 and SEQ ID NO:233; SEQ ID NO: 202 and SEQ ID NO:233; SEQ ID NO:203 and SEQ ID NO: 233; SEQ ID NO: 204 and SEQ ID NO:233; SEQ ID NO:205 and SEQ ID NO:233; SEQ ID NO: 206 and SEQ ID NO:233; SEQ ID NO:207 and SEQ ID NO:233; SEQ ID NO:208 and SEQ ID NO:233; SEQ ID NO:209 and SEQ ID NO:233; SEQ ID NO:210 and SEQ ID NO:233; SEQ ID NO:211 and SEQ ID NO:233; SEQ ID NO:212 and SEQ ID NO:233; SEQ ID NO:213 and SEQ ID NO:233; SEQ ID NO:214 and SEQ ID NO:233; SEQ ID NO:215 and SEQ ID NO: 233; and SEQ ID NO:216 and SEQ ID NO:233.

In some aspects, the V_{H}-V_{L} pairs are selected from SEQ ID NO: 167 and SEQ ID NO: 234; SEQ ID NO: 168 and SEQ ID NO:234; SEQ ID NO: 169 and SEQ ID NO:234; SEQ ID NO: 170 and SEQ ID NO:234; SEQ ID NO: 171 and SEQ ID NO:234; SEQ ID NO: 172 and SEQ ID NO:234; SEQ ID NO: 173 and SEQ ID NO:234; SEQ ID NO: 174 and SEQ ID NO:234; SEQ ID NO: 175 and SEQ ID NO:234; SEQ ID NO: 176 and SEQ ID NO:234; SEQ ID NO:177 and SEQ ID NO:234; SEQ ID NO: 178 and SEQ ID NO:234; SEQ ID NO: 179 and SEQ ID NO: 234; SEQ ID NO: 180 and SEQ ID NO:234; SEQ ID NO: 181 and SEQ ID NO:234; SEQ ID NO: 182 and SEQ ID NO:234; SEQ ID NO: 183 and SEQ ID NO:234; SEQ ID NO: 184 and SEQ ID NO:234; SEQ ID NO: 185 and SEQ ID NO: 234; SEQ ID NO: 186 and SEQ ID NO:234; SEQ ID NO: 187 and SEQ ID NO: 234; SEQ ID NO: 188 and SEQ ID NO:234; SEQ ID NO: 189 and SEQ ID NO:234; SEQ ID NO: 190 and SEQ ID NO:234; SEQ ID NO: 191 and SEQ ID NO: 234; SEQ ID NO: 192 and SEQ ID NO: 234; SEQ ID NO: 193 and SEQ ID NO: 234; SEQ ID NO: 194 and SEQ ID NO:234; SEQ ID NO: 195 and SEQ ID NO:234; SEQ ID NO: 196 and SEQ ID NO: 234; SEQ ID NO: 197 and SEQ ID NO:234; SEQ ID NO: 198 and SEQ ID NO:234; SEQ ID NO: 199 and SEQ ID NO:234; SEQ ID NO: 200 and SEQ ID NO:234; SEQ ID NO:201 and SEQ ID NO:234; SEQ ID NO:202 and SEQ ID NO:234; SEQ ID NO:203 and SEQ ID NO: 234; SEQ ID NO:204 and SEQ ID NO:234; SEQ ID NO:205 and SEQ ID NO:234; SEQ ID NO: 206 and SEQ ID NO:234; SEQ ID NO:207 and SEQ ID NO:234; SEQ ID NO:208 and SEQ ID NO: 234; SEQ ID NO:209 and SEQ ID NO:234; SEQ ID NO:210 and SEQ ID NO:234; SEQ ID NO:211 and SEQ ID NO:234; SEQ ID NO:212 and SEQ ID NO:234; SEQ ID NO:213 and SEQ ID NO:234; SEQ ID NO:214 and SEQ ID NO:234; SEQ ID NO:215 and SEQ ID NO: 234; and SEQ ID NO:216 and SEQ ID NO:234.

In some aspects, the V_{H}-V_{L} pairs are selected from SEQ ID NO: 167 and SEQ ID NO: 235; SEQ ID NO: 168 and SEQ ID NO:235; SEQ ID NO: 169 and SEQ ID NO:235; SEQ ID NO: 170 and SEQ ID NO:235; SEQ ID NO: 171 and SEQ ID NO:235; SEQ ID NO: 172 and SEQ ID NO:235; SEQ ID NO: 173 and SEQ ID NO:235; SEQ ID NO: 174 and SEQ ID NO:235; SEQ ID NO: 175 and SEQ ID NO:235; SEQ ID NO: 176 and SEQ ID NO:235; SEQ ID NO: 177 and SEQ ID NO:235; SEQ ID NO: 178 and SEQ ID NO:235; SEQ ID NO:179 and SEQ ID NO: 235; SEQ ID NO: 180 and SEQ ID NO:235; SEQ ID NO: 181 and SEQ ID NO:235; SEQ ID NO: 182 and SEQ ID NO:235; SEQ ID NO: 183 and SEQ ID NO:235; SEQ ID NO: 184 and SEQ ID NO:235; SEQ ID NO: 185 and SEQ ID NO:235; SEQ ID NO: 186 and SEQ ID NO:235; SEQ ID NO: 187 and SEQ ID NO:235; SEQ ID NO: 188 and SEQ ID NO:235; SEQ ID NO: 189 and SEQ ID NO:235; SEQ ID NO: 190 and SEQ ID NO:235; SEQ ID NO: 191 and SEQ ID NO: 235; SEQ ID NO: 192 and SEQ ID NO: 235; SEQ ID NO: 193 and SEQ ID NO:235; SEQ ID NO: 194 and SEQ ID NO:235; SEQ ID NO: 195 and SEQ ID NO:235; SEQ ID NO: 196 and SEQ ID NO: 235; SEQ ID NO: 197 and SEQ ID NO: 235; SEQ ID NO: 198 and SEQ ID NO:235; SEQ ID NO: 199 and SEQ ID NO:235; SEQ ID NO:200 and SEQ ID NO:235; SEQ ID NO:201 and SEQ ID NO:235; SEQ ID NO:202 and SEQ ID NO:235; SEQ ID NO:203 and SEQ ID NO: 235; SEQ ID NO:204 and SEQ ID NO:235; SEQ ID NO:205 and SEQ ID NO:235; SEQ ID NO: 206 and SEQ ID NO:235; SEQ ID NO:207 and SEQ ID NO:235; SEQ ID NO:208 and SEQ ID NO:235; SEQ ID NO:209 and SEQ ID NO:235; SEQ ID NO:210 and SEQ ID NO:235; SEQ ID NO:211 and SEQ ID NO:235; SEQ ID NO:212 and SEQ ID NO:235; SEQ ID NO:213 and SEQ ID NO:235; SEQ ID NO:214 and SEQ ID NO:235; SEQ ID NO:215 and SEQ ID NO: 235; and SEQ ID NO:216 and SEQ ID NO:235.

In some aspects, the V_{H}-V_{L} pairs are selected from SEQ ID NO: 167 and SEQ ID NO: 236; SEQ ID NO: 168 and SEQ ID NO:236; SEQ ID NO: 169 and SEQ ID NO:236; SEQ ID NO: 170 and SEQ ID NO:236; SEQ ID NO: 171 and SEQ ID NO:236; SEQ ID NO: 172 and SEQ ID NO:236; SEQ ID NO: 173 and SEQ ID NO:236; SEQ ID NO: 174 and SEQ ID NO:236; SEQ ID NO: 175 and SEQ ID NO:236; SEQ ID NO: 176 and SEQ ID NO:236; SEQ ID NO: 177 and SEQ ID NO:236; SEQ ID NO:178 and SEQ ID NO:236; SEQ ID NO: 179 and SEQ ID NO: 236; SEQ ID NO: 180 and SEQ ID NO:236; SEQ ID NO: 181 and SEQ ID NO:236; SEQ ID NO: 182 and SEQ ID NO:236; SEQ ID NO: 183 and SEQ ID NO:236; SEQ ID NO: 184 and SEQ ID NO:236; SEQ ID NO: 185 and SEQ ID NO:236; SEQ ID NO: 186 and SEQ ID NO:236; SEQ ID NO: 187 and SEQ ID NO: 236; SEQ ID NO: 188 and SEQ ID NO: 236; SEQ ID NO: 189 and SEQ ID NO:236; SEQ ID NO: 190 and SEQ ID NO:236; SEQ ID NO: 191 and SEQ ID NO: 236; SEQ ID NO: 192 and SEQ ID NO:236; SEQ ID NO: 193 and SEQ ID NO:236; SEQ ID NO: 194 and SEQ ID NO:236; SEQ ID NO: 195 and SEQ ID NO:230; SEQ ID NO: 196 and SEQ ID NO: 230; SEQ ID NO: 197 and SEQ ID NO: 236; SEQ ID NO: 198 and SEQ ID NO:236; SEQ ID NO: 199 and SEQ ID NO:236; SEQ ID NO:200 and SEQ ID NO:236; SEQ ID NO:201 and SEQ ID NO:236; SEQ ID NO:202 and SEQ ID NO:236; SEQ ID NO:203 and SEQ ID NO: 236; SEQ ID NO:204 and SEQ ID NO:236; SEQ ID NO:205 and SEQ ID NO:236; SEQ ID NO: 206 and SEQ ID NO:236; SEQ ID NO:207 and SEQ ID NO:236; SEQ ID NO:208 and SEQ ID NO:236; SEQ ID NO:209 and SEQ ID NO:236; SEQ ID NO:210 and SEQ ID NO:236; SEQ ID NO:211 and SEQ ID NO:236; SEQ ID NO:212 and SEQ ID NO:236; SEQ ID NO:213 and SEQ ID NO:236; SEQ ID NO:214 and SEQ ID NO:236; SEQ ID NO:215 and SEQ ID NO: 236; and SEQ ID NO:216 and SEQ ID NO:236.

In some aspects, the V_{H}-V_{L} pairs are selected from SEQ ID NO: 167 and SEQ ID NO: 237; SEQ ID NO: 168 and SEQ ID NO:237; SEQ ID NO: 169 and SEQ ID NO:237; SEQ ID NO: 170 and SEQ ID NO:237; SEQ ID NO: 171 and SEQ ID NO:237; SEQ ID NO: 172 and SEQ ID NO:237; SEQ ID NO: 173 and SEQ ID NO:237; SEQ ID NO: 174 and SEQ ID NO:237; SEQ ID NO: 175 and SEQ ID NO:237; SEQ ID NO: 176 and SEQ ID NO:237; SEQ ID NO: 177 and SEQ ID NO:237; SEQ ID NO:178 and SEQ ID NO:237; SEQ ID NO: 179 and SEQ ID NO: 237; SEQ ID NO: 180 and SEQ ID NO: 237; SEQ ID NO: 181 and SEQ ID NO:237; SEQ ID NO: 182 and SEQ ID NO:237; SEQ ID NO: 183 and SEQ ID NO:237; SEQ ID NO: 184 and SEQ ID NO:237; SEQ ID NO: 185 and SEQ ID NO:237; SEQ ID NO: 186 and SEQ ID NO:237; SEQ ID NO: 187 and SEQ ID NO: 237; SEQ ID NO: 188 and SEQ ID NO: 237; SEQ ID NO: 189 and SEQ ID NO:237; SEQ ID NO: 190 and SEQ ID NO:237; SEQ ID NO: 191 and SEQ ID NO: 237; SEQ ID NO: 192 and SEQ ID NO:237; SEQ ID NO: 193 and SEQ ID NO:237; SEQ ID NO: 194 and SEQ ID NO:237; SEQ ID NO: 195 and SEQ ID NO:237; SEQ ID NO: 196 and SEQ ID NO: 237; SEQ ID NO: 197 and SEQ ID NO:237; SEQ ID NO: 198 and SEQ ID NO:237; SEQ ID NO: 199 and SEQ ID NO:237; SEQ ID NO: 200 and SEQ ID NO:237; SEQ ID NO:201 and SEQ ID NO:237; SEQ ID NO:202 and SEQ ID NO:237; SEQ ID NO:203 and SEQ ID NO: 237; SEQ ID NO:204 and SEQ ID NO:237; SEQ ID NO:205 and SEQ ID NO:237; SEQ ID NO: 206 and SEQ ID NO:237; SEQ ID NO:207 and SEQ ID NO:237; SEQ ID NO:208 and SEQ ID NO:237; SEQ ID NO:209 and SEQ ID NO:237; SEQ ID NO:210 and SEQ ID NO:237; SEQ ID NO:211 and SEQ ID NO:237; SEQ ID NO:212 and SEQ ID NO:237; SEQ ID NO:213 and SEQ ID NO:237; SEQ ID NO:214 and SEQ ID NO:237; SEQ ID NO:215 and SEQ ID NO: 237; and SEQ ID NO:216 and SEQ ID NO:237.

In some aspects, the V_{H}-V_{L} pairs are selected from SEQ ID NO: 167 and SEQ ID NO: 238; SEQ ID NO: 168 and SEQ ID NO:238; SEQ ID NO: 169 and SEQ ID NO:238; SEQ ID NO: 170 and SEQ ID NO:238; SEQ ID NO:171 and SEQ ID NO:238; SEQ ID NO:172 and SEQ ID NO: 238; SEQ ID NO: 173 and SEQ ID NO:238; SEQ ID NO: 174 and SEQ ID NO:238; SEQ ID NO:175 and SEQ ID NO:238; SEQ ID NO: 176 and SEQ ID NO:238; SEQ ID NO:177 and SEQ ID NO:238; SEQ ID NO: 178 and SEQ ID NO:238; SEQ ID NO: 179 and SEQ ID NO: 238; SEQ ID NO:180 and SEQ ID NO:238; SEQ ID NO: 181 and SEQ ID NO:238; SEQ ID NO: 182 and SEQ ID NO:238; SEQ ID NO:183 and SEQ ID NO:238; SEQ ID NO:184 and SEQ ID NO:238; SEQ ID NO: 185 and SEQ ID NO:238; SEQ ID NO:186 and SEQ ID NO:238; SEQ ID NO: 187 and SEQ ID NO:238; SEQ ID NO: 188 and SEQ ID NO:238; SEQ ID NO: 189 and SEQ ID NO:238; SEQ ID NO:190 and SEQ ID NO:238; SEQ ID NO: 191 and SEQ ID NO: 238; SEQ ID NO: 192 and SEQ ID NO:238; SEQ ID NO: 193 and SEQ ID NO:238; SEQ ID NO: 194 and SEQ ID NO:238; SEQ ID NO: 195 and SEQ ID NO:238; SEQ ID NO: 196 and SEQ ID NO:238; SEQ ID NO: 197 and SEQ ID NO:238; SEQ ID NO: 198 and SEQ ID NO:238; SEQ ID NO: 199 and SEQ ID NO:238; SEQ ID NO:200 and SEQ ID NO:238; SEQ ID NO:201 and SEQ ID NO:238; SEQ ID NO:202 and SEQ ID NO:238; SEQ ID NO:203 and SEQ ID NO: 238; SEQ ID NO:204 and SEQ ID NO:238; SEQ ID NO:205 and SEQ ID NO:238; SEQ ID NO: 206 and SEQ ID NO:238; SEQ ID NO:207 and SEQ ID NO:238; SEQ ID NO:208 and SEQ ID NO: 238; SEQ ID NO: 209 and SEQ ID NO:238; SEQ ID NO:210 and SEQ ID NO:238; SEQ ID NO:211 and SEQ ID NO:238; SEQ ID NO:212 and SEQ ID NO:238; SEQ ID NO:213 and SEQ ID NO:238; SEQ ID NO:214 and SEQ ID NO:238; SEQ ID NO:215 and SEQ ID NO: 238; and SEQ ID NO:216 and SEQ ID NO:238.

2.7.4.1. Variants of V_{H} V_{L} Pairs

In some embodiments, the V_{H}-V_{L} pairs provided herein comprise a variant of an illustrative V_{H} and/or V_{L} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.1% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 2.7.5. Heavy Chain Light Chain Pairs

In some embodiments, the antibody comprises a heavy chain sequence of an antibody disclosed herein and a light chain sequence of a suitable antibody. In some embodiments, the antibody comprises a heavy chain sequence of an antibody disclosed herein and a light chain sequence of an antibody disclosed herein.

In some embodiments, the heavy chain comprises a V_{H} sequence comprising, consisting of, or consisting essentially of any one of SEQ ID NOs: 167-216, and the light chain comprises a light chain sequence of any suitable antibody. Techniques for determining whether a particular light chain will pair with a heavy chain as described herein are well known to those of skill in the art. For example, a cell-free protein synthesis reaction comprising a nucleic acid encoding the heavy chain of interest and a nucleic acid encoding the light chain to be assessed may be performed as described, for example, in Example 1.

In some embodiments, the heavy chain comprises a V_{H} sequence comprising, consisting of, or consisting essentially of any one of SEQ ID NOs: 167-216, and the light chain comprises a V_{L} sequence comprising, consisting of, or consisting essentially of any one of SEQ ID NOs: 217-238.

In some embodiments, the heavy chain comprises a V_{H} sequence comprising, consisting of, or consisting essentially of any one of SEQ ID NOs: 167-179, and the light chain comprises a V_{L} sequence comprising, consisting of, or consisting essentially of SEQ ID NO: 217.

In some embodiments, the heavy chain comprises a V_{H} sequence comprising, consisting of, or consisting essentially of any one of SEQ ID NOs: 180-181, and the light chain comprises a V_{L} sequence comprising, consisting of, or consisting essentially of SEQ ID NO: 217.

In some embodiments, the heavy chain comprises a V_{H} sequence comprising, consisting of, or consisting essentially of any one of SEQ ID NOs: 182-188, and the light chain comprises a V_{L} sequence comprising, consisting of, or consisting essentially of SEQ ID NO: 217.

In some embodiments, the heavy chain comprises a V_{H} sequence comprising, consisting of, or consisting essentially of any one of SEQ ID NOs: 189-195, and the light chain comprises a V_{L} sequence comprising, consisting of, or consisting essentially of SEQ ID NO: 217.

In some embodiments, the heavy chain comprises a V_{H} sequence comprising, consisting of, or consisting essentially of any one of SEQ ID NOs: 196-202, and the light chain comprises a V_{L} sequence comprising, consisting of, or consisting essentially of any one of SEQ ID NOs: 218-224.

In some embodiments, the heavy chain comprises a V_{H} sequence comprising, consisting of, or consisting essentially of any one of SEQ ID NOs: 203-207, and the light chain comprises a V_{L} sequence comprising, consisting of, or consisting essentially of any one of SEQ ID NOs: 225-229.

In some embodiments, the heavy chain comprises a V_{H} sequence comprising, consisting of, or consisting essentially of any one of SEQ ID NOs: 208-216, and the light chain comprises a V_{L} sequence comprising, consisting of, or consisting essentially of any one of SEQ ID NO: 230-238.

### 2.8. Antibodies Comprising Six CDRs

In some embodiments, the antibody comprises a CDR-H1 sequence, a CDR-H2 sequence, a CDR-H3 sequence, a CDR-L1 sequence, a CDR-L2 sequence, and a CDR-L3 sequence. In some aspects, the CDR sequences are part of a V_{H} (for CDR-H) or V_{L} (for CDR-L).

In some aspects, the CDR-H1 sequence is a Chothia CDR-H1 sequence comprising, consisting of, or consisting essentially of SEQ ID NOs: 5-31; the CDR-H2 sequence is a Chothia CDR-H2 sequence comprising, consisting of, or consisting essentially of SEQ ID NOs: 57-78; the CDR-H3 sequence is a CDR-H3 sequence comprising, consisting of, or consisting essentially of SEQ ID NOs: 116-145; the CDR-L1 sequence is a CDR-L1 sequence comprising, consisting of, or consisting essentially of SEQ ID NOs: 146-154; the CDR-L2 sequence is a CDR-L2 sequence comprising, consisting of, or consisting essentially of SEQ ID NOs: 155-160; and the CDR-L3 sequence is a CDR-L3 sequence comprising, consisting of, or consisting essentially of SEQ ID NOs: 161-166.

In some aspects, the CDR-H1 sequence is a Kabat CDR-H1 sequence comprising, consisting of, or consisting essentially of SEQ ID NOs: 32-56; the CDR-H2 sequence is a Kabat CDR-H2 sequence comprising, consisting of, or consisting essentially of SEQ ID NOs: 79-115; the CDR-H3 sequence is a CDR-H3 sequence comprising, consisting of, or consisting essentially of SEQ ID NOs: 116-145; the CDR-L1 sequence is a CDR-L1 sequence comprising, consisting of, or consisting essentially of SEQ ID NOs: 146-154; the CDR-L2 sequence is a CDR-L2 sequence comprising, consisting of, or consisting essentially of SEQ ID NOs: 155-160; and the CDR-L3 sequence is a CDR-L3 sequence comprising, consisting of, or consisting essentially of SEQ ID NOs: 161-166.

In some embodiments, the antibody comprises one, two, or three of: a CDR-H1 comprising one of SEQ ID NOs: 5 and 32; a CDR-H2 comprising one of SEQ ID NOs: 57 and 79; a CDR-H3 comprising SEQ ID NO: 116; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 5 and 32; a CDR-H2 comprising one of SEQ ID NOs: 57 and 79; a CDR-H3 comprising SEQ ID NO: 116; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 5 and 32; a CDR-H2 comprising one of SEQ ID NOs: 57 and 79; a CDR-H3 comprising SEQ ID NO: 116; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 6 and 33; a CDR-H2 comprising one of SEQ ID NOs: 57 and 79; a CDR-H3 comprising SEQ ID NO: 117; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 7 and 34; a CDR-H2 comprising one of SEQ ID NOs: 57 and 79; a CDR-H3 comprising SEQ ID NO: 118; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 8 and 34; a CDR-H2 comprising one of SEQ ID NOs: 57 and 79; a CDR-H3 comprising SEQ ID NO: 119; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 9 and 35; a CDR-H2 comprising one of SEQ ID NOs: 57 and 79; a CDR-H3 comprising SEQ ID NO: 120; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 10 and 36; a CDR-H2 comprising one of SEQ ID NOs: 57 and 79; a CDR-H3 comprising SEQ ID NO: 121; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 11 and 36; a CDR-H2 comprising one of SEQ ID NOs: 58 and 80; a CDR-H3 comprising SEQ ID NO: 122; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 12 and 35; a CDR-H2 comprising one of SEQ ID NOs: 57 and 79; a CDR-H3 comprising SEQ ID NO: 116; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 13 and 36; a CDR-H2 comprising one of SEQ ID NOs: 59 and 81; a CDR-H3 comprising SEQ ID NO: 123; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 13 and 36; a CDR-H2 comprising one of SEQ ID NOs: 59 and 81; a CDR-H3 comprising SEQ ID NO: 124; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 14 and 37; a CDR-H2 comprising one of SEQ ID NOs: 57 and 79; a CDR-H3 comprising SEQ ID NO: 125; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 15 and 35; a CDR-H2 comprising one of SEQ ID NOs: 57 and 79; a CDR-H3 comprising SEQ ID NO: 126; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 16 and 38; a CDR-H2 comprising one of SEQ ID NOs: 57 and 79; a CDR-H3 comprising SEQ ID NO: 125; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 17 and 39; a CDR-H2 comprising one of SEQ ID NOs: 60 and 82; a CDR-H3 comprising SEQ ID NO: 127; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 17 and 40; a CDR-H2 comprising one of SEQ ID NOs: 61 and 83; a CDR-H3 comprising SEQ ID NO: 128; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 18 and 41; a CDR-H2 comprising one of SEQ ID NOs: 62 and 84; a CDR-H3 comprising SEQ ID NO: 129; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 19 and 42; a CDR-H2 comprising one of SEQ ID NOs: 63 and 85; a CDR-H3 comprising SEQ ID NO: 130; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 19 and 41; a CDR-H2 comprising one of SEQ ID NOs: 64 and 86; a CDR-H3 comprising SEQ ID NO: 131; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 20 and 43; a CDR-H2 comprising one of SEQ ID NOs: 65 and 87; a CDR-H3 comprising SEQ ID NO: 132; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 21 and 44; a CDR-H2 comprising one of SEQ ID NOs: 66 and 88; a CDR-H3 comprising SEQ ID NO: 133; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 22 and 42; a CDR-H2 comprising one of SEQ ID NOs: 67 and 89; a CDR-H3 comprising SEQ ID NO: 134; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 23 and 45; a CDR-H2 comprising one of SEQ ID NOs: 66 and 90; a CDR-H3 comprising SEQ ID NO: 135; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 18 and 46; a CDR-H2 comprising one of SEQ ID NOs: 68 and 91; a CDR-H3 comprising SEQ ID NO: 136; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 24 and 47; a CDR-H2 comprising one of SEQ ID NOs: 68 and 92; a CDR-H3 comprising SEQ ID NO: 137; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 19 and 48; a CDR-H2 comprising one of SEQ ID NOs: 69 and 93; a CDR-H3 comprising SEQ ID NO: 138; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 25 and 49; a CDR-H2 comprising one of SEQ ID NOs: 70 and 94; a CDR-H3 comprising SEQ ID NO: 136; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 26 and 48; a CDR-H2 comprising one of SEQ ID NOs: 70 and 95; a CDR-H3 comprising SEQ ID NO: 139; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 18 and 50; a CDR-H2 comprising one of SEQ ID NOs: 70 and 96; a CDR-H3 comprising SEQ ID NO: 140; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 26 and 48; a CDR-H2 comprising one of SEQ ID NOs: 70 and 97; a CDR-H3 comprising SEQ ID NO: 136; and/or one, two, or three of: a CDR-L1 comprising SEQ ID NO: 146; a CDR-L2 comprising SEQ ID NO: 155; and a CDR-L3 comprising SEQ ID NO: 161. In some embodiments, the antibody comprises one, two, or three of: a CDR-H1 comprising one of SEQ ID NOs: 27 and 51; a CDR-H2 comprising one of SEQ ID NOs: 71 and 98; a CDR-H3 comprising SEQ ID NO: 141; and/or one, two, or three of: a CDR-L1 comprising SEQ ID NO: 147; a CDR-L2 comprising SEQ ID NO: 156; and a CDR-L3 comprising SEQ ID NO: 162. In some embodiments, the antibody comprises one, two, or three of: a CDR-H1 comprising one of SEQ ID NOs: 27 and 51; a CDR-H2 comprising one of SEQ ID NOs: 72 and 99; a CDR-H3 comprising SEQ ID NO: 142; and/or one, two, or three of: a CDR-L1 comprising SEQ ID NO: 148; a CDR-L2 comprising SEQ ID NO: 158; and a CDR-L3 comprising SEQ ID NO: 163. In some embodiments, the antibody comprises one, two, or three of: a CDR-H1 comprising one of SEQ ID NOs: 27 and 51; a CDR-H2 comprising one of SEQ ID NOs: 73 and 100; a CDR-H3 comprising SEQ ID NO: 142; and/or one, two, or three of: a CDR-L1 comprising SEQ ID NO: 149; a CDR-L2 comprising SEQ ID NO: 158; and a CDR-L3 comprising SEQ ID NO: 164. In some embodiments, the antibody comprises one, two, or three of: a CDR-H1 comprising one of SEQ ID NOs: 27 and 51; a CDR-H2 comprising one of SEQ ID NOs: 73 and 101; and a CDR-H3 comprising SEQ ID NO: 142; and/or one, two, or three of: a CDR-L1 comprising SEQ ID NO: 149; a CDR-L2 comprising SEQ ID NO: 158; and a CDR-L3 comprising SEQ ID NO: 165. In some embodiments, the antibody comprises one, two, or three of: a CDR-H1 comprising one of SEQ ID NOs: 27 and 51; a CDR-H2 comprising one of SEQ ID NOs: 73 and 102; and a CDR-H3 comprising SEQ ID NO: 142; and/or one, two, or three of: a CDR-L1 comprising SEQ ID NO: 149; a CDR-L2 comprising SEQ ID NO: 158; and a CDR-L3 comprising SEQ ID NO: 165. In some embodiments, the antibody comprises one, two, or three of: a CDR-H1 comprising one of SEQ ID NOs: 27 and 51; a CDR-H2 comprising one of SEQ ID NOs: 73 and 102; and a CDR-H3 comprising SEQ ID NO: 142; and/or one, two, or three of: a CDR-L1 comprising SEQ ID NO: 149; a CDR-L2 comprising SEQ ID NO: 158; and a CDR-L3 comprising SEQ ID NO: 165. In some embodiments, the antibody comprises one, two, or three of: a CDR-H1 comprising one of SEQ ID NOs: 27 and 51; a CDR-H2 comprising one of SEQ ID NOs: 73 and 103; and a CDR-H3 comprising SEQ ID NO: 142; and/or one, two, or three of: a CDR-L1 comprising SEQ ID NO: 149; a CDR-L2 comprising SEQ ID NO: 158; and a CDR-L3 comprising SEQ ID NO: 165. In some embodiments, the antibody comprises one, two, or three of: a CDR-H1 comprising one of SEQ ID NOs: 28 and 52; a CDR-H2 comprising one of SEQ ID NOs: 74 and 104; and a CDR-H3 comprising SEQ ID NO: 143; and/or one, two, or three of: a CDR-L1 comprising SEQ ID NO: 150; a CDR-L2 comprising SEQ ID NO: 159; and a CDR-L3 comprising SEQ ID NO: 166. In some embodiments, the antibody comprises one, two, or three of: a CDR-H1 comprising one of SEQ ID NOs: 28 and 52; a CDR-H2 comprising one of SEQ ID NOs: 74 and 105; and a CDR-H3 comprising SEQ ID NO: 143; and/or one, two, or three of: a CDR-L1 comprising SEQ ID NO: 150; a CDR-L2 comprising SEQ ID NO: 159; and a CDR-L3 comprising SEQ ID NO: 166. In some embodiments, the antibody comprises one, two, or three of: a CDR-H1 comprising one of SEQ ID NOs: 28 and 52; a CDR-H2 comprising one of SEQ ID NOs: 74 and 106; and a CDR-H3 comprising SEQ ID NO: 143; and/or one, two, or three of: a CDR-L1 comprising SEQ ID NO: 150; a CDR-L2 comprising SEQ ID NO: 159; and a CDR-L3 comprising SEQ ID NO: 166. In some embodiments, the antibody comprises one, two, or three of: a CDR-H1 comprising one of SEQ ID NOs: 28 and 52; a CDR-H2 comprising one of SEQ ID NOs: 74 and 106; and a CDR-H3 comprising SEQ ID NO: 143; and/or one, two, or three of: a CDR-L1 comprising SEQ ID NO: 150; a CDR-L2 comprising SEQ ID NO: 159; and a CDR-L3 comprising SEQ ID NO: 166. In some embodiments, the antibody comprises one, two, or three of: a CDR-H1 comprising one of SEQ ID NOs: 28 and 52; a CDR-H2 comprising one of SEQ ID NOs: 74 and 107; and a CDR-H3 comprising SEQ ID NO: 143; and/or one, two, or three of: a CDR-L1 comprising SEQ ID NO: 150; a CDR-L2 comprising SEQ ID NO: 159; and a CDR-L3 comprising SEQ ID NO: 166. In some embodiments, the antibody comprises one, two, or three of: a CDR-H1 comprising one of SEQ ID NOs: 29 and 53; a CDR-H2 comprising one of SEQ ID NOs: 75 and 108; and a CDR-H3 comprising SEQ ID NO: 144; and/or one, two, or three of: a CDR-L1 comprising SEQ ID NO: 151; a CDR-L2 comprising SEQ ID NO: 159; and a CDR-L3 comprising SEQ ID NO: 166. In some embodiments, the antibody comprises one, two, or three of: a CDR-H1 comprising one of SEQ ID NOs: 30 and 54; a CDR-H2 comprising one of SEQ ID NOs: 76 and 109; and a CDR-H3 comprising SEQ ID NO: 145; and/or one, two, or three of: a CDR-L1 comprising SEQ ID NO: 152; a CDR-L2 comprising SEQ ID NO: 159; and a CDR-L3 comprising SEQ ID NO: 166. In some embodiments, the antibody comprises one, two, or three of: a CDR-H1 comprising one of SEQ ID NOs: 30 and 53; a CDR-H2 comprising one of SEQ ID NOs: 77 and 110; and a CDR-H3 comprising SEQ ID NO: 145; and/or one, two, or three of: a CDR-L1 comprising SEQ ID NO: 152; a CDR-L2 comprising SEQ ID NO: 159; and a CDR-L3 comprising SEQ ID NO: 166. In some embodiments, the antibody comprises one, two, or three of: a CDR-H1 comprising one of SEQ ID NOs: 28 and 55; a CDR-H2 comprising one of SEQ ID NOs: 77 and 111; and a CDR-H3 comprising SEQ ID NO: 145; and/or one, two, or three of: a CDR-L1 comprising SEQ ID NO: 153; a CDR-L2 comprising SEQ ID NO: 159; and a CDR-L3 comprising SEQ ID NO: 166. In some embodiments, the antibody comprises one, two, or three of: a CDR-H1 comprising one of SEQ ID NOs: 31 and 56; a CDR-H2 comprising one of SEQ ID NOs: 78 and 112; and a CDR-H3 comprising SEQ ID NO: 145; and/or one, two, or three of: a CDR-L1 comprising SEQ ID NO: 154; a CDR-L2 comprising SEQ ID NO: 160; and a CDR-L3 comprising SEQ ID NO: 166. In some embodiments, the antibody comprises one, two, or three of: a CDR-H1 comprising one of SEQ ID NOs: 28 and 55; a CDR-H2 comprising one of SEQ ID NOs: 77 and 113; and a CDR-H3 comprising SEQ ID NO: 145; and/or one, two, or three of: a CDR-L1 comprising SEQ ID NO: 153; a CDR-L2 comprising SEQ ID NO: 159; and a CDR-L3 comprising SEQ ID NO: 166. In some embodiments, the antibody comprises one, two, or three of: a CDR-H1 comprising one of SEQ ID NOs: 28 and 55; a CDR-H2 comprising one of SEQ ID NOs: 77 and 114; and a CDR-H3 comprising SEQ ID NO: 145; and/or one, two, or three of: a CDR-L1 comprising SEQ ID NO: 153; a CDR-L2 comprising SEQ ID NO: 159; and a CDR-L3 comprising SEQ ID NO: 166. In some embodiments, the antibody comprises one, two, or three of: a CDR-H1 comprising one of SEQ ID NOs: 28 and 55; a CDR-H2 comprising one of SEQ ID NOs: 77 and 114; and a CDR-H3 comprising SEQ ID NO: 145; and/or one, two, or three of: a CDR-L1 comprising SEQ ID NO: 153; a CDR-L2 comprising SEQ ID NO: 159; and a CDR-L3 comprising SEQ ID NO: 166. In some embodiments, the antibody comprises one, two, or three of: a CDR-H1 comprising one of SEQ ID NOs: 28 and 55; a CDR-H2 comprising one of SEQ ID NOs: 77 and 115; and a CDR-H3 comprising SEQ ID NO: 145; and/or one, two, or three of: a CDR-L1 comprising SEQ ID NO: 153; a CDR-L2 comprising SEQ ID NO: 159; and a CDR-L3 comprising SEQ ID NO: 166.

In certain embodiments, each antibody comprises one, two, three, four, five, or six of the listed CDRs. In certain embodiments, each antibody comprises one of the listed heavy chain CDRs. In certain embodiments, each antibody comprises two of the listed heavy chain CDRs. In certain embodiments, each antibody comprises three of the listed heavy chain CDRs. In certain embodiments, each antibody comprises one of the listed light chain CDRs. In certain embodiments, each antibody comprises two of the listed light chain CDRs. In certain embodiments, each antibody comprises three of the listed light chain CDRs. In certain embodiments, each antibody comprises the listed CDR-H3 and CDR-L3. In certain embodiments, each antibody comprises the listed CDR-H2 and CDR-L2. In certain embodiments, each antibody comprises the listed CDR-H1 and CDR-L1. In certain embodiments, each antibody comprises the listed CDR-H3, CDR-H2, CDR-L3, and CDR-L2. In certain embodiments, each antibody comprises six of the listed CDRs. In particular embodiments, the CDRs are according to Chothia. In particular embodiments, the CDRs are according to Kabat.

### 2.8.1. Variants of Antibodies Comprising All Six CDRs

In some embodiments, the CDR-H1, CDR-H2, CDR-H3, CDR-L1, CDR-L2, and CDR-L3 provided herein comprise a variant of an illustrative CDR-H1, CDR-H2, CDR-H3, CDR-L1, CDR-L2, and/or CDR-L3 sequence provided in this disclosure.

In some aspects, the CDR-H1 sequence comprises, consists of, or consists essentially of a variant of an illustrative Chothia or Kabat CDR-H1 sequence provided in this disclosure. In some aspects, the CDR-H1 sequence comprises, consists of, or consists essentially of a sequence having at least 70%, 75%, 80%, 85%, 90%, or 95% identity with any of the illustrative Chothia or Kabat CDR-H1 sequences provided in this disclosure. In some aspects, the CDR-H1 sequence comprises, consists of, or consists essentially of any of the illustrative Chothia or Kabat CDR-H1 sequences provided in this disclosure, with 1, 2, or 3 amino acid substitutions. In some aspects, the amino acid substitutions are conservative amino acid substitutions.

In some aspects, the CDR-H2 sequence comprises, consists of, or consists essentially of a variant of an illustrative Chothia or Kabat CDR-H2 sequence provided in this disclosure. In some aspects, the CDR-H2 sequence comprises, consists of, or consists essentially of a sequence having at least 70%, 75%, 80%, 85%, 90%, or 95% identity with any of the illustrative Chothia or Kabat CDR-H2 sequences provided in this disclosure. In some aspects, the CDR-H2 sequence comprises, consists of, or consists essentially of any of the illustrative Chothia or Kabat CDR-H2 sequences provided in this disclosure, with 1, 2, or 3 amino acid substitutions. In some aspects, the amino acid substitutions are conservative amino acid substitutions.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

### 2.9. Consensus Sequences

In some embodiments, provided herein are anti-BCMA antibodies comprising one or more sequences defined by consensus sequences. Each consensus sequence is based, at least in part, on one or more alignments of two or more useful anti-BCMA CDR sequences provided in this disclosure. Based on such alignments, a person of skill in the art would recognize that different amino acid residues may useful in certain positions of the CDRs. Accordingly, each consensus sequence encompasses two or more useful anti-BCMA CDR sequences.

In some embodiments, the antibodies comprise one to six of the consensus CDR sequences provided herein. In some embodiments, the antibodies comprise two to six of the consensus CDR sequences provided herein. In some embodiments, the antibodies comprise three to six of the consensus CDR sequences provided herein. In some embodiments, the antibodies comprise four to six of the consensus CDR sequences provided herein. In some embodiments, the antibodies comprise five to six of the consensus CDR sequences provided herein. In some embodiments, the antibodies comprise six of the consensus CDR sequences provided herein. In some embodiments, the antibodies comprise a V_{L} comprising the CDR-L consensus sequence(s). In some embodiments, the antibodies comprise a V_{H} comprising the CDR-H consensus sequence(s). In some embodiments, the antibodies comprise a V_{H} comprising the CDR-H consensus sequence(s) and a V_{L} comprising the CDR-L consensus sequence(s).

### 2.9.1. CDR-H3 Consensus Sequences

In some embodiments, the antibody comprises a CDR-H3 sequence defined by the consensus sequence D-α₂-α₃-α₄-α₅-Y-W-T-Y-V-L-D-Y (SEQ ID NO: 248), where α₂ is Y or F; α₃ is V or I; α₄ is Y, L, N, R, Q, or P; and α₅ is Q, A, N, or S.

In some embodiments, the antibody comprises a CDR-H3 sequence defined by the consensus sequence D-Y-α₃-α₄-α₅-Y-α₇-T-G-V-L-D-Y (SEQ ID NO: 249), where α₃ is G or D; α₄ is P or L; α₅ is W or R; and α₇ is G or L.

In some embodiments, the antibody comprises a CDR-H3 sequence defined by the consensus sequence D-α₂-G-α₄-α₅—Y-W-V-G-α₁₀-α₁₁-D-Y (SEQ ID NO: 250), where α₂ is L, M, or W; α₄ is G, V, H, Y, or S; α₅ is G or R; α₁₀ is For V; and α₁₁ is For S.

In some embodiments, the antibody comprises a CDR-H3 sequence defined by the consensus sequence D-α₂-α₃-D-R-Y-α₇-T-α₉-V-L-D-Y (SEQ ID NO: 251), where α₂ is For Y; α₃ is Y, H, or N; α₇ is S, A, or F; and α₉ is Y or F.

In some embodiments, the antibody comprises a CDR-H3 sequence defined by the consensus sequence T-T-C-α₄-G-S-G-G-C-I-D-T (SEQ ID NO: 252), where α₄ is I or V.

In some embodiments, the antibody comprises a CDR-H3 sequence defined by the consensus sequence G-G-G-L-N-S-Y-G-C-S-G-A-N-I-D-A (SEQ ID NO: 143).

In some embodiments, the antibody comprises a CDR-H3 sequence defined by the consensus sequence G-G-G-α₄-A-S-I-D-α₉ (SEQ ID NO: 253), where α₄ is A or G, and α₉ is T or G.

### 2.9.2. Chothia CDR-H1 Consensus Sequences

In some embodiments, the antibody comprises a Chothia CDR-H1 sequence defined by the consensus sequence G-F-N-I-γ₅-γ₆-γ₇ (SEQ ID NO: 254), where γ₅ is S, I, R, Y, or G; γ₆ is G, Y, A, V, or R; and γ₇ is S or P.

In some embodiments, the antibody comprises a Chothia CDR-H1 sequence defined by the consensus sequence G-F-N-I-N-N-S(SEQ ID NO: 17).

In some embodiments, the antibody comprises a Chothia CDR-H1 sequence defined by the consensus sequence G-F-N-I-γ₅-γ₆-γ₇ (SEQ ID NO: 255), where γ₅ is S, T, A, or Q; γ₆ is S, P, Y, or T; and γ₇ is Y, D, or R.

In some embodiments, the antibody comprises a Chothia CDR-H1 sequence defined by the consensus sequence G-F-N-I-γ₅-γ₆-γ₇ (SEQ ID NO: 256), where γ₅ is S, A, K, or D; γ₆ is S, A, P, or D; and γ₇ is Y or T.

In some embodiments, the antibody comprises a Chothia CDR-H1 sequence defined by the consensus sequence G-F-T-F-S-S-F (SEQ ID NO: 27).

In some embodiments, the antibody comprises a Chothia CDR-H1 sequence defined by the consensus sequence G-F-T-F-S-G-Y (SEQ ID NO: 28).

In some embodiments, the antibody comprises a Chothia CDR-H1 sequence defined by the consensus sequence G-F-γ₃-γ₄-S-γ₆-Y (SEQ ID NO: 257), where γ₃ is S or T; γ₄ is I or F; and γ₆ is D, G, or S.

### 2.9.3. Chothia CDR-H2 Consensus Sequences

In some embodiments, the antibody comprises a Chothia CDR-H2 sequence defined by the consensus sequence ε₁-P-ε₃-A-ε₅-G-Y (SEQ ID NO: 258), where ε₁ is N or S; ε₃ is absent; and ε₅ is G or A.

In some embodiments, the antibody comprises a Chothia CDR-H2 sequence defined by the consensus sequence Y-P-ε₃-Y-ε₅-G-ε₇ (SEQ ID NO: 259), where ε₃ is absent; ε₅ is S or I; and ε₇ is Y or F.

In some embodiments, the antibody comprises a Chothia CDR-H2 sequence defined by the consensus sequence T-P-ε₃-ε₄-ε₅-G-ε₇ (SEQ ID NO: 260), where ε₃ is absent; &4 is S, P, A, or F; ε5 is G, S, A, or D; and ε₇ is Y or F.

In some embodiments, the antibody comprises a Chothia CDR-H2 sequence defined by the consensus sequence ε₁-P-ε₃-ε₄-ε₅-G-Y (SEQ ID NO: 261), where ε₁ is S or F; ε₃ is absent; ε₄ is Y or S; and ε₅ is G or D.

In some embodiments, the antibody comprises a Chothia CDR-H2 sequence defined by the consensus sequence ε₁-N-D-ε₄-G-ε₆-S(SEQ ID NO: 262), where ε₁ is R or S; ε₄ is absent; and ε₆ is N, S, or R.

In some embodiments, the antibody comprises a Chothia CDR-H2 sequence defined by the consensus sequence T-Y-G-T-G-S-Y (SEQ ID NO: 74).

In some embodiments, the antibody comprises a Chothia CDR-H2 sequence defined by the consensus sequence ε₁-ε₂-ε₃-ε₄-ε₅-ε₆-ε₇, where ε₁ is D or N; ε₂ is H or S; ε₃ is D, A, G, or absent; ε₄ is G, A, or absent; ε₅ is G or S; ε₆ is R or S; and ε₇ is Y, G, or D.

### 2.9.4. Kabat CDR-H1 Consensus Sequences

In some embodiments, the antibody comprises a Kabat CDR-H1 sequence defined by the consensus sequence ζ₁-ζ₂-G-I-H, where ζ₁ is G, Y, A, V, or R; and ζ₂ is S or P.

In some embodiments, the antibody comprises a Kabat CDR-H1 sequence defined by the consensus sequence N-S-ζ₃-I-H (SEQ ID NO: 263), where ζ₃ is Y or W.

In some embodiments, the antibody comprises a Kabat CDR-H1 sequence defined by the consensus sequence ζ₁-ζ₂-W-I-H, where ζ₁ is S, P, Y, or T; and ζ₂ is Y, D, or R.

In some embodiments, the antibody comprises a Kabat CDR-H1 sequence defined by the consensus sequence ζ₁-ζ₂-ζ₃-I-H, where ζ₁ is S, A, P, or D; ζ₂ is Y or T; and ζ₃ is A, T, Y, or G.

In some embodiments, the antibody comprises a Kabat CDR-H1 sequence defined by the consensus sequence S-F-N-M-F (SEQ ID NO: 51).

In some embodiments, the antibody comprises a Kabat CDR-H1 sequence defined by the consensus sequence G-Y-N-M-G (SEQ ID NO: 52).

In some embodiments, the antibody comprises a Kabat CDR-H1 sequence defined by the consensus sequence ζ₁-Y-G-ζ₄-G, where ζ₁ is D, G, or S; and ζ₄ is M or L.

### 2.9.5. Kabat CDR-H2 Consensus Sequences

In some embodiments, the antibody comprises a Kabat CDR-H2 sequence defined by the consensus sequence F-I-θ₃-P-A-θ₆-G-Y-T-D-Y-A-θ₁₃-S-V-K-G (SEQ ID NO: 264), where θ₃ is N or S; θ₆ is G or A; and θ₁₃ is D or G.

In some embodiments, the antibody comprises a Kabat CDR-H2 sequence defined by the consensus sequence W-I-Y-P-Y-θ₆-G-θ₈-T-θ₁₀-Y-A-D-S-V-K-G (SEQ ID NO: 265), where θ₆ is S or I; θ₈ is Y or F; and θ₁₀ is N or E.

In some embodiments, the antibody comprises a Kabat CDR-H2 sequence defined by the consensus sequence V-I-T-P-θ₅-θ₆-G-θ₈-T-θ₁₀-Y-A-D-S-V-K-G (SEQ ID NO: 266), where θ₅ is S, P, A, or F; θ₆ is G, S, A, or D; θ₈ is Y or F; and θ₁₀ is Y or H.

In some embodiments, the antibody comprises a Kabat CDR-H2 sequence defined by the consensus sequence Jan. 1, 2003-P-θ₅-θ₆-G-Y-T-θ₁₀-Y-A-D-S-V-K-G (SEQ ID NO: 267), where θ₁ is V, W, H, or F; θ₃ is S or F; θ₅ is Y or S; θ₆ is G or D; and θ₁₀ is E or D.

In some embodiments, the antibody comprises a Kabat CDR-H2 sequence defined by the consensus sequence Y-I-θ₃-N-D-G-θ₇-S-θ₉-S-Y-θ₁₂-θ₁₃-θ₁₄-V-K-G (SEQ ID NO: 268), where θ₃ is R or S; θ₇ is N, S, or R; θ₉ is A or T; θ₁₂ is G, V, or A; θ₁₃ is P, D, or A; and θ₁₄ is A, S, or P.

In some embodiments, the antibody comprises a Kabat CDR-H2 sequence defined by the consensus sequence G-I-T-Y-G-T-G-S-Y-T-A-Y-θ₁₃-θ₁₄-θ₁₅-V-K-G (SEQ ID NO: 269), where θ₁₃ is G, V, or A; θ₁₄ is A or D; and θ₁₅ is A, S, or P.

In some embodiments, the antibody comprises a Kabat CDR-H2 sequence defined by the consensus sequence R-I-θ₃-θ₄-θ₅-θ₆-θ₇-θ₈-θ₉-T-θ₁₁-Y-θ₁₃-θ₁₄-θ₁₅-V-θ₁₇-G (SEQ ID NO: 270), where θ₃ is D or N; θ₄ is H or S; θ₅ is D, A, G, or absent; θ₆ is G, A, or absent; θ₇ is G or S; θ₈ is R or S; θ₉ is Y, G, or D; On is D, Y, or N; θ₁₃ is G, V, or A; θ₁₄ is A, S, or D; θ₁₅ is V, A, S, or P; and θ₁₇ is K or D.

### 2.9.6. CDR-L3 Consensus Sequences

In some embodiments, the antibody comprises a CDR-L3 sequence defined by the consensus sequence Q-Q-H-Y-T-T-P-P-T (SEQ ID NO: 161).

In some embodiments, In some embodiments, the antibody comprises a CDR-L3 sequence defined by the consensus sequence σ₁-σ₂-σ₃-D-σ₅-σ₆-σ₇-D-σ₉-σ₁₀, where: σ₁ is A or G; σ₂ is N or G; σ₃ is V or F; σ₅ is absent or S; σ₆ is Y, S, or F; σ₇ is T or S; σ₉ is D or A; and σ₁₀ is V or I.

In some embodiments, the antibody comprises a CDR-L3 sequence defined by the consensus sequence G-G-F-D-S-S-T-D-A-I (SEQ ID NO: 166).

In some embodiments, the antibody comprises a CDR-L3 sequence defined by the consensus sequence G-σ₂-F-D-S-S-σ₇-D-A-I (SEQ ID NO: 271), where: σ₂ is S or G; and σ₇ is T or S.

### 2.9.7. CDR-L2 Consensus Sequences

In some embodiments, the antibody comprises a CDR-L2 sequence defined by the consensus sequence S-A-S-F-L-Y-S(SEQ ID NO: 155).

In some embodiments, the antibody comprises a CDR-L2 sequence defined by the consensus sequence π₁-N-N-π₄-R-P-S(SEQ ID NO: 272), where: π₁ is S, Y, or R; and π₄ is Q or K.

In some embodiments, the antibody comprises a CDR-L2 sequence defined by the consensus sequence N-N-N-N-R-P-S(SEQ ID NO: 159).

In some embodiments, the antibody comprises a CDR-L2 sequence defined by the consensus sequence N-π₂-N-N-R-P-S(SEQ ID NO: 273), where: π₂ is N or S.

### 2.9.8. CDR-L1 Consensus Sequences

In some embodiments, the antibody comprises a CDR-L1 sequence defined by the consensus sequence R-A-S-Q-D-V-N-T-A-V-A (SEQ ID NO: 146).

In some embodiments, the antibody comprises a CDR-L1 sequence defined by the consensus sequence S-G-G-μ₄-μ₅-D-Y-G (SEQ ID NO: 274), where: μ₄ is S or N; and μ₅ is S or Y.

In some embodiments, the antibody comprises a CDR-L1 sequence defined by the consensus sequence S-G-G-G-N-Y-F-G-S-Y-Y-Y-G (SEQ ID NO: 150).

In some embodiments, the antibody comprises a CDR-L1 sequence defined by the consensus sequence S-G-G-G-μ₅-Y-μ₇-G-μ₉-Y-Y-Y-G (SEQ ID NO: 275), where: μ₅ is S or N; μ₇ is V, Y, or A; and μ₉ is G or S.

### 3. Germline

One of skill in the art would recognize that the CDR sequences provided herein may also be useful when combined with variable regions encoded by other variable region germline genes, or variants thereof. In particular, the CDR sequences provided herein may be useful when combined with variable regions encoded by variable region germline genes, or variants thereof, that are structurally similar to the variable region germline genes recited above. For example, in some embodiments, a CDR-H sequence provided herein may be combined with a variable region encoded by a variable region germline gene selected from the V_{H} 1, V_{H} 2, V_{H} 3, or V_{H} 4 families, or a variant thereof. In some embodiments, a CDR-L sequence provided herein may be combined with a variable region encoded by a variable region germline gene selected from the Vκ1, Vκ2, or Vκ3, or a variant thereof.

### 4. Affinity

In some embodiments, the affinity of the antibody for BCMA as indicated by K_{D}, is less than about 10⁻⁵ M, less than about 10⁻⁶ M, less than about 10⁻⁷ M, less than about 10⁻⁸ M, less than about 10⁻⁹ M, or less than about 10⁻¹⁰ M. In some embodiments, the affinity of the antibody is between about 10⁻⁷ M and 10⁻¹¹ M. In some embodiments, the affinity of the antibody is between about 10⁻⁷ M and 10⁻¹⁰ M. In some embodiments, the affinity of the antibody is between about 10⁻⁷ M and 10⁻⁹ M. In some embodiments, the affinity of the antibody is between about 10⁻⁷ M and 10⁻⁸ M. In some embodiments, the affinity of the antibody is between about 10⁻⁸ M and 10⁻¹¹ M. In some embodiments, the affinity of the antibody is between about 10⁻⁸ M and 10⁻¹⁰ M. In some embodiments, the affinity of the antibody is between about 10⁻⁹ M and 10⁻¹¹ M. In some embodiments, the affinity of the antibody is between about 10⁻⁹ M and 10⁻¹⁰ M.

In some embodiments, the affinity of the antibody for human BCMA, human BCMA extracellular domain, or for individual domains within human BCMA, as determined by surface plasmon resonance at 25° C., and as indicated by K_{D}, is from about 4.38×10⁻¹¹ M to about 5.23×10⁻⁹ M. In some embodiments, the affinity of the antibody for human BCMA, as determined by surface plasmon resonance at 25° C., and as indicated by K_{D}, is from about 2.76×10⁻¹⁰ M to about 2.36×10⁻⁹ M. In some embodiments, the affinity of the antibody for human BCMA, as determined by surface plasmon resonance at 25° C., and as indicated by K_{D}, is from about 3.78×10⁻¹⁰ M to about 2.08×10⁻⁹ M. In some embodiments, the affinity of the antibody for human BCMA, as determined by surface plasmon resonance at 25° C., and as indicated by K_{D}, is from about 5.57×10⁻¹⁰ M to about 1.63×10⁻⁹ M. In some embodiments, the affinity of the antibody for human BCMA is about any of the K_{D} values reported for human BCMA in the examples below.

In some embodiments, the affinity of the antibody for cynomolgous BCMA, cynomolgous BCMA extracellular domain, or for individual domains within cynomolgous BCMA, as determined by surface plasmon resonance at 25° C., and as indicated by K_{D}, is from about 3.24×10⁻⁹ M to about 7.90×10⁻⁹ M. In some embodiments, the affinity of the antibody for cynomolgous BCMA is about any of the K_{D} values reported for cynomolgous BCMA in the examples below.

In some embodiments the antibody has a kₐ of at least about 10⁴ M⁻¹×sec⁻¹. In some embodiments the antibody has a kₐ of at least about 10⁵ M⁻¹×sec⁻¹. In some embodiments the antibody has a kₐ of at least about 10⁻⁶ M⁻¹×sec⁻¹. In some embodiments the antibody has a kₐ of between about 10⁴ M⁻¹×sec⁻¹ and about 10⁷ M⁻¹×sec⁻¹. In some embodiments the antibody has a kₐ of between about 10⁵ M⁻¹×sec⁻¹ and about 10⁷ M⁻¹×sec⁻¹. In some embodiments the antibody has a kₐ of between about 10⁶ M⁻¹×sec⁻¹ and about 10⁷ M⁻¹×sec⁻¹.

In some embodiments the antibody has a kₐ when associating with human BCMA, human BCMA extracellular domain, or for individual domains within human BCMA, as determined by surface plasmon resonance at 25° C., of from about 1.36×10⁵ M⁻¹×sec⁻¹ to about 1.41×10⁶ M⁻¹×sec⁻¹. In some embodiments the antibody has a kₐ when associating with human BCMA, as determined by surface plasmon resonance at 25° C., of from about 4.37×10⁵ M⁻¹×sec-1 to about 1.36×10⁶ M⁻¹×sec⁻¹. In some embodiments the antibody has a kₐ when associating with human BCMA, as determined by surface plasmon resonance at 25° C., of from about 4.57×10⁵ M⁻¹×sec⁻¹ to about 9.27×10⁵ M⁻¹×sec⁻¹. In some embodiments the antibody has a kₐ when associating with human BCMA, as determined by surface plasmon resonance at 25° C., of from about 7.14×10⁵ M⁻¹×sec⁻¹ to about 7.66×10⁵ M⁻¹×sec⁻¹. In some embodiments the antibody has a kₐ when associating with human BCMA of about any of the kₐ values reported for human BCMA in the examples below.

In some embodiments the antibody has a kₐ when associating with cynomolgous BCMA, cynomolgous BCMA extracellular domain, or for individual domains within cynomolgous BCMA, as determined by surface plasmon resonance at 25° C., of from about 2.49×10⁵ M⁻¹×sec⁻¹ to about 3.58×10⁶ M⁻¹×sec⁻¹. In some embodiments the antibody has a kₐ when associating with cynomolgous BCMA of about any of the kₐ values reported for cynomolgous BCMA in the examples below.

In some embodiments the antibody has a k_{d} of about 10⁻⁵ sec⁻¹ or less. In some embodiments the antibody has a k_{d} of about 10⁴ sec⁻¹ or less. In some embodiments the antibody has a k_{d} of about 10⁻³ sec⁻¹ or less. In some embodiments the antibody has a k_{d} of between about 10⁻² sec⁻¹ and about 10⁻⁵ sec⁻¹. In some embodiments the antibody has a k_{d} of between about 10⁻² sec⁻¹ and about 10⁴ sec⁻¹. In some embodiments the antibody has a k_{d} of between about 10⁻³ sec⁻¹ and about 10⁻⁵ sec⁻¹.

In some embodiments the antibody has a k_{d} when dissociating from human BCMA, human BCMA extracellular domain, or for individual domains within human BCMA, as determined by surface plasmon resonance at 25° C., of from about 2.82×10⁻⁵ sec⁻¹ to about 3.32×10⁻³ sec⁻¹. In some embodiments the antibody has a k_{d} when dissociating from human BCMA, as determined by surface plasmon resonance at 25° C., of from about 1.31×10⁴ sec⁻¹ to about 2.83×10⁻³ sec⁻¹. In some embodiments the antibody has a k_{d} when dissociating from human BCMA, as determined by surface plasmon resonance at 25° C., of from about 1.93×10⁻⁴ sec⁻¹ to about 7.45×10⁻⁴ sec⁻¹. In some embodiments the antibody has a k_{d} when dissociating from human BCMA, as determined by surface plasmon resonance at 25° C., of from about 5.16×10⁴ sec⁻¹ to about 7.12×10⁴ sec⁻¹. In some embodiments the antibody has a k_{d} when dissociating from human BCMA of about any of the k_{d} values reported for human BCMA in the examples below.

In some embodiments the antibody has a k_{d} when dissociating from cynomolgous BCMA, cynomolgous BCMA extracellular domain, or for individual domains within cynomolgous BCMA, as determined by surface plasmon resonance at 25° C., of from about 1.14×10⁻³ sec⁻¹ to about 2.74×10⁻³ sec⁻¹. In some embodiments the antibody has a k_{d} when dissociating from cynomolgous BCMA of about any of the k_{d} values reported for cynomolgous BCMA in the examples below.

In some aspects, the K_{D}, kₐ, and k_{d} are determined at 25° C. In some embodiments, the K_{D}, kₐ, and k_{d} are determined by surface plasmon resonance. In some embodiments, the K_{D}, kₐ, and k_{d} are determined according to the methods described in the Examples provided herein.

### 5. Epitope Bins

In some embodiments, the antibody binds the same epitope as an antibody encompassing any of SEQ ID NOs: 167-216. In some embodiments, the antibody binds the same epitope as an antibody comprising (a) a V_{H} sequence comprising, consisting of, or consisting essentially of SEQ ID NOs: 167-216, and (b) a V_{L} sequence comprising, consisting of, or consisting essentially of SEQ ID NOs: 217-238. For example, in some embodiments, the antibody binds the same epitope as an antibody comprising any of the V_{H}-V_{L} pairs, above. In some embodiments, the antibody competes for epitope binding with an antibody encompassing any of SEQ ID NOs: 167-216. In some embodiments, the antibody competes for epitope binding with an antibody comprising (a) a V_{H} sequence comprising, consisting or, or consisting essentially of SEQ ID NOs: 167-216, and (b) a V_{L} sequence comprising, consisting of, or consisting essentially of SEQ ID NOs: 217-238. For example, in some embodiments, the antibody competes for epitope binding with an antibody comprising any of the V_{H}-V_{L} pairs, above.

In some embodiments, the antibody binds the same epitope as an antibody comprising (a) a V_{H} sequence comprising, consisting of, or consisting essentially of SEQ ID NOs: 167-179, and (b) a V_{L} sequence comprising, consisting of, or consisting essentially of SEQ ID NO: 217. In some embodiments, the antibody competes for epitope binding with an antibody comprising (a) a V_{H} sequence comprising, consisting or, or consisting essentially of SEQ ID NOs: 167-179, and (b) a V_{L} sequence comprising, consisting of, or consisting essentially of SEQ ID NO: 217.

In some embodiments, the antibody binds the same epitope as an antibody comprising (a) a V_{H} sequence comprising, consisting of, or consisting essentially of SEQ ID NOs: 180-181, and (b) a V_{L} sequence comprising, consisting of, or consisting essentially of SEQ ID NO: 217. In some embodiments, the antibody competes for epitope binding with an antibody comprising (a) a V_{H} sequence comprising, consisting or, or consisting essentially of SEQ ID NOs: 180-181, and (b) a V_{L} sequence comprising, consisting of, or consisting essentially of SEQ ID NO: 217.

In some embodiments, the antibody binds the same epitope as an antibody comprising (a) a V_{H} sequence comprising, consisting of, or consisting essentially of SEQ ID NOs: 182-188, and (b) a V_{L} sequence comprising, consisting of, or consisting essentially of SEQ ID NO: 217. In some embodiments, the antibody competes for epitope binding with an antibody comprising (a) a V_{H} sequence comprising, consisting or, or consisting essentially of SEQ ID NOs: 182-188, and (b) a V_{L} sequence comprising, consisting of, or consisting essentially of SEQ ID NO: 217.

In some embodiments, the antibody binds the same epitope as an antibody comprising (a) a V_{H} sequence comprising, consisting of, or consisting essentially of SEQ ID NOs: 189-195, and (b) a V_{L} sequence comprising, consisting of, or consisting essentially of SEQ ID NO: 217. In some embodiments, the antibody competes for epitope binding with an antibody comprising (a) a V_{H} sequence comprising, consisting or, or consisting essentially of SEQ ID NOs: 189-195, and (b) a V_{L} sequence comprising, consisting of, or consisting essentially of SEQ ID NO: 217.

In some embodiments, the antibody binds the same epitope as an antibody comprising (a) a V_{H} sequence comprising, consisting of, or consisting essentially of SEQ ID NOs: 196-202, and (b) a V_{L} sequence comprising, consisting of, or consisting essentially of SEQ ID NO: 218-224. In some embodiments, the antibody competes for epitope binding with an antibody comprising (a) a V_{H} sequence comprising, consisting or, or consisting essentially of SEQ ID NOs: 196-202, and (b) a V_{L} sequence comprising, consisting of, or consisting essentially of SEQ ID NO: 218-224.

In some embodiments, the antibody binds the same epitope as an antibody comprising (a) a V_{H} sequence comprising, consisting of, or consisting essentially of SEQ ID NOs: 203-207, and (b) a V_{L} sequence comprising, consisting of, or consisting essentially of SEQ ID NO: 225-229. In some embodiments, the antibody competes for epitope binding with an antibody comprising (a) a V_{H} sequence comprising, consisting or, or consisting essentially of SEQ ID NOs: 203-207, and (b) a V_{L} sequence comprising, consisting of, or consisting essentially of SEQ ID NO: 225-229.

In some embodiments, the antibody binds the same epitope as an antibody comprising (a) a V_{H} sequence comprising, consisting of, or consisting essentially of SEQ ID NOs: 208-216, and (b) a V_{L} sequence comprising, consisting of, or consisting essentially of SEQ ID NO: 230-238. In some embodiments, the antibody competes for epitope binding with an antibody comprising (a) a V_{H} sequence comprising, consisting or, or consisting essentially of SEQ ID NOs: 208-216, and (b) a V_{L} sequence comprising, consisting of, or consisting essentially of SEQ ID NO: 230-238.

### 6. Glycosylation Variants

In certain embodiments, an antibody may be altered to increase, decrease or eliminate the extent to which it is glycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to the antibody may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

### 7. Fc Variants

In certain embodiments, amino acid modifications may be introduced into the Fc region of an antibody provided herein to generate an Fc region variant. In certain embodiments, the Fc region variant possesses some, but not all, effector functions. Such antibodies may be useful, for example, in applications in which the half-life of the antibody in vivo is important, yet certain effector functions are unnecessary or deleterious. Examples of effector functions include complement-dependent cytotoxicity (CDC) and antibody-directed complement-mediated cytotoxicity (ADCC). Numerous substitutions or substitutions or deletions with altered effector function are known in the art.

In some embodiments, the Fc comprises one or more modifications in at least one of the CH3 sequences. In some embodiments, the Fc comprises one or more modifications in at least one of the CH2 sequences. For example, the Fc can include one or modifications selected from the group consisting of: V262E, V262D, V262K, V262R, V262S, V264S, V303R, and V305R. In some embodiments, an Fc is a single polypeptide. In some embodiments, an Fc is multiple peptides, e.g., two polypeptides. Exemplary modifications in the Fc region are described, for example, in International Patent Application No. PCT/US2017/037545, filed Jun. 14, 2017.

An alteration in in CDC and/or ADCC activity can be confirmed using in vitro and/or in vivo assays. For example, Fc receptor (FcR) binding assays can be conducted to measure FcγR binding. The primary cells for mediating ADCC, NK cells, express FcγRIII only, whereas monocytes express FcγRI, FcγRII and FcγRIII. FcR expression on hematopoietic cells is summarized in Ravetch and Kinet, Ann. Rev. Immunol., 1991, 9:457-492, incorporated by reference in its entirety.

Non-limiting examples of in vitro assays to assess ADCC activity of a molecule of interest are provided in U.S. Pat. Nos. 5,500,362 and 5,821,337; Hellstrom et al., Proc. Natl. Acad. Sci. U.S.A., 1986, 83:7059-7063; Hellstrom et al., Proc. Natl. Acad. Sci. U.S.A., 1985, 82:1499-1502; and Bruggemann et al., J. Exp. Med., 1987, 166:1351-1361; each of which is incorporated by reference in its entirety. Useful effector cells for such assays include peripheral blood mononuclear cells (PBMC) and Natural Killer (NK) cells. Alternatively, or additionally, ADCC activity of the molecule of interest may be assessed in vivo, using an animal model such as that disclosed in Clynes et al. Proc. Natl. Acad. Sci. U.S.A., 1998, 95:652-656, incorporated by reference in its entirety.

C1q binding assays may also be carried out to confirm that the antibody is unable to bind C1q and hence lacks CDC activity. Examples of C1q binding assays include those described in WO 2006/029879 and WO 2005/100402, each of which is incorporated by reference in its entirety.

Complement activation assays include those described, for example, in Gazzano-Santoro et al., J. Immunol. Methods, 1996, 202:163-171; Cragg et al., Blood, 2003, 101:1045-1052; and Cragg and Glennie, Blood, 2004, 103:2738-2743; each of which is incorporated by reference in its entirety.

FcRn binding and in vivo clearance (half-life determination) can also be measured, for example, using the methods described in Petkova et al., Intl. Immunol., 2006, 18:1759-1769, incorporated by reference in its entirety.

### 8. Preparation of Antibodies

### 8.1. Antigen Preparation

The BCMA protein to be used for isolation of the antibodies may be intact BCMA or a fragment of BCMA. The intact BCMA protein, or fragment of BCMA, may be in the form of an isolated protein or protein expressed by a cell. Other forms of BCMA useful for generating antibodies will be apparent to those skilled in the art.

### 8.2 Monoclonal Antibodies

Monoclonal antibodies may be obtained, for example, using the hybridoma method first described by Kohler et al., Nature, 1975, 256:495-497 (incorporated by reference in its entirety), and/or by recombinant DNA methods (see e.g., U.S. Pat. No. 4,816,567, incorporated by reference in its entirety). Monoclonal antibodies may also be obtained, for example, using phage or yeast-based libraries. See e.g., U.S. Pat. Nos. 8,258,082 and 8,691,730, each of which is incorporated by reference in its entirety.

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice 3^{rd} ed. (1986) Academic Press, San Diego, CA, incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, CA), and SP-2 or X63-Ag8-653 cells (available from the American Type Culture Collection, Rockville, MD). Human myeloma and mouse-human heteromyeloma cell lines also have been described for the production of human monoclonal antibodies. See e.g., Kozbor, J. Immunol., 1984, 133:3001, incorporated by reference in its entirety.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

### 8.3. Humanized Antibodies

Humanized antibodies may be generated by replacing most, or all, of the structural portions of a non-human monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad. Sci. U.S.A., 1998, 95:8910-8915; Steinberger et al., J. Biol. Chem., 2000, 275:36073-36078; Queen et al., Proc. Natl. Acad. Sci. U.S.A., 1989, 86:10029-10033; and U.S. Pat. Nos. 5,585,089, 5,693,761, 5,693,762, and 6,180,370; each of which is incorporated by reference in its entirety.

### 8.4. Human Antibodies

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807; each of which is incorporated by reference in its entirety. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905; each of which is incorporated by reference in its entirety). Human antibodies may also be generated by in vitro activated B cells (see e.g., U.S. Pat. Nos. 5,567,610 and 5,229,275, each of which is incorporated by reference in its entirety). Human antibodies may also be derived from yeast-based libraries (see e.g., U.S. Pat. No. 8,691,730, incorporated by reference in its entirety).

### 9. Vectors, Host Cells, and Recombinant Methods

The invention also provides isolated nucleic acids encoding anti-BCMA antibodies, vectors and host cells comprising the nucleic acids, and recombinant techniques for the production of the antibodies.

For recombinant production of the antibody, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615, incorporated by reference in its entirety.

Illustrative examples of suitable host cells are provided below. These host cells are not meant to be limiting.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W3110 are suitable.

In addition to prokaryotes, eukaryotic microbes such as filamentous fungi or yeast are also suitable cloning or expression hosts for anti-BCMA antibody-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the anti-BCMA antibody of this invention may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. For example, Carter et al. (Bio/Technology, 1992, 10:163-167) describes a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the antibody is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the antibody may be useful, for example, where the antibody accumulates in a cell as an insoluble aggregate, or where yields from periplasmic expression are low.

Where the antibody is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth., 1983, 62:1-13, incorporated by reference in its entirety). Protein G is useful for all mouse isotypes and for human γ3 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly (styrenedivinyl) benzene allow for faster flow rates and shorter processing times than can be achieved with agarose. Where the antibody comprises a CH3 domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the antibody of interest and contaminants may be subjected to low pH hydrophobic interaction chromatography using an elution buffer at a pH between about 2.5 to about 4.5, generally performed at low salt concentrations (e.g., from about 0 to about 0.25 M salt).

### 10. Pharmaceutical Compositions and Methods of Administration

Any of the antibodies provided herein can be provided in any appropriate pharmaceutical composition and be administered by any suitable route of administration. Suitable routes of administration include, but are not limited to, the inhalation, intraarterial, intradermal, intramuscular, intraperitoneal, intravenous, nasal, parenteral, pulmonary, and subcutaneous routes.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, olcyl alcohol, simethicone, and combinations thereof.

In some embodiments, the pharmaceutical composition comprises a cosolvent. Illustrative examples of cosolvents include ethanol, poly (ethylene) glycol, butylene glycol, dimethylacetamide, glycerin, and propylene glycol.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In some embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl bchenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene (glycol) succinate.

In some embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press, incorporated by reference in its entirety.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects, the microparticles or nanoparticles are micelles, liposomes, or polymersomes.

Further provided herein are anhydrous pharmaceutical compositions and dosage forms comprising an antibody, since water can facilitate the degradation of some antibodies.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

In some embodiments lactose-free compositions are provided herein which comprise excipients that are well known in the art and are listed, for example, in the U.S. Pharmocopia (USP) SP (XXI)/NF (XVI). In general, lactose-free compositions comprise an active ingredient, a binder/filler, and a lubricant in pharmaceutically compatible and pharmaceutically acceptable amounts. Exemplary lactose-free dosage forms comprise an active ingredient, microcrystalline cellulose, pre gelatinized starch, and magnesium stearate.

Also provided are pharmaceutical compositions and dosage forms that comprise one or more excipients that reduce the rate by which an antibody will decompose. Such excipients, which are referred to herein as “stabilizers,” include, but are not limited to, antioxidants such as ascorbic acid, pH buffers, or salt buffers.

### 10.1. Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the antibodies disclosed herein can also be incorporated into the parenteral dosage forms.

### 10.2. Dosage and Unit Dosage Forms

In human therapeutics, the doctor will determine the posology which he considers most appropriate according to a preventive or curative treatment and according to the age, weight, condition and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibodies.

The amount of the antibody or composition which will be effective in the prevention or treatment of a disorder or one or more symptoms thereof will vary with the nature and severity of the disease or condition, and the route by which the antibody is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the antibody per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). In certain embodiment, the dosage of the antibody provided herein, based on weight of the antibody, administered to prevent, treat, manage, or ameliorate a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the composition or a composition provided herein administered to prevent, treat, manage, or ameliorate a disorder, or one or more symptoms thereof in a subject is 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.25 mg to 2.5 mg, 0.5 mg to 20 mg, 0.5 to 15 mg, 0.5 to 12 mg, 0.5 to 10 mg, 0.5 mg to 7.5 mg, 0.5 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

The dose can be administered according to a suitable schedule, for example, once, two times, three times, or for times weekly. It may be necessary to use dosages of the antibody outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the antibodies provided herein are also encompassed by the herein described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of an antibody or composition provided herein followed by one or more maintenance doses.

In certain embodiments, a dose of an antibody or composition provided herein can be administered to achieve a steady-state concentration of the antibody in blood or serum of the subject. The steady-state concentration can be determined by measurement according to techniques available to those of skill or can be based on the physical characteristics of the subject such as height, weight and age.

In certain embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least 1 day, 2 days, 3 days, 5 days, 10 days, 15 days, 30 days, 45 days, 2 months, 75 days, 3 months, or 6 months.

### 11. Therapeutic Applications

For therapeutic applications, the antibodies of the invention are administered to a mammal, generally a human, in a pharmaceutically acceptable dosage form such as those known in the art and those discussed above. For example, the antibodies of the invention may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The antibodies also are suitably administered by peritumoral, intralesional, or perilesional routes, to exert local as well as systemic therapeutic effects. The intraperitoneal route may be particularly useful, for example, in the treatment of ovarian tumors.

The antibodies provided herein may be useful for the treatment of any disease or condition involving BCMA. In some embodiments, the disease or condition is a disease or condition that can be diagnosed by overexpression of BCMA. In some embodiments, the disease or condition is a disease or condition that can benefit from treatment with an anti-BCMA antibody. In some embodiments, the disease or condition is a cancer. In some embodiments, the disease or condition is a leukemia, a lymphoma, or multiple myeloma.

Any suitable cancer may be treated with the antibodies provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In some embodiments, the disease to be treated with the antibodies provided herein is gastric cancer, colorectal cancer, renal cell carcinoma, cervical cancer, non-small cell lung carcinoma, ovarian cancer, uterine cancer, endometrial carcinoma, prostate cancer, breast cancer, head and neck cancer, brain carcinoma, liver cancer, pancreatic cancer, mesothelioma, and/or a cancer of epithelial origin. In particular embodiments, the disease is colorectal cancer. In some embodiments, the disease is ovarian cancer. In some embodiments, the disease is breast cancer. In some embodiments, the disease is lung cancer. In some embodiments, the disease is head and neck cancer. In some embodiments, the disease is renal cell carcinoma. In some embodiments, the disease is brain carcinoma. In some embodiments, the disease is endometrial carcinoma.

### 12. Diagnostic Applications

In some embodiments, the antibodies provided herein are used in diagnostic applications. For example, an anti-BCMA antibody may be useful in assays for BCMA protein. In some aspects the antibody can be used to detect the expression of BCMA in various cells and tissues. These assays may be useful, for example, in making a diagnosis and/or prognosis for a disease, such as a cancer, a leukemia, a lymphoma, or multiple myeloma.

In some diagnostic and prognostic applications, the antibody may be labeled with a detectable moiety. Suitable detectable moieties include, but are not limited to radioisotopes, fluorescent labels, and enzyme-substrate labels. In another embodiment, the anti-BCMA antibody need not be labeled, and the presence of the antibody can be detected using a labeled antibody which specifically binds to the anti-BCMA antibody.

### 13. Affinity Purification Reagents

The antibodies of the invention may be used as affinity purification agents. In this process, the antibodies may be immobilized on a solid phase such a resin or filter paper, using methods well known in the art. The immobilized antibody is contacted with a sample containing the BCMA protein (or fragment thereof) to be purified, and thereafter the support is washed with a suitable solvent that will remove substantially all the material in the sample except the BCMA protein, which is bound to the immobilized antibody. Finally, the support is washed with another suitable solvent, such as glycine buffer, pH 5.0 that will release the BCMA protein from the antibody.

### 14. Kits

In some embodiments, an anti-BCMA antibody provided herein is provided in the form of a kit, i.e., a packaged combination of reagents in predetermined amounts with instructions for performing a procedure. In some embodiments, the procedure is a diagnostic assay. In other embodiments, the procedure is a therapeutic procedure.

In some embodiments, the kit further comprises a solvent for the reconstitution of the anti-BCMA antibody. In some embodiments, the anti-BCMA antibody is provided in the form of a pharmaceutical composition.

### EXAMPLES

### Example 1

### Generation of Anti-BCMA Antibodies

### Generation and Phage Display Selection

Phage display was used to discover initial human antibody leads 2190-B01 and 2213-A06. Antibody Fab libraries were constructed using an optimized trastuzumab Fab sequence codon optimized in a modified, commercially available p3 phagemid vector (Antibody Design Labs). Briefly, the phagemid vector was modified to express Fab heavy chains as C-terminal p3 fusion proteins, and regulatory regions (start codons, restriction enzyme sites, periplasmic leader sequences) were optimized for Fab display levels. Libraries were constructed using a standard overlap extension PCR protocol with mutagenic primers targeting heavy chain complementary determining regions (CDRs). See Heckman and Pease, Nat. Protoc., 2007, 2:924-932. Libraries were rescued through electroporation in M13-K07 infected SS320 E. coli cells. Library selections were performed using standard phage display protocols. See Rajan & Sidhu, Methods Enzymol., 2012, 502:3-23; Marks & Bradbury, Methods Mol Biol., 2004, 248:161-76. Following multiple selection rounds, Fab heavy chain pools were transferred into cell-free expression vectors for expression as His6 and FLAG-tagged IgG1.

### Ribosome Display Selections

Ribosome display was used to discover initial human antibody leads 2137-A05 and 2137-C07. Ribosome display was also used to affinity mature 2137-A05, 2137-C05, 2190-B01, and 2213-A06 to generate improved derivatives 2265, 2288, 2290, and 2291 families, respectively.

Antibody Fab libraries were constructed using a standard overlap extension PCR protocol with mutagenic primers targeting complementary determining regions (CDRs). See Heckman & Pease, supra. Selections for novel antibodies were performed using standard ribosome display protocols. See Hanes & Pluckthun, Proc. Natl. Acad. Sci. U.S.A, 1997, 94:4937-4942. Specifically, Fab-based ribosome display selections were performed according to published protocols. See Stafford et al., 2014, Protein Eng. Des. Sel. 27:97-109; Dreier and Pluckthun, 2011, Methods Mol Biol 687:283-306. After multiple rounds of selection, the DNA from RT-PCR output was cloned into an optimized vector for cell-free expression using standard molecular biology techniques. See Yin et al., 2012, mAbs 4:217-225. All constructs were HIS- and FLAG-tagged to streamline purification and testing during screening.

### Chicken HybriFree and Humanization

HybriFree methods were performed as published by Kivi et al. to discover antibodies 9A8, 10G5, 11D6, 10F4, 11D11, 9A5, 9E12, 9H1, 10H1, and 10E10. See Kivi et al., 2014, BMC Biotech 16:2 (14 pages). Briefly, human BCMA extracellular domain fused to chicken Fc and a C-terminal His tag was cloned and expressed and purified using standard methods. Two chickens were immunized until an antibody positive titer was detected in the egg yolk as determined by an ELISA. After boosting, the spleens were isolated and used to extract mRNA. Antibodies were screened and sequenced using methods described by Kivi et al. (supra).

The CDRs for 11D6 were grafted onto human antibody frameworks VH3-30, VH3-7, Vk1-6, V11-51, V13-1, and V13-21 by standard methodology to yield h11D6 humanized antibodies. The CDRs for 10F4 were grafted onto human antibody frameworks VH3-23, VH3-30, VH3-21, Vk1-33, V11-51, V13-1, and V13-21 by standard methodology to yield h10F4 humanized antibodies. The CDRs for 10H1 were grafted onto human antibody frameworks VH3-15, VH3-23, VH3-30, VH3-74, Vk1-33, V11-51, V13-1, and V13-21 by standard methodology to yield h10H1 humanized antibodies. See, e.g., Kuramochi et al., 2014, Method in Molecular Biology 1060:123-137.

Exemplary antibodies are reported in Tables 5 and 6, below.

TABLE 5
Antibodies produced by ribosome and phage-display

| Antibody | V_{H} | SEQ ID NO. | V_{L} | SEQ ID NO. |
|---|---|---|---|---|
| 1 | 2137-C07 | 167 | Trastuzumab | 217 |
| 2 | 2265-F06 | 168 | Trastuzumab | 217 |
| 3 | 2265-F05 | 169 | Trastuzumab | 217 |
| 4 | 2265-F02 | 170 | Trastuzumab | 217 |
| 5 | 2265-B06 | 171 | Trastuzumab | 217 |
| 6 | 2265-A09 | 172 | Trastuzumab | 217 |
| 7 | 2265-F03 | 173 | Trastuzumab | 217 |
| 8 | 2265-E02 | 174 | Trastuzumab | 217 |
| 9 | 2265-D11 | 175 | Trastuzumab | 217 |
| 10 | 2265-D05 | 176 | Trastuzumab | 217 |
| 11 | 2265-C03 | 177 | Trastuzumab | 217 |
| 12 | 2265-C02 | 178 | Trastuzumab | 217 |
| 13 | 2265-A06 | 179 | Trastuzumab | 217 |
| 14 | 2137-A05 | 180 | Trastuzumab | 217 |
| 15 | 2288-A03 | 181 | Trastuzumab | 217 |
| 16 | 2190-B01 | 182 | Trastuzumab | 217 |
| 17 | 2290-G01 | 183 | Trastuzumab | 217 |
| 18 | 2290-D02 | 184 | Trastuzumab | 217 |
| 19 | 2290-C07 | 185 | Trastuzumab | 217 |
| 20 | 2290-D05 | 186 | Trastuzumab | 217 |
| 21 | 2290-C08 | 187 | Trastuzumab | 217 |
| 22 | 2290-A02 | 188 | Trastuzumab | 217 |
| 23 | 2213-A06 | 189 | Trastuzumab | 217 |
| 24 | 2291-G05 | 190 | Trastuzumab | 217 |
| 25 | 2291-E06 | 191 | Trastuzumab | 217 |
| 26 | 2291-D07 | 192 | Trastuzumab | 217 |
| 27 | 2291-F10 | 193 | Trastuzumab | 217 |
| 28 | 2291-A04 | 194 | Trastuzumab | 217 |
| 29 | 2291-A01 | 195 | Trastuzumab | 217 |

TABLE 6
Chicken Antibodies and Humanized Chicken Antibodies

| Antibody | V_{H} | SEQ ID NO. | V_{L} | SEQ ID NO. |
|---|---|---|---|---|
| 30 | 9A8 | 196 | 9A8 | 218 |
| 31 | 10G5 | 197 | 10G5 | 219 |
| 32 | 11D6 | 198 | 11D6 | 220 |
| 33 | h11D6-HC4 | 199 | h11D6-LC4 | 221 |
| 34 | h11D6-HC3 | 200 | h11D6-LC3 | 222 |
| 35 | h11D6-HC2 | 201 | h11D6-LC2 | 223 |
| 36 | h11D6-HC1 | 202 | h11D6-LC1 | 224 |
| 37 | 10F4 | 203 | 10F4 | 225 |
| 38 | h10F4-HC4 | 204 | h10F4-LC4 | 226 |
| 39 | h10F4-HC3 | 205 | h10F4-LC3 | 227 |
| 40 | h10F4-HC2 | 206 | h10F4-LC2 | 228 |
| 41 | h10F4-HC1 | 207 | h10F4-LC1 | 229 |
| 42 | 9A5 | 208 | 9A5 | 230 |
| 43 | 9E12 | 209 | 9E12 | 231 |
| 44 | 9H1 | 210 | 9H1 | 232 |
| 45 | 10H1 | 211 | 10H1 | 233 |
| 46 | 10E10 | 212 | 10E10 | 234 |
| 47 | h10H1-HC4 | 213 | h10H1-LC4 | 235 |
| 48 | h10H1-HC3 | 214 | h10H1-LC3 | 236 |
| 49 | h10H1-HC2 | 215 | h10H1-LC2 | 237 |
| 50 | h10H1-HC1 | 216 | h10H1-LC1 | 238 |

### Example 2

### Primary Screening of Antibodies

### Primary ELISA Screening of Antibody Variants

Libraries of antibody variants generated by selection workflow were transformed into E. coli and grown on agar plates with antibiotic (Kanamycin). Individual colonies were grown in liquid broth (TB+antibiotic Kanamycin), and used as a template for DNA amplification via rolling circle amplification (RCA). The variants were then expressed in a cell-free protein synthesis reaction as described. See Yin et al., mAbs, 2012, 4:217-225. Briefly, cell-free extracts were treated with 50 UM iodoacetamide for 30 min at RT (20° C.) and added to a premix containing cell-free components (see Cai et al., Biotechnol Prg, 2015, 3:823-831), 10% (v/v) RCA DNA template (approximately 10 μg/mL DNA) for HC variants of interest, and 2.5 μg/mL of the trastuzumab LC. 60 μL cell free (CF) reactions were incubated at 30° C. for 12 hr on a shaker at 650 rpm in 96-well plates. 400-1500 colonies were screened, depending on the predicted diversity of different selection campaigns. Following synthesis, each reaction was diluted 1:200 and tested for binding to human or cynomolgus BCMA-Fc protein by ELISA. Briefly, BCMA-Fc (R&D Systems, Minneapolis, MN) was coated to 384-well Maxisorp plates in 0.1M bicarbonate (pH 8.9) and blocked with 1% BSA in PBST. Antibodies from a 1:200 diluted CF reaction were incubated on the plates, washed, and detected with HRP-conjugated anti-human Fab antibodies (Jackson ImmunoResearch, West Grove, PA) and Pierce Pico Supersignal ELISA substrate (ThermoFisher Scientific).

### High-Throughput Cell Binding

A high-throughput primary screen was performed to rapidly assess cell binding of antibodies produced in small-scale (60 μL) cell-free reactions. In this screen, four components were combined in equal volumes to a final volume of 100 μL/well in a U-bottom 96-well plate (Greiner Cat #650201) or flat bottom 384-well plate (Greiner Cat #781201). These components are: 1) BCMA-expressing NCI-H929 cells diluted in assay buffer (1×PBS+0.2% BSA, sterile filtered) to achieve a final concentration of 500,000 cells/well, 2) BCMA-negative MOLT-4 cells stained with CellTrace Oregon Green (Invitrogen Cat #34555) and diluted in assay buffer to achieve a final concentration of 500,000 cells/well, 3) a 1:50 dilution of cell-free reaction producing the antibody of interest diluted in assay buffer, and 4) a secondary anti-human antibody (AlexaFluor 647 AffiniPure F(ab′)₂ Donkey anti-human IgG, Fc specific; Jackson ImmunoResearch Cat #709-606-098) diluted 1:100 in assay buffer. Plates were then incubated on ice for one hour. Cells were pelleted by spinning at 1500×g for 5 minutes and resuspended in assay buffer. High-throughput flow cytometry was then performed on resuspended cells on a FACS instrument (BD Biosciences FACSCanto II or BD Biosciences LSR II), and data was analyzed with FlowJo software. Antibody binding was assessed by the proportional level of secondary antibody signal (presumably due to binding to the antibody of interest) on NCIH929 BCMA-positive cells compared to the signal on MOLT-4 BCMA-negative cells.

### Example 3

### Secondary Screening of Antibodies

### Preparation of IgGs

The top leads from the initial round of screening were cultured and miniprepped via the Qiaprep 96 Turbo miniprep kit (Qiagen) according to manufacturer's instructions. 7.5 μg/mL miniprepped HC DNA and 2.5 μg/mL of the trastuzumab LC was added to 4 mL cell-free reactions and incubated overnight for 12 hr at 30° C., 650 rpm. Expressed variants from clarified cell-free reactions were purified via IMAC purification using a semi-automated high throughput batch purification method. Briefly, purifications were performed in a 96-well plate format where 50 μL/well of IMAC resin (Ni Sepharose High Performance, GE Healthcare) was equilibrated in IMAC binding buffer (50 mM Tris pH 8.0, 300 mM NaCl, 10 mM imidazole), incubated with 1 mL cell-free reaction for 15 minutes followed by two washes in IMAC binding buffer. His-tagged antibody variants were then eluted using 200 μL IMAC elution buffer (50 mM Tris pH 8.0, 300 mM NaCl, 500 mM imidazole) and buffer exchanged into PBS using a 96-well Zeba plate (7 kD MWCO, Thermofisher). Purified antibodies were quantified via high throughput capillary electrophoresis using the Labchip GXII (Perkin Elmer) against a Herceptin standard curve, according to manufacturer's instructions.

Preparation of scFvs

A single-chain antibody is made in either the V_{H}V_{L} or V_{L}V_{H} orientation with a linker sequence between the V_{H} and V_{L} domains. Typically scFv linkers are composed of (GGGGS) n repeats where n=3, 4, 5, or 6 for linkers of 15, 20, 25, or 30 residues respectively. For cell-free expression, an N-terminal Met is added, but for mammalian expression a leader peptide is added. On the C-terminal end of the scFv, an Fc sequence can be added to extend in vivo half-life or the scFv can be used directly. An optional linker sequence can be incorporated between the scFv and the Fc. An exemplary scFv-Fc linker sequence is AAGSDQEPKSS (SEQ ID NO: 247). C-terminal affinity tags can optionally be added to facilitate purification and assay development. An exemplary affinity tag is a C-terminal FlagHis tag GSGDYKDDDDKGSGHHHHHH (SEQ ID NO: 245). A stop codon is typically inserted at the end of the sequence. An exemplary scFv can include an N-terminal Met residue, a V_{H} domain, a GGGGGGGGSGGGGS (SEQ ID NO: 246) linker, a V_{L} domain, an AAGSDQEPKSS (SEQ ID NO: 247) linker, an Fc domain, a FlagHis tag, and a stop codon.

### Differential Scanning Fluorimetry

A protein thermal shift assay was carried out by mixing the protein to be assayed with an environmentally sensitive dye (SYPRO Orange, Life Technologies Cat #S-6650) in a phosphate buffered solution (PBS), and monitoring the fluorescence of the mixture in real time as it underwent controlled thermal denaturation. Protein solutions between 0.2-2 mg/mL were mixed at a 1:1 volumetric ratio with a 1:500 PBS-diluted solution of SYPRO Orange (SYPRO Orange stock dye is 5000×in DMSO). 10 μL aliquots of the protein-dye mixture were dispensed in quadruplicate in a 384-well microplate (Bio-Rad Cat #MSP-3852), and the plate was sealed with an optically clear sealing film (Bio-Rad Cat #MSB-1001) and placed in a 384-well plate real-time thermocycler (Bio-Rad CFX384 Real Time System). The protein-dye mixture was heated from 25° C. to 95° C., at increments of 0.1° C. per cycle (˜1.5° C. per minute), allowing 3 seconds of equilibration at each temperature before taking a fluorescence measurement. At the end of the experiment, the transition melting temperatures (TM1 and TM2) were determined using the Bio-Rad CFX manager software. TM1 represents the melting temperature of the Fc domain. TM2 represents the melting temperature of the Fab domain.

### Biacore Off-Rate and Kinetic Analysis

Anti-Fab or anti-Fc polyclonal antibodies were immobilized onto a CM5 chip (GE Life Sciences) using amine coupling chemistry (from Amine Coupling Kit, GE Life Sciences). The immobilization steps were carried out at a flow rate of 25 L/min in 1×HBS-EP+ buffer (GE Life Sciences; 10×Stock diluted before use). The sensor surfaces were activated for 7 min with a mixture of NHS (0.05 M) and EDC (0.2 M). The anti-Fab or anti-Fc antibodies were injected over all 4 flow cells at a concentration of 25 μg/ml in 10 mM sodium acetate, pH 4.5, for 7 min. Ethanolamine (1 M, pH 8.5) was injected for 7 min to block any remaining activated groups. An average of 12,000 response units (RU) of capture antibody was immobilized on each flow cell.

Off-rate and kinetic binding experiments were performed at 25° C. using 1×HBS-EP+ buffer. Test and control antibodies were injected over the anti-Fab or anti-Fc surface at concentrations of 5-10 μg/mL for 12 seconds at a flow rate of 10 L/min on flow cells 2, 3 and 4, followed by a buffer wash for 30 seconds at the same flow rate. Kinetic characterization of antibody samples was carried out with a range of antigen concentrations from 1-100 nM and 1 injection of 0 nM antigen (for example, 100, 50, 25, 6.25, 1.56 and 0 nM). After capturing ligand (antibody) on the anti-Fab or anti-Fc surface, the analyte (human BCMA-Fc, cyno BCMA-Fc, or human BCMA from R&D Systems, custom protein production, or Sigma Aldrich, respectively) was bound for 180 seconds, followed by a 600 second dissociation phase at a flow rate of 50 μL/min. Between each ligand capture and analyte binding cycle, regeneration was carried out using 2 injections of 10 mM glycine pH 2.0 for 30 seconds at 30 μL/min, followed by a 30 second buffer wash step.

The data was fit with the Biacore T200 Evaluation software, using a 1-1 Langmuir binding model. K_{D}) (affinity, nM) was determined as a ratio of the kinetic rate constants calculated from the fits of the association and dissociation phases.

### Cell Lines and Cell Culture Conditions

NCI-H929, U266B1, MOLT-4 and ARP-1, were obtained from ATCC and the Keats Lab (Tgen, Phoenix, AZ). 293T-cynoBCMA and 293T-ratBCMA recombinant cells were generated by transfecting 293T cells with a plasmid containing cynomolgus or rat BCMA cDNA sequences and selecting for the highest stable expression of cynomolgus BCMA or rat BCMA on the cell surface. NCI-H929, U266B1, and MOLT-4 cells were maintained in RPMI-1640 (Cellgro-Mediatech; Manassas, VA) supplemented with 20% heat-inactivated fetal bovine serum (Hyclone; Thermo Scientific; Waltham, MA), 1% Penicillin/Streptomycin (Cellgro-Mediatech; Manassas, VA), and 2 mmol/L-glutamax (Life Technology; Carlsbad, CA). 293T-cynoBCMA and 293T-ratBCMA cells were maintained in Ham's F-12-high glucose DMEM (50-50) (Cellgro-Mediatech; Manassas, VA) supplemented with 10% heat-inactivated fetal bovine serum (Hyclone; Thermo Scientific; Waltham, MA), 1% Penicillin/Streptomycin (Cellgro-Mediatech; Manassas, VA), and 2 mmol/L-glutamax (Life Technology; Carlsbad, CA).

### Cell Binding Experiments

Variants for which sufficient protein was purified in secondary screening were tested in a fluorescence-activated cell sorting (FACS) cell-binding assay. BCMA positive NCI-H929 and 293T-cynoBCMA cells and BCMA negative 293T cells were used to screen for FACS binders. 293T cells were treated with 1 μM DAPT 24 hours prior to cell binding to prevent BCMA shedding. 6-12 point dilutions of anti-BCMA variants starting from concentrations of about 100-200 nM antibody were dispensed into each well using a BioMekFX (Beckman Coulter). Cells were then incubated on ice for 1 hr, washed with FACS buffer and incubated for 1 hr on ice with 50 mL FACS buffer containing 2.5 g/ml Alexa647-conjugated Goat Anti-Human IgG dispensed using BioMekFX (Beckman Coulter). Cells were then washed 2× with FACS buffer and fixed for 10 minutes in 200 ml PBS with 2% PFA prior to fluorescence detection. Samples were acquired using a Beckton Dickinson LSRII FACS. Geometric Mean Fluorescence Intensity of BCMA antibody binding was analyzed using FlowJo® software (Trec Star, Inc.).

### Cell-Killing Analysis

The internalization of the antibodies was evaluated by drugs conjugated to secondary antibodies in a cell killing assay on BCMA positive cells. BCMA-positive cell lines ARP-1 and U266B1 were used to screen for internalizing leads. Cells were washed twice with calcium and magnesium-free Dulbecco's phosphate-buffered saline (DPBS), harvested with Accutase® (Innovative Cell Technologies; San Diego, CA) and counted by the Vi-CELL Cell Viability Analyzers (Beckman Coulter, Brea, CA). A total of 625 cells in a volume of 25 μL were seeded in each well of a 384-well half area flat bottom tissue culture-coated white polystyrene plate (Greiner Bio-One, Monroe, NC). Lead antibodies were formulated at 4×starting concentration in the cell culture medium and filtered through MultiScreenHTS 96-Well Filter Plates (Millipore; Billerica, MA). 12.5 μL of the serial diluted antibody (1:3 serial dilution starting from 100 nM) was added into treatment wells and 12.5 μL of an anti-human nanobody conjugated to SC239 (hemiasterlin via a cleavable linker) or SC225 (maytansinoid via a non-cleavable linker) was then added into each well at a fixed final concentration of 20 nM. Assay plates were cultured at 37° C. in a CO2 incubator for 72 hours before assay. For cell viability measurement, 30 μL of Cell Titer-Glo® reagent (Promega Corp. Madison, WI) was added into each well, and plates were processed as per product instructions. Relative luminescence was measured on an ENVISION® plate reader (Perkin-Elmer; Waltham, MA). Relative luminescence readings were converted to % viability using untreated cells as controls. Data was fitted with non-linear regression analysis, using a log (inhibitor) vs. response-variable slope, 4 parameter fit with GraphPad Prism (GraphPad v 5.0, Software; San Diego, CA). Data was expressed as relative cell viability (ATP content) % vs. dose of antibody.

### Example 4

### Characteristics of Illustrative Anti-BCMA Antibodies

Tables 7 through 9 show results obtained using the illustrative antibodies described herein. Tables 7 and 8 show results obtained with antibodies produced by ribosome and phage-display of initial leads. Table 9 shows results obtained with antibodies isolated from affinity maturation of initial antibody leads constructed with a trastuzumab light chain.

TABLE 7A
Antibodies from ribosome and phage-display.

| | | | | | | | U266B1, | |
|---|---|---|---|---|---|---|---|---|
| | NCI-H929 | | 293T- | | ARP-1, | | SC225-conjugated | |
| | (BCMA + cells) | | cynoBCMA | | SC225-conjugated 2° | | 2° antibody cell | |
| Fab-HC | cell binding | | cell binding | | antibody cell killing | | killing | |
| Variant | Bₘₐₓ | Kd | Bₘₐₓ | Kd | EC₅₀ | Span | EC₅₀ | Span |
| ID | (MFI) | (nM) | (MFI) | (nM) | (nM) | (%) | (nM) | (%) |
| SRP2137-C07 | — | — | 18519 | 10.1 | NK | NK | NK | NK |
| SRP2265-F06 | 11434 | 4.5 | 25786 | 3.4 | 1.2 | 62 | 0.9 | 59 |
| SRP2265-F05 | 12685 | 10.3 | 18484 | 3.0 | 1.8 | 52 | 1.2 | 61 |
| SRP2265-F02 | 12211 | 4.5 | 21843 | 5.0 | 1.6 | 62 | 1.0 | 62 |
| SRP2265-B06 | 11856 | 6.2 | 26591 | 3.4 | 1.6 | 67 | 0.6 | 59 |
| SRP2265-A09 | 11728 | 5.2 | 23759 | 6.2 | 1.9 | 55 | 0.9 | 58 |
| SRP2265-F03 | 12522 | 5.3 | — | — | 2.3 | 57 | 0.8 | 56 |
| SRP2265-E02 | 11629 | 4.5 | 26694 | 5.1 | 1.5 | 55 | 0.8 | 53 |
| SRP2265-D11 | 9617 | 4.4 | 21925 | 2.5 | 1.9 | 51 | 1.0 | 54 |
| SRP2265-D05 | 10944 | 4.0 | 21324 | 3.8 | 1 | 50 | 0.8 | 53 |
| SRP2265-C03 | 11519 | 4.4 | 24116 | 4.9 | 2.2 | 70 | 1.1 | 61 |
| SRP2265-C02 | 11248 | 4.3 | 17431 | 2.5 | 1.5 | 55 | 0.7 | 51 |
| SRP2265-A06 | 11072 | 5.7 | 22100 | 3.8 | 2.9 | 68 | 1.3 | 59 |

NK = no killing

TABLE 7B
Antibodies from ribosome and phage-display.

| | Thermo- | | | | | | |
|---|---|---|---|---|---|---|---|
| | stability | Biacore, human BCMA-Fc | | | Biacore, cyno BCMA-Fc | | |
| Fab-HC | Fab TM2 | kₐ | | K_{D} | kₐ | | K_{D} |
| Variant ID | (° C.) | (1/Ms) | k_{d} (1/s) | (M) | (1/Ms) | k_{d} (1/s) | (M) |
| SRP2137-C07 | 88.9 | 4.57E+05 | 7.45E−04 | 1.63E−09 | ND | ND | ND |
| SRP2265-F06 | 87.0 | 6.99E+05 | 1.93E−04 | 2.76E−10 | 3.47E+05 | 1.33E−03 | 3.84E−09 |
| SRP2265-F05 | 86.9 | 5.12E+05 | 2.40E−04 | 4.69E−10 | 2.60E+05 | 1.14E−03 | 4.39E−09 |
| SRP2265-F02 | 85.0 | 5.87E+05 | 2.93E−04 | 4.99E−10 | 3.15E+05 | 1.16E−03 | 3.68E−09 |
| SRP2265-B06 | 86.7 | 7.05E+05 | 3.85E−04 | 5.46E−10 | 3.54E+05 | 1.30E−03 | 3.67E−09 |
| SRP2265-A09 | 86.0 | 5.39E+05 | 3.18E−04 | 5.89E−10 | 2.70E+05 | 1.73E−03 | 6.43E−09 |
| SRP2265-F03 | 83.3 | 7.49E+05 | 4.74E−04 | 6.33E−10 | 3.46E+05 | 2.74E−03 | 7.90E−09 |
| SRP2265-E02 | 87.3 | 5.71E+05 | 3.83E−04 | 6.70E−10 | 2.66E+05 | 1.53E−03 | 5.75E−09 |
| SRP2265-D11 | 87.5 | 7.66E+05 | 5.37E−04 | 7.01E−10 | 3.58E+05 | 1.16E−03 | 3.24E−09 |
| SRP2265-D05 | 87.0 | 6.91E+05 | 4.85E−04 | 7.02E−10 | 3.25E+05 | 1.47E−03 | 4.53E−09 |
| SRP2265-C03 | 88.0 | 5.87E+05 | 4.25E−04 | 7.24E−10 | 3.04E+05 | 1.77E−03 | 5.83E−09 |
| SRP2265-C02 | 87.3 | 7.47E+05 | 5.67E−04 | 7.58E−10 | 3.25E+05 | 1.37E−03 | 4.22E−09 |
| SRP2265-A06 | 86.6 | 4.97E+05 | 3.92E−04 | 7.89E−10 | 2.49E+05 | 1.56E−03 | 6.24E−09 |

ND = not detected

TABLE 8A
Antibodies from ribosome and phage-display.

| | | | | | U266B1, | |
|---|---|---|---|---|---|---|
| | NCI-H929 (BCMA + | | 293T-cynoBCMA cell | | SC225-conjugated 2° | |
| | cells) cell binding | | binding | | antibody cell killing | |
| Fab-HC | Bₘₐₓ | Kd | Bₘₐₓ | Kd | EC₅₀ | |
| Variant ID | (MFI) | (nM) | (MFI) | (nM) | (nM) | Span (%) |
| SRP2137-A05 | 2689 | 10.0 | 1906 | 18.3 | NK | NK |
| SRP2288-A03 | 16291 | 4.8 | 46759 | 1.8 | 2.3 | 42 |
| SRP2190-B01 | NSB | NSB | NSB | NSB | NK | NK |
| SRP2290-C08 | 37810 | 11.0 | 70959 | 2.3 | 1.1 | 73 |
| SRP2290-G01 | NSB | NSB | 77116 | 4.7 | 1.4 | 70 |
| SRP2290-A02 | 33585 | 4.9 | 70302 | 2.3 | 1.6 | 70 |
| SRP2290-C07 | NSB | NSB | 78098 | 6.7 | 0.93 | 75 |
| SRP2290-D05 | 21841 | 4.6 | 57580 | 1.7 | 0.96 | 50 |
| SRP2290-D02 | 33647 | 19.2 | 75785 | 3.5 | 1.4 | 60 |
| SRP2213-A06 | 2506 | 5.3 | 3622 | 15.1 | NK | NK |
| SRP2291-D07 | 28671 | 3.8 | 67897 | 0.5 | 16 | 2.0 |
| SRP2291-G05 | 23164 | 1.4 | 62738 | 1.0 | 20 | 0.8 |
| SRP2291-E06 | 34417 | 5.8 | 68318 | 1.4 | 27 | 1.1 |
| SRP2291-F10 | 33846 | 5.4 | 66502 | 0.7 | 46 | 1.0 |
| SRP2291-A04 | 33916 | 5.0 | 63767 | 1.2 | 49 | 0.8 |
| SRP2291-A01 | 32503 | 4.9 | 67229 | 1.0 | 58 | 1.1 |

NK = no killing
NSB = non-saturating binding

TABLE 8B
Antibodies from ribosome and phage-display.

| | huBCMA-Fc | | cynoBCMA-Fc | | Biacore, human BCMA-Fc | | | Thermo- |
|---|---|---|---|---|---|---|---|---|
| | ELISA | | ELISA | | kinetics | | | stability |
| Fab-HC | | EC₅₀ | | EC₅₀ | kₐ | | K_{D} | Fab TM2 |
| Variant ID | Bₘₐₓ | (nM) | Bₘₐₓ | (nM) | (1/Ms) | k_{d} (1/s) | (M) | (° C.) |
| SRP2137-A05 | 3302000 | 0.52 | 2198000 | 419.2 | 1.41E+06 | 3.32E−03 | 2.36E−09 | 85.6 |
| SRP2288-A03 | 4245000 | 0.56 | 3022000 | 0.57 | 9.27E+05 | 5.16E−04 | 5.57E−10 | 81.1 |
| SRP2190-B01 | 3678000 | 0.3 | 2154000 | 19.3 | 1.36E+05 | 7.12E−04 | 5.23E−09 | 75.3 |
| SRP2290-C08 | 4334000 | 0.3 | 4135000 | 0.2 | 6.66E+05 | 2.52E−04 | 3.78E−10 | 80.3 |
| SRP2290-G01 | 4828000 | 0.3 | 4599000 | 0.3 | 7.14E+05 | 3.18E−04 | 4.45E−10 | 70.9 |
| SRP2290-A02 | 4871000 | 0.4 | 4471000 | 0.4 | 2.31E+05 | 1.31E−04 | 5.67E−10 | 84.8 |
| SRP2290-C07 | 4116000 | 0.2 | 3810000 | 0.2 | 3.61E+05 | 2.21E−04 | 6.12E−10 | 74.3 |
| SRP2290-D05 | 3943000 | 0.3 | 3606000 | 0.2 | 4.76E+05 | 4.46E−04 | 9.37E−10 | 81.2 |
| SRP2290-D02 | 4539000 | 0.3 | 3913000 | 0.2 | 2.92E+05 | 2.77E−04 | 9.49E−10 | 85.6 |
| SRP2213-A06 | 3685000 | 0.2 | 2230000 | 39.04 | 1.36E+06 | 2.83E−03 | 2.08E−09 | 87 |
| SRP2291-D07 | 4080000 | 0.03 | 3942000 | 0.03 | 4.37E+05 | 4.14E−04 | 9.48E−10 | 84.1 |
| SRP2291-G05 | 3906000 | 0.13 | 3385000 | 0.09 | 4.40E+05 | 4.23E−04 | 9.62E−10 | 85.7 |
| SRP2291-E06 | 4107000 | 0.21 | 3360000 | 0.13 | 4.68E+05 | 3.57E−04 | 7.63E−10 | 84.6 |
| SRP2291-F10 | 3724000 | 0.1 | 3432000 | 0.06 | 5.41E+05 | 9.65E−05 | 1.78E−10 | 84.7 |
| SRP2291-A04 | 4604000 | 0.23 | 4227000 | 0.2 | 4.87E+05 | 3.49E−04 | 7.16E−10 | 86.7 |
| SRP2291-A01 | 4999000 | 0.39 | 4772000 | 0.33 | 6.44E+05 | 2.82E−05 | 4.38E−11 | 82.3 |

TABLE 9A
Chicken HybriFree-derived antibodies.

| | NCI-H929 | | | | | | | | |
|---|---|---|---|---|---|---|---|---|---|
| | (BCMA + cells) | | 293T-cynoBCMA | | 293T-ratBCMA | | | | |
| | cell binding | | cell binding | | cell binding | | Biacore, human BCMA-Fc kinetics | | |
| Fab-HC | Bₘₐₓ | Kd | Bₘₐₓ | Kd | Bₘₐₓ | Kd | kₐ | | K_{D} |
| Variant ID | (MFI) | (nM) | (MFI) | (nM) | (MFI) | (nM) | (1/Ms) | k_{d} (1/s) | (M) |
| 9A8 | 21148 | 32.99 | 17799 | 8.87 | NB | NB | not determined | not determined | not determined |
| 10G5 | 25759 | 65.84 | 24439 | 7.33 | NB | NB | not determined | not determined | not determined |
| 11D6 | 26365 | 47.55 | 27672 | 12.17 | NB | NB | 1.02E+06 | 3.18E−04 | 3.13E−10 |
| 10F4 | 18715 | 10.51 | 12768 | 5.94 | 410 | 5.47 | not detected | not detected | not detected |
| 9A5 | 19686 | 1.01 | 18234 | 0.64 | 2780 | 7.13 | not determined | not determined | not determined |
| 9E12 | 8137 | 0.52 | 13204 | 0.34 | 4692 | 2.61 | not determined | not determined | not determined |
| 9H1 | 6156 | 1 | 11443 | 0.39 | 3452 | 1.32 | not determined | not determined | not determined |
| 10H1 | 22498 | 0.36 | 21201 | 0.25 | 4353 | 0.39 | 6.14E+09 | 1.79E+01 | 2.92E−09 |
| 10E10 | 3435 | 4.67 | 4543 | 18.91 | 906 | 6.83 | not determined | not determined | not determined |

TABLE 9B
Chicken HybriFree-derived antibodies.

| | ARP-1, | | U266B1, | |
|---|---|---|---|---|
| | SC239-conjugated 2° | | SC239-conjugated 2° | |
| | antibody cell killing | | antibody cell killing | |
| Fab-HC | EC50 | Span | EC50 | Span |
| Variant ID | (nM) | (%) | (nM) | (%) |
| 9A8 | 2.4 | 82 | 2.3 | 42 |
| 10G5 | 3.5 | 82 | 1.9 | 63 |
| 11D6 | 3.8 | 83 | 1.1 | 67 |
| 10F4 | 1 | 87 | 0.36 | 81 |
| 9A5 | 0.7 | 93 | 0.29 | 84 |
| 9E12 | 1.2 | 80 | 1.4 | 47 |
| 9H1 | 1.3 | 74 | 1.1 | 50 |
| 10H1 | 0.86 | 88 | 0.29 | 89 |
| 10E10 | 3 | 46 | 2.5 | 20 |

### Example 5

### Sequences

Table 10 provides sequences referred to herein.

TABLE 10
Sequences

| SEQ ID | | | | |
|---|---|---|---|---|
| NO: | Molecule | Region | Scheme | Sequence |
| 1 | Human BCMA | | | MLQMAGQCSQNEYFDSLLHACIPCQLRC |
| | (Isoform 1, | | | SSNTPPLTCQRYCNASVTNSVKGTNAIL |
| | UniprotKB- | | | WTCLGLSLIISLAVFVLMFLLRKINSEP |
| | Q02223) | | | LKDEFKNTGSGLLGMANIDLEKSRTGDE |
| | | | | IILPRGLEYTVEECTCEDCIKSKPKVDS |
| | | | | DHCFPLPAMEEGATILVTTKTNDYCKSL |
| | | | | PAALSATEIEKSISAR |
| 2 | Human BCMA | | | MLQMAGQCSQNEYFDSLLHACIPCQLRC |
| | (Isoform 2, | | | SSNTPPLTCQRYCNARSGLLGMANIDLE |
| | UniprotKB- | | | KSRTGDEIILPRGLEYTVEECTCEDCIK |
| | Q02223) | | | SKPKVDSDHCFPLPAMEEGATILVTTKT |
| | | | | NDYCKSLPAALSATEIEKSISAR |
| 3 | Cynomolgus | | | MLQMARQCSQNEYFDSLLHDCKPCQLRC |
| | BCMA (Predicted | | | SSTPPLTCQRYCNASMTNSVKGMNAILW |
| | NCBI Reference | | | TCLGLSLIISLAVFVLTFLLRKMSSEPL |
| | Sequence: | | | KDEFKNTGSGLLGMANIDLEKGRTGDEI |
| | XP_001106892.1) | | | VLPRGLEYTVEECTCEDCIKNKPKVDSD |
| | | | | HCFPLPAMEEGATILVTTKTNDYCNSLS |
| | | | | AALSVTEIEKSISAR |
| 4 | Murine BCMA | | | MAQQCFHSEYFDSLLHACKPCHLRCSNP |
| | (NBCI Reference | | | PATCQPYCDPSVTSSVKGTYTVLWIFLG |
| | Sequence: | | | LTLVLSLALFTISFLLRKMNPEALKDEP |
| | NP_035738.1) | | | QSPGQLDGSAQLDKADTELTRIRAGDDR |
| | | | | IFPRSLEYTVEECTCEDCVKSKPKGDSD |
| | | | | HFFPLPAMEEGATILVTTKTGDYGKSSV |
| | | | | PTALQSVMGMEKPTHTR |
| 5 | 2137-C07 | CDR-H1 | Chothia | GFNISGS |
| 6 | 2265-F06 | CDR-H1 | Chothia | GFNISYP |
| 7 | 2265-F05 | CDR-H1 | Chothia | GFNIIAP |
| 8 | 2265-F02 | CDR-H1 | Chothia | GFNISAP |
| 9 | 2265-B06 | CDR-H1 | Chothia | GFNIRVS |
| 10 | 2265-A09 | CDR-H1 | Chothia | GFNIIGP |
| 11 | 2265-F03 | CDR-H1 | Chothia | GFNIRGP |
| 12 | 2265-E02 | CDR-H1 | Chothia | GFNIYVS |
| 13 | 2265-D11 | CDR-H1 | Chothia | GFNISGP |
| 14 | 2265-C03 | CDR-H1 | Chothia | GFNISVP |
| 15 | 2265-C02 | CDR-H1 | Chothia | GFNIGVS |
| 16 | 2265-A06 | CDR-H1 | Chothia | GFNIYRS |
| 17 | 2137-A05 | CDR-H1 | Chothia | GFNINNS |
| 18 | 2190-B01 | CDR-H1 | Chothia | GFNISSY |
| 19 | 2290-G01 | CDR-H1 | Chothia | GFNISPY |
| 20 | 2290-C07 | CDR-H1 | Chothia | GFNITYD |
| 21 | 2290-D05 | CDR-H1 | Chothia | GFNIASR |
| 22 | 2290-C08 | CDR-H1 | Chothia | GFNIQPY |
| 23 | 2290-A02 | CDR-H1 | Chothia | GFNISTR |
| 24 | 2291-G05 | CDR-H1 | Chothia | GFNIAAY |
| 25 | 2291-D07 | CDR-H1 | Chothia | GFNIKDT |
| 26 | 2291-F10 | CDR-H1 | Chothia | GFNIDPY |
| 27 | 9A8 | CDR-H1 | Chothia | GFTFSSF |
| 28 | 10F4 | CDR-H1 | Chothia | GFTFSGY |
| 29 | 9A5 | CDR-H1 | Chothia | GFSISDY |
| 30 | 9E12 | CDR-H1 | Chothia | GFTFSDY |
| 31 | 10E10 | CDR-H1 | Chothia | GFTFSSY |
| 32 | 2137-C07 | CDR-H1 | Kabat | GSGIH |
| 33 | 2265-F06 | CDR-H1 | Kabat | YPGIH |
| 34 | 2265-F05 | CDR-H1 | Kabat | APGIH |
| 35 | 2265-B06 | CDR-H1 | Kabat | VSGIH |
| 36 | 2265-A09 | CDR-H1 | Kabat | GPGIH |
| 37 | 2265-C03 | CDR-H1 | Kabat | VPGIH |
| 38 | 2265-A06 | CDR-H1 | Kabat | RSGIH |
| 39 | 2137-A05 | CDR-H1 | Kabat | NSYIH |
| 40 | 2288-A03 | CDR-H1 | Kabat | NSWIH |
| 41 | 2190-B01 | CDR-H1 | Kabat | SYWIH |
| 42 | 2290-G01 | CDR-H1 | Kabat | PYWIH |
| 43 | 2290-C07 | CDR-H1 | Kabat | YDWIH |
| 44 | 2290-D05 | CDR-H1 | Kabat | SRWIH |
| 45 | 2290-A02 | CDR-H1 | Kabat | TRWIH |
| 46 | 2213-A06 | CDR-H1 | Kabat | SYAIH |
| 47 | 2291-G05 | CDR-H1 | Kabat | AYTIH |
| 48 | 2291-E06 | CDR-H1 | Kabat | PYTIH |
| 49 | 2291-D07 | CDR-H1 | Kabat | DTYIH |
| 50 | 2291-A04 | CDR-H1 | Kabat | SYGIH |
| 51 | 9A8 | CDR-H1 | Kabat | SFNMF |
| 52 | 10F4 | CDR-H1 | Kabat | GYNMG |
| 53 | 9A5 | CDR-H1 | Kabat | DYGMG |
| 54 | 9E12 | CDR-H1 | Kabat | DYGLG |
| 55 | 10H1 | CDR-H1 | Kabat | GYGMG |
| 56 | 10E10 | CDR-H1 | Kabat | SYGMG |
| 57 | 2137-C07 | CDR-H2 | Chothia | NPAGGY |
| 58 | 2265-F03 | CDR-H2 | Chothia | SPAAGY |
| 59 | 2265-D11 | CDR-H2 | Chothia | NPAAGY |
| 60 | 2137-A05 | CDR-H2 | Chothia | YPYSGY |
| 61 | 2288-A03 | CDR-H2 | Chothia | YPYIGF |
| 62 | 2190-B01 | CDR-H2 | Chothia | TPSGGY |
| 63 | 2290-G01 | CDR-H2 | Chothia | TPPSGF |
| 64 | 2290-D02 | CDR-H2 | Chothia | TPAAGY |
| 65 | 2290-C07 | CDR-H2 | Chothia | TPFDGY |
| 66 | 2290-D05 | CDR-H2 | Chothia | TPSAGY |
| 67 | 2290-C08 | CDR-H2 | Chothia | TPPSGY |
| 68 | 2213-A06 | CDR-H2 | Chothia | SPYGGY |
| 69 | 2291-E06 | CDR-H2 | Chothia | FPSGGY |
| 70 | 2291-D07 | CDR-H2 | Chothia | SPYDGY |
| 71 | 9A8 | CDR-H2 | Chothia | RNDGNS |
| 72 | 10G5 | CDR-H2 | Chothia | SNDGSS |
| 73 | 11D6 | CDR-H2 | Chothia | RNDGRS |
| 74 | 10F4 | CDR-H2 | Chothia | TYGTGSY |
| 75 | 9A5 | CDR-H2 | Chothia | DHDGRY |
| 76 | 9E12 | CDR-H2 | Chothia | NSAGSG |
| 77 | 9H1 | CDR-H2 | Chothia | NSAGSD |
| 78 | 10E10 | CDR-H2 | Chothia | NSGGSSY |
| 79 | 2137-C07 | CDR-H2 | Kabat | FINPAGGYTDYADSVKG |
| 80 | 2265-F03 | CDR-H2 | Kabat | FISPAAGYTDYADSVKG |
| 81 | 2265-D11 | CDR-H2 | Kabat | FINPAAGYTDYADSVKG |
| 82 | 2137-A05 | CDR-H2 | Kabat | WIYPYSGYTNYADSVKG |
| 83 | 2288-A03 | CDR-H2 | Kabat | WIYPYIGFTEYADSVKG |
| 84 | 2190-B01 | CDR-H2 | Kabat | VITPSGGYTYYADSVKG |
| 85 | 2290-G01 | CDR-H2 | Kabat | VITPPSGFTYYADSVKG |
| 86 | 2290-D02 | CDR-H2 | Kabat | vITPAAGYTYYADSVKG |
| 87 | 2290-C07 | CDR-H2 | Kabat | VITPFDGYTYYADSVKG |
| 88 | 2290-D05 | CDR-H2 | Kabat | VITPSAGYTYYADSVKG |
| 89 | 2290-C08 | CDR-H2 | Kabat | VITPPSGYTYYADSVKG |
| 90 | 2290-A02 | CDR-H2 | Kabat | VITPSAGYTHYADSVKG |
| 91 | 2213-A06 | CDR-H2 | Kabat | VISPYGGYTEYADSVKG |
| 92 | 2291-G05 | CDR-H2 | Kabat | WISPYGGYTEYADSVKG |
| 93 | 2291-E06 | CDR-H2 | Kabat | HIFPSGGYTDYADSVKG |
| 94 | 2291-D07 | CDR-H2 | Kabat | v1SPYDGYTEYADSVKG |
| 95 | 2291-F10 | CDR-H2 | Kabat | WISPYDGYTEYADSVKG |
| 96 | 2291-A04 | CDR-H2 | Kabat | FISPYDGYTEYADSVKG |
| 97 | 2291-A01 | CDR-H2 | Kabat | HISPYDGYTDYADSVKG |
| 98 | 9A8 | CDR-H2 | Kabat | YIRNDGNSASYGPAVKG |
| 99 | 10G5 | CDR-H2 | Kabat | YISNDGSSTSYGPAVKG |
| 100 | 11D6 | CDR-H2 | Kabat | YIRNDGRSTSYGPAVKG |
| 101 | h11D6-Hc4 | CDR-H2 | Kabat | YIRNDGRSTSYVDSVKG |
| 102 | h11D6-Hc3 | CDR-H2 | Kabat | YIRNDGRSTSYADSVKG |
| 103 | h11D6-Hc1 | CDR-H2 | Kabat | YIRNDGRSTSYAAPVKG |
| 104 | 10F4 | CDR-H2 | Kabat | GITYGTGSYTAYGAAVKG |
| 105 | h10F4-Hc4 | CDR-H2 | Kabat | GITYGTGSYTAYVDSVKG |
| 106 | h10F4-Hc3 | CDR-H2 | Kabat | GITYGTGSYTAYADSVKG |
| 107 | h10F4-Hc1 | CDR-H2 | Kabat | GITYGTGSYTAYAAPVKG |
| 108 | 9A5 | CDR-H2 | Kabat | RIDHDGRYTDYGAVVKG |
| 109 | 9E12 | CDR-H2 | Kabat | RINSAGSGTYYGSAVDG |
| 110 | 9H1 | CDR-H2 | Kabat | RINSAGSDTNYGSAVKG |
| 111 | 11H1 | CDR-H2 | Kabat | RINSAGSDTDYGAAVKG |
| 112 | 10E10 | CDR-H2 | Kabat | RINSGGSSYTDYGSAVKG |
| 113 | h10H1-Hc4 | CDR-H2 | Kabat | RINSAGSDTDYVDSVKG |
| 114 | h10H1-Hc3 | CDR-H2 | Kabat | RINSAGSDTDYADSVKG |
| 115 | h10H1-Hc1 | CDR-H2 | Kabat | RINSAGSDTDYAAPVKG |
| 116 | 2137-C07 | CDR-H3 | | DYVYQYWTYVLDY |
| 117 | 2265-F06 | CDR-H3 | | DYILQYWTYVLDY |
| 118 | 2265-F05 | CDR-H3 | | DYVNAYWTYVLDY |
| 119 | 2265-F02 | CDR-H3 | | DYIRQYWTYVLDY |
| 120 | 2265-B06 | CDR-H3 | | DYVQAYWTYVLDY |
| 121 | 2265-A09 | CDR-H3 | | DYVYNYWTYVLDY |
| 122 | 2265-F03 | CDR-H3 | | DFVQSYWTYVLDY |
| 123 | 2265-D11 | CDR-H3 | | DYIYQYWTYVLDY |
| 124 | 2265-D05 | CDR-H3 | | DFVYAYWTYVLDY |
| 125 | 2265-C03 | CDR-H3 | | DYVPQYWTYVLDY |
| 126 | 2265-C02 | CDR-H3 | | DYIYSYWTYVLDY |
| 127 | 2137-A05 | CDR-H3 | | DYGPWYGTGVLDY |
| 128 | 2288-A03 | CDR-H3 | | DYDLRYLTGVLDY |
| 129 | 2190-B01 | CDR-H3 | | DLGGGYWVGFFDY |
| 130 | 2290-G01 | CDR-H3 | | DLGVGYWVGFSDY |
| 131 | 2290-D02 | CDR-H3 | | DLGHRYWVGVFDY |
| 132 | 2290-C07 | CDR-H3 | | DMGVGYWVGFSDY |
| 133 | 2290-D05 | CDR-H3 | | DLGYGYWVGFSDY |
| 134 | 2290-C08 | CDR-H3 | | DWGVGYWVGFSDY |
| 135 | 2290-A02 | CDR-H3 | | DLGSRYWVGVFDY |
| 136 | 2213-A06 | CDR-H3 | | DFYDRYSTYVLDY |
| 137 | 2291-G05 | CDR-H3 | | DFHDRYATFVLDY |
| 138 | 2291-E06 | CDR-H3 | | DFYDRYATYVLDY |
| 139 | 2291-F10 | CDR-H3 | | DYYDRYSTYVLDY |
| 140 | 2291-A04 | CDR-H3 | | DFNDRYFTYVLDY |
| 141 | 9A8 | CDR-H3 | | TTCIGSGGCIDT |
| 142 | 11D6 | CDR-H3 | | TTCVGSGGCIDT |
| 143 | 10F4 | CDR-H3 | | GGGLNSYGCSGANIDA |
| 144 | 9A5 | CDR-H3 | | GGGAASIDT |
| 145 | 9E12 | CDR-H3 | | GGGGASIDG |
| 146 | trastuzumab | CDR-L1 | | RASQDVNTAVA |
| 147 | 9A8 | CDR-L1 | | SGGSSDYG |
| 148 | 10G5 | CDR-L1 | | SGGNYDYG |
| 149 | 11D6 | CDR-L1 | | SGGNSDYG |
| 150 | 10F4 | CDR-L1 | | SGGGNYFGSYYYG |
| 151 | 9A5 | CDR-L1 | | SGGGNYVGGYYYG |
| 152 | 9E12 | CDR-L1 | | SGGGSYYGSYYYG |
| 153 | 10H1 | CDR-L1 | | SGGGNYYGSYYYG |
| 154 | 10E10 | CDR-L1 | | SGGGNYAGSYYYG |
| 155 | trastuzumab | CDR-L2 | | SASFLYS |
| 156 | 9A8 | CDR-L2 | | SNNQRPS |
| 157 | 10G5 | CDR-L2 | | YNNKRPS |
| 158 | 11D6 | CDR-L2 | | RNNQRPS |
| 159 | 10F4 | CDR-L2 | | NNNNRPS |
| 160 | 10E10 | CDR-L2 | | NSNNRPS |
| 161 | trastuzumab | CDR-L3 | | QQHYTTPPT |
| 162 | 9A8 | CDR-L3 | | ANVDYTDDV |
| 163 | 10G5 | CDR-L3 | | ANVDSTDDV |
| 164 | 11D6 | CDR-L3 | | GNVDFTDDV |
| 165 | h11D6-Lc4 | CDR-L3 | | GGFDSSSDAI |
| 166 | 10F4 | CDR-L3 | | GGFDSSTDAI |
| 167 | 2137-C07 | V_{H} | | EVQLVESGGGLVQPGGSLRLSCAASGFN |
| | | | | ISGSGIHWVRQAPGKGLEWVGFINPAGG |
| | | | | YTDYADSVKGRFTISADTSKNTAYLQMN |
| | | | | SLRAEDTAVYYCARDYVYQYWTYVLDYW |
| | | | | GQGTLVTVSS |
| 168 | 2265-F06 | V_{H} | | EVQLVESGGGLVQPGGSLRLSCAASGFN |
| | | | | ISYPGIHWVRQAPGKGLEWVGFINPAGG |
| | | | | YTDYADSVKGRFTISADTSKNTAYLQMN |
| | | | | SLRAEDTAVYYCARDYILQYWTYVLDYW |
| | | | | GQGTLVTVSS |
| 169 | 2265-F05 | V_{H} | | EVQLVESGGGLVQPGGSLRLSCAASGFN |
| | | | | IIAPGIHWVRQAPGKGLEWVGFINPAGG |
| | | | | YTDYADSVKGRFTISADTSKNTAYLQMN |
| | | | | SLRAEDTAVYYCARDYVNAYWTYVLDYW |
| | | | | GQGTLVTVSS |
| 170 | 2265-F02 | V_{H} | | EVQLVESGGGLVQPGGSLRLSCAASGFN |
| | | | | ISAPGIHWVRQAPGKGLEWVGFINPAGG |
| | | | | YTDYADSVKGRFTISADTSKNTAYLQMN |
| | | | | SLRAEDTAVYYCARDYIRQYWTYVLDYW |
| | | | | GQGTLVTVSS |
| 171 | 2265-B06 | V_{H} | | EVQLVESGGGLVQPGGSLRLSCAASGFN |
| | | | | IRVSGIHWVRQAPGKGLEWVGFINPAGG |
| | | | | YTDYADSVKGRFTISADTSKNTAYLQMN |
| | | | | SLRAEDTAVYYCARDYVQAYWTYVLDYW |
| | | | | GQGTLVTVSS |
| 172 | 2265-A09 | V_{H} | | EVQLVESGGGLVQPGGSLRLSCAASGFN |
| | | | | IIGPGIHWVRQAPGKGLEWVGFINPAGG |
| | | | | YTDYADSVKGRFTISADTSKNTAYLQMN |
| | | | | SLRAEDTAVYYCARDYVYNYWTYVLDYW |
| | | | | GQGTLVTVSS |
| 173 | 2265-F03 | V_{H} | | EVQLVESGGGLVQPGGSLRLSCAASGFN |
| | | | | IRGPGIHWVRQAPGKGLEWVGFISPAAG |
| | | | | YTDYADSVKGRFTISADTSKNTAYLQMN |
| | | | | SLRAEDTAVYYCARDFVQSYWTYVLDYW |
| | | | | GQGTLVTVSS |
| 174 | 2265-E02 | V_{H} | | EVQLVESGGGLVQPGGSLRLSCAASGFN |
| | | | | IYVSGIHWVRQAPGKGLEWVGFINPAGG |
| | | | | YTDYADSVKGRFTISADTSKNTAYLQTN |
| | | | | SLRAEDTAVYYCARDYVYQYWTYVLDYW |
| | | | | GQGTLVTVSS |
| 175 | 2265-D11 | V_{H} | | EVQLVESGGGLVQPGGSLRLSCAASGFN |
| | | | | ISGPGIHWVRQAPGKGLEWVGFINPAAG |
| | | | | YTDYADSVKGRFTISADTSKNTAYLQMN |
| | | | | SLRAEDTAVYYCARDYIYQYWTYVLDYW |
| | | | | GQGTLVTVSS |
| 176 | 2265-D05 | V_{H} | | EVQLVESGGGLVQPGGSLRLSCAASGFN |
| | | | | ISGPGIHWVRQAPGKGLEWVGFINPAAG |
| | | | | YTDYADSVKGRFAISADTSKNTAYLQMN |
| | | | | SLRAEDTAVYYCARDFVYAYWTYVLDYW |
| | | | | GQGTLVTVSS |
| 177 | 2265-C03 | V_{H} | | EVQLVESGGGLVQPGGSLRLSCAAPGFN |
| | | | | ISVPGIHWVRQAPGKGLEWVGFINPAGG |
| | | | | YTDYADSVKGRFTISADTSKNTAYLQMN |
| | | | | SLRAEDTAVYYCARDYVPQYWTYVLDYW |
| | | | | GQGTLVTVSS |
| 178 | 2265-C02 | V_{H} | | EVQLVESGGGLVQPGGSLRLSCAASGFN |
| | | | | IGVSGIHWVRQAPGKGLEWVGFINPAGG |
| | | | | YTDYAGSVKGRFTISADTSKNTAYLQMN |
| | | | | SLRAEDTAVYYCARDYTYSYWTYVLDYW |
| | | | | GQGTLVTVSS |
| 179 | 2265-A06 | V_{H} | | EVQLVESGGGLVQPGGSLRLSCAASGFN |
| | | | | IYRSGIHWVRQAPGKGLEWVGFINPAGG |
| | | | | YTDYADSVKGRFTISADTSKNTAYLQMN |
| | | | | SLRAEDTAVYYCARDYVPQYWTYVLDYW |
| | | | | GQGTLVTVSS |
| 180 | 2137-A05 | V_{H} | | EVQLVESGGGLVQPGGSLRLSCAASGFN |
| | | | | INNSYIHWVRQAPGKGLEWVGWIYPYSG |
| | | | | YTNYADSVKGRFTISADTSKNTAYLQMN |
| | | | | SLRAEDTAVYYCARDYGPWYGTGVLDYW |
| | | | | GQGTLVTVSS |
| 181 | 2288-A03 | V_{H} | | EVQLVESGGGLVQPGGSLRLSCAASGFN |
| | | | | INNSWIHWVRQAPGKGLEWVGWIYPYIG |
| | | | | FTEYADSVKGRFTISADTSKNTAYLQMN |
| | | | | SLRAEDTAVYYCARDYDLRYLTGVLDYW |
| | | | | GQGTLVTVSS |
| 182 | 2190-B01 | V_{H} | | EVQLVESGGGLVQPGGSLRLSCAASGFN |
| | | | | ISSYWIHWVRQAPGKGLEWVGVITPSGG |
| | | | | YTYYADSVKGRFTISADTSKNTAYLQMN |
| | | | | SLRAEDTAVYYCARDLGGGYWVGFFDYW |
| | | | | GQGTLVTVSS |
| 183 | 2290-G01 | V_{H} | | EVQLVESGGGLVQPGGSLRLSCAASGFN |
| | | | | ISPYWIHWVRQAPGKGLEWVGVITPPSG |
| | | | | FTYYADSVKGRFTISADTSKNTAYLQVN |
| | | | | SLRAEDTAVYYCARDLGVGYWVGFSDYW |
| | | | | GQGTLVTVSS |
| 184 | 2290-D02 | V_{H} | | EVQLVESGGGLVQPGGSLRLSCAASGFN |
| | | | | ISSYWIHWVRQAPGKGLEWMGVITPAAG |
| | | | | YTYYADSVKGRFTISADTSKNTAYLQMN |
| | | | | SLRAEDTAVYYCARDLGHRYWVGVFDYW |
| | | | | GQGTLVTVSS |
| 185 | 2290-C07 | V_{H} | | EVQLVESGGGLVQPGGSLRLSCAASGFN |
| | | | | ITYDWIHWVRQAPGKGLEWVGVITPFDG |
| | | | | YTYYADSVKGHFTISADTSKNTAYLQMN |
| | | | | SLRAEDTAVYYCARDMGVGYWVGFSDYW |
| | | | | GQGTLVTVSS |
| 186 | 2290-D05 | V_{H} | | EVQLVESGGGLVQPGGSLRLSCAASGFN |
| | | | | IASRWIHWVRQAPGKGLEWVGVITPSAG |
| | | | | YTYYADSVKGRFTISADTSKNTAYLQMN |
| | | | | SLRAEDTAVYYCARDLGYGYWVGFSDYW |
| | | | | GQGTLVTVSS |
| 187 | 2290-C08 | V_{H} | | EVQLVESGGGLVQPGGSLRLSCAASGFN |
| | | | | IQPYWIHWVRQAPGKGLEWVGVITPPSG |
| | | | | YTYYADSVKGRFTISADTSKNTAYLQMN |
| | | | | SLRAEDTAVYYCARDWGVGYWVGFSDYW |
| | | | | GQGTLVTVSS |
| 188 | 2290-A02 | V_{H} | | EVQLVESGGGLVQPGGSLRLSCAASGFN |
| | | | | ISTRWIHWVRQAPGKGLEWVGVITPSAG |
| | | | | YTHYADSVKGRFTISAGTSKNTAYLQMN |
| | | | | SLRAEDTAVYYCARDLGSRYWVGVFDYW |
| | | | | GQGTLVTVSS |
| 189 | 2213-A06 | V_{H} | | EVQLVESGGGLVQPGGSLRLSCAASGFN |
| | | | | ISSYAIHWVRQAPGKGLEWVGVISPYGG |
| | | | | YTEYADSVKGRFTISADTSKNTAYLQMN |
| | | | | SLRAEDTAVYYCARDFYDRYSTYVLDYW |
| | | | | GQGTLVTVSS |
| 190 | 2291-G05 | V_{H} | | EVQLVESGGGLVQPGGSLRLSCAASGFN |
| | | | | IAAYTIHWVRQAPGKGLEWVGWISPYGG |
| | | | | YTEYADSVKGRFTISADTSKNTAYLQMN |
| | | | | SLRAEDTAVYYCARDFHDRYATFVLDYW |
| | | | | GQGTLVTVSS |
| 191 | 2291-E06 | V_{H} | | EVQLVESGGGLVQPGGSLRLSCAASGFN |
| | | | | ISPYTIHWVRQAPGKGLEWVAHIFPSGG |
| | | | | YTDYADSVKGRFTISADTSKNTAYLQMN |
| | | | | SLRAEDTAVYYCARDFYDRYATYVLDYW |
| | | | | GQGTLVTVSS |
| 192 | 2291-D07 | V_{H} | | EVQLVESGGGLVQPGGSLRLSCAASGFN |
| | | | | IKDTYIHWVRQAPGKGLEWVGVISPYDG |
| | | | | YTEYADSVKGRFTISADTSKNTAYLQMN |
| | | | | SLRAEDTAVYYCAHDFYDRYSTYVLDYW |
| | | | | GQGTLVTVSS |
| 193 | 2291-F10 | V_{H} | | EVQLVESGGGLVQPGGSLRLSCAASGFN |
| | | | | IDPYTIHWVRQAPGKGLEWVGWISPYDG |
| | | | | YTEYADSVKGRFTISADTSKNTAYLQMN |
| | | | | SLRAEDTAVYYCARDYYDRYSTYVLDYW |
| | | | | GRGTLVTVSS |
| 194 | 2291-A04 | V_{H} | | EVQLVESGGGLVQPGGSLRLSCAASGFN |
| | | | | ISSYGIHWVRQAPGKGLEWVGFISPYDG |
| | | | | YTEYADSVKGRFTISAGTSKNTAYLQMN |
| | | | | SLRAEDTAVYYCARDFNDRYFTYVLDYW |
| | | | | GQGTLVTVSS |
| 195 | 2291-A01 | V_{H} | | EVQLVESGGGLVQPGGSLRLSCAASGFN |
| | | | | IDPYTIHWARQAPGKGLEWVAHISPYDG |
| | | | | YTDYADSVKGRFTISADTSKNTAYLQMN |
| | | | | SLRAEDTAVYYCARDFYDRYSTYVLDYW |
| | | | | GQGTLVTVSS |
| 196 | 9A8 | V_{H} | | AVTLDESGGGLQTPGGTLSLVCKASGFT |
| | | | | FSSFNMFWVRQAPGKGLEWVAYIRNDGN |
| | | | | SASYGPAVKGRATISRDNGQSTVRLQLN |
| | | | | NLRAEDTATYYCAKTTCIGSGGCIDTWG |
| | | | | HGTEVIVSS |
| 197 | 10G5 | V_{H} | | AVTLDESGGGLQTPGGVLSLVCKASGFT |
| | | | | FSSFNMFWVRQAPGKGLEWVAYISNDGS |
| | | | | STSYGPAVKGRATISRDNGQSTVRLQLN |
| | | | | NLRAEDTATYFCAKTTCIGSGGCIDTWG |
| | | | | HGTEVIVSS |
| 198 | 11D6 | V_{H} | | AVTLDESGGGLQTPGGTLSLVCKASGFT |
| | | | | FSSFNMFWVRQAPGEGLEWVAYIRNDGR |
| | | | | STSYGPAVKGRATISRDNGQSTVRLQLN |
| | | | | NLRAEDTGTYFCAKTTCVGSGGCIDTWG |
| | | | | HGTEVIVSS |
| 199 | h11D6-HC4 | V_{H} | | EVQLVESGGGLVQPGGSLRLSCAASGFT |
| | | | | FSSFNMFWVRQAPGKGLEWVAYIRNDGR |
| | | | | STSYVDSVKGRFTISRDNAKSSVYLQMN |
| | | | | SLRAEDTAVYYCAKTTCVGSGGCIDTWG |
| | | | | QGTLVTVSS |
| 200 | h11D6-HC3 | V_{H} | | QVQLVESGGGVVQPGRSLRLSCAASGFT |
| | | | | FSSFNMFWVRQAPGKGLEWVAYIRNDGR |
| | | | | STSYADSVKGRFTISRDNSKSTVYLQMN |
| | | | | SLRAEDTAVYYCAKTTCVGSGGCIDTWG |
| | | | | QGTLVTVSS |
| 201 | h11D6-HC2 | V_{H} | | EVQLLESGGGLVQPGGSLRLSCAASGFT |
| | | | | FSSFNMFWVRQAPGKGLEWVAYIRNDGR |
| | | | | STSYADSVKGRFTISRDNSKSTVYLQMN |
| | | | | SLRAEDTAVYYCAKTTCVGSGGCIDTWG |
| | | | | QGTLVTVSS |
| 202 | h11D6-HC1 | V_{H} | | EVQLVESGGGLVKPGGSLRLSCAASGFT |
| | | | | FSSFNMFWVRQAPGKGLEWVAYIRNDGR |
| | | | | STSYAAPVKGRFTISRDNSKSTVYLQMN |
| | | | | SLKTEDTAVYYCAKTTCVGSGGCIDTWG |
| | | | | QGTLVTVSS |
| 203 | 10F4 | V_{H} | | AVTLDESGGGLQTPGGALSLVCKASGFT |
| | | | | FSGYNMGWVRQAPGKGLEYVAGITYGTG |
| | | | | SYTAYGAAVKGRATISRDNGQSTLRLQL |
| | | | | NNLRAEDTATYYCARGGGLNSYGCSGAN |
| | | | | IDAWGHGTEVIVSS |
| 204 | h10F4-HC4 | V_{H} | | EVQLVESGGGLVQPGGSLRLSCAASGFT |
| | | | | FSGYNMGWVRQAPGKGLEWVAGITYGTG |
| | | | | SYTAYVDSVKGRFTISRDNAKSSLYLQM |
| | | | | NSLRAEDTAVYYCARGGGLNSYGCSGAN |
| | | | | IDAWGQGTLVTVSS |
| 205 | h10F4-HC3 | V_{H} | | QVQLVESGGGVVQPGRSLRLSCAASGFT |
| | | | | FSGYNMGWVRQAPGKGLEWVAGITYGTG |
| | | | | SYTAYADSVKGRFTISRDNSKSTLYLQM |
| | | | | NSLRAEDTAVYYCARGGGLNSYGCSGAN |
| | | | | IDAWGQGTLVTVSS |
| 206 | h10F4-HC2 | V_{H} | | EVQLLESGGGLVQPGGSLRLSCAASGFT |
| | | | | FSGYNMGWVRQAPGKGLEWVAGITYGTG |
| | | | | SYTAYADSVKGRFTISRDNSKSTLYLQM |
| | | | | NSLRAEDTAVYYCARGGGLNSYGCSGAN |
| | | | | IDAWGQGTLVTVSS |
| 207 | h10F4-HC1 | V_{H} | | EVQLVESGGGLVKPGGSLRLSCAASGFT |
| | | | | FSGYNMGWVRQAPGKGLEWVAGITYGTG |
| | | | | SYTAYAAPVKGRFTISRDNSKSTLYLQM |
| | | | | NSLKTEDTAVYYCARGGGLNSYGCSGAN |
| | | | | IDAWGQGTLVTVSS |
| 208 | 9A5 | V_{H} | | AVTLDESGGGLQTPGGAVSLVCKASGFS |
| | | | | ISDYGMGWMRQAPGKGLQYVARIDHDGR |
| | | | | YTDYGAVVKGRATISRDNGQSTVRLQLN |
| | | | | NLRAEDTGTYYCTRGGGAASIDTWGHGT |
| | | | | EVIVSS |
| 209 | 9E12 | V_{H} | | AVTLDESGGGLQTPGGGLSLVCKASGFT |
| | | | | FSDYGLGWMRQAPGKGLEYVARINSAGS |
| | | | | GTYYGSAVDGRATISRDNGQSTVRLQLN |
| | | | | NLRAEDTGTYYCTRGGGGASIDGWGHGT |
| | | | | EVIVSS |
| 210 | 9H1 | V_{H} | | AVTLDESGGGLQTPGGALSLVCKGSGFT |
| | | | | FSDYGMGWMRQAPGKGLQYVARINSAGS |
| | | | | DTNYGSAVKGRATISRDDGQSTVRLQLS |
| | | | | SLRAEDTGIYYCTRGGGGASIDGWGHGT |
| | | | | EVIVSS |
| 211 | 10H1 | V_{H} | | AVTLDESGGGLQTPGGALSLVCKASGFT |
| | | | | FSGYGMGWMRQAPGKGLEYVARINSAGS |
| | | | | DTDYGAAVKGRATISRDNGQSTVRLQLN |
| | | | | NLRAEDTATYFCTRGGGGASIDGWGHGT |
| | | | | EVIVSS |
| 212 | 10E10 | V_{H} | | AVTLDESGGGLQTPGGGLSLVCKASGFT |
| | | | | FSSYGMGWMRQAPGKGLEFVARINSGGS |
| | | | | SYTDYGSAVKGRATISRDDGQSTVRLQL |
| | | | | NNLRAEDTGIYYCTRGGGGASIDGWGHG |
| | | | | TEVIVSS |
| 213 | h10H1-HC4 | V_{H} | | EVQLVESGGGLVQPGGSLRLSCAASGFT |
| | | | | FSGYGMGWVRQAPGKGLEWVARINSAGS |
| | | | | DTDYVDSVKGRFTISRDNAKSSVYLQMN |
| | | | | SLRAEDTAVYYCTRGGGGASIDGWGQGT |
| | | | | LVTVSS |
| 214 | h10H1-HC3 | V_{H} | | QVQLVESGGGVVQPGRSLRLSCAASGFT |
| | | | | FSGYGMGWVRQAPGKGLEWVARINSAGS |
| | | | | DTDYADSVKGRFTISRDNSKSTVYLQMN |
| | | | | SLRAEDTAVYYCTRGGGGASIDGWGQGT |
| | | | | LVTVSS |
| 215 | h10H1-HC2 | V_{H} | | EVQLLESGGGLVQPGGSLRLSCAASGFT |
| | | | | FSGYGMGWVRQAPGKGLEWVARINSAGS |
| | | | | DTDYADSVKGRFTISRDNSKSTVYLQMN |
| | | | | SLRAEDTAVYYCTRGGGGASIDGWGQGT |
| | | | | LVTVSS |
| 216 | h10H1-HC1 | V_{H} | | EVQLVESGGGLVKPGGSLRLSCAASGFT |
| | | | | FSGYGMGWVRQAPGKGLEWVARINSAGS |
| | | | | DTDYAAPVKGRFTISRDNSKSTVYLQMN |
| | | | | SLKTEDTAVYYCTRGGGGASIDGWGQGT |
| | | | | LVTVSS |
| 217 | trastuzumab | V_{L} | | DIQMTQSPSSLSASVGDRVTITCRASQD |
| | | | | VNTAVAWYQQKPGKAPKLLIYSASFLYS |
| | | | | GVPSRFSGSRSGTDFTLTISSLQPEDFA |
| | | | | TYYCQQHYTTPPTFGQGTKVEIK |
| 218 | 9A8 | V_{L} | | ALTQPSSVSANPGETVKITCSGGSSDYG |
| | | | | WFQQKSPGSAPVTVIYSNNQRPSGIPSR |
| | | | | FSGSKSGSTGTLTITGVQAEDEAIYYCA |
| | | | | NVDYTDDVFGAGTTLTVL |
| 219 | 10G5 | V_{L} | | ALTQPSSVSANPGETVKITCSGGNYDYG |
| | | | | WYQQKSPGSAPVTLIYYNNKRPSDIPSR |
| | | | | FSGSKSGSTGTLTITGVQAEDEAIYYCA |
| | | | | NVDSTDDVFGAGTTLTVL |
| 220 | 11D6 | V_{L} | | ALTQPSSVSANPGETVEITCSGGNSDYG |
| | | | | WFQQKSPGSAPVTVIYRNNQRPSDIPSR |
| | | | | FSGSGSGSTNTLTITGVQAEDEAIYYCG |
| | | | | NVDFTDDVFGAGTTLTVL |
| 221 | h11D6-LC4 | V_{L} | | SYVLTQPPSVSVAPGKTARITCSGGNSD |
| | | | | YGWYQQKPGQAPVLVVYRNNQRPSGIPE |
| | | | | RFSGSGSGSTNTLTISGTQAMDEADYYC |
| | | | | GGFDSSSDAIFGGGTKLTVL |
| 222 | h11D6-LC3 | V_{L} | | SYELTQPPSVSVSPGQTASITCSGGNSD |
| | | | | YGWYQQKPGQSPVLVIYRNNQRPSGIPE |
| | | | | RFSGSGSGSTNTLTISGTQAMDEADYYC |
| | | | | GGFDSSSDAIFGGGTKLTVL |
| 223 | h11D6-LC2 | V_{L} | | QSVLTQPPSVSAAPGQKVTISCSGGNSD |
| | | | | YGWYQQLPGTAPKLLIYRNNQRPSGIPD |
| | | | | RFSGSGSGSTNTLGITGLQTGDEADYYC |
| | | | | GGFDSSSDAIFGGGTKLTVL |
| 224 | h11D6-LC1 | V_{L} | | DIQMTQSPSSVSASVGDRVTITCSGGNS |
| | | | | DYGWYQQKPGKAPKLLIYRNNQRPSGVP |
| | | | | SRFSGSGSGSTNTLTISSLQPEDFATYY |
| | | | | CGGFDSSSDAIFGQGTKVEIK |
| 225 | 10F4 | V_{L} | | ALTQPSSVSANLGGTVKITCSGGGNYFG |
| | | | | SYYYGWYQQKAPGSAPVTVIYNNNNRPS |
| | | | | DIPSRFSGSTSGSTSTLTISGVRAEDEA |
| | | | | VYFCGGFDSSTDAIFGAGTTLTVL |
| 226 | h10F4-LC4 | V_{L} | | SYVLTQPPSVSVAPGKTARITCSGGGNY |
| | | | | FGSYYYGWYQQKPGQAPVLVVYNNNNRP |
| | | | | SGIPERFSGSTSGSTSTLTISGTQAMDE |
| | | | | ADYYCGGFDSSTDAIFGGGTKLTVL |
| 227 | h10F4-LC3 | V_{L} | | SYELTQPPSVSVSPGQTASITCSGGGNY |
| | | | | FGSYYYGWYQQKPGQSPVLVIYNNNNRP |
| | | | | SGIPERFSGSTSGSTSTLTISGTQAMDE |
| | | | | ADYYCGGFDSSTDAIFGGGTKLTVL |
| 228 | h10F4-LC2 | V_{L} | | QSVLTQPPSVSAAPGQKVTISCSGGGNY |
| | | | | FGSYYYGWYQQLPGTAPKLLIYNNNNRP |
| | | | | SGIPDRFSGSTSGSTSTLGITGLQTGDE |
| | | | | ADYYCGGFDSSTDAIFGGGTKLTVL |
| 229 | h10F4-LC1 | V_{L} | | DIQMTQSPSSVSASVGDRVTITCSGGGN |
| | | | | YFGSYYYGWYQQKPGKAPKLLIYNNNNR |
| | | | | PSGVPSRFSGSTSGSTSTLTISSLQPED |
| | | | | FATYYCGGFDSSTDAIFGQGTKVEIK |
| 230 | 9A5 | V_{L} | | ALTQPSSVSANPGETVKITCSGGGNYVG |
| | | | | GYYYGWYQQKAPGSALVTLIYNNNNRPS |
| | | | | NIPSRFSGSTSGSTSTLTITGVRAEDEA |
| | | | | VYFCGSFDSSTDAIFGAGTTLTVL |
| 231 | 9E12 | V_{L} | | ALTQPSSVSANPGETVKITCSGGGSYYG |
| | | | | SYYYGWYQQKSPGSAPVTLIYNNNNRPS |
| | | | | DIPSRFSGSTSGSTGTLTITGVRAEDEA |
| | | | | VYYCGSFDSSTDAIFGAGTTLTVL |
| 232 | 9H1 | V_{L} | | ALTQPSSVSANPGETVKITCSGGGSYYG |
| | | | | SYYYGWYQQKSPGSAPVTLIYNNNNRPS |
| | | | | DIPSRFSGSTSGSTGTLTITGVRAEDEA |
| | | | | VYYCGSFDSSTDAIFGAGTTLTVL |
| 233 | 10H1 | V_{L} | | ALTQPSSVSANPGETVKITCSGGGNYYG |
| | | | | SYYYGWYQQKAPGSAPVTVIYNNNNRPS |
| | | | | NIPSRFSGSKSGSTGTLTITGVQAEDEA |
| | | | | VYFCGGFDSSSDAIFGAGTTLTVL |
| 234 | 10E10 | V_{L} | | ALTQPSSVSANPGETVKITCSGGGNYAG |
| | | | | SYYYGWYQQKSPGSAPLTVIYNSNNRPS |
| | | | | DIPSRFSGSLSGSTGTLTITGVRAEDEA |
| | | | | VYFCGGFDSSTDAIFGAGTTLTVL |
| 235 | h10H1-LC4 | V_{L} | | SYVLTQPPSVSVAPGKTARITCSGGGNY |
| | | | | YGSYYYGWYQQKPGQAPVLVVYNNNNRP |
| | | | | SGIPERFSGSKSGSTGTLTISGTQAMDE |
| | | | | ADYYCGGFDSSSDAIFGGGTKLTVL |
| 236 | h10H1-LC3 | V_{L} | | SYELTQPPSVSVSPGQTASITCSGGGNY |
| | | | | YGSYYYGWYQQKPGQSPVLVIYNNNNRP |
| | | | | SGIPERFSGSKSGSTGTLTISGTQAMDE |
| | | | | ADYYCGGFDSSSDAIFGGGTKLTVL |
| 237 | h10H1-LC2 | V_{L} | | QSVLTQPPSVSAAPGQKVTISCSGGGNY |
| | | | | YGSYYYGWYQQLPGTAPKLLIYNNNNRP |
| | | | | SGIPDRFSGSKSGSTGTLGITGLQTGDE |
| | | | | ADYYCGGFDSSSDAIFGGGTKLTVL |
| 238 | h10H1-LC1 | V_{L} | | DIQMTQSPSSVSASVGDRVTITCSGGGN |
| | | | | YYGSYYYGWYQQKPGKAPKLLIYNNNNR |
| | | | | PSGVPSRFSGSKSGSTGTLTISSLQPED |
| | | | | FATYYCGGFDSSSDAIFGQGTKVEIK |
| 239 | Human IgG1 HC | | | ASTKGPSVFPLAPSSKSTSGGTAALGCL |
| | Constant | | | VKDYFPEPVTVSWNSGALTSGVHTFPAV |
| | | | | LQSSGLYSLSSVVTVPSSSLGTQTYICN |
| | | | | VNHKPSNTKVDKKVEPKSCDKTHTCPPC |
| | | | | PAPELLGGPSVFLFPPKPKDTLMISRTP |
| | | | | EVTCVVVDVSHEDPEVKFNWYVDGVEVH |
| | | | | NAKTKPREEQYNSTYRVVSVLTVLHQDW |
| | | | | LNGKEYKCKVSNKALPAPIEKTISKAKG |
| | | | | QPREPQVYTLPPSREEMTKNQVSLTCLV |
| | | | | KGFYPSDIAVEWESNGQPENNYKTTPPV |
| | | | | LDSDGSFFLYSKLTVDKSRWQQGNVFSC |
| | | | | SVMHEALHNHYTQKSLSLSPGK |
| 240 | Human IgG LC | | | RTVAAPSVFIFPPSDEQLKSGTASVVCL |
| | Constant Ckappa | | | LNNFYPREAKVQWKVDNALQSGNSQESV |
| | | | | TEQDSKDSTYSLSSTLTLSKADYEKHKV |
| | | | | YACEVTHQGLSSPVTKSFNRGEC |
| 241 | Mouse IgG1 HC | | | AKTTPPSVYPLAPGSAAQTNSMVTLGCL |
| | Constant | | | VKGYFPEPVTVTWNSGSLSSGVHTFPAV |
| | | | | LQSDLYTLSSSVTVPSSTWPSETVTCNV |
| | | | | AHPASSTKVDKKIVPRDCGCKPCICTVP |
| | | | | EVSSVFIFPPKPKDVLTITLTPKVTCVV |
| | | | | VDISKDDPEVQFSWFVDDVEVHTAQTQP |
| | | | | REEQFNSTFRSVSELPIMHQDWLNGKEF |
| | | | | KCRVNSAAFPAPIEKTISKTKGRPKAPQ |
| | | | | VYTIPPPKEQMAKDKVSLTCMITDFFPE |
| | | | | DITVEWQWNGQPAENYKNTQPIMDTDGS |
| | | | | YFVYSKLNVQKSNWEAGNTFTCSVLHEG |
| | | | | LHNHHTEKSLSHSPG |
| 242 | Mouse IgG LC | | | RADAAPTVSIFPPSSEQLTSGGASVVCF |
| | Constant Ckappa | | | LNNFYPKDINVKWKIDGSERQNGVLNSW |
| | | | | TDQDSKDSTYSMSSTLTLTKDEYERHNS |
| | | | | YTCEATHKTSTSPIVKSFNRNEC |
| 243 | Kappa LC | | | HMTVAAPSVFIFPPSDEQLKSGTASVVC |
| | | | | LLNNFYPREAKVQWKVDNALQSGNSQES |
| | | | | VTEQDSKDSTYSLSSTLTLSKADYEKHK |
| | | | | VYACEVTHQGLSSPVTKSFNRGEC |
| 244 | Lambda LD | | | GQPKAAPSVTLFPPSSEELQANKATLVC |
| | | | | LISDFYPGAVTVAWKADSSPVKAGVETT |
| | | | | TPSKQSNNKYAASSYLSLTPEQWKSHRS |
| | | | | YSCQVTHEGSTVEKTVAPTECS |
| 245 | FlagHis Tag | | | GSGDYKDDDDKGSGHHHHHH |
| 246 | Linker | | | GGGGSGGGGSGGGGS |
| 247 | Linker | | | AAGSDQEPKSS |

Table 11 provides sequences referred to herein.

| | SEQ | | SEQ | | SEQ | | SEQ | | SEQ | |
|---|---|---|---|---|---|---|---|---|---|---|
| | ID | CDR H1 | ID | CDR H1 | ID | CDR H2 | ID | CDR H2 | ID | |
| | NO | Chothia | NO | Kabat | NO | Chothia | NO | Kabat | NO | CDR H3 |
| 2137-C07 | 5 | GFNISGS | 32 | GSGIH | 57 | NPAGGY | 79 | FINPAGGYTDYADSVKG | 116 | DYVYQYWTYVLDY |
| 2265-F06 | 6 | GFNISYP | 33 | YPGIH | 57 | NPAGGY | 79 | FINPAGGYTDYADSVKG | 117 | DYILQYWTYVLDY |
| 2265-F05 | 7 | GFNIIAP | 34 | APGIH | 57 | NPAGGY | 79 | FINPAGGYTDYADSVKG | 118 | DYVNAYWTYVLDY |
| 2265-F02 | 8 | GFNISAP | 34 | APGIH | 57 | NPAGGY | 79 | FINPAGGYTDYADSVKG | 119 | DYIRQYWTYVLDY |
| 2265-B06 | 9 | GFNIRVS | 35 | VSGIH | 57 | NPAGGY | 79 | FINPAGGYTDYADSVKG | 120 | DYVQAYWTYVLDY |
| 2265-A09 | 10 | GFNIIGP | 36 | GPGIH | 57 | NPAGGY | 79 | FINPAGGYTDYADSVKG | 121 | DYVYNYWTYVLDY |
| 2265-F03 | 11 | GFNIRGP | 36 | GPGIH | 58 | SPAAGY | 80 | FISPAAGYTDYADSVKG | 122 | DFVQSYWTYVLDY |
| 2265-E02 | 12 | GFNIYVS | 35 | VSGIH | 57 | NPAGGY | 79 | FINPAGGYTDYADSVKG | 116 | DYVYQYWTYVLDY |
| 2265-D11 | 13 | GFNISGP | 36 | GPGIH | 59 | NPAAGY | 81 | FINPAAGYTDYADSVKG | 123 | DYIYQYWTYVLDY |
| 2265-D05 | 13 | GFNISGP | 36 | GPGIH | 59 | NPAAGY | 81 | FINPAAGYTDYADSVKG | 124 | DFVYAYWTYVLDY |
| 2265-C03 | 14 | GFNISVP | 37 | VPGIH | 57 | NPAGGY | 79 | FINPAGGYTDYADSVKG | 125 | DYVPQYWTYVLDY |
| 2265-C02 | 15 | GFNIGVS | 35 | VSGIH | 57 | NPAGGY | 79 | FINPAGGYTDYADSVKG | 126 | DYIYSYWTYVLDY |
| 2265-A06 | 16 | GFNIYRS | 38 | RSGIH | 57 | NPAGGY | 79 | FINPAGGYTDYADSVKG | 125 | DYVPQYWTYVLDY |
| 2137-A05 | 17 | GFNINNS | 39 | NSYIH | 60 | YPYSGY | 82 | WIYPYSGYTNYADSVKG | 127 | DYGPWYGTGVLDY |
| 2288-A03 | 17 | GFNINNS | 40 | NSWIH | 61 | YPYIGF | 83 | WIYPYIGFTEYADSVKG | 128 | DYDLRYLTGVLDY |
| 2190-B01 | 18 | GFNISSY | 41 | SYWIH | 62 | TPSGGY | 84 | VITPSGGYTYYADSVKG | 129 | DLGGGYWVGFFDY |
| 2290-G01 | 19 | GFNISPY | 42 | PYWIH | 63 | TPPSGF | 85 | VITPPSGFTYYADSVKG | 130 | DLGVGYWVGFSDY |
| 2290-D02 | 19 | GFNISSY | 41 | SYWIH | 64 | TPAAGY | 86 | VITPAAGYTYYADSVKG | 131 | DLGHRYWVGVFDY |
| 2290-C07 | 20 | GFNITYD | 43 | YDWIH | 65 | TPFDGY | 87 | VITPFDGYTYYADSVKG | 132 | DMGVGYWVGFSDY |
| 2290-D05 | 21 | GFNIASR | 44 | SRWIH | 66 | TPSAGY | 88 | VITPSAGYTYYADSVKG | 133 | DLGYGYWVGFSDY |
| 2290-C08 | 22 | GFNIQPY | 42 | PYWIH | 67 | TPPSGY | 89 | VITPPSGYTYYADSVKG | 134 | DWGVGYWVGFSDY |
| 2290-A02 | 23 | GFNISTR | 45 | TRWIH | 66 | TPSAGY | 90 | VITPSAGYTHYADSVKG | 135 | DLGSRYWVGVFDY |
| 2213-A06 | 18 | GFNISSY | 46 | SYAIH | 68 | SPYGGY | 91 | VISPYGGYTEYADSVKG | 136 | DFYDRYSTYVLDY |
| 2291-G05 | 24 | GFNIAAY | 47 | AYTIH | 68 | SPYGGY | 92 | WISPYGGYTEYADSVKG | 137 | DFHDRYATFVLDY |
| 2291-E06 | 19 | GFNISPY | 48 | PYTIH | 69 | FPSGGY | 93 | HIFPSGGYTDYADSVKG | 138 | DFYDRYATYVLDY |
| 2291-D07 | 25 | GFNIKDT | 49 | DTYIH | 70 | SPYDGY | 94 | VISPYDGYTEYADSVKG | 136 | DFYDRYSTYVLDY |
| 2291-F10 | 26 | GFNIDPY | 48 | PYTIH | 70 | SPYDGY | 95 | WISPYDGYTEYADSVKG | 139 | DYYDRYSTYVLDY |
| 2291-A04 | 18 | GFNISSY | 50 | SYGIH | 70 | SPYDGY | 96 | FISPYDGYTEYADSVKG | 140 | DFNDRYFTYVLDY |
| 2291-A01 | 26 | GFNIDPY | 48 | PYTIH | 70 | SPYDGY | 97 | HISPYDGYTDYADSVKG | 136 | DFYDRYSTYVLDY |
| 9A8 | 27 | GFTFSSF | 51 | SFNMF | 71 | RNDGNS | 98 | YIRNDGNSASYGPAVKG | 141 | TTCIGSGGCIDT |
| 10g5 | 27 | GFTFSSF | 51 | SFNMF | 72 | SNDGSS | 99 | YISNDGSSTSYGPAVKG | 142 | TTCVGSGGCIDT |
| 11D6 | 27 | GFTFSSF | 51 | SFNMF | 73 | RNDGRS | 100 | YIRNDGRSTSYGPAVKG | 142 | TTCVGSGGCIDT |
| H11D6- | 27 | GFTFSSF | 51 | SFNMF | 73 | RNDGRS | 101 | YIRNDGRSTSYVDSVKG | 142 | TTCVGSGGCIDT |
| HC4 | | | | | | | | | | |
| H11D6- | 27 | GFTFSSF | 51 | SFNMF | 73 | RNDGRS | 102 | YIRNDGRSTSYADSVKG | 142 | TTCVGSGGCIDT |
| HC3 | | | | | | | | | | |
| H11D6- | 27 | GFTFSSF | 51 | SFNMF | 73 | RNDGRS | 102 | YIRNDGRSTSYADSVKG | 142 | TTCVGSGGCIDT |
| HC2 | | | | | | | | | | |
| H11D6- | 27 | GFTFSSF | 51 | SFNMF | 73 | RNDGRS | 103 | YIRNDGRSTSYAAPVKG | 142 | TTCVGSGGCIDT |
| HC1 | | | | | | | | | | |
| 10F4 | 28 | GFTFSGY | 52 | GYNMG | 74 | TYGTGSY | 104 | GITYGTGSYTAYGAAVKG | 143 | GGGLNSYGCSGANIDA |
| H10F4- | 28 | GFTFSGY | 52 | GYNMG | 74 | TYGTGSY | 105 | GITYGTGSYTAYVDSVKG | 143 | GGGLNSYGCSGANIDA |
| HC4 | | | | | | | | | | |
| H10F4- | 28 | GFTFSGY | 52 | GYNMG | 74 | TYGTGSY | 106 | GITYGTGSYTAYADSVKG | 143 | GGGLNSYGCSGANIDA |
| HC3 | | | | | | | | | | |
| H10F4- | 28 | GFTFSGY | 52 | GYNMG | 74 | TYGTGSY | 106 | GITYGTGSYTAYADSVKG | 143 | GGGLNSYGCSGANIDA |
| HC2 | | | | | | | | | | |
| H10F4- | 28 | GFTFSGY | 52 | GYNMG | 74 | TYGTGSY | 107 | GITYGTGSYTAYAAPVKG | 143 | GGGLNSYGCSGANIDA |
| HC1 | | | | | | | | | | |
| 9A5 | 29 | GFSISDY | 53 | DYGMG | 75 | DHDGRY | 108 | RIDHDGRYTDYGAVVKG | 144 | GGGAASIDT |
| 9E12 | 30 | GFTFSDY | 54 | DYGLG | 76 | NSAGSG | 109 | RINSAGSGTYYGSAVDG | 145 | GGGGASIDG |
| 9H1 | 30 | GFTFSDY | 53 | DYGMG | 77 | NSAGSD | 110 | RINSAGSDTNYGSAVKG | 145 | GGGGASIDG |
| 10H1 | 28 | GFTFSGY | 55 | GYGMG | 77 | NSAGSD | 111 | RINSAGSDTDYGAAVKG | 145 | GGGGASIDG |
| 10E10 | 31 | GFTFSSY | 56 | SYGMG | 78 | NSGGSSY | 112 | RINSGGSSYTDYGSAVKG | 145 | GGGGASIDG |
| H10H1- | 28 | GFTFSGY | 55 | GYGMG | 77 | NSAGSD | 113 | RINSAGSDTDYVDSVKG | 145 | GGGGASIDG |
| HC4 | | | | | | | | | | |
| H10H1- | 28 | GFTFSGY | 55 | GYGMG | 77 | NSAGSD | 114 | RINSAGSDTDYADSVKG | 145 | GGGGASIDG |
| HC3 | | | | | | | | | | |
| H10H1- | 28 | GFTFSGY | 55 | GYGMG | 77 | NSAGSD | 114 | RINSAGSDTDYADSVKG | 145 | GGGGASIDG |
| HC2 | | | | | | | | | | |
| H10H1- | 28 | GFTFSGY | 55 | GYGMG | 77 | NSAGSD | 115 | RINSAGSDTDYAAPVKG | 145 | GGGGASIDG |
| HC1 | | | | | | | | | | |

Table 12 provides sequences referred to herein.

| | SEQ ID | | SEQ ID | | SEQ ID | |
|---|---|---|---|---|---|---|
| | NO | CDR L1 | NO | CDR L2 | NO | CDR L3 |
| trastuzumab | 146 | RASQDVNTAVA | 155 | SASFLYS | 161 | QQHYTTPPT |
| 9A8 | 147 | SGGSSDYG | 156 | SNNQRPS | 162 | ANVDYTDDV |
| 10G5 | 148 | SGGNYDYG | 157 | YNNKRPS | 163 | ANVDSTDDV |
| 11D6 | 149 | SGGNSDYG | 158 | RNNQRPS | 164 | GNVDFTDDV |
| H11D6-LC4 | 149 | SGGNSDYG | 158 | RNNQRPS | 165 | GGFDSSSDAI |
| H11D6-LC3 | 149 | SGGNSDYG | 158 | RNNQRPS | 165 | GGFDSSSDAI |
| H11D6-LC2 | 149 | SGGNSDYG | 158 | RNNQRPS | 165 | GGFDSSSDAI |
| H11D6-LC1 | 149 | SGGNSDYG | 158 | RNNQRPS | 165 | GGFDSSSDAI |
| 10F4 | 150 | SGGGNYFGSYYYG | 159 | NNNNRPS | 166 | GGFDSSTDAI |
| H10F4-LC4 | 150 | SGGGNYFGSYYYG | 159 | NNNNRPS | 166 | GGFDSSTDAI |
| H10F4-LC3 | 150 | SGGGNYFGSYYYG | 159 | NNNNRPS | 166 | GGFDSSTDAI |
| H10F4-LC2 | 150 | SGGGNYFGSYYYG | 159 | NNNNRPS | 166 | GGFDSSTDAI |
| H10F4-LC1 | 150 | SGGGNYFGSYYYG | 159 | NNNNRPS | 166 | GGFDSSTDAI |
| 9A5 | 151 | SGGGNYVGGYYYG | 159 | NNNNRPS | 166 | GGFDSSTDAI |
| 9E12 | 152 | SGGGSYYGSYYYG | 159 | NNNNRPS | 166 | GGFDSSTDAI |
| 9H1 | 152 | SGGGSYYGSYYYG | 159 | NNNNRPS | 166 | GGFDSSTDAI |
| 10H1 | 153 | SGGGNYYGSYYYG | 159 | NNNNRPS | 166 | GGFDSSTDAI |
| 10E10 | 154 | SGGGNYAGSYYYG | 160 | NSNNRPS | 166 | GGFDSSTDAI |
| H10H1-LC4 | 153 | SGGGNYYGSYYYG | 159 | NNNNRPS | 166 | GGFDSSTDAI |
| H10H1-LC3 | 153 | SGGGNYYGSYYYG | 159 | NNNNRPS | 166 | GGFDSSTDAI |
| H10H1-LC2 | 153 | SGGGNYYGSYYYG | 159 | NNNNRPS | 166 | GGFDSSTDAI |
| H10H1-LC1 | 153 | SGGGNYYGSYYYG | 159 | NNNNRPS | 166 | GGFDSSTDAI |

Table 13 provides sequences referred to herein.

| | CDR H1 | CDR H1 | CDR H2 | CDR H2 | | | | |
|---|---|---|---|---|---|---|---|---|
| | Chothia | Kabat | Chothia | Kabat | CDR H3 | CDR L1 | | |
| | SEQ ID | SEQ ID | SEQ ID | SEQ ID | SEQ ID | SEQ ID | CDR 12 | CDR 13 |
| | NO | NO | NO | NO | NO | NO | SEQ ID NO | SEQ ID NO |
| 2137-C07 | 5 | 32 | 57 | 79 | 116 | 146 | 155 | 161 |
| 2265-F06 | 6 | 33 | 57 | 79 | 117 | 146 | 155 | 161 |
| 2265-F05 | 7 | 34 | 57 | 79 | 118 | 146 | 155 | 161 |
| 2265-F02 | 8 | 34 | 57 | 79 | 119 | 146 | 155 | 161 |
| 2265-B06 | 9 | 35 | 57 | 79 | 120 | 146 | 155 | 161 |
| 2265-A09 | 10 | 36 | 57 | 79 | 121 | 146 | 155 | 161 |
| 2265-F03 | 11 | 36 | 58 | 80 | 122 | 146 | 155 | 161 |
| 2265-E02 | 12 | 35 | 57 | 79 | 116 | 146 | 155 | 161 |
| 2265-D11 | 13 | 36 | 59 | 81 | 123 | 146 | 155 | 161 |
| 2265-D05 | 13 | 36 | 59 | 81 | 124 | 146 | 155 | 161 |
| 2265-C03 | 14 | 37 | 57 | 79 | 125 | 146 | 155 | 161 |
| 2265-C02 | 15 | 35 | 57 | 79 | 126 | 146 | 155 | 161 |
| 2265-A06 | 16 | 38 | 57 | 79 | 125 | 146 | 155 | 161 |
| 2137-A05 | 17 | 39 | 60 | 82 | 127 | 146 | 155 | 161 |
| 2288-A03 | 17 | 40 | 61 | 83 | 128 | 146 | 155 | 161 |
| 2190-B01 | 18 | 41 | 62 | 84 | 129 | 146 | 155 | 161 |
| 2290-G01 | 19 | 42 | 63 | 85 | 130 | 146 | 155 | 161 |
| 2290-D02 | 19 | 41 | 64 | 86 | 131 | 146 | 155 | 161 |
| 2290-C07 | 20 | 43 | 65 | 87 | 132 | 146 | 155 | 161 |
| 2290-D05 | 21 | 44 | 66 | 88 | 133 | 146 | 155 | 161 |
| 2290-C08 | 22 | 42 | 67 | 89 | 134 | 146 | 155 | 161 |
| 2290-A02 | 23 | 45 | 66 | 90 | 135 | 146 | 155 | 161 |
| 2213-A06 | 18 | 46 | 68 | 91 | 136 | 146 | 155 | 161 |
| 2291-G05 | 24 | 47 | 68 | 92 | 137 | 146 | 155 | 161 |
| 2291-E06 | 19 | 48 | 69 | 93 | 138 | 146 | 155 | 161 |
| 2291-D07 | 25 | 49 | 70 | 94 | 136 | 146 | 155 | 161 |
| 2291-F10 | 26 | 48 | 70 | 95 | 139 | 146 | 155 | 161 |
| 2291-A04 | 18 | 50 | 70 | 96 | 140 | 146 | 155 | 161 |
| 2291-A01 | 26 | 48 | 70 | 97 | 136 | 146 | 155 | 161 |
| 9A8 | 27 | 51 | 71 | 98 | 141 | 147 | 156 | 162 |
| 10g5 | 27 | 51 | 72 | 99 | 142 | 148 | 157 | 163 |
| 11D6 | 27 | 51 | 73 | 100 | 142 | 149 | 158 | 164 |
| H11D6-HC4-LC4 | 27 | 51 | 73 | 101 | 142 | 149 | 158 | 165 |
| H11D6-HC3-LC3 | 27 | 51 | 73 | 102 | 142 | 149 | 158 | 165 |
| H11D6-HC2-LC2 | 27 | 51 | 73 | 102 | 142 | 149 | 158 | 165 |
| H11D6-HC1-LC1 | 27 | 51 | 73 | 103 | 142 | 149 | 158 | 165 |
| 10F4 | 28 | 52 | 74 | 104 | 143 | 150 | 159 | 166 |
| H10F4-HC4-LC4 | 28 | 52 | 74 | 105 | 143 | 150 | 159 | 166 |
| H10F4-HC3-LC3 | 28 | 52 | 74 | 106 | 143 | 150 | 159 | 166 |
| H10F4-HC2-LC2 | 28 | 52 | 74 | 106 | 143 | 150 | 159 | 166 |
| H10F4-HC1-LC1 | 28 | 52 | 74 | 107 | 143 | 150 | 159 | 166 |
| 9A5 | 29 | 53 | 75 | 108 | 144 | 151 | 159 | 166 |
| 9E12 | 30 | 54 | 76 | 109 | 145 | 152 | 159 | 166 |
| 9H1 | 30 | 53 | 77 | 110 | 145 | 152 | 159 | 166 |
| 10H1 | 28 | 55 | 77 | 111 | 145 | 153 | 159 | 166 |
| 10E10 | 31 | 56 | 78 | 112 | 145 | 154 | 160 | 166 |
| H10H1-HC4-LC4 | 28 | 55 | 77 | 113 | 145 | 153 | 159 | 166 |
| H10H1-HC3-LC3 | 28 | 55 | 77 | 114 | 145 | 153 | 159 | 166 |
| H10H1-HC2-LC2 | 28 | 55 | 77 | 114 | 145 | 153 | 159 | 166 |
| H10H1-HC1-LC1 | 28 | 55 | 77 | 115 | 145 | 153 | 159 | 166 |

### EQUIVALENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

One or more features from any embodiments described herein or in the figures may be combined with one or more features of any other embodiments described herein or in the figures without departing from the scope of the invention.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. An isolated antibody of the IgG class that specifically binds to B-cell maturation antigen (BCMA), wherein the antibody comprises a CDR-H3 sequence selected from:
(a) a sequence defined by the consensus sequence consensus sequence D-α₂-α₃-α₄-α₅-Y-W-T-Y-V-L-D-Y, where α₂ is Y or F; α₃ is V or I; α₄ is Y, L, N, R, Q, or P; and α₅ is Q, A, N, or S;
(b) a sequence defined by the consensus sequence D-Y-α₃-α₄-α₅-Y-α₇-T-G-V-L-D-Y, where as is G or D; α₄ is P or L; α₅ is W or R; and α₇ is G or L;
(c) a sequence defined by the consensus sequence D-α₂-G-α₄-α₅-Y-W-V-G-α₁₀-α₁₁-D-Y, where α₂ is L, M, or W; α₄ is G, V, H, Y, or S; α₅ is G or R; α₁₀ is F or V; and α₁₁ is F or S;
(d) a sequence defined by the consensus sequence D-α₂-α₃-D-R-Y-α₇-T-α₉-V-L-D-Y, where α₂ is F or Y; α₃ is Y, H, or N; α₇ is S, A, or F; and α₉ is Y or F;
(e) a sequence defined by the consensus sequence T-T-C-α₄-G-S-G-G-C-I-D-T, where as is I or V;
(f) a sequence defined by the consensus sequence G-G-G-L-N-S-Y-G-C-S-G-A-N-I-D-A;
(g) a sequence defined by the consensus sequence G-G-G-α₄-A-S-I-D-α₉, where α₄ is A or G, and α₉ is T or G; (h)
(h) a sequence selected from any one of SEQ ID NOs: 116-145, or a variant thereof having three, two, or one amino acid substitution(s);
a CDR-H2 sequence consisting of:
a Chothia CDR-H2 sequence selected from:
(i) a sequence defined by the consensus sequence ε₁-P-ε₃-A-ε₅-G-Y, where si is N or S; ε₃ is absent; and ε₅ is G or A;
(i) a sequence defined by the consensus sequence Y-P-ε₃-Y-ε₅-G-ε₇, where ε₃ is absent; ε₅ is S or I; and ε₇ is Y or F;
(k) a sequence defined by the consensus sequence T-P-ε₃-ε₄-ε₅-G-ε₇, where ε₃ is absent; ε₄ is S, P, A, or F; ε₅ is G, S, A, or D; and ε₇ is Y or F;
(l) a sequence defined by the consensus sequence ε₁-P-ε₃-ε₄-ε₅-G-Y, where ε₁ is S or F; ε₃ is absent; ε₄ is Y or S; and ε₅ is G or D;
(m) a sequence defined by the consensus sequence ε₁-N-D-ε₄-G-ε₆-S, where ε₁ is R or S; ε₄ is absent; and ε₆ is N, S, or R;
(n) a sequence defined by the consensus sequence T-Y-G-T-G-S-Y;
(o) a sequence defined by the consensus sequence ε₁-ε₂-ε₃-ε₄-ε₅-ε₆-ε₇, where ε₁ is D or N; ε₂ is H or S; ε₃ is D, A, G, or absent; ε₄ is G, A, or absent; ε₅ is G or S; ε₆ is R or S;
and ε₇ is Y, G, or D;
(p) a sequence selected from any one of SEQ ID NOs: 57-78, or a variant thereof having two or one amino acid substitutions(s); or
a Kabat CDR-H2 sequence selected from:
(g) a sequence defined by the consensus sequence F-I-θ₃-P-A-θ₆-G-Y-T-D-Y-A-θ₁₃-S-V-K-G, where θ₃ is N or S; θ₆ is G or A; and θ₁₃ is D or G;
(r) a sequence defined by the consensus sequence W-I-Y-P-Y-θ₆-G-θ₈-T-θ₁₀-Y-A-D-S-V-K-G, where θ₆ is S or I; θ₈ is Y or F; and θ₁₀ is N or E;
(s) a sequence defined by the consensus sequence V-I-T-P-θ₅-θ₆-G-θ₈-T-θ₁₀-Y-A-D-S-V-K-G, where θ₅ is S, P, A, or F; θ₆ is G, S, A, or D; θ₈ is Y or F; and θ₁₀ is Y or H;
(t) a sequence defined by the consensus sequence θ₁-I-θ₃-P-θ₅-θ₆-G-Y-T-θ₁₀-Y-A-D-S-V-K-G, where θ₁ is V, W, H, or F; θ₃ is S or F; θ₅ is Y or S; θ₆ is G or D; and θ₁₀ is E or D;
(u) a sequence defined by the consensus sequence Y-I-θ₃-N-D-G-θ₇-S-θ₉-S-Y-θ₁₂-θ₁₃-θ₁₄-V-K-G, where θ₃ is R or S; θ₇ is N, S, or R; θ₉ is A or T; θ₁₂ is G, V, or A; θ₁₃ is P, D, or A; and θ₁₄ is A, S, or P;
(v) a sequence defined by the consensus sequence G-I-T-Y-G-T-G-S-Y-T-A-Y-θ₁₃-θ₁₄-θ₁₅-V-K-G, where θ₁₃ is G, V, or A; θ₁₄ is A or D; and θ₁₅ is A, S, or P;
(w) a sequence defined by the consensus sequence R-I-θ₃-θ₄-θ₅-θ₆-θ₇-θ₈-θ₉-T-θ₁₁-Y-θ₁₃-θ₁₄-θ₁₅-V-θ₁₇-G, where θ₃ is D or N; θ₄ is H or S; θ₅ is D, A, G, or absent; θ₆ is G, A, or absent; θ₇ is G or S; θ₈ is R or S; θ₉ is Y, G, or D; θ₁₁ is D, Y, or N; θ₁₃ is G, V, or A; θ₁₄ is A, S, or D; θ₁₅ is V, A, S, or P; and θ₁₇ is K or D;
(x) a sequence selected from any one of SEQ ID NOs: 79-115, or a variant thereof having three, two, or one amino acid substitutions(s);
a CDR-H1 sequence consisting of:
a Chothia CDR-H1 sequence selected from:
(y) a sequence defined by the consensus sequence G-F-N-I-γ₅-γ₆-γ₇, where γ₅ is S, I, R, Y, or G; γ₆ is G, Y, A, V, or R; and γ₇ is S or P;
(z) a sequence defined by the consensus sequence G-F-N-I-N-N-S;
(aa) a sequence defined by the consensus sequence G-F-N-I-γ₅-γ₆-γ₇, where γ₅ is S, T, A, or Q; γ₆ is S, P, Y, or T; and γ₇ is Y, D, or R;
(bb) a sequence defined by the consensus sequence G-F-N-I-γ₅-γ₆-γ₇, where γ₅ is S, A, K, or D; γ₆ is S, A, P, or D; and γ₇ is Y or T;
(cc) a sequence defined by the consensus sequence G-F-T-F-S-S-F;
(dd) G-F-T-F-S-G-Y;
(ee) G-F-γ₃-γ₄-S-γ₆-Y, where γ₃ is S or T; γ₄ is I or F; and γ₆ is D, G, or S;
(ff) a sequence selected from any one of SEQ ID NOs: 5-31, or a variant thereof having two or one amino acid substitutions(s); or
a Kabat CDR-H1 sequence selected from:
(gg) a sequence defined by the consensus sequence ζ₁-ζ₂-G-I-H, where ζ₁ is G, Y, A, V, or R; and ζ₂ is S or P;
(hh) a sequence defined by the consensus sequence N-S-ζ₃-I-H, where ζ₃ is Y or W;
(ii) a sequence defined by the consensus sequence ζ₁-ζ₂-W-I-H, where ζ₁ is S, P, Y, or T; and ζ₂ is Y, D, or R;
(jj) a sequence defined by the consensus sequence ζ₁-ζ₂-ζ₃-I-H, where ζ₁ is S, A, P, or D; ζ₂ is Y or T; and ζ₃ is A, T, Y, or G;
(kk) a sequence defined by the consensus sequence S-F-N-M-F;
(ll) a sequence defined by the consensus sequence G-Y-N-M-G;
(mm) a sequence defined by the consensus sequence ζ₁-Y-G-ζ₄-G, where ζ₁ is D, G, or S; and ζ₄ is M or L; and
(nn) a sequence selected from any one of SEQ ID NOs: 32-56, or a variant thereof having three, two, or one amino acid substitutions(s);
a CDR-L3 sequence selected from any one of SEQ ID NOs: 161-166, or a variant thereof having three, two, or one amino acid substitution(s);
a CDR-L2 sequence selected from a sequence selected from any one of SEQ ID NOs: 155-160, or a variant thereof having two or one amino acid substitution(s); and
a CDR-L1 sequence selected from a sequence selected from any one of SEQ ID NOs: 146-154, or a variant thereof having three, two, or one amino acid substitution(s).

2. The antibody of claim 1, wherein the antibody comprises
a CDR-H1, a CDR-H2, a CDR-H3, a CDR-L1, a CDR-L2, and a CDR-L3, of a VH-VL pair selected from the list of SEQ ID NO:
167 and 217; 168 and 217; 169 and 217; 171 and 217; 172 and 217; 173 and 217; 174 and 217; 175 and 217; 176 and 217; 177 and 217; 178 and 217; 179 and 217; 180 and 217; 181 and 217; 182 and 217; 183 and 217; 184 and 217; 185 and 217; 186 and 217; 187 and 217; 188 and 217; 189 and 217; 190 and 217; 191 and 217; 192 and 217; 193 and 217; 194 and 217; 195 and 217; 196 and 218; 197 and 219; 198 and 220; 199 and 221; 200 and 222; 201 and 223; 202 and 224; 203 and 225; 204 and 226; 205 and 227; 206 and 228; 207 and 229; 208 and 230; 209 and 231; 210 and 232; 211 and 233; 212 and 234; 213 and 235; 214 and 236; 215 and 237; and 216 and 238.

3. The antibody of claim 2, wherein the antibody comprises a V_{H}-V_{L} pair selected from the list of SEQ ID NO:
167 and 217; 168 and 217; 169 and 217; 171 and 217; 172 and 217; 173 and 217; 174 and 217; 175 and 217; 176 and 217; 177 and 217; 178 and 217; 179 and 217; 180 and 217; 181 and 217; 182 and 217; 183 and 217; 184 and 217; 185 and 217; 186 and 217; 187 and 217; 188 and 217; 189 and 217; 190 and 217; 191 and 217; 192 and 217; 193 and 217; 194 and 217; 195 and 217; 196 and 218; 197 and 219; 198 and 220; 199 and 221; 200 and 222; 201 and 223; 202 and 224; 203 and 225; 204 and 226; 205 and 227; 206 and 228; 207 and 229; 208 and 230; 209 and 231; 210 and 232; 211 and 233; 212 and 234; 213 and 235; 214 and 236; 215 and 237; and 216 and 238.

4.-20. (canceled)

21. The antibody of claim 1 comprising a V_{H} region selected from any one of SEQ ID NOs: 167-216, or a variant thereof having 20 or fewer amino acid substitutions, and a V_{L} region selected from any one of SEQ ID NOs: 217-238, or a variant thereof having 20 or fewer amino acid substitutions.

22.-24. (canceled)

25. The antibody of claim 1, wherein the antibody binds to the same epitope as a second antibody with a V_{H} region selected from any one of SEQ ID NOs: 167-216 and a V_{L} region selected from any one of SEQ ID NOs: 217-238.

26. The antibody of claim 25, wherein the isolated antibody inhibits binding of the second antibody to BCMA by at least 50%, or wherein the second antibody inhibits binding of the isolated antibody to BCMA by at least 50%.

27. The antibody of claim 1, wherein the antibody comprises at least one constant region domain.

28. The antibody of claim 27, wherein the constant region comprises a sequence selected from SEQ ID NOs: 239-242.

29. The antibody of claim 1, wherein the antibody is a monoclonal antibody.

30. (canceled)

31. The antibody of claim 1, wherein the antibody is humanized or human.

32. The antibody of claim 1, wherein the antibody is aglycosylated.

33. The antibody of claim 1, wherein the antibody is an antibody fragment.

34. The antibody of claim 33, wherein the antibody fragment is selected from an Fv fragment, a Fab fragment, a F(ab′)₂ fragment, a Fab′ fragment, an scFv (sFv) fragment, and an scFv-Fc fragment.

35. The antibody of claim 34, wherein the antibody is an scFv fragment.

36. The antibody conjugate of claim 1, wherein the antibody comprises:
(i) a V_{H} comprising: a CDR-H1 comprising one of SEQ ID NOs: 5 and 32; a CDR-H2 comprising one of SEQ ID NOs: 57 and 79; and a CDR-H3 comprising SEQ ID NO: 116;
(ii) a V_{H} comprising: a CDR-H1 comprising one of SEQ ID NOs: 6 and 33; a CDR-H2 comprising one of SEQ ID NOs: 57 and 79; and a CDR-H3 comprising SEQ ID NO: 117;
(iii) a V_{H} comprising: a CDR-H1 comprising one of SEQ ID NOs: 7 and 34; a CDR-H2 comprising one of SEQ ID NOs: 57 and 79; and a CDR-H3 comprising SEQ ID NO: 118;
(v) a V_{H} comprising: a CDR-H1 comprising one of SEQ ID NOs: 9 and 35; a CDR-H2 comprising one of SEQ ID NOs: 57 and 79; and a CDR-H3 comprising SEQ ID NO: 120;
(vi) a V_{H} comprising: a CDR-H1 comprising one of SEQ ID NOs: 10 and 36; a CDR-H2 comprising one of SEQ ID NOs: 57 and 79; and a CDR-H3 comprising SEQ ID NO: 121;
(vii) a V_{H} comprising: a CDR-H1 comprising one of SEQ ID NOs: 11 and 36; a CDR-H2 comprising one of SEQ ID NOs: 58 and 80; and a CDR-H3 comprising SEQ ID NO: 122;
(viii) a V_{H} comprising: a CDR-H1 comprising one of SEQ ID NOs: 12 and 35; a CDR-H2 comprising one of SEQ ID NOs: 57 and 79; and a CDR-H3 comprising SEQ ID NO: 116;
(ix) a V_{H} comprising: a CDR-H1 comprising one of SEQ ID NOs: 13 and 36; a CDR-H2 comprising one of SEQ ID NOs: 59 and 81; and a CDR-H3 comprising SEQ ID NO: 123;
(x) a V_{H} comprising: a CDR-H1 comprising one of SEQ ID NOs: 13 and 36; a CDR-H2 comprising one of SEQ ID NOs: 59 and 81; and a CDR-H3 comprising SEQ ID NO: 124;
(xi) a V_{H} comprising: a CDR-H1 comprising one of SEQ ID NOs: 14 and 37; a CDR-H2 comprising one of SEQ ID NOs: 57 and 79; and a CDR-H3 comprising SEQ ID NO: 125;
(xii) a V_{H} comprising: a CDR-H1 comprising one of SEQ ID NOs: 15 and 35; a CDR-H2 comprising one of SEQ ID NOs: 57 and 79; and a CDR-H3 comprising SEQ ID NO: 126;
(xiii) a V_{H} comprising: a CDR-H1 comprising one of SEQ ID NOs: 16 and 38; a CDR-H2 comprising one of SEQ ID NOs: 57 and 79; and a CDR-H3 comprising SEQ ID NO: 125;
(xiv) a V_{H} comprising: a CDR-H1 comprising one of SEQ ID NOs: 17 and 39; a CDR-H2 comprising one of SEQ ID NOs: 60 and 82; and a CDR-H3 comprising SEQ ID NO: 127;
(xv) a V_{H} comprising: a CDR-H1 comprising one of SEQ ID NOs: 17 and 40; a CDR-H2 comprising one of SEQ ID NOs: 61 and 83; and a CDR-H3 comprising SEQ ID NO: 128;
(xvi) a V_{H} comprising: a CDR-H1 comprising one of SEQ ID NOs: 18 and 41; a CDR-H2 comprising one of SEQ ID NOs: 62 and 84; and a CDR-H3 comprising SEQ ID NO: 129;
(xvii) a V_{H} comprising: a CDR-H1 comprising one of SEQ ID NOs: 19 and 42; a CDR-H2 comprising one of SEQ ID NOs: 63 and 85; and a CDR-H3 comprising SEQ ID NO: 130;
(xviii) a V_{H} comprising: a CDR-H1 comprising one of SEQ ID NOs: 19 and 41; a CDR-H2 comprising one of SEQ ID NOs: 64 and 86; and a CDR-H3 comprising SEQ ID NO: 131;
(xix) a V_{H} comprising: a CDR-H1 comprising one of SEQ ID NOs: 20 and 43; a CDR-H2 comprising one of SEQ ID NOs: 65 and 87; and a CDR-H3 comprising SEQ ID NO: 132;
(xx) a V_{H} comprising: a CDR-H1 comprising one of SEQ ID NOs: 21 and 44; a CDR-H2 comprising one of SEQ ID NOs: 66 and 88; and a CDR-H3 comprising SEQ ID NO: 133;
(xxi) a V_{H} comprising: a CDR-H1 comprising one of SEQ ID NOs: 22 and 42; a CDR-H2 comprising one of SEQ ID NOs: 67 and 89; and a CDR-H3 comprising SEQ ID NO: 134;
(xxii) a V_{H} comprising: a CDR-H1 comprising one of SEQ ID NOs: 23 and 45; a CDR-H2 comprising one of SEQ ID NOs: 66 and 90; and a CDR-H3 comprising SEQ ID NO: 135;
(xxiii) a V_{H} comprising: a CDR-H1 comprising one of SEQ ID NOs: 18 and 46; a CDR-H2 comprising one of SEQ ID NOs: 68 and 91; and a CDR-H3 comprising SEQ ID NO: 136;
(xxiv) a V_{H} comprising: a CDR-H1 comprising one of SEQ ID NOs: 24 and 47; a CDR-H2 comprising one of SEQ ID NOs: 68 and 92; and a CDR-H3 comprising SEQ ID NO: 137;
(xxv) a V_{H} comprising: a CDR-H1 comprising one of SEQ ID NOs: 19 and 48; a CDR-H2 comprising one of SEQ ID NOs: 69 and 93; and a CDR-H3 comprising SEQ ID NO: 138;
(xxvi) a V_{H} comprising: a CDR-H1 comprising one of SEQ ID NOs: 25 and 49; a CDR-H2 comprising one of SEQ ID NOs: 70 and 94; and a CDR-H3 comprising SEQ ID NO: 136;
(xxvii) a V_{H} comprising: a CDR-H1 comprising one of SEQ ID NOs: 26 and 48; a CDR-H2 comprising one of SEQ ID NOs: 70 and 95; and a CDR-H3 comprising SEQ ID NO: 139;
(xxviii) a V_{H} comprising: a CDR-H1 comprising one of SEQ ID NOs: 18 and 50; a CDR-H2 comprising one of SEQ ID NOs: 70 and 96; and a CDR-H3 comprising SEQ ID NO: 140;
(xxix) a V_{H} comprising: a CDR-H1 comprising one of SEQ ID NOs: 26 and 48; a CDR-H2 comprising one of SEQ ID NOs: 70 and 97; and a CDR-H3 comprising SEQ ID NO: 136;
(xxx) a V_{H} comprising: a CDR-H1 comprising one of SEQ ID NOs: 27 and 51; a CDR-H2 comprising one of SEQ ID NOs: 71 and 98; and a CDR-H3 comprising SEQ ID NO: 141;
(xxxi) a V_{H} comprising: a CDR-H1 comprising one of SEQ ID NOs: 27 and 51; a CDR-H2 comprising one of SEQ ID NOs: 72 and 99; and a CDR-H3 comprising SEQ ID NO: 142;
(xxxii) a V_{H} comprising: a CDR-H1 comprising one of SEQ ID NOs: 27 and 51; a CDR-H2 comprising one of SEQ ID NOs: 73 and 100; and a CDR-H3 comprising SEQ ID NO: 142;
(xxxiii) a V_{H} comprising: a CDR-H1 comprising one of SEQ ID NOs: 27 and 51; a CDR-H2 comprising one of SEQ ID NOs: 73 and 101; and a CDR-H3 comprising SEQ ID NO: 142;
(xxxiv) a V_{H} comprising: a CDR-H1 comprising one of SEQ ID NOs: 27 and 51; a CDR-H2 comprising one of SEQ ID NOs: 73 and 102; and a CDR-H3 comprising SEQ ID NO: 142;
(xxxv) a V_{H} comprising: a CDR-H1 comprising one of SEQ ID NOs: 27 and 51; a CDR-H2 comprising one of SEQ ID NOs: 73 and 102; and a CDR-H3 comprising SEQ ID NO: 142;
(xxxvi) a V_{H} comprising: a CDR-H1 comprising one of SEQ ID NOs: 27 and 51; a CDR-H2 comprising one of SEQ ID NOs: 73 and 103; and a CDR-H3 comprising SEQ ID NO: 142;
(xxxvii) a V_{H} comprising: a CDR-H1 comprising one of SEQ ID NOs: 28 and 52; a CDR-H2 comprising one of SEQ ID NOs: 74 and 104; and a CDR-H3 comprising SEQ ID NO: 143;
(xxxviii) a V_{H} comprising: a CDR-H1 comprising one of SEQ ID NOs: 28 and 52; a CDR-H2 comprising one of SEQ ID NOs: 74 and 105; and a CDR-H3 comprising SEQ ID NO: 143;
(xxxix) a V_{H} comprising: a CDR-H1 comprising one of SEQ ID NOs: 28 and 52; a CDR-H2 comprising one of SEQ ID NOs: 74 and 106; and a CDR-H3 comprising SEQ ID NO: 143;
(x1) a V_{H} comprising: a CDR-H1 comprising one of SEQ ID NOs: 28 and 52; a CDR-H2 comprising one of SEQ ID NOs: 74 and 106; and a CDR-H3 comprising SEQ ID NO: 143;
(xli) a V_{H} comprising: a CDR-H1 comprising one of SEQ ID NOs: 28 and 52; a CDR-H2 comprising one of SEQ ID NOs: 74 and 107; and a CDR-H3 comprising SEQ ID NO: 143;
(xlii) a V_{H} comprising: a CDR-H1 comprising one of SEQ ID NOs: 29 and 53; a CDR-H2 comprising one of SEQ ID NOs: 75 and 108; and a CDR-H3 comprising SEQ ID NO: 144;
(xliii) a V_{H} comprising: a CDR-H1 comprising one of SEQ ID NOs: 30 and 54; a CDR-H2 comprising one of SEQ ID NOs: 76 and 109; and a CDR-H3 comprising SEQ ID NO: 145;
(xliv) a V_{H} comprising: a CDR-H1 comprising one of SEQ ID NOs: 30 and 53; a CDR-H2 comprising one of SEQ ID NOs: 77 and 110; and a CDR-H3 comprising SEQ ID NO: 145;
(xlv) a V_{H} comprising: a CDR-H1 comprising one of SEQ ID NOs: 28 and 55; a CDR-H2 comprising one of SEQ ID NOs: 77 and 111; and a CDR-H3 comprising SEQ ID NO: 145;
(xlvi) a V_{H} comprising: a CDR-H1 comprising one of SEQ ID NOs: 31 and 56; a CDR-H2 comprising one of SEQ ID NOs: 78 and 112; and a CDR-H3 comprising SEQ ID NO: 145;
(xlvii) a V_{H} comprising: a CDR-H1 comprising one of SEQ ID NOs: 28 and 55; a CDR-H2 comprising one of SEQ ID NOs: 77 and 113; and a CDR-H3 comprising SEQ ID NO: 145;
(xlviii) a V_{H} comprising: a CDR-H1 comprising one of SEQ ID NOs: 28 and 55; a CDR-H2 comprising one of SEQ ID NOs: 77 and 114; and a CDR-H3 comprising SEQ ID NO: 145;
(xlix) a V_{H} comprising: a CDR-H1 comprising one of SEQ ID NOs: 28 and 55; a CDR-H2 comprising one of SEQ ID NOs: 77 and 114; and a CDR-H3 comprising SEQ ID NO: 145;
(l) a V_{H} comprising: a CDR-H1 comprising one of SEQ ID NOs: 28 and 55; a CDR-H2 comprising one of SEQ ID NOs: 77 and 115; and a CDR-H3 comprising SEQ ID NO: 145.

37. The antibody conjugate of claim 1, wherein the antibody comprises:
(a) a V_{L} comprising: a CDR-L1 comprising SEQ ID NO: 146; a CDR-L2 comprising SEQ ID NO: 155; and a CDR-L3 comprising SEQ ID NO: 161; or
(b) a V_{L} comprising: a CDR-L1 comprising SEQ ID NO: 147; a CDR-L2 comprising SEQ ID NO: 156; and a CDR-L3 comprising SEQ ID NO: 162; or
(c) a V_{L} comprising: a CDR-L1 comprising SEQ ID NO: 148; a CDR-L2 comprising SEQ ID NO: 158; and a CDR-L3 comprising SEQ ID NO: 163; or
(d) a V_{L} comprising: a CDR-L1 comprising SEQ ID NO: 149; a CDR-L2 comprising SEQ ID NO: 158; and a CDR-L3 comprising SEQ ID NO: 164; or
(e) a V_{L} comprising: a CDR-L1 comprising SEQ ID NO: 149; a CDR-L2 comprising SEQ ID NO: 158; and a CDR-L3 comprising SEQ ID NO: 165; or
(f) a V_{L} comprising: a CDR-L1 comprising SEQ ID NO: 149; a CDR-L2 comprising SEQ ID NO: 158; and a CDR-L3 comprising SEQ ID NO: 165; or
(g) a V_{L} comprising: a CDR-L1 comprising SEQ ID NO: 149; a CDR-L2 comprising SEQ ID NO: 158; and a CDR-L3 comprising SEQ ID NO: 165; or
(h) a V_{L} comprising: a CDR-L1 comprising SEQ ID NO: 149; a CDR-L2 comprising SEQ ID NO: 158; and a CDR-L3 comprising SEQ ID NO: 165; or
(i) a V_{L} comprising: a CDR-L1 comprising SEQ ID NO: 150; a CDR-L2 comprising SEQ ID NO: 159; and a CDR-L3 comprising SEQ ID NO: 166; or
(j) a V_{L} comprising: a CDR-L1 comprising SEQ ID NO: 150; a CDR-L2 comprising SEQ ID NO: 159; and a CDR-L3 comprising SEQ ID NO: 166; or
(k) a V_{L} comprising: a CDR-L1 comprising SEQ ID NO: 151; a CDR-L2 comprising SEQ ID NO: 159; and a CDR-L3 comprising SEQ ID NO: 166; or
(l) a V_{L} comprising: a CDR-L1 comprising SEQ ID NO: 152; a CDR-L2 comprising SEQ ID NO: 159; and a CDR-L3 comprising SEQ ID NO: 166; or
(m) a V_{L} comprising: a CDR-L1 comprising SEQ ID NO: 152; a CDR-L2 comprising SEQ ID NO: 159; and a CDR-L3 comprising SEQ ID NO: 166; or
(n) a V_{L} comprising: a CDR-L1 comprising SEQ ID NO: 153; a CDR-L2 comprising SEQ ID NO: 159; and a CDR-L3 comprising SEQ ID NO: 166; or
(o) a V_{L} comprising: a CDR-L1 comprising SEQ ID NO: 146; a CDR-L2 comprising SEQ ID NO: 155; and a CDR-L3 comprising SEQ ID NO; or
(p) a V_{L} comprising: a CDR-L1 comprising SEQ ID NO: 154; a CDR-L2 comprising SEQ ID NO: 160; and a CDR-L3 comprising SEQ ID NO: 166.

38. The antibody conjugate of claim 1, wherein the antibody comprises:
(i) a V_{H} comprising: a CDR-H1 comprising one of SEQ ID NOs: 5 and 32; a CDR-H2 comprising one of SEQ ID NOs: 57 and 79; a CDR-H3 comprising SEQ ID NO: 116; a CDR-L1 comprising SEQ ID NO: 146; a CDR-L2 comprising SEQ ID NO: 155; and a CDR-L3 comprising SEQ ID NO: 161;
(ii) a V_{H} comprising: a CDR-H1 comprising one of SEQ ID NOs: 6 and 33; a CDR-H2 comprising one of SEQ ID NOs: 57 and 79; a CDR-H3 comprising SEQ ID NO: 117; a CDR-L1 comprising SEQ ID NO: 146; a CDR-L2 comprising SEQ ID NO: 155; and a CDR-L3 comprising SEQ ID NO: 161;
(iii) a V_{H} comprising: a CDR-H1 comprising one of SEQ ID NOs: 7 and 34; a CDR-H2 comprising one of SEQ ID NOs: 57 and 79; a CDR-H3 comprising SEQ ID NO: 118; a CDR-L1 comprising SEQ ID NO: 146; a CDR-L2 comprising SEQ ID NO: 155; and a CDR-L3 comprising SEQ ID NO: 161;
(v) a V_{H} comprising: a CDR-H1 comprising one of SEQ ID NOs: 9 and 35; a CDR-H2 comprising one of SEQ ID NOs: 57 and 79; a CDR-H3 comprising SEQ ID NO: 120; a CDR-L1 comprising SEQ ID NO: 146; a CDR-L2 comprising SEQ ID NO: 155; and a CDR-L3 comprising SEQ ID NO: 161;
(vi) a V_{H} comprising: a CDR-H1 comprising one of SEQ ID NOs: 10 and 36; a CDR-H2 comprising one of SEQ ID NOs: 57 and 79; a CDR-H3 comprising SEQ ID NO: 121; a CDR-L1 comprising SEQ ID NO: 146; a CDR-L2 comprising SEQ ID NO: 155; and a CDR-L3 comprising SEQ ID NO: 161;
(vii) a V_{H} comprising: a CDR-H1 comprising one of SEQ ID NOs: 11 and 36; a CDR-H2 comprising one of SEQ ID NOs: 58 and 80; a CDR-H3 comprising SEQ ID NO: 122; a CDR-L1 comprising SEQ ID NO: 146; a CDR-L2 comprising SEQ ID NO: 155; and a CDR-L3 comprising SEQ ID NO: 161;
(viii) a V_{H} comprising: a CDR-H1 comprising one of SEQ ID NOs: 12 and 35; a CDR-H2 comprising one of SEQ ID NOs: 57 and 79; a CDR-H3 comprising SEQ ID NO: 116; a CDR-L1 comprising SEQ ID NO: 146; a CDR-L2 comprising SEQ ID NO: 155; and a CDR-L3 comprising SEQ ID NO: 161;
(ix) a V_{H} comprising: a CDR-H1 comprising one of SEQ ID NOs: 13 and 36; a CDR-H2 comprising one of SEQ ID NOs: 59 and 81; a CDR-H3 comprising SEQ ID NO: 123; a CDR-L1 comprising SEQ ID NO: 146; a CDR-L2 comprising SEQ ID NO: 155; and a CDR-L3 comprising SEQ ID NO: 161;
(x) a V_{H} comprising: a CDR-H1 comprising one of SEQ ID NOs: 13 and 36; a CDR-H2 comprising one of SEQ ID NOs: 59 and 81; a CDR-H3 comprising SEQ ID NO: 124; a CDR-L1 comprising SEQ ID NO: 146; a CDR-L2 comprising SEQ ID NO: 155; and a CDR-L3 comprising SEQ ID NO: 161;
(xi) a V_{H} comprising: a CDR-H1 comprising one of SEQ ID NOs: 14 and 37; a CDR-H2 comprising one of SEQ ID NOs: 57 and 79; a CDR-H3 comprising SEQ ID NO: 125; a CDR-L1 comprising SEQ ID NO: 146; a CDR-L2 comprising SEQ ID NO: 155; and a CDR-L3 comprising SEQ ID NO: 161;
(xii) a V_{H} comprising: a CDR-H1 comprising one of SEQ ID NOs: 15 and 35; a CDR-H2 comprising one of SEQ ID NOs: 57 and 79; a CDR-H3 comprising SEQ ID NO: 126; a CDR-L1 comprising SEQ ID NO: 146; a CDR-L2 comprising SEQ ID NO: 155; and a CDR-L3 comprising SEQ ID NO: 161;
(xiii) a V_{H} comprising: a CDR-H1 comprising one of SEQ ID NOs: 16 and 38; a CDR-H2 comprising one of SEQ ID NOs: 57 and 79; a CDR-H3 comprising SEQ ID NO: 125; a CDR-L1 comprising SEQ ID NO: 146; a CDR-L2 comprising SEQ ID NO: 155; and a CDR-L3 comprising SEQ ID NO: 161;
(xiv) a V_{H} comprising: a CDR-H1 comprising one of SEQ ID NOs: 17 and 39; a CDR-H2 comprising one of SEQ ID NOs: 60 and 82; a CDR-H3 comprising SEQ ID NO: 127; a CDR-L1 comprising SEQ ID NO: 146; a CDR-L2 comprising SEQ ID NO: 155; and a CDR-L3 comprising SEQ ID NO: 161;
(xv) a V_{H} comprising: a CDR-H1 comprising one of SEQ ID NOs: 17 and 40; a CDR-H2 comprising one of SEQ ID NOs: 61 and 83; a CDR-H3 comprising SEQ ID NO: 128; a CDR-L1 comprising SEQ ID NO: 146; a CDR-L2 comprising SEQ ID NO: 155; and a CDR-L3 comprising SEQ ID NO: 161;
(xvi) a V_{H} comprising: a CDR-H1 comprising one of SEQ ID NOs: 18 and 41; a CDR-H2 comprising one of SEQ ID NOs: 62 and 84; a CDR-H3 comprising SEQ ID NO: 129; a CDR-L1 comprising SEQ ID NO: 146; a CDR-L2 comprising SEQ ID NO: 155; and a CDR-L3 comprising SEQ ID NO: 161;
(xvii) a V_{H} comprising: a CDR-H1 comprising one of SEQ ID NOs: 19 and 42; a CDR-H2 comprising one of SEQ ID NOs: 63 and 85; a CDR-H3 comprising SEQ ID NO: 130; a CDR-L1 comprising SEQ ID NO: 146; a CDR-L2 comprising SEQ ID NO: 155; and a CDR-L3 comprising SEQ ID NO: 161;
(xviii) a V_{H} comprising: a CDR-H1 comprising one of SEQ ID NOs: 19 and 41; a CDR-H2 comprising one of SEQ ID NOs: 64 and 86; a CDR-H3 comprising SEQ ID NO: 131; a CDR-L1 comprising SEQ ID NO: 146; a CDR-L2 comprising SEQ ID NO: 155; and a CDR-L3 comprising SEQ ID NO: 161;
(xix) a V_{H} comprising: a CDR-H1 comprising one of SEQ ID NOs: 20 and 43; a CDR-H2 comprising one of SEQ ID NOs: 65 and 87; a CDR-H3 comprising SEQ ID NO: 132; a CDR-L1 comprising SEQ ID NO: 146; a CDR-L2 comprising SEQ ID NO: 155; and a CDR-L3 comprising SEQ ID NO: 161;
(xx) a V_{L} comprising: a CDR-H1 comprising one of SEQ ID NOs: 21 and 44; a CDR-H2 comprising one of SEQ ID NOs: 66 and 88; a CDR-H3 comprising SEQ ID NO: 133; a CDR-L1 comprising SEQ ID NO: 146; a CDR-L2 comprising SEQ ID NO: 155; and a CDR-L3 comprising SEQ ID NO: 161;
(xxi) a V_{H} comprising: a CDR-H1 comprising one of SEQ ID NOs: 22 and 42; a CDR-H2 comprising one of SEQ ID NOs: 67 and 89; a CDR-H3 comprising SEQ ID NO: 134; a CDR-L1 comprising SEQ ID NO: 146; a CDR-L2 comprising SEQ ID NO: 155; and a CDR-L3 comprising SEQ ID NO: 161;
(xxii) a V_{H} comprising: a CDR-H1 comprising one of SEQ ID NOs: 23 and 45; a CDR-H2 comprising one of SEQ ID NOs: 66 and 90; a CDR-H3 comprising SEQ ID NO: 135; a CDR-L1 comprising SEQ ID NO: 146; a CDR-L2 comprising SEQ ID NO: 155; and a CDR-L3 comprising SEQ ID NO: 161;
(xxiii) a V_{H} comprising: a CDR-H1 comprising one of SEQ ID NOs: 18 and 46; a CDR-H2 comprising one of SEQ ID NOs: 68 and 91; a CDR-H3 comprising SEQ ID NO: 136; a CDR-L1 comprising SEQ ID NO: 146; a CDR-L2 comprising SEQ ID NO: 155; and a CDR-L3 comprising SEQ ID NO: 161;
(xxiv) a V_{H} comprising: a CDR-H1 comprising one of SEQ ID NOs: 24 and 47; a CDR-H2 comprising one of SEQ ID NOs: 68 and 92; a CDR-H3 comprising SEQ ID NO: 137; a CDR-L1 comprising SEQ ID NO: 146; a CDR-L2 comprising SEQ ID NO: 155; and a CDR-L3 comprising SEQ ID NO: 161;
(xxv) a V_{H} comprising: a CDR-H1 comprising one of SEQ ID NOs: 19 and 48; a CDR-H2 comprising one of SEQ ID NOs: 69 and 93; a CDR-H3 comprising SEQ ID NO: 138; a CDR-L1 comprising SEQ ID NO: 146; a CDR-L2 comprising SEQ ID NO: 155; and a CDR-L3 comprising SEQ ID NO: 161;
(xxvi) a V_{H} comprising: a CDR-H1 comprising one of SEQ ID NOs: 25 and 49; a CDR-H2 comprising one of SEQ ID NOs: 70 and 94; a CDR-H3 comprising SEQ ID NO: 136; a CDR-L1 comprising SEQ ID NO: 146; a CDR-L2 comprising SEQ ID NO: 155; and a CDR-L3 comprising SEQ ID NO: 161;
(xxvii) a V_{H} comprising: a CDR-H1 comprising one of SEQ ID NOs: 26 and 48; a CDR-H2 comprising one of SEQ ID NOs: 70 and 95; a CDR-H3 comprising SEQ ID NO: 139; a CDR-L1 comprising SEQ ID NO: 146; a CDR-L2 comprising SEQ ID NO: 155; and a CDR-L3 comprising SEQ ID NO: 161;
(xxviii) a V_{H} comprising: a CDR-H1 comprising one of SEQ ID NOs: 18 and 50; a CDR-H2 comprising one of SEQ ID NOs: 70 and 96; a CDR-H3 comprising SEQ ID NO: 140; a CDR-L1 comprising SEQ ID NO: 146; a CDR-L2 comprising SEQ ID NO: 155; and a CDR-L3 comprising SEQ ID NO: 161;
(xxix) a V_{H} comprising: a CDR-H1 comprising one of SEQ ID NOs: 26 and 48; a CDR-H2 comprising one of SEQ ID NOs: 70 and 97; a CDR-H3 comprising SEQ ID NO: 136; a CDR-L1 comprising SEQ ID NO: 146; a CDR-L2 comprising SEQ ID NO: 155; and a CDR-L3 comprising SEQ ID NO: 161;
(b) a V_{H} comprising: a CDR-H1 comprising one of SEQ ID NOs: 27 and 51; a CDR-H2 comprising one of SEQ ID NOs: 71 and 98; a CDR-H3 comprising SEQ ID NO: 141; a CDR-L1 comprising SEQ ID NO: 147; a CDR-L2 comprising SEQ ID NO: 156; and a CDR-L3 comprising SEQ ID NO: 162;
(i) a V_{H} comprising: a CDR-H1 comprising one of SEQ ID NOs: 27 and 51; a CDR-H2 comprising one of SEQ ID NOs: 72 and 99; a CDR-H3 comprising SEQ ID NO: 142; a CDR-L1 comprising SEQ ID NO: 148; a CDR-L2 comprising SEQ ID NO: 158; and a CDR-L3 comprising SEQ ID NO: 163;
(ii) a V_{H} comprising: a CDR-H1 comprising one of SEQ ID NOs: 27 and 51; a CDR-H2 comprising one of SEQ ID NOs: 73 and 100; a CDR-H3 comprising SEQ ID NO: 142; a CDR-L1 comprising SEQ ID NO: 149; a CDR-L2 comprising SEQ ID NO: 158; and a CDR-L3 comprising SEQ ID NO: 164;
(iii) a V_{H} comprising: a CDR-H1 comprising one of SEQ ID NOs: 27 and 51; a CDR-H2 comprising one of SEQ ID NOs: 73 and 101; and a CDR-H3 comprising SEQ ID NO: 142; a CDR-L1 comprising SEQ ID NO: 149; a CDR-L2 comprising SEQ ID NO: 158; and a CDR-L3 comprising SEQ ID NO: 165;
(iv) a V_{H} comprising: a CDR-H1 comprising one of SEQ ID NOs: 27 and 51; a CDR-H2 comprising one of SEQ ID NOs: 73 and 102; and a CDR-H3 comprising SEQ ID NO: 142; a CDR-L1 comprising SEQ ID NO: 149; a CDR-L2 comprising SEQ ID NO: 158; and a CDR-L3 comprising SEQ ID NO: 165;
(v) a V_{H} comprising: a CDR-H1 comprising one of SEQ ID NOs: 27 and 51; a CDR-H2 comprising one of SEQ ID NOs: 73 and 102; and a CDR-H3 comprising SEQ ID NO: 142; a CDR-L1 comprising SEQ ID NO: 149; a CDR-L2 comprising SEQ ID NO: 158; and a CDR-L3 comprising SEQ ID NO: 165;
(vi) a V_{H} comprising: a CDR-H1 comprising one of SEQ ID NOs: 27 and 51; a CDR-H2 comprising one of SEQ ID NOs: 73 and 103; and a CDR-H3 comprising SEQ ID NO: 142; a CDR-L1 comprising SEQ ID NO: 149; a CDR-L2 comprising SEQ ID NO: 158; and a CDR-L3 comprising SEQ ID NO: 165;
(vii) a V_{H} comprising: a CDR-H1 comprising one of SEQ ID NOs: 28 and 52; a CDR-H2 comprising one of SEQ ID NOs: 74 and 104; and a CDR-H3 comprising SEQ ID NO: 143; a CDR-L1 comprising SEQ ID NO: 150; a CDR-L2 comprising SEQ ID NO: 159; and a CDR-L3 comprising SEQ ID NO: 166;
(viii) a V_{H} comprising: a CDR-H1 comprising one of SEQ ID NOs: 28 and 52; a CDR-H2 comprising one of SEQ ID NOs: 74 and 105; and a CDR-H3 comprising SEQ ID NO: 143; a CDR-L1 comprising SEQ ID NO: 150; a CDR-L2 comprising SEQ ID NO: 159; and a CDR-L3 comprising SEQ ID NO: 166;
(ix) a V_{H} comprising: a CDR-H1 comprising one of SEQ ID NOs: 28 and 52; a CDR-H2 comprising one of SEQ ID NOs: 74 and 106; and a CDR-H3 comprising SEQ ID NO: 143; a CDR-L1 comprising SEQ ID NO: 150; a CDR-L2 comprising SEQ ID NO: 159; and a CDR-L3 comprising SEQ ID NO: 166;
(x) a V_{L} comprising: a CDR-H1 comprising one of SEQ ID NOs: 28 and 52; a CDR-H2 comprising one of SEQ ID NOs: 74 and 106; and a CDR-H3 comprising SEQ ID NO: 143; a CDR-L1 comprising SEQ ID NO: 150; a CDR-L2 comprising SEQ ID NO: 159; and a CDR-L3 comprising SEQ ID NO: 166;
(xi) a V_{H} comprising: a CDR-H1 comprising one of SEQ ID NOs: 28 and 52; a CDR-H2 comprising one of SEQ ID NOs: 74 and 107; and a CDR-H3 comprising SEQ ID NO: 143; a CDR-L1 comprising SEQ ID NO: 150; a CDR-L2 comprising SEQ ID NO: 159; and a CDR-L3 comprising SEQ ID NO: 166;
(xii) a V_{H} comprising: a CDR-H1 comprising one of SEQ ID NOs: 29 and 53; a CDR-H2 comprising one of SEQ ID NOs: 75 and 108; and a CDR-H3 comprising SEQ ID NO: 144; a CDR-L1 comprising SEQ ID NO: 151; a CDR-L2 comprising SEQ ID NO: 159; and a CDR-L3 comprising SEQ ID NO: 166;
(xiii) a V_{H} comprising: a CDR-H1 comprising one of SEQ ID NOs: 30 and 54; a CDR-H2 comprising one of SEQ ID NOs: 76 and 109; and a CDR-H3 comprising SEQ ID NO: 145; a CDR-L1 comprising SEQ ID NO: 152; a CDR-L2 comprising SEQ ID NO: 159; and a CDR-L3 comprising SEQ ID NO: 166;
(xiv) a V_{H} comprising: a CDR-H1 comprising one of SEQ ID NOs: 30 and 53; a CDR-H2 comprising one of SEQ ID NOs: 77 and 110; and a CDR-H3 comprising SEQ ID NO: 145; a CDR-L1 comprising SEQ ID NO: 152; a CDR-L2 comprising SEQ ID NO: 159; and a CDR-L3 comprising SEQ ID NO: 166;
(xv) a V_{H} comprising: a CDR-H1 comprising one of SEQ ID NOs: 28 and 55; a CDR-H2 comprising one of SEQ ID NOs: 77 and 111; and a CDR-H3 comprising SEQ ID NO: 145; a CDR-L1 comprising SEQ ID NO: 153; a CDR-L2 comprising SEQ ID NO: 159; and a CDR-L3 comprising SEQ ID NO: 166;
(xvi) a V_{H} comprising: a CDR-H1 comprising one of SEQ ID NOs: 31 and 56; a CDR-H2 comprising one of SEQ ID NOs: 78 and 112; and a CDR-H3 comprising SEQ ID NO: 145; a CDR-L1 comprising SEQ ID NO: 154; a CDR-L2 comprising SEQ ID NO: 160; and a CDR-L3 comprising SEQ ID NO: 166;
(xvii) a V_{H} comprising: a CDR-H1 comprising one of SEQ ID NOs: 28 and 55; a CDR-H2 comprising one of SEQ ID NOs: 77 and 113; and a CDR-H3 comprising SEQ ID NO: 145; a CDR-L1 comprising SEQ ID NO: 153; a CDR-L2 comprising SEQ ID NO: 159; and a CDR-L3 comprising SEQ ID NO: 166;
(xviii) a V_{H} comprising: a CDR-H1 comprising one of SEQ ID NOs: 28 and 55; a CDR-H2 comprising one of SEQ ID NOs: 77 and 114; and a CDR-H3 comprising SEQ ID NO: 145; a CDR-L1 comprising SEQ ID NO: 153; a CDR-L2 comprising SEQ ID NO: 159; and a CDR-L3 comprising SEQ ID NO: 166;
(xix) a V_{H} comprising: a CDR-H1 comprising one of SEQ ID NOs: 28 and 55; a CDR-H2 comprising one of SEQ ID NOs: 77 and 114; and a CDR-H3 comprising SEQ ID NO: 145; a CDR-L1 comprising SEQ ID NO: 153; a CDR-L2 comprising SEQ ID NO: 159; and a CDR-L3 comprising SEQ ID NO: 166;
(xx) a V_{H} comprising: a CDR-H1 comprising one of SEQ ID NOs: 28 and 55; a CDR-H2 comprising one of SEQ ID NOs: 77 and 115; and a CDR-H3 comprising SEQ ID NO: 145; a CDR-L1 comprising SEQ ID NO: 153; a CDR-L2 comprising SEQ ID NO: 159; and a CDR-L3 comprising SEQ ID NO: 166.

39. The antibody of claim 1, wherein the antibody has a kₐ of about 4.57×10⁵ M⁻¹×sec⁻¹ to about 7.66×10⁵ M⁻¹×sec⁻¹ when associating with human BCMA at a temperature of 25° C.

40. The antibody of claim 1, wherein the antibody has a k_{d} of about 1.93×10⁻⁴ sec⁻¹ to about 7.45×10⁻⁴ sec⁻¹ when dissociating from human BCMA at a temperature of 25° C.

41. The antibody of claim 1, wherein the antibody has a K_{D} of about 2.76×10⁻¹⁰ M to about 1.63×10⁻⁹ M when bound to human BCMA at a temperature of 25° C.

42.-50. (canceled)

51. A pharmaceutical composition comprising the antibody claim 1 and a pharmaceutically acceptable carrier.

52. A method of treating or preventing a disease or condition in a subject in need thereof, comprising administering to the subject an effective amount of an antibody of claim 1, or a pharmaceutical composition, wherein the disease or condition is a BCMA-expressing cancer.

53. A method of diagnosing a disease or condition in a subject in need thereof, comprising administering to the subject an effective amount of an antibody of claim 1, or a pharmaceutical composition wherein the disease or condition is a BCMA-expressing cancer.

54. The method of claim 52, wherein the BCMA-expressing cancer is leukemia, lymphoma, multiple myeloma, a plasmacytoid dendritic cell tumor, a B-cell lineage malignancy, a plasma cell neoplasm, diffuse large B-cell lymophoma (DLBCL), a low-grade B-cell lymphoma, Burkitt's lymphoma, a plasmablastic lymphoma, or a follicular lymphoma.

55.-57. (canceled)


SEQUENCE LISTING (275 sequences)

SEQ ID NO: 1 - protein, 184 aa, Homo sapiens
  REGION 1..184: note = misc_feature - Human BCMA (Isoform 1)
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 2 - protein, 135 aa, Homo sapiens
  REGION 1..135: note = misc_feature - Human BCMA (Isoform 2)
MLQMAGQCSQNEYFDSLLHACIPCQLRCSSNTPPLTCQRYCNARSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 3 - protein, 183 aa, Macaca mulatta
  REGION 1..183: note = misc_feature - Cynomolgus BCMA
MLQMARQCSQNEYFDSLLHDCKPCQLRCSSTPPLTCQRYCNASMTNSVKGMNAILWTCLGLSLIISLAVFVLTFLLRKMSSEPLKDEFKNTGSGLLGMANIDLEKGRTGDEIVLPRGLEYTVEECTCEDCIKNKPKVDSDHCFPLPAMEEGATILVTTKTNDYCNSLSAALSVTEIEKSISAR

SEQ ID NO: 4 - protein, 185 aa, Mus musculus
  REGION 1..185: note = misc_feature - Murine BCMA
MAQQCFHSEYFDSLLHACKPCHLRCSNPPATCQPYCDPSVTSSVKGTYTVLWIFLGLTLVLSLALFTISFLLRKMNPEALKDEPQSPGQLDGSAQLDKADTELTRIRAGDDRIFPRSLEYTVEECTCEDCVKSKPKGDSDHFFPLPAMEEGATILVTTKTGDYGKSSVPTALQSVMGMEKPTHTR

SEQ ID NO: 5 - protein, 7 aa, synthetic construct
  REGION 1..7: note = Synthetic: 108843.00220_ST25 - v1
GFNISGS

SEQ ID NO: 6 - protein, 7 aa, synthetic construct
  REGION 1..7: note = Synthetic: 2265-F06; CDR-H1; Chothia
GFNISYP

SEQ ID NO: 7 - protein, 7 aa, synthetic construct
  REGION 1..7: note = Synthetic: 2265-F05; CDR-H1; Chothia
GFNIIAP

SEQ ID NO: 8 - protein, 7 aa, synthetic construct
  REGION 1..7: note = Synthetic: 2265-F02; CDR-H1; Chothia
GFNISAP

SEQ ID NO: 9 - protein, 7 aa, synthetic construct
  REGION 1..7: note = Synthetic: 2265-B06; CDR-H1; Chothia
GFNIRVS

SEQ ID NO: 10 - protein, 7 aa, synthetic construct
  REGION 1..7: note = Synthetic: 2265-A09; CDR-H1; Chothia
GFNIIGP

SEQ ID NO: 11 - protein, 7 aa, synthetic construct
  REGION 1..7: note = Synthetic: 2265-F03; CDR-H1; Chothia
GFNIRGP

SEQ ID NO: 12 - protein, 7 aa, synthetic construct
  REGION 1..7: note = Synthetic: 2265-E02; CDR-H1; Chothia
GFNIYVS

SEQ ID NO: 13 - protein, 7 aa, synthetic construct
  REGION 1..7: note = Synthetic: 2265-D11; CDR-H1; Chothia
GFNISGP

SEQ ID NO: 14 - protein, 7 aa, synthetic construct
  REGION 1..7: note = Synthetic: 2265-C03; CDR-H1; Chothia
GFNISVP

SEQ ID NO: 15 - protein, 7 aa, synthetic construct
  REGION 1..7: note = Synthetic: 2265-C02; CDR-H1; Chothia
GFNIGVS

SEQ ID NO: 16 - protein, 7 aa, synthetic construct
  REGION 1..7: note = Synthetic: 2265-A06; CDR-H1; Chothia
GFNIYRS

SEQ ID NO: 17 - protein, 7 aa, synthetic construct
  REGION 1..7: note = Synthetic: 2137-A05; CDR-H1; Chothia
GFNINNS

SEQ ID NO: 18 - protein, 7 aa, synthetic construct
  REGION 1..7: note = Synthetic: 2190-B01; CDR-H1; Chothia
GFNISSY

SEQ ID NO: 19 - protein, 7 aa, synthetic construct
  REGION 1..7: note = Synthetic: 2290-G01; CDR-H1; Chothia
GFNISPY

SEQ ID NO: 20 - protein, 7 aa, synthetic construct
  REGION 1..7: note = Synthetic: 2290-C07; CDR-H1; Chothia
GFNITYD

SEQ ID NO: 21 - protein, 7 aa, synthetic construct
  REGION 1..7: note = Synthetic: 2290-D05; CDR-H1; Chothia
GFNIASR

SEQ ID NO: 22 - protein, 7 aa, synthetic construct
  REGION 1..7: note = Synthetic: 2290-C08; CDR-H1; Chothia
GFNIQPY

SEQ ID NO: 23 - protein, 7 aa, synthetic construct
  REGION 1..7: note = Synthetic: 2290-A02; CDR-H1; Chothia
GFNISTR

SEQ ID NO: 24 - protein, 7 aa, synthetic construct
  REGION 1..7: note = Synthetic: 2291-G05; CDR-H1; Chothia
GFNIAAY

SEQ ID NO: 25 - protein, 7 aa, synthetic construct
  REGION 1..7: note = Synthetic: 2291-D07; CDR-H1; Chothia
GFNIKDT

SEQ ID NO: 26 - protein, 7 aa, synthetic construct
  REGION 1..7: note = Synthetic: 2291-F10; CDR-H1; Chothia
GFNIDPY

SEQ ID NO: 27 - protein, 7 aa, synthetic construct
  REGION 1..7: note = Synthetic: 9A8; CDR-H1; Chothia
GFTFSSF

SEQ ID NO: 28 - protein, 7 aa, synthetic construct
  REGION 1..7: note = Synthetic: 10F4; CDR-H1; Chothia
GFTFSGY

SEQ ID NO: 29 - protein, 7 aa, synthetic construct
  REGION 1..7: note = Synthetic: 9A5; CDR-H1; Chothia
GFSISDY

SEQ ID NO: 30 - protein, 7 aa, synthetic construct
  REGION 1..7: note = Synthetic: 9E12; CDR-H1; Chothia
GFTFSDY

SEQ ID NO: 31 - protein, 7 aa, synthetic construct
  REGION 1..7: note = Synthetic: 10E10; CDR-H1; Chothia
GFTFSSY

SEQ ID NO: 32 - protein, 5 aa, synthetic construct
  REGION 1..5: note = Synthetic: 2137-C07; CDR-H1; Kabat
GSGIH

SEQ ID NO: 33 - protein, 5 aa, synthetic construct
  REGION 1..5: note = Synthetic: 2265-F06; CDR-H1; Kabat
YPGIH

SEQ ID NO: 34 - protein, 5 aa, synthetic construct
  REGION 1..5: note = Synthetic: 2265-F05; CDR-H1; Kabat
APGIH

SEQ ID NO: 35 - protein, 5 aa, synthetic construct
  REGION 1..5: note = Synthetic: 2265-B06; CDR-H1; Kabat
VSGIH

SEQ ID NO: 36 - protein, 5 aa, synthetic construct
  REGION 1..5: note = Synthetic: 2265-A09; CDR-H1; Kabat
GPGIH

SEQ ID NO: 37 - protein, 5 aa, synthetic construct
  REGION 1..5: note = Synthetic: 2265-C03; CDR-H1; Kabat
VPGIH

SEQ ID NO: 38 - protein, 5 aa, synthetic construct
  REGION 1..5: note = Synthetic: 2265-A06; CDR-H1; Kabat
RSGIH

SEQ ID NO: 39 - protein, 5 aa, synthetic construct
  REGION 1..5: note = Synthetic: 2137-A05; CDR-H1; Kabat
NSYIH

SEQ ID NO: 40 - protein, 5 aa, synthetic construct
  REGION 1..5: note = Synthetic: 2288-A03; CDR-H1; Kabat
NSWIH

SEQ ID NO: 41 - protein, 5 aa, synthetic construct
  REGION 1..5: note = Synthetic: 2190-B01; CDR-H1; Kabat
SYWIH

SEQ ID NO: 42 - protein, 5 aa, synthetic construct
  REGION 1..5: note = Synthetic: 2290-G01; CDR-H1; Kabat
PYWIH

SEQ ID NO: 43 - protein, 5 aa, synthetic construct
  REGION 1..5: note = Synthetic: 2290-C07; CDR-H1; Kabat
YDWIH

SEQ ID NO: 44 - protein, 5 aa, synthetic construct
  REGION 1..5: note = Synthetic: 2290-D05; CDR-H1; Kabat
SRWIH

SEQ ID NO: 45 - protein, 5 aa, synthetic construct
  REGION 1..5: note = Synthetic: 2290-A02; CDR-H1; Kabat
TRWIH

SEQ ID NO: 46 - protein, 5 aa, synthetic construct
  REGION 1..5: note = Synthetic: 2213-A06; CDR-H1; Kabat
SYAIH

SEQ ID NO: 47 - protein, 5 aa, synthetic construct
  REGION 1..5: note = Synthetic: 2291-G05; CDR-H1; Kabat
AYTIH

SEQ ID NO: 48 - protein, 5 aa, synthetic construct
  REGION 1..5: note = Synthetic: 2291-E06; CDR-H1; Kabat
PYTIH

SEQ ID NO: 49 - protein, 5 aa, synthetic construct
  REGION 1..5: note = Synthetic: 2291-D07; CDR-H1; Kabat
DTYIH

SEQ ID NO: 50 - protein, 5 aa, synthetic construct
  REGION 1..5: note = Synthetic: 2291-A04; CDR-H1; Kabat
SYGIH

SEQ ID NO: 51 - protein, 5 aa, synthetic construct
  REGION 1..5: note = Synthetic: 9A8; CDR-H1; Kabat
SFNMF

SEQ ID NO: 52 - protein, 5 aa, synthetic construct
  REGION 1..5: note = Synthetic: 10F4; CDR-H1; Kabat
GYNMG

SEQ ID NO: 53 - protein, 5 aa, synthetic construct
  REGION 1..5: note = Synthetic: 9A5; CDR-H1; Kabat
DYGMG

SEQ ID NO: 54 - protein, 5 aa, synthetic construct
  REGION 1..5: note = Synthetic: 9E12; CDR-H1; Kabat
DYGLG

SEQ ID NO: 55 - protein, 5 aa, synthetic construct
  REGION 1..5: note = Synthetic: 10H1; CDR-H1; Kabat
GYGMG

SEQ ID NO: 56 - protein, 5 aa, synthetic construct
  REGION 1..5: note = Synthetic: 10E10; CDR-H1; Kabat
SYGMG

SEQ ID NO: 57 - protein, 6 aa, synthetic construct
  REGION 1..6: note = Synthetic: 2137-C07; CDR-H2; Chothia
NPAGGY

SEQ ID NO: 58 - protein, 6 aa, synthetic construct
  REGION 1..6: note = Synthetic: 2265-F03; CDR-H2; Chothia
SPAAGY

SEQ ID NO: 59 - protein, 6 aa, synthetic construct
  REGION 1..6: note = Synthetic: 2265-D11; CDR-H2; Chothia
NPAAGY

SEQ ID NO: 60 - protein, 6 aa, synthetic construct
  REGION 1..6: note = Synthetic: 2137-A05; CDR-H2; Chothia
YPYSGY

SEQ ID NO: 61 - protein, 6 aa, synthetic construct
  REGION 1..6: note = Synthetic: 2288-A03; CDR-H2; Chothia
YPYIGF

SEQ ID NO: 62 - protein, 6 aa, synthetic construct
  REGION 1..6: note = Synthetic: 2190-B01; CDR-H2; Chothia
TPSGGY

SEQ ID NO: 63 - protein, 6 aa, synthetic construct
  REGION 1..6: note = Synthetic: 2290-G01; CDR-H2; Chothia
TPPSGF

SEQ ID NO: 64 - protein, 6 aa, synthetic construct
  REGION 1..6: note = Synthetic: 2290-D02; CDR-H2; Chothia
TPAAGY

SEQ ID NO: 65 - protein, 6 aa, synthetic construct
  REGION 1..6: note = Synthetic: 2290-C07; CDR-H2; Chothia
TPFDGY

SEQ ID NO: 66 - protein, 6 aa, synthetic construct
  REGION 1..6: note = Synthetic: 2290-D05; CDR-H2; Chothia
TPSAGY

SEQ ID NO: 67 - protein, 6 aa, synthetic construct
  REGION 1..6: note = Synthetic: 2290-C08; CDR-H2; Chothia
TPPSGY

SEQ ID NO: 68 - protein, 6 aa, synthetic construct
  REGION 1..6: note = Synthetic: 2213-A06; CDR-H2; Chothia
SPYGGY

SEQ ID NO: 69 - protein, 6 aa, synthetic construct
  REGION 1..6: note = Synthetic: 2291-E06; CDR-H2; Chothia
FPSGGY

SEQ ID NO: 70 - protein, 6 aa, synthetic construct
  REGION 1..6: note = Synthetic: 2291-D07; CDR-H2; Chothia
SPYDGY

SEQ ID NO: 71 - protein, 6 aa, synthetic construct
  REGION 1..6: note = Synthetic: 9A8; CDR-H2; Chothia
RNDGNS

SEQ ID NO: 72 - protein, 6 aa, synthetic construct
  REGION 1..6: note = Synthetic: 10G5; CDR-H2; Chothia
SNDGSS

SEQ ID NO: 73 - protein, 6 aa, synthetic construct
  REGION 1..6: note = Synthetic: 11D6; CDR-H2; Chothia
RNDGRS

SEQ ID NO: 74 - protein, 7 aa, synthetic construct
  REGION 1..7: note = Synthetic: 10F4; CDR-H2; Chothia
TYGTGSY

SEQ ID NO: 75 - protein, 6 aa, synthetic construct
  REGION 1..6: note = Synthetic: 9A5; CDR-H2; Chothia
DHDGRY

SEQ ID NO: 76 - protein, 6 aa, synthetic construct
  REGION 1..6: note = Synthetic: 9E12; CDR-H2; Chothia
NSAGSG

SEQ ID NO: 77 - protein, 6 aa, synthetic construct
  REGION 1..6: note = Synthetic: 9H1; CDR-H2; Chothia
NSAGSD

SEQ ID NO: 78 - protein, 7 aa, synthetic construct
  REGION 1..7: note = Synthetic: 10E10; CDR-H2; Chothia
NSGGSSY

SEQ ID NO: 79 - protein, 17 aa, synthetic construct
  REGION 1..17: note = Synthetic: 2137-C07; CDR-H2; Kabat
FINPAGGYTDYADSVKG

SEQ ID NO: 80 - protein, 17 aa, synthetic construct
  REGION 1..17: note = Synthetic: 2265-F03; CDR-H2; Kabat
FISPAAGYTDYADSVKG

SEQ ID NO: 81 - protein, 17 aa, synthetic construct
  REGION 1..17: note = Synthetic: 2265-D11; CDR-H2; Kabat
FINPAAGYTDYADSVKG

SEQ ID NO: 82 - protein, 17 aa, synthetic construct
  REGION 1..17: note = Synthetic: 2137-A05; CDR-H2; Kabat
WIYPYSGYTNYADSVKG

SEQ ID NO: 83 - protein, 17 aa, synthetic construct
  REGION 1..17: note = Synthetic: 2288-A03; CDR-H2; Kabat
WIYPYIGFTEYADSVKG

SEQ ID NO: 84 - protein, 17 aa, synthetic construct
  REGION 1..17: note = Synthetic: 2190-B01; CDR-H2; Kabat
VITPSGGYTYYADSVKG

SEQ ID NO: 85 - protein, 17 aa, synthetic construct
  REGION 1..17: note = Synthetic: 2290-G01; CDR-H2; Kabat
VITPPSGFTYYADSVKG

SEQ ID NO: 86 - protein, 17 aa, synthetic construct
  REGION 1..17: note = Synthetic: 2290-D02; CDR-H2; Kabat
VITPAAGYTYYADSVKG

SEQ ID NO: 87 - protein, 17 aa, synthetic construct
  REGION 1..17: note = Synthetic: 2290-C07; CDR-H2; Kabat
VITPFDGYTYYADSVKG

SEQ ID NO: 88 - protein, 17 aa, synthetic construct
  REGION 1..17: note = Synthetic: 2290-D05; CDR-H2; Kabat
VITPSAGYTYYADSVKG

SEQ ID NO: 89 - protein, 17 aa, synthetic construct
  REGION 1..17: note = Synthetic: 2290-C08; CDR-H2; Kabat
VITPPSGYTYYADSVKG

SEQ ID NO: 90 - protein, 17 aa, synthetic construct
  REGION 1..17: note = Synthetic: 2290-A02; CDR-H2; Kabat
VITPSAGYTHYADSVKG

SEQ ID NO: 91 - protein, 17 aa, synthetic construct
  REGION 1..17: note = Synthetic: 2213-A06; CDR-H2; Kabat
VISPYGGYTEYADSVKG

SEQ ID NO: 92 - protein, 17 aa, synthetic construct
  REGION 1..17: note = Synthetic: 2291-G05; CDR-H2; Kabat
WISPYGGYTEYADSVKG

SEQ ID NO: 93 - protein, 17 aa, synthetic construct
  REGION 1..17: note = Synthetic: 2291-E06; CDR-H2; Kabat
HIFPSGGYTDYADSVKG

SEQ ID NO: 94 - protein, 17 aa, synthetic construct
  REGION 1..17: note = Synthetic: 2291-D07; CDR-H2; Kabat
VISPYDGYTEYADSVKG

SEQ ID NO: 95 - protein, 17 aa, synthetic construct
  REGION 1..17: note = Synthetic: 2291-F10; CDR-H2; Kabat
WISPYDGYTEYADSVKG

SEQ ID NO: 96 - protein, 17 aa, synthetic construct
  REGION 1..17: note = Synthetic: 2291-A04; CDR-H2; Kabat
FISPYDGYTEYADSVKG

SEQ ID NO: 97 - protein, 17 aa, synthetic construct
  REGION 1..17: note = Synthetic: 2291-A01; CDR-H2; Kabat
HISPYDGYTDYADSVKG

SEQ ID NO: 98 - protein, 17 aa, synthetic construct
  REGION 1..17: note = Synthetic: 9A8; CDR-H2; Kabat
YIRNDGNSASYGPAVKG

SEQ ID NO: 99 - protein, 17 aa, synthetic construct
  REGION 1..17: note = Synthetic: 10G5; CDR-H2; Kabat
YISNDGSSTSYGPAVKG

SEQ ID NO: 100 - protein, 17 aa, synthetic construct
  REGION 1..17: note = Synthetic: 11D6; CDR-H2; Kabat
YIRNDGRSTSYGPAVKG

SEQ ID NO: 101 - protein, 17 aa, synthetic construct
  REGION 1..17: note = Synthetic: h11D6-Hc4; CDR-H2; Kabat
YIRNDGRSTSYVDSVKG

SEQ ID NO: 102 - protein, 17 aa, synthetic construct
  REGION 1..17: note = Synthetic: h11D6-Hc3; CDR-H2; Kabat
YIRNDGRSTSYADSVKG

SEQ ID NO: 103 - protein, 17 aa, synthetic construct
  REGION 1..17: note = Synthetic: h11D6-Hc1; CDR-H2; Kabat
YIRNDGRSTSYAAPVKG

SEQ ID NO: 104 - protein, 18 aa, synthetic construct
  REGION 1..18: note = Synthetic: 10F4; CDR-H2; Kabat
GITYGTGSYTAYGAAVKG

SEQ ID NO: 105 - protein, 18 aa, synthetic construct
  REGION 1..18: note = Synthetic: h10F4-Hc4; CDR-H2; Kabat
GITYGTGSYTAYVDSVKG

SEQ ID NO: 106 - protein, 18 aa, synthetic construct
  REGION 1..18: note = Synthetic: h10F4-Hc3; CDR-H2; Kabat
GITYGTGSYTAYADSVKG

SEQ ID NO: 107 - protein, 18 aa, synthetic construct
  REGION 1..18: note = Synthetic: h10F4-Hc1; CDR-H2; Kabat
GITYGTGSYTAYAAPVKG

SEQ ID NO: 108 - protein, 17 aa, synthetic construct
  REGION 1..17: note = Synthetic: 9A5; CDR-H2; Kabat
RIDHDGRYTDYGAVVKG

SEQ ID NO: 109 - protein, 17 aa, synthetic construct
  REGION 1..17: note = Synthetic: 9E12; CDR-H2; Kabat
RINSAGSGTYYGSAVDG

SEQ ID NO: 110 - protein, 17 aa, synthetic construct
  REGION 1..17: note = Synthetic: 9H1; CDR-H2; Kabat
RINSAGSDTNYGSAVKG

SEQ ID NO: 111 - protein, 17 aa, synthetic construct
  REGION 1..17: note = Synthetic: 10H1; CDR-H2; Kabat
RINSAGSDTDYGAAVKG

SEQ ID NO: 112 - protein, 18 aa, synthetic construct
  REGION 1..18: note = Synthetic: 10E10; CDR-H2; Kabat
RINSGGSSYTDYGSAVKG

SEQ ID NO: 113 - protein, 17 aa, synthetic construct
  REGION 1..17: note = Synthetic: h10H1-Hc4; CDR-H2; Kabat
RINSAGSDTDYVDSVKG

SEQ ID NO: 114 - protein, 17 aa, synthetic construct
  REGION 1..17: note = Synthetic: h10H1-Hc3; CDR-H2; Kabat
RINSAGSDTDYADSVKG

SEQ ID NO: 115 - protein, 17 aa, synthetic construct
  REGION 1..17: note = Synthetic: h10H1-Hc1; CDR-H2; Kabat
RINSAGSDTDYAAPVKG

SEQ ID NO: 116 - protein, 13 aa, synthetic construct
  REGION 1..13: note = Synthetic: 2137-C07; CDR-H3
DYVYQYWTYVLDY

SEQ ID NO: 117 - protein, 13 aa, synthetic construct
  REGION 1..13: note = Synthetic: 2265-F06; CDR-H3
DYILQYWTYVLDY

SEQ ID NO: 118 - protein, 13 aa, synthetic construct
  REGION 1..13: note = Synthetic: 2265-F05; CDR-H3
DYVNAYWTYVLDY

SEQ ID NO: 119 - protein, 13 aa, synthetic construct
  REGION 1..13: note = Synthetic: 2265-F02; CDR-H3
DYIRQYWTYVLDY

SEQ ID NO: 120 - protein, 13 aa, synthetic construct
  REGION 1..13: note = Synthetic: 2265-B06; CDR-H3
DYVQAYWTYVLDY

SEQ ID NO: 121 - protein, 13 aa, synthetic construct
  REGION 1..13: note = Synthetic: 2265-A09; CDR-H3
DYVYNYWTYVLDY

SEQ ID NO: 122 - protein, 13 aa, synthetic construct
  REGION 1..13: note = Synthetic: 2265-F03; CDR-H3
DFVQSYWTYVLDY

SEQ ID NO: 123 - protein, 13 aa, synthetic construct
  REGION 1..13: note = Synthetic: 2265-D11; CDR-H3
DYIYQYWTYVLDY

SEQ ID NO: 124 - protein, 13 aa, synthetic construct
  REGION 1..13: note = Synthetic: 2265-D05; CDR-H3
DFVYAYWTYVLDY

SEQ ID NO: 125 - protein, 13 aa, synthetic construct
  REGION 1..13: note = Synthetic: 2265-C03; CDR-H3
DYVPQYWTYVLDY

SEQ ID NO: 126 - protein, 13 aa, synthetic construct
  REGION 1..13: note = Synthetic: 2265-C02; CDR-H3
DYIYSYWTYVLDY

SEQ ID NO: 127 - protein, 13 aa, synthetic construct
  REGION 1..13: note = Synthetic: 2137-A05; CDR-H3
DYGPWYGTGVLDY

SEQ ID NO: 128 - protein, 13 aa, synthetic construct
  REGION 1..13: note = Synthetic: 2288-A03; CDR-H3
DYDLRYLTGVLDY

SEQ ID NO: 129 - protein, 13 aa, synthetic construct
  REGION 1..13: note = Synthetic: 2190-B01; CDR-H3
DLGGGYWVGFFDY

SEQ ID NO: 130 - protein, 13 aa, synthetic construct
  REGION 1..13: note = Synthetic: 2290-G01; CDR-H3
DLGVGYWVGFSDY

SEQ ID NO: 131 - protein, 13 aa, synthetic construct
  REGION 1..13: note = Synthetic: 2290-D02; CDR-H3
DLGHRYWVGVFDY

SEQ ID NO: 132 - protein, 13 aa, synthetic construct
  REGION 1..13: note = Synthetic: 2290-C07; CDR-H3
DMGVGYWVGFSDY

SEQ ID NO: 133 - protein, 13 aa, synthetic construct
  REGION 1..13: note = Synthetic: 2290-D05; CDR-H3
DLGYGYWVGFSDY

SEQ ID NO: 134 - protein, 13 aa, synthetic construct
  REGION 1..13: note = Synthetic: 2290-C08; CDR-H3
DWGVGYWVGFSDY

SEQ ID NO: 135 - protein, 13 aa, synthetic construct
  REGION 1..13: note = Synthetic: 2290-A02; CDR-H3
DLGSRYWVGVFDY

SEQ ID NO: 136 - protein, 13 aa, synthetic construct
  REGION 1..13: note = Synthetic: 2213-A06; CDR-H3
DFYDRYSTYVLDY

SEQ ID NO: 137 - protein, 13 aa, synthetic construct
  REGION 1..13: note = Synthetic: 2291-G05; CDR-H3
DFHDRYATFVLDY

SEQ ID NO: 138 - protein, 13 aa, synthetic construct
  REGION 1..13: note = Synthetic: 2291-E06; CDR-H3
DFYDRYATYVLDY

SEQ ID NO: 139 - protein, 13 aa, synthetic construct
  REGION 1..13: note = Synthetic: 2291-F10; CDR-H3
DYYDRYSTYVLDY

SEQ ID NO: 140 - protein, 13 aa, synthetic construct
  REGION 1..13: note = Synthetic: 2291-A04; CDR-H3
DFNDRYFTYVLDY

SEQ ID NO: 141 - protein, 12 aa, synthetic construct
  REGION 1..12: note = Synthetic: 9A8; CDR-H3
TTCIGSGGCIDT

SEQ ID NO: 142 - protein, 12 aa, synthetic construct
  REGION 1..12: note = Synthetic: 11D6; CDR-H3
TTCVGSGGCIDT

SEQ ID NO: 143 - protein, 16 aa, synthetic construct
  REGION 1..16: note = Synthetic: 10F4; CDR-H3
GGGLNSYGCSGANIDA

SEQ ID NO: 144 - protein, 9 aa, synthetic construct
  REGION 1..9: note = Synthetic: 9A5; CDR-H3
GGGAASIDT

SEQ ID NO: 145 - protein, 9 aa, synthetic construct
  REGION 1..9: note = Synthetic: 9E12; CDR-H3
GGGGASIDG

SEQ ID NO: 146 - protein, 11 aa, synthetic construct
  REGION 1..11: note = Synthetic: trastuzumab; CDR-L1
RASQDVNTAVA

SEQ ID NO: 147 - protein, 8 aa, synthetic construct
  REGION 1..8: note = Synthetic: 9A8; CDR-L1
SGGSSDYG

SEQ ID NO: 148 - protein, 8 aa, synthetic construct
  REGION 1..8: note = Synthetic: 10G5; CDR-L1
SGGNYDYG

SEQ ID NO: 149 - protein, 8 aa, synthetic construct
  REGION 1..8: note = Synthetic: 11D6; CDR-L1
SGGNSDYG

SEQ ID NO: 150 - protein, 13 aa, synthetic construct
  REGION 1..13: note = Synthetic: 10F4; CDR-L1
SGGGNYFGSYYYG

SEQ ID NO: 151 - protein, 13 aa, synthetic construct
  REGION 1..13: note = Synthetic: 9A5; CDR-L1
SGGGNYVGGYYYG

SEQ ID NO: 152 - protein, 13 aa, synthetic construct
  REGION 1..13: note = Synthetic: 9E12; CDR-L1
SGGGSYYGSYYYG

SEQ ID NO: 153 - protein, 13 aa, synthetic construct
  REGION 1..13: note = Synthetic: 10H1; CDR-L1
SGGGNYYGSYYYG

SEQ ID NO: 154 - protein, 13 aa, synthetic construct
  REGION 1..13: note = Synthetic: 10E10; CDR-L1
SGGGNYAGSYYYG

SEQ ID NO: 155 - protein, 7 aa, synthetic construct
  REGION 1..7: note = Synthetic: trastuzumab; CDR-L2
SASFLYS

SEQ ID NO: 156 - protein, 7 aa, synthetic construct
  REGION 1..7: note = Synthetic: 9A8; CDR-L2
SNNQRPS

SEQ ID NO: 157 - protein, 7 aa, synthetic construct
  REGION 1..7: note = Synthetic: 10G5; CDR-L2
YNNKRPS

SEQ ID NO: 158 - protein, 7 aa, synthetic construct
  REGION 1..7: note = Synthetic: 11D6; CDR-L2
RNNQRPS

SEQ ID NO: 159 - protein, 7 aa, synthetic construct
  REGION 1..7: note = Synthetic: 10F4; CDR-L2
NNNNRPS

SEQ ID NO: 160 - protein, 7 aa, synthetic construct
  REGION 1..7: note = Synthetic: 10E10; CDR-L2
NSNNRPS

SEQ ID NO: 161 - protein, 9 aa, synthetic construct
  REGION 1..9: note = Synthetic: trastuzumab; CDR-L3
QQHYTTPPT

SEQ ID NO: 162 - protein, 9 aa, synthetic construct
  REGION 1..9: note = Synthetic: 9A8; CDR-L3
ANVDYTDDV

SEQ ID NO: 163 - protein, 9 aa, synthetic construct
  REGION 1..9: note = Synthetic: 10G5; CDR-L3
ANVDSTDDV

SEQ ID NO: 164 - protein, 9 aa, synthetic construct
  REGION 1..9: note = Synthetic: 11D6; CDR-L3
GNVDFTDDV

SEQ ID NO: 165 - protein, 10 aa, synthetic construct
  REGION 1..10: note = Synthetic: h11D6-Lc4; CDR-L3
GGFDSSSDAI

SEQ ID NO: 166 - protein, 10 aa, synthetic construct
  REGION 1..10: note = Synthetic: 10F4; CDR-L3
GGFDSSTDAI

SEQ ID NO: 167 - protein, 122 aa, synthetic construct
  REGION 1..122: note = Synthetic: 2137-C07; VH
EVQLVESGGGLVQPGGSLRLSCAASGFNISGSGIHWVRQAPGKGLEWVGFINPAGGYTDYADSVKGRFTISADTSKNTAYLQMNSLRAEDTAVYYCARDYVYQYWTYVLDYWGQGTLVTVSS

SEQ ID NO: 168 - protein, 122 aa, synthetic construct
  REGION 1..122: note = Synthetic: 2265-F06; VH
EVQLVESGGGLVQPGGSLRLSCAASGFNISYPGIHWVRQAPGKGLEWVGFINPAGGYTDYADSVKGRFTISADTSKNTAYLQMNSLRAEDTAVYYCARDYILQYWTYVLDYWGQGTLVTVSS

SEQ ID NO: 169 - protein, 122 aa, synthetic construct
  REGION 1..122: note = Synthetic: 2265-F05; VH
EVQLVESGGGLVQPGGSLRLSCAASGFNIIAPGIHWVRQAPGKGLEWVGFINPAGGYTDYADSVKGRFTISADTSKNTAYLQMNSLRAEDTAVYYCARDYVNAYWTYVLDYWGQGTLVTVSS

SEQ ID NO: 170 - protein, 122 aa, synthetic construct
  REGION 1..122: note = Synthetic: 2265-F02; VH
EVQLVESGGGLVQPGGSLRLSCAASGFNISAPGIHWVRQAPGKGLEWVGFINPAGGYTDYADSVKGRFTISADTSKNTAYLQMNSLRAEDTAVYYCARDYIRQYWTYVLDYWGQGTLVTVSS

SEQ ID NO: 171 - protein, 122 aa, synthetic construct
  REGION 1..122: note = Synthetic: 2265-B06; VH
EVQLVESGGGLVQPGGSLRLSCAASGFNIRVSGIHWVRQAPGKGLEWVGFINPAGGYTDYADSVKGRFTISADTSKNTAYLQMNSLRAEDTAVYYCARDYVQAYWTYVLDYWGQGTLVTVSS

SEQ ID NO: 172 - protein, 122 aa, synthetic construct
  REGION 1..122: note = Synthetic: 2265-A09; VH
EVQLVESGGGLVQPGGSLRLSCAASGFNIIGPGIHWVRQAPGKGLEWVGFINPAGGYTDYADSVKGRFTISADTSKNTAYLQMNSLRAEDTAVYYCARDYVYNYWTYVLDYWGQGTLVTVSS

SEQ ID NO: 173 - protein, 122 aa, synthetic construct
  REGION 1..122: note = Synthetic: 2265-F03; VH
EVQLVESGGGLVQPGGSLRLSCAASGFNIRGPGIHWVRQAPGKGLEWVGFISPAAGYTDYADSVKGRFTISADTSKNTAYLQMNSLRAEDTAVYYCARDFVQSYWTYVLDYWGQGTLVTVSS

SEQ ID NO: 174 - protein, 122 aa, synthetic construct
  REGION 1..122: note = Synthetic: 2265-E02; VH
EVQLVESGGGLVQPGGSLRLSCAASGFNIYVSGIHWVRQAPGKGLEWVGFINPAGGYTDYADSVKGRFTISADTSKNTAYLQTNSLRAEDTAVYYCARDYVYQYWTYVLDYWGQGTLVTVSS

SEQ ID NO: 175 - protein, 122 aa, synthetic construct
  REGION 1..122: note = Synthetic: 2265-D11; VH
EVQLVESGGGLVQPGGSLRLSCAASGFNISGPGIHWVRQAPGKGLEWVGFINPAAGYTDYADSVKGRFTISADTSKNTAYLQMNSLRAEDTAVYYCARDYIYQYWTYVLDYWGQGTLVTVSS

SEQ ID NO: 176 - protein, 122 aa, synthetic construct
  REGION 1..122: note = Synthetic: 2265-D05; VH
EVQLVESGGGLVQPGGSLRLSCAASGFNISGPGIHWVRQAPGKGLEWVGFINPAAGYTDYADSVKGRFAISADTSKNTAYLQMNSLRAEDTAVYYCARDFVYAYWTYVLDYWGQGTLVTVSS

SEQ ID NO: 177 - protein, 122 aa, synthetic construct
  REGION 1..122: note = Synthetic: 2265-C03; VH
EVQLVESGGGLVQPGGSLRLSCAAPGFNISVPGIHWVRQAPGKGLEWVGFINPAGGYTDYADSVKGRFTISADTSKNTAYLQMNSLRAEDTAVYYCARDYVPQYWTYVLDYWGQGTLVTVSS

SEQ ID NO: 178 - protein, 122 aa, synthetic construct
  REGION 1..122: note = Synthetic: 2265-C02; VH
EVQLVESGGGLVQPGGSLRLSCAASGFNIGVSGIHWVRQAPGKGLEWVGFINPAGGYTDYAGSVKGRFTISADTSKNTAYLQMNSLRAEDTAVYYCARDYIYSYWTYVLDYWGQGTLVTVSS

SEQ ID NO: 179 - protein, 122 aa, synthetic construct
  REGION 1..122: note = Synthetic: 2265-A06; VH
EVQLVESGGGLVQPGGSLRLSCAASGFNIYRSGIHWVRQAPGKGLEWVGFINPAGGYTDYADSVKGRFTISADTSKNTAYLQMNSLRAEDTAVYYCARDYVPQYWTYVLDYWGQGTLVTVSS

SEQ ID NO: 180 - protein, 122 aa, synthetic construct
  REGION 1..122: note = Synthetic: 2137-A05; VH
EVQLVESGGGLVQPGGSLRLSCAASGFNINNSYIHWVRQAPGKGLEWVGWIYPYSGYTNYADSVKGRFTISADTSKNTAYLQMNSLRAEDTAVYYCARDYGPWYGTGVLDYWGQGTLVTVSS

SEQ ID NO: 181 - protein, 122 aa, synthetic construct
  REGION 1..122: note = Synthetic: 2288-A03; VH
EVQLVESGGGLVQPGGSLRLSCAASGFNINNSWIHWVRQAPGKGLEWVGWIYPYIGFTEYADSVKGRFTISADTSKNTAYLQMNSLRAEDTAVYYCARDYDLRYLTGVLDYWGQGTLVTVSS

SEQ ID NO: 182 - protein, 122 aa, synthetic construct
  REGION 1..122: note = Synthetic: 2190-B01; VH
EVQLVESGGGLVQPGGSLRLSCAASGFNISSYWIHWVRQAPGKGLEWVGVITPSGGYTYYADSVKGRFTISADTSKNTAYLQMNSLRAEDTAVYYCARDLGGGYWVGFFDYWGQGTLVTVSS

SEQ ID NO: 183 - protein, 122 aa, synthetic construct
  REGION 1..122: note = Synthetic: 2290-G01; VH
EVQLVESGGGLVQPGGSLRLSCAASGFNISPYWIHWVRQAPGKGLEWVGVITPPSGFTYYADSVKGRFTISADTSKNTAYLQVNSLRAEDTAVYYCARDLGVGYWVGFSDYWGQGTLVTVSS

SEQ ID NO: 184 - protein, 122 aa, synthetic construct
  REGION 1..122: note = Synthetic: 2290-D02; VH
EVQLVESGGGLVQPGGSLRLSCAASGFNISSYWIHWVRQAPGKGLEWMGVITPAAGYTYYADSVKGRFTISADTSKNTAYLQMNSLRAEDTAVYYCARDLGHRYWVGVFDYWGQGTLVTVSS

SEQ ID NO: 185 - protein, 122 aa, synthetic construct
  REGION 1..122: note = Synthetic: 2290-C07; VH
EVQLVESGGGLVQPGGSLRLSCAASGFNITYDWIHWVRQAPGKGLEWVGVITPFDGYTYYADSVKGHFTISADTSKNTAYLQMNSLRAEDTAVYYCARDMGVGYWVGFSDYWGQGTLVTVSS

SEQ ID NO: 186 - protein, 122 aa, synthetic construct
  REGION 1..122: note = Synthetic: 2290-D05; VH
EVQLVESGGGLVQPGGSLRLSCAASGFNIASRWIHWVRQAPGKGLEWVGVITPSAGYTYYADSVKGRFTISADTSKNTAYLQMNSLRAEDTAVYYCARDLGYGYWVGFSDYWGQGTLVTVSS

SEQ ID NO: 187 - protein, 122 aa, synthetic construct
  REGION 1..122: note = Synthetic: 2290-C08; VH
EVQLVESGGGLVQPGGSLRLSCAASGFNIQPYWIHWVRQAPGKGLEWVGVITPPSGYTYYADSVKGRFTISADTSKNTAYLQMNSLRAEDTAVYYCARDWGVGYWVGFSDYWGQGTLVTVSS

SEQ ID NO: 188 - protein, 122 aa, synthetic construct
  REGION 1..122: note = Synthetic: 2290-A02; VH
EVQLVESGGGLVQPGGSLRLSCAASGFNISTRWIHWVRQAPGKGLEWVGVITPSAGYTHYADSVKGRFTISAGTSKNTAYLQMNSLRAEDTAVYYCARDLGSRYWVGVFDYWGQGTLVTVSS

SEQ ID NO: 189 - protein, 122 aa, synthetic construct
  REGION 1..122: note = Synthetic: 2213-A06; VH
EVQLVESGGGLVQPGGSLRLSCAASGFNISSYAIHWVRQAPGKGLEWVGVISPYGGYTEYADSVKGRFTISADTSKNTAYLQMNSLRAEDTAVYYCARDFYDRYSTYVLDYWGQGTLVTVSS

SEQ ID NO: 190 - protein, 122 aa, synthetic construct
  REGION 1..122: note = Synthetic: 2291-G05; VH
EVQLVESGGGLVQPGGSLRLSCAASGFNIAAYTIHWVRQAPGKGLEWVGWISPYGGYTEYADSVKGRFTISADTSKNTAYLQMNSLRAEDTAVYYCARDFHDRYATFVLDYWGQGTLVTVSS

SEQ ID NO: 191 - protein, 122 aa, synthetic construct
  REGION 1..122: note = Synthetic: 2291-E06; VH
EVQLVESGGGLVQPGGSLRLSCAASGFNISPYTIHWVRQAPGKGLEWVAHIFPSGGYTDYADSVKGRFTISADTSKNTAYLQMNSLRAEDTAVYYCARDFYDRYATYVLDYWGQGTLVTVSS

SEQ ID NO: 192 - protein, 122 aa, synthetic construct
  REGION 1..122: note = Synthetic: 2291-D07; VH
EVQLVESGGGLVQPGGSLRLSCAASGFNIKDTYIHWVRQAPGKGLEWVGVISPYDGYTEYADSVKGRFTISADTSKNTAYLQMNSLRAEDTAVYYCAHDFYDRYSTYVLDYWGQGTLVTVSS

SEQ ID NO: 193 - protein, 122 aa, synthetic construct
  REGION 1..122: note = Synthetic: 2291-F10; VH
EVQLVESGGGLVQPGGSLRLSCAASGFNIDPYTIHWVRQAPGKGLEWVGWISPYDGYTEYADSVKGRFTISADTSKNTAYLQMNSLRAEDTAVYYCARDYYDRYSTYVLDYWGRGTLVTVSS

SEQ ID NO: 194 - protein, 122 aa, synthetic construct
  REGION 1..122: note = Synthetic: 2291-A04; VH
EVQLVESGGGLVQPGGSLRLSCAASGFNISSYGIHWVRQAPGKGLEWVGFISPYDGYTEYADSVKGRFTISAGTSKNTAYLQMNSLRAEDTAVYYCARDFNDRYFTYVLDYWGQGTLVTVSS

SEQ ID NO: 195 - protein, 122 aa, synthetic construct
  REGION 1..122: note = Synthetic: 2291-A01; VH
EVQLVESGGGLVQPGGSLRLSCAASGFNIDPYTIHWARQAPGKGLEWVAHISPYDGYTDYADSVKGRFTISADTSKNTAYLQMNSLRAEDTAVYYCARDFYDRYSTYVLDYWGQGTLVTVSS

SEQ ID NO: 196 - protein, 121 aa, synthetic construct
  REGION 1..121: note = Synthetic: 9A8; VH
AVTLDESGGGLQTPGGTLSLVCKASGFTFSSFNMFWVRQAPGKGLEWVAYIRNDGNSASYGPAVKGRATISRDNGQSTVRLQLNNLRAEDTATYYCAKTTCIGSGGCIDTWGHGTEVIVSS

SEQ ID NO: 197 - protein, 121 aa, synthetic construct
  REGION 1..121: note = Synthetic: 10G5; VH
AVTLDESGGGLQTPGGVLSLVCKASGFTFSSFNMFWVRQAPGKGLEWVAYISNDGSSTSYGPAVKGRATISRDNGQSTVRLQLNNLRAEDTATYFCAKTTCIGSGGCIDTWGHGTEVIVSS

SEQ ID NO: 198 - protein, 121 aa, synthetic construct
  REGION 1..121: note = Synthetic: 11D6; VH
AVTLDESGGGLQTPGGTLSLVCKASGFTFSSFNMFWVRQAPGEGLEWVAYIRNDGRSTSYGPAVKGRATISRDNGQSTVRLQLNNLRAEDTGTYFCAKTTCVGSGGCIDTWGHGTEVIVSS

SEQ ID NO: 199 - protein, 121 aa, synthetic construct
  REGION 1..121: note = Synthetic: h11D6-HC4; VH
EVQLVESGGGLVQPGGSLRLSCAASGFTFSSFNMFWVRQAPGKGLEWVAYIRNDGRSTSYVDSVKGRFTISRDNAKSSVYLQMNSLRAEDTAVYYCAKTTCVGSGGCIDTWGQGTLVTVSS

SEQ ID NO: 200 - protein, 121 aa, synthetic construct
  REGION 1..121: note = Synthetic: h11D6-HC3; VH
QVQLVESGGGVVQPGRSLRLSCAASGFTFSSFNMFWVRQAPGKGLEWVAYIRNDGRSTSYADSVKGRFTISRDNSKSTVYLQMNSLRAEDTAVYYCAKTTCVGSGGCIDTWGQGTLVTVSS

SEQ ID NO: 201 - protein, 121 aa, synthetic construct
  REGION 1..121: note = Synthetic: h11D6-HC2; VH
EVQLLESGGGLVQPGGSLRLSCAASGFTFSSFNMFWVRQAPGKGLEWVAYIRNDGRSTSYADSVKGRFTISRDNSKSTVYLQMNSLRAEDTAVYYCAKTTCVGSGGCIDTWGQGTLVTVSS

SEQ ID NO: 202 - protein, 121 aa, synthetic construct
  REGION 1..121: note = Synthetic: h11D6-HC1; VH
EVQLVESGGGLVKPGGSLRLSCAASGFTFSSFNMFWVRQAPGKGLEWVAYIRNDGRSTSYAAPVKGRFTISRDNSKSTVYLQMNSLKTEDTAVYYCAKTTCVGSGGCIDTWGQGTLVTVSS

SEQ ID NO: 203 - protein, 126 aa, synthetic construct
  REGION 1..126: note = Synthetic: 10F4; VH
AVTLDESGGGLQTPGGALSLVCKASGFTFSGYNMGWVRQAPGKGLEYVAGITYGTGSYTAYGAAVKGRATISRDNGQSTLRLQLNNLRAEDTATYYCARGGGLNSYGCSGANIDAWGHGTEVIVSS

SEQ ID NO: 204 - protein, 126 aa, synthetic construct
  REGION 1..126: note = Synthetic: h10F4-HC4; VH
EVQLVESGGGLVQPGGSLRLSCAASGFTFSGYNMGWVRQAPGKGLEWVAGITYGTGSYTAYVDSVKGRFTISRDNAKSSLYLQMNSLRAEDTAVYYCARGGGLNSYGCSGANIDAWGQGTLVTVSS

SEQ ID NO: 205 - protein, 126 aa, synthetic construct
  REGION 1..126: note = Synthetic: h10F4-HC3; VH
QVQLVESGGGVVQPGRSLRLSCAASGFTFSGYNMGWVRQAPGKGLEWVAGITYGTGSYTAYADSVKGRFTISRDNSKSTLYLQMNSLRAEDTAVYYCARGGGLNSYGCSGANIDAWGQGTLVTVSS

SEQ ID NO: 206 - protein, 126 aa, synthetic construct
  REGION 1..126: note = Synthetic: h10F4-HC2; VH
EVQLLESGGGLVQPGGSLRLSCAASGFTFSGYNMGWVRQAPGKGLEWVAGITYGTGSYTAYADSVKGRFTISRDNSKSTLYLQMNSLRAEDTAVYYCARGGGLNSYGCSGANIDAWGQGTLVTVSS

SEQ ID NO: 207 - protein, 126 aa, synthetic construct
  REGION 1..126: note = Synthetic: h10F4-HC1; VH
EVQLVESGGGLVKPGGSLRLSCAASGFTFSGYNMGWVRQAPGKGLEWVAGITYGTGSYTAYAAPVKGRFTISRDNSKSTLYLQMNSLKTEDTAVYYCARGGGLNSYGCSGANIDAWGQGTLVTVSS

SEQ ID NO: 208 - protein, 118 aa, synthetic construct
  REGION 1..118: note = Synthetic: 9A5; VH
AVTLDESGGGLQTPGGAVSLVCKASGFSISDYGMGWMRQAPGKGLQYVARIDHDGRYTDYGAVVKGRATISRDNGQSTVRLQLNNLRAEDTGTYYCTRGGGAASIDTWGHGTEVIVSS

SEQ ID NO: 209 - protein, 118 aa, synthetic construct
  REGION 1..118: note = Synthetic: 9E12; VH
AVTLDESGGGLQTPGGGLSLVCKASGFTFSDYGLGWMRQAPGKGLEYVARINSAGSGTYYGSAVDGRATISRDNGQSTVRLQLNNLRAEDTGTYYCTRGGGGASIDGWGHGTEVIVSS

SEQ ID NO: 210 - protein, 118 aa, synthetic construct
  REGION 1..118: note = Synthetic: 9H1; VH
AVTLDESGGGLQTPGGALSLVCKGSGFTFSDYGMGWMRQAPGKGLQYVARINSAGSDTNYGSAVKGRATISRDDGQSTVRLQLSSLRAEDTGIYYCTRGGGGASIDGWGHGTEVIVSS

SEQ ID NO: 211 - protein, 118 aa, synthetic construct
  REGION 1..118: note = Synthetic: 10H1; VH
AVTLDESGGGLQTPGGALSLVCKASGFTFSGYGMGWMRQAPGKGLEYVARINSAGSDTDYGAAVKGRATISRDNGQSTVRLQLNNLRAEDTATYFCTRGGGGASIDGWGHGTEVIVSS

SEQ ID NO: 212 - protein, 119 aa, synthetic construct
  REGION 1..119: note = Synthetic: 10E10; VH
AVTLDESGGGLQTPGGGLSLVCKASGFTFSSYGMGWMRQAPGKGLEFVARINSGGSSYTDYGSAVKGRATISRDDGQSTVRLQLNNLRAEDTGIYYCTRGGGGASIDGWGHGTEVIVSS

SEQ ID NO: 213 - protein, 118 aa, synthetic construct
  REGION 1..118: note = Synthetic: h10H1-HC4; VH
EVQLVESGGGLVQPGGSLRLSCAASGFTFSGYGMGWVRQAPGKGLEWVARINSAGSDTDYVDSVKGRFTISRDNAKSSVYLQMNSLRAEDTAVYYCTRGGGGASIDGWGQGTLVTVSS

SEQ ID NO: 214 - protein, 118 aa, synthetic construct
  REGION 1..118: note = Synthetic: h10H1-HC3; VH
QVQLVESGGGVVQPGRSLRLSCAASGFTFSGYGMGWVRQAPGKGLEWVARINSAGSDTDYADSVKGRFTISRDNSKSTVYLQMNSLRAEDTAVYYCTRGGGGASIDGWGQGTLVTVSS

SEQ ID NO: 215 - protein, 118 aa, synthetic construct
  REGION 1..118: note = Synthetic: h10H1-HC2; VH
EVQLLESGGGLVQPGGSLRLSCAASGFTFSGYGMGWVRQAPGKGLEWVARINSAGSDTDYADSVKGRFTISRDNSKSTVYLQMNSLRAEDTAVYYCTRGGGGASIDGWGQGTLVTVSS

SEQ ID NO: 216 - protein, 118 aa, synthetic construct
  REGION 1..118: note = Synthetic: h10H1-HC1; VH
EVQLVESGGGLVKPGGSLRLSCAASGFTFSGYGMGWVRQAPGKGLEWVARINSAGSDTDYAAPVKGRFTISRDNSKSTVYLQMNSLKTEDTAVYYCTRGGGGASIDGWGQGTLVTVSS

SEQ ID NO: 217 - protein, 107 aa, synthetic construct [sequence omitted]

SEQ ID NO: 218 - protein, 102 aa, synthetic construct
  REGION 1..102: note = Synthetic: 9A8; VL
ALTQPSSVSANPGETVKITCSGGSSDYGWFQQKSPGSAPVTVIYSNNQRPSGIPSRFSGSKSGSTGTLTITGVQAEDEAIYYCANVDYTDDVFGAGTTLTVL

SEQ ID NO: 219 - protein, 102 aa, synthetic construct
  REGION 1..102: note = Synthetic: 10G5; VL
ALTQPSSVSANPGETVKITCSGGNYDYGWYQQKSPGSAPVTLIYYNNKRPSDIPSRFSGSKSGSTGTLTITGVQAEDEAIYYCANVDSTDDVFGAGTTLTVL

SEQ ID NO: 220 - protein, 102 aa, synthetic construct
  REGION 1..102: note = Synthetic: 11D6; VL
ALTQPSSVSANPGETVEITCSGGNSDYGWFQQKSPGSAPVTVIYRNNQRPSDIPSRFSGSGSGSTNTLTITGVQAEDEAIYYCGNVDFTDDVFGAGTTLTVL

SEQ ID NO: 221 - protein, 104 aa, synthetic construct
  REGION 1..104: note = Synthetic: h11D6-LC4; VL
SYVLTQPPSVSVAPGKTARITCSGGNSDYGWYQQKPGQAPVLVVYRNNQRPSGIPERFSGSGSGSTNTLTISGTQAMDEADYYCGGFDSSSDAIFGGGTKLTVL

SEQ ID NO: 222 - protein, 104 aa, synthetic construct
  REGION 1..104: note = Synthetic: h11D6-LC3; VL
SYELTQPPSVSVSPGQTASITCSGGNSDYGWYQQKPGQSPVLVIYRNNQRPSGIPERFSGSGSGSTNTLTISGTQAMDEADYYCGGFDSSSDAIFGGGTKLTVL

SEQ ID NO: 223 - protein, 104 aa, synthetic construct
  REGION 1..104: note = Synthetic: h11D6-LC2; VL
QSVLTQPPSVSAAPGQKVTISCSGGNSDYGWYQQLPGTAPKLLIYRNNQRPSGIPDRFSGSGSGSTNTLGITGLQTGDEADYYCGGFDSSSDAIFGGGTKLTVL

SEQ ID NO: 224 - protein, 105 aa, synthetic construct
  REGION 1..105: note = Synthetic: h11D6-LC1; VL
DIQMTQSPSSVSASVGDRVTITCSGGNSDYGWYQQKPGKAPKLLIYRNNQRPSGVPSRFSGSGSGSTNTLTISSLQPEDFATYYCGGFDSSSDAIFGQGTKVEIK

SEQ ID NO: 225 - protein, 108 aa, synthetic construct
  REGION 1..108: note = Synthetic: 10F4; VL
ALTQPSSVSANLGGTVKITCSGGGNYFGSYYYGWYQQKAPGSAPVTVIYNNNNRPSDIPSRFSGSTSGSTSTLTISGVRAEDEAVYFCGGFDSSTDAIFGAGTTLTVL

SEQ ID NO: 226 - protein, 109 aa, synthetic construct
  REGION 1..109: note = Synthetic: h10F4-LC4; VL
SYVLTQPPSVSVAPGKTARITCSGGGNYFGSYYYGWYQQKPGQAPVLVVYNNNNRPSGIPERFSGSTSGSTSTLTISGTQAMDEADYYCGGFDSSTDAIFGGGTKLTVL

SEQ ID NO: 227 - protein, 109 aa, synthetic construct
  REGION 1..109: note = Synthetic: h10F4-LC3; VL
SYELTQPPSVSVSPGQTASITCSGGGNYFGSYYYGWYQQKPGQSPVLVIYNNNNRPSGIPERFSGSTSGSTSTLTISGTQAMDEADYYCGGFDSSTDAIFGGGTKLTVL

SEQ ID NO: 228 - protein, 109 aa, synthetic construct
  REGION 1..109: note = Synthetic: h10F4-LC2; VL
QSVLTQPPSVSAAPGQKVTISCSGGGNYFGSYYYGWYQQLPGTAPKLLIYNNNNRPSGIPDRFSGSTSGSTSTLGITGLQTGDEADYYCGGFDSSTDAIFGGGTKLTVL

SEQ ID NO: 229 - protein, 110 aa, synthetic construct
  REGION 1..110: note = Synthetic: h10F4-LC1; VL
DIQMTQSPSSVSASVGDRVTITCSGGGNYFGSYYYGWYQQKPGKAPKLLIYNNNNRPSGVPSRFSGSTSGSTSTLTISSLQPEDFATYYCGGFDSSTDAIFGQGTKVEIK

SEQ ID NO: 230 - protein, 108 aa, synthetic construct
  REGION 1..108: note = Synthetic: 9A5; VL
ALTQPSSVSANPGETVKITCSGGGNYVGGYYYGWYQQKAPGSALVTLIYNNNNRPSNIPSRFSGSTSGSTSTLTITGVRAEDEAVYFCGSFDSSTDAIFGAGTTLTVL

SEQ ID NO: 231 - protein, 108 aa, synthetic construct
  REGION 1..108: note = Synthetic: 9E12; VL
ALTQPSSVSANPGETVKITCSGGGSYYGSYYYGWYQQKSPGSAPVTLIYNNNNRPSDIPSRFSGSTSGSTGTLTITGVRAEDEAVYYCGSFDSSTDAIFGAGTTLTVL

SEQ ID NO: 232 - protein, 108 aa, synthetic construct; identical to SEQ ID NO: 231
  REGION 1..108: note = Synthetic: 9H1; VL

SEQ ID NO: 233 - protein, 108 aa, synthetic construct
  REGION 1..108: note = Synthetic: 10H1; VL
ALTQPSSVSANPGETVKITCSGGGNYYGSYYYGWYQQKAPGSAPVTVIYNNNNRPSNIPSRFSGSKSGSTGTLTITGVQAEDEAVYFCGGFDSSSDAIFGAGTTLTVL

SEQ ID NO: 234 - protein, 108 aa, synthetic construct
  REGION 1..108: note = Synthetic: 10E10; VL
ALTQPSSVSANPGETVKITCSGGGNYAGSYYYGWYQQKSPGSAPLTVIYNSNNRPSDIPSRFSGSLSGSTGTLTITGVRAEDEAVYFCGGFDSSTDAIFGAGTTLTVL

SEQ ID NO: 235 - protein, 109 aa, synthetic construct
  REGION 1..109: note = Synthetic: h10H1-LC4; VL
SYVLTQPPSVSVAPGKTARITCSGGGNYYGSYYYGWYQQKPGQAPVLVVYNNNNRPSGIPERFSGSKSGSTGTLTISGTQAMDEADYYCGGFDSSSDAIFGGGTKLTVL

SEQ ID NO: 236 - protein, 109 aa, synthetic construct
  REGION 1..109: note = Synthetic: h10H1-LC3; VL
SYELTQPPSVSVSPGQTASITCSGGGNYYGSYYYGWYQQKPGQSPVLVIYNNNNRPSGIPERFSGSKSGSTGTLTISGTQAMDEADYYCGGFDSSSDAIFGGGTKLTVL

SEQ ID NO: 237 - protein, 109 aa, synthetic construct
  REGION 1..109: note = Synthetic: h10H1-LC2; VL
QSVLTQPPSVSAAPGQKVTISCSGGGNYYGSYYYGWYQQLPGTAPKLLIYNNNNRPSGIPDRFSGSKSGSTGTLGITGLQTGDEADYYCGGFDSSSDAIFGGGTKLTVL

SEQ ID NO: 238 - protein, 110 aa, synthetic construct
  REGION 1..110: note = Synthetic: h10H1-LC1; VL
DIQMTQSPSSVSASVGDRVTITCSGGGNYYGSYYYGWYQQKPGKAPKLLIYNNNNRPSGVPSRFSGSKSGSTGTLTISSLQPEDFATYYCGGFDSSSDAIFGQGTKVEIK

SEQ ID NO: 239 - protein, 330 aa, Homo sapiens
  REGION 1..330: note = misc_feature - Human IgG1 HC Constant
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 240 - protein, 107 aa, Homo sapiens [sequence omitted]

SEQ ID NO: 241 - protein, 323 aa, Mus musculus
  REGION 1..323: note = misc_feature - Mouse IgG1 HC Constant
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 242 - protein, 107 aa, Mus musculus
  REGION 1..107: note = misc_feature - Mouse IgG LC Constant Ckappa
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 243 - protein, 108 aa, synthetic construct
  REGION 1..108: note = Synthetic: Kappa LC
HMTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 106 aa, synthetic construct
  REGION 1..106: note = Synthetic: Lambda LD
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 245 - protein, 20 aa, synthetic construct
  REGION 1..20: note = Synthetic: FlagHis Tag
GSGDYKDDDDKGSGHHHHHH

SEQ ID NO: 246 - protein, 15 aa, synthetic construct
  REGION 1..15: note = Synthetic: Linker
GGGGSGGGGSGGGGS

SEQ ID NO: 247 - protein, 11 aa, synthetic construct
  REGION 1..11: note = Synthetic: Linker
AAGSDQEPKSS

SEQ ID NO: 248 - protein, 13 aa, synthetic construct
  REGION 1..13: note = Synthetic: Consensus sequence
  VARIANT 2: note = X is Y or F
  VARIANT 3: note = X is V or I
  VARIANT 4: note = X is Y, L, N, R, Q, or P
  VARIANT 5: note = X is Q, A, N, or S
DXXXXYWTYVLDY

SEQ ID NO: 249 - protein, 13 aa, synthetic construct
  REGION 1..13: note = Synthetic: Consensus sequence
  VARIANT 3: note = X is G or D
  VARIANT 4: note = X is P or L
  VARIANT 5: note = X is W or R
  VARIANT 7: note = X is G or L
DYXXXYXTGVLDY

SEQ ID NO: 250 - protein, 13 aa, synthetic construct
  REGION 1..13: note = Synthetic: Consensus sequence
  VARIANT 2: note = X is L, M, or W
  VARIANT 4: note = X is G, V, H, Y, or S
  VARIANT 5: note = X is G or R
  VARIANT 10: note = X is F or V
  VARIANT 11: note = X is F or S
DXGXXYWVGXXDY

SEQ ID NO: 251 - protein, 13 aa, synthetic construct
  REGION 1..13: note = Synthetic: Consensus sequence
  VARIANT 2: note = X is F or Y
  VARIANT 3: note = X is Y, H, or N
  VARIANT 7: note = X is S, A, or F
  VARIANT 9: note = X is Y or F
DXXDRYXTXVLDY

SEQ ID NO: 252 - protein, 12 aa, synthetic construct
  REGION 1..12: note = Synthetic: Consensus sequence
  VARIANT 4: note = X is I or V
TTCXGSGGCIDT

SEQ ID NO: 253 - protein, 9 aa, synthetic construct
  REGION 1..9: note = Synthetic: Consensus sequence
  VARIANT 4: note = X is A or G
  VARIANT 9: note = X is T or G
GGGXASIDX

SEQ ID NO: 254 - protein, 7 aa, synthetic construct
  REGION 1..7: note = Synthetic: Consensus sequence
  VARIANT 5: note = X is S, I, R, Y, or G
  VARIANT 6: note = X is G, Y, A, V, or R
  VARIANT 7: note = X is S or P
GFNIXXX

SEQ ID NO: 255 - protein, 7 aa, synthetic construct
  REGION 1..7: note = Synthetic: Consensus sequence
  VARIANT 5: note = X is S, T, A, or Q
  VARIANT 6: note = X is S, P, Y, or T
  VARIANT 7: note = X is Y, D, or R
GFNIXXX

SEQ ID NO: 256 - protein, 7 aa, synthetic construct
  REGION 1..7: note = Synthetic: Consensus sequence
  VARIANT 5: note = X is S, A, K, or D
  VARIANT 6: note = X is S, A, P, or D
  VARIANT 7: note = X is Y or T
GFNIXXX

SEQ ID NO: 257 - protein, 7 aa, synthetic construct
  REGION 1..7: note = Synthetic: Consensus sequence
  VARIANT 3: note = X is S or T
  VARIANT 4: note = X is I or F
  VARIANT 6: note = X is D, G, or S
GFXXSXY

SEQ ID NO: 258 - protein, 7 aa, synthetic construct
  REGION 1..7: note = Synthetic: Consensus sequence
  VARIANT 1: note = X is N or S
  VARIANT 3: note = X is absent
  VARIANT 5: note = X is G or A
XPXAXGY

SEQ ID NO: 259 - protein, 7 aa, synthetic construct
  REGION 1..7: note = Synthetic: Consensus sequence
  VARIANT 3: note = X is absent
  VARIANT 5: note = X is S or I
  VARIANT 7: note = X is Y or F
YPXYXGX

SEQ ID NO: 260-261 - skipped (2 entries)

SEQ ID NO: 262 - protein, 7 aa, synthetic construct
  REGION 1..7: note = Synthetic: Consensus sequence
  VARIANT 1: note = X is R or S
  VARIANT 4: note = X is absent
  VARIANT 6: note = X is N, S, or R
XNDXGXS

SEQ ID NO: 263 - protein, 5 aa, synthetic construct
  REGION 1..5: note = Synthetic: Consensus sequence
  VARIANT 3: note = X is Y or W
NSXIH

SEQ ID NO: 264 - protein, 17 aa, synthetic construct
  REGION 1..17: note = Synthetic: Consensus sequence
  VARIANT 3: note = X is N or S
  VARIANT 6: note = X is G or A
  VARIANT 13: note = X is D or G
FIXPAXGYTDYAXSVKG

SEQ ID NO: 265 - protein, 17 aa, synthetic construct
  REGION 1..17: note = Synthetic: Consensus sequence
  VARIANT 6: note = X is S or I
  VARIANT 8: note = X is Y or F
  VARIANT 10: note = X is N or E
WIYPYXGXTXYADSVKG

SEQ ID NO: 266 - protein, 17 aa, synthetic construct
  REGION 1..17: note = Synthetic: Consensus sequence
  VARIANT 5: note = X is S, P, A, or F
  VARIANT 6: note = X is G, S, A, or D
  VARIANT 8: note = X is Y or F
  VARIANT 10: note = X is Y or H
VITPXXGXTXYADSVKG

SEQ ID NO: 267 - protein, 17 aa, synthetic construct
  REGION 1..17: note = Synthetic: Consensus sequence
  VARIANT 1: note = X is V, W, H, or F
  VARIANT 3: note = X is S or F
  VARIANT 5: note = X is Y or S
  VARIANT 6: note = X is G or D
  VARIANT 10: note = X is E or D
XIXPXXGYTXYADSVKG

SEQ ID NO: 268 - protein, 17 aa, synthetic construct
  REGION 1..17: note = Synthetic: Consensus sequence
  VARIANT 3: note = X is R or S
  VARIANT 7: note = X is N, S, or R
  VARIANT 9: note = X is A or T
  VARIANT 12: note = X is G, V, or A
  VARIANT 13: note = X is P, D, or A
  VARIANT 14: note = X is A, S, or P
YIXNDGXSXSYXXXVKG

SEQ ID NO: 269 - protein, 18 aa, synthetic construct
  REGION 1..18: note = Synthetic: Consensus sequence
  VARIANT 13: note = X is G, V, or A
  VARIANT 14: note = X is A or D
  VARIANT 15: note = X is A, S, or P
GITYGTGSYTAYXXXVKG

SEQ ID NO: 270 - protein, 18 aa, synthetic construct
  REGION 1..18: note = Synthetic: Consensus sequence
  VARIANT 3: note = X is D or N
  VARIANT 4: note = X is H or S
  VARIANT 5: note = X is D, A, G, or absent
  VARIANT 6: note = X is G, A, or absent
  VARIANT 7: note = X is G or S
  VARIANT 8: note = X is R or S
  VARIANT 9: note = X is Y, G, or D
  VARIANT 11: note = X is D, Y, or N
  VARIANT 13: note = X is G, V, or A
  VARIANT 14: note = X is A, S, or D
  VARIANT 15: note = X is V, A, S, or P
  VARIANT 17: note = X is K or D
RIXXXXXXXTXYXXXVXG

SEQ ID NO: 271 - protein, 10 aa, synthetic construct
  REGION 1..10: note = Synthetic: Consensus sequence
  VARIANT 2: note = X is S or G
  VARIANT 7: note = X is T or S
GXFDSSXDAI

SEQ ID NO: 272 - protein, 7 aa, synthetic construct
  REGION 1..7: note = Synthetic: Consensus sequence
  VARIANT 1: note = X is S, Y, or R
  VARIANT 4: note = X is Q or K
XNNXRPS

SEQ ID NO: 273 - protein, 7 aa, synthetic construct
  REGION 1..7: note = Synthetic: Consensus sequence
  VARIANT 2: note = X is N or S
NXNNRPS

SEQ ID NO: 274 - protein, 8 aa, synthetic construct
  REGION 1..8: note = Synthetic: Consensus sequence
  VARIANT 4: note = X is S or N
  VARIANT 5: note = X is S or Y
SGGXXDYG

SEQ ID NO: 275 - protein, 13 aa, synthetic construct
  REGION 1..13: note = Synthetic: Consensus sequence
  VARIANT 5: note = X is S or N
  VARIANT 7: note = X is V, Y, or A
  VARIANT 9: note = X is G or S
SGGGXYXGXYYYG